SEQUENCE LISTING (3 sequences)

SEQ ID NO: 1 - protein, 4 aa, synthetic construct
  SITE 1: note = Further connected to X
  SITE 3: note = Further connected to T
  SITE 4: note = Further connected to Z
FRKY

SEQ ID NO: 2 - protein, 4 aa, synthetic construct
  SITE 1: note = Further connected to X
  SITE 3: note = Further connected to T
  SITE 4: note = Further connected to Z
FKKF

SEQ ID NO: 3 - protein, 4 aa, synthetic construct
  SITE 1: note = Further connected to X
  SITE 4: note = Further connected to Z; note = Further connected to T
FRFK

(19) United States
(12) Patent Application Publication
(10) Pub. No.: US 2025/0249113 A1
(43) Pub. Date: Aug. 7, 2025

(54) LIGAND-DRUG-CONJUGATES WITH IMPROVED PHARMACOKINETIC AND DRUG RELEASE PROPERTIES

(71) Applicant: Debiopharm Research & Manufacturing S.A., Martigny (CH)

(72) Inventors: Léo Marx, Martigny (CH); Mathilde Lucile Colette Pantin, Thonon-les-baines (FR); Aurélien Simon Denis Adam, Muraz (CH)

(21) Appl. No.: 18/855,834

(22) PCT Filed: Apr. 14, 2023
(86) PCT No.: PCT/EP2023/059781

§ 371 Date: Oct. 10, 2024

(30) Foreign Application Priority Data
Apr. 14, 2022 (WO) PCT/EP2022/060099
Dec. 7, 2022 (WO) PCT/EP2022/084848
Mar. 10, 2023 (WO) PCT/EP2023/056237

(51) Int. Cl. A61K 47/68; A61K 45/06; A61P 35/02; C07K 16/28; C07K 16/32

(52) U.S. Cl. CPC A61K 47/68033; A61K 45/06; A61K 47/68031; A61K 47/6849; A61K 47/6855; A61K 47/6889; A61P 35/02; C07K 16/2896; C07K 16/32

(57) ABSTRACT

The present invention relates to ligand-drug-conjugates for the treatment of disease. In particular, the present invention relates to ligand-drug-conjugates comprising a linker system, which allows for improved delivery of a drug to a target cell while retaining the favorable pharmacokinetic properties of antibodies. The present invention also relates to ligand-drug-conjugates, which achieve high drug-antibody-ratio and exhibit excellent pharmacokinetic properties, thus resulting in significantly improved efficacy. In certain aspects, the present invention also relates to ligand-drug-conjugates for the intracellular delivery of cytotoxic drugs to tumor or cancer cells.

The present invention relates to ligand-drug-conjugates (LDCs) for the treatment of disease. In particular, the present invention relates to ligand-drug-conjugates comprising a linker system, which allows for improved delivery of a drug to a target cell while retaining the favorable pharmacokinetic properties of antibodies. The present invention also relates to ligand-drug-conjugates, which achieve a high drug-antibody-ratio (DAR) and exhibit excellent pharmacokinetic properties, thus resulting in significantly improved efficacy. In certain aspects, the present invention also relates to ligand-drug-conjugates for the intracellular delivery of cytotoxic drugs to tumor or cancer cells.

### BACKGROUND OF THE INVENTION

Recently, a great deal of interest has surrounded the use of enzyme-triggered drug release systems such as antibody-drug-conjugates (ADCs) for the targeted delivery of cytotoxic agents to tumor cells. Antibody-drug-conjugates generally consist of three components: an antibody that targets an antigen highly expressed on tumor cells, a cytotoxic agent (sometimes called “toxin” or “payload”), and a linker system which may release the cytotoxic agent from the antibody upon internalization into cancer cells.

Ideally, antibody-drug-conjugates should retain the favorable pharmacokinetic and functional properties of antibodies, remain intact and nontoxic in systemic circulation (blood), and become active at the target site with drug released in sufficient amount to kill the target cell. Thus, one of the biggest challenges in the development of antibody-drug-conjugates represents the design of linker systems for the conjugation of antibody and drug, which are nontoxic and stable in systemic circulation, but which are nevertheless capable of releasing the drug inside the target cell at a high rate and in sufficient amount while retaining the favorable pharmacokinetic properties of antibodies.

Many linker systems have been developed for the specific intracellular release of cytotoxic drugs. There are two main families of linkers: cleavable and non-cleavable. Cleavable linkers usually utilize an inherent property of the target cell, such as protease-sensitivity, for selectively releasing the drug, e.g., a cytotoxic agent, from the conjugate. Non-cleavable linkers usually rely on the complete degradation of the antibody after internalization of the conjugate into the target cell. An example of antibody-drug-conjugate using a non-cleavable linker is the humanized anti-HER2 (anti-ErbB2) antibody-maytansine conjugate trastuzumab-emtansine (T-DM1, or Kadcyla®; LoRusso et al. Clin. Cancer Res. 2011, 17, 6437-6447), which comprises the monoclonal antibody trastuzumab conjugated to the tubulin polymerisation inhibitor mertansine (DM1) via a non-cleavable maleimidomethyl cyclohexane-1-carboxylate (MCC) linker.

While non-cleavable antibody-drug-conjugates, such as T-DM1 (Kadcyla®), are considered advantageous for their stability in systemic circulation, they may nevertheless not exhibit the desired efficacy. Especially, the drug may not be released in sufficient amount to achieve the desired pharmacological effect, e.g., due to slow degradation of the antibody moiety, and/or may be released in a less potent modified form, e.g., as a “linker-drug” construct. For example, lysosomal degradation of the antibody component of T-DM1 releases the moiety lysine-MCC-DM1, which efficiently binds to tubulin but has only limited ability to induce cytotoxic bystander effect (Ogitani et al. Cancer Sci. 2016, 107, 1039-1046). Treatment failure due to intrinsic and acquired resistance to T-DM1 has also remained a major clinical challenge (Hunter et al. Br. J. Cancer 2020, 122(5), 603-612).

Peptide linkers have also been proposed as they combine good stability in systemic circulation with rapid intracellular drug release by specific enzymes, e.g, proteases. Especially, peptide linkers comprising a valine-citrulline (Val-Cit) dipeptide as substrate for intracellular cleavage by Cathepsin B (Cat B) have been described (Lu et al. Int. J. Mol. Sci. 2016, 17, 561-582; Jain et al. Pharm. Res. 2015, 32(11), 3526-3540; Dubowchik et al. Bioconj. Chem. 2002, 13, 855-859). Cat B is a lysosomal cysteine protease implicated in a number of physiological processes, which differs from other cysteine proteases in that it possesses endopeptidase activity and also exopeptidase (carboxydipeptidase) activity, meaning that it can remove dipeptide units from the C-termini of proteins and peptides (Turk et al. Biochim. Biophys. Acta 2012, 1824(1), 68-88).

Typically, enzymatic cleavage of a conjugate releases the antibody and a linker-drug conjugate at the target site. The linker must, in turn, allow rapid release of the drug from the linker-drug conjugate. Thus, “self-immolative” spacers between linker and drug have been proposed for enhancing drug release rate after enzymatic cleavage. Self-immolative spacers usually release a drug by elimination- or cyclization-based mechanisms.

An example of linker system comprising a self-immolative spacer is the para-amino benzyloxycarbonyl (PABC) linker as used, e.g., in the bremtuximab-vedotin conjugate Adcetris® (Younes et al. N. Engl. J. Med. 2010, 363, 1812-1821; Jain et al. Pharm. Res. 2015, 32(11), 3526-3540). The PABC linker system as used in antibody-drug-conjugates utilizes a protease-sensitive Val-Cit-PABC dipeptide linker, which can be recognized and cleaved by Cathepsin B. A maleimidocaproyl (MC) moiety is typically used for attaching the linker unit to the antibody and serves as a spacer between drug and antibody for avoiding steric conflicts in substrate recognition by Cathepsin B. After enzymatic cleavage of the citrulline-PABC amide bond, the resulting PABC-substituted drug, e.g., monomethyl auristatin E (MMAE) spontaneously undergoes a 1,6-elimination that releases the free drug (e.g., MMAE) as the product. Nonetheless, the efficacy of the PABC linker system may be limited due to slow intracellular drug release and the tendency of the Val-Cit-PABC moiety to cleave in systemic circulation (Dorywalska et al. Mol. Cancer Ther. 2016, 15(5), 958-970).

Furthermore, in vivo studies indicate that the pharmacokinetic properties, such as distribution and hepatic clearance, of Val-Cit-PABC-based conjugates depend on the number of drug molecules attached to the antibody moiety (Strop et al. Chem. & Biol. 2013, 20, 161-167). Thus, an important parameter of antibody-drug-conjugates is the drug-antibody-ratio (DAR) (or drug load(ing)) referring to the average number of drug molecule(s) attached to one antibody moiety. The DAR not only affects efficacy, but also the pharmacokinetic properties and toxicity of the conjugates. A high DAR has been associated with decreased pharmacokinetic properties and/or higher toxicity due to increased tendency of the conjugate molecules for aggregation and/or premature cleavage.

To overcome these problems, it has been proposed to employ hydrophilic linker systems containing negatively charged sulfonate groups, polyethylene glycol groups or pyrophosphate diester groups in order to reduce conjugate aggregation. Likewise, WO 2015/057699 A2 discloses antibody-drug-conjugates based on the combination of a drug, such as MMAE, with a linker system comprising a cleavable Val-Cit-PABC moiety and a hydrophilic untethered polyethylene glycol group. The conjugates disclosed in WO 2015/057699 A2 are said to exhibit good pharmacokinetic properties in an in vivo model even at high DAR (e.g., 8). However, the efficacy of the linker systems e.g. as disclosed in WO 2015/057699 A2 may be limited due to unspecific enzymatic and/or premature cleavage, slow intracellular drug release and possibly also increased lysosomal trapping.

There is thus a need for novel compounds comprising a linker system, which is stable in systemic circulation, and which can rapidly release and deliver a drug to a target cell in sufficient amount to kill the target cell while allowing to retain favorable pharmacokinetic properties.

It is hence an object of the present invention to provide compounds comprising a linker system, which is stable in systemic circulation and allows rapid release and delivery of a drug to a target cell in a traceless manner while allowing to retain favorable pharmacokinetic properties. It is a further object of the present invention to provide pharmaceutical compositions comprising such compounds. The present invention also relates to ligand-drug-conjugates, which are characterized by a high DAR and exhibit excellent pharmacokinetic properties, thus resulting in significantly improved efficacy.

It is yet another object of the present invention to provide compounds comprising a linker system, which can release multiple drug molecules while allowing to retain favorable pharmacokinetic properties, wherein the individual drug molecules may be the same or different.

A further object of the present invention is to provide compounds or compositions that can be used in methods of treating or preventing a cancer, an autoimmune disease or inflammatory disease and/or an infectious disease.

### SUMMARY OF THE PRESENT INVENTION

The present invention provides a new hydrophilic cleavable linker system which can be used in ligand-drug-conjugates. The linker system is preferably characterized by the presence of a C-terminal peptide unit carrying a drug covalently attached to its N-terminus via a specific spacer group and a solubilizing group on a side chain thereof.

The C-terminal peptide unit acts as highly specific substrate for Cathepsin B and preferably for the exopeptidase (i.e., carboxydipeptidase) activity of Cathepsin B, resulting in improved intracellular cleavage and drug release. The linker system is stable in systemic circulation and enables to achieve high DAR while retaining excellent pharmacokinetic properties, thus resulting in significantly improved efficacy.

The present invention thus relates to a compound represented by the following general formula (I):

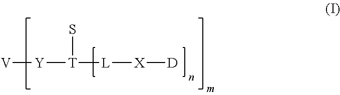

wherein,
D represents a moiety derived from a drug, the drug being selected from a carboxyl-containing drug, a thiol-containing drug, an amino-containing drug, and a hydroxyl-containing drug; if more than one (D) is present, each (D) is independently selected from a carboxyl-containing drug, a thiol-containing drug, an amino-containing drug and a hydroxyl-containing drug, the moieties (D) being preferably identical to each other;
X represents a divalent group comprising one to seven backbone atoms independently selected from C, N, O, and S; X being covalently attached to (D) via an atom selected from C, S, N and O derived from the carboxyl, thiol, amino, or hydroxyl functional group comprised in (D);
Y is a divalent group comprising one or more atoms selected from C, N, O, P and S;
L represents a linker capable of being cleaved by Cathepsin B;
T represents a (2+n)-valent branching group;
S represents a moiety derived from a compound comprising one or more, e.g., two, three or four, solubilizing groups;
V represents a moiety derived from a vector group capable of interacting with a target cell;
n is an integer of 1 to 4; and
m is an integer of 1 to 12.

The present invention also relates to a compound as hereinbefore described or composition thereof for use in a method of treating or preventing a cancer, an autoimmune disease or inflammatory disease and/or an infectious disease.

The present invention in particular includes the following embodiments (“Items”):
1. A compound represented by the general formula (I):

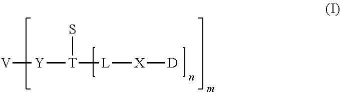

wherein,
D represents a moiety derived from a drug, the drug being selected from a carboxyl-containing drug such as auristatin F (AF), a thiol-containing drug such as mertansine (DM1) or ravtansine (DM4), an amino-containing drug such as monomethyl auristatin F (MMAF) or exatecan, and a hydroxyl-containing drug such as Maaa-1181a, preferably from a thiol-containing drug, an amino-containing drug and a hydroxyl-containing drug; if more than one (D) is present, each (D) is independently selected from a carboxyl-containing drug, a thiol-containing drug, an amino-containing drug and a hydroxyl-containing drug, the moieties (D) being preferably identical to each other;
X represents a divalent group comprising one to seven, preferably two to six, more preferably two to five, backbone atoms independently selected from C, N, O, and S; X being covalently attached to (D) via an atom selected from C, S, N and O derived from the carboxyl, thiol, amino, or hydroxyl functional group comprised in (D);
Y is a divalent group comprising one or more atoms selected from C, N, O, P and S, preferably a divalent group derived from a compound selected from maleimides, triazoles, hydrazones, carbonyl-containing compounds and derivatives thereof, more preferably a divalent group derived from maleimides and derivatives thereof such as opened hydrolyzed maleimides, and most preferably a group derived from an opened hydrolyzed maleimide;
L represents a linker capable of being cleaved by Cathepsin B;
T represents a (2+n)-valent branching group;
S represents a moiety derived from a compound comprising one or more, e.g., two, three or four, solubilizing groups;
V represents a moiety derived from a vector group capable of interacting with a target cell;
n is an integer of 1 to 4, preferably 1 or 2, more preferably 1; and
m is an integer of 1 to 12, preferably 2 to 10, more preferably 4 to 8.
2. The compound of item 1, wherein (L) is represented by the general formula (II) or (II′):

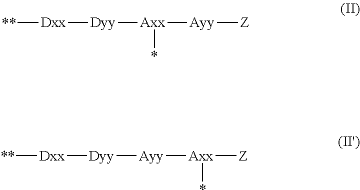

wherein,
Axx represents a moiety derived from a trifunctional amino acid, with the proviso that Axx in formula (II) is not a moiety derived from an amino acid in the (D) configuration;
Ayy represents a moiety derived from an amino acid selected from Phe, Ala, Trp, Tyr, Phenylglycine (Phg), Met, Val, His, Lys, Arg, Citrulline (Cit), 2-amino-butyric acid (Abu), Ornithine (Orn), Ser, Thr, Leu and Ile; or Ayy in formula (II) represents a moiety derived from an amino acid selected from homo-tyrosine (homo-Tyr), homo-phenylalanine (homo-Phe), beta-phenylalanine (beta-Phe) and beta-homo-phenylalanine (beta-homo-Phe), Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24; with the proviso that Ayy in formula (II′) is not a moiety derived from an amino acid in the (D) configuration;
Dxx represents a single covalent bond or a moiety derived from an amino acid having a hydrophobic side chain, preferably a single covalent bond or a moiety derived from an amino acid selected from Phe, Val, Tyr, homo-Phe and Ala, more preferably a single covalent bond or a moiety derived from Phe or Val;
Dyy represents a single covalent bond, a moiety derived from Phe or a moiety derived from an amino acid having a basic side chain, preferably a moiety derived from an amino acid selected from Arg, Lys, Cit, Orn, 2,3-diamino-propionic acid (Dap), 2,4-diamino-butyric acid (Dab), more preferably a moiety derived from Arg or Cit; with the proviso that if Dxx is a moiety derived from an amino acid having a hydrophobic side chain, Dyy is a moiety derived from Phe or a moiety derived from an amino acid having a basic side chain, and if Dxx is a single covalent bond, Dyy is a single covalent bond, a moiety derived from Phe or a moiety derived from an amino acid having a basic side chain;
Z represents a group covalently bonded to the C-terminus of Ayy or Axx selected from —OH and —N(H)(R) wherein R represents a hydrogen atom, an alkyl group or a cycloalkyl group, preferably —OH;
* indicates covalent attachment to (T); and
** indicates covalent attachment to (X).
3. The compound of item 2, wherein at least one of Axx and Ayy is defined as follows:
   Axx represents a moiety derived from an amino acid selected from Glu, 2-amino-pimelic acid (Apa), 2-amino adipic acid (Aaa), Dap, Dab, Lys, Orn, Ser, Ama, and homo-lysine (homo-Lys), preferably a moiety derived from an amino acid selected from Dap, Dab, Lys, Orn and homo-Lys, more preferably a moiety derived from Orn or Lys, most preferably a moiety derived from Lys;
   Ayy in formula (II) represents a moiety derived from an amino acid selected from Phe, homo-Phe, Ala, Trp, Phg, Leu, Val, Tyr, homo-Tyr, Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24, preferably a moiety derived from Phe, homo-Phe, Tyr, homo-Tyr, Tyr(OR₁) and homo-Tyr(OR₁), more preferably a moiety derived Phe or Tyr, most preferably a moiety derived from Tyr;
   Ayy in formula (II′) represents a moiety derived from an amino acid selected from Phe, homo-Phe, Ala, Trp, Phg, Leu, Val, Tyr and Ser, preferably a moiety derived from Phe, home-Phe and Ser, more preferably a moiety derived from Phe or Ser, most preferably a moiety derived from Phe.
4. The compound of any of items 1 to 3, wherein (X) represents a divalent carbonyl- or thiocarbonyl-containing group, preferably a group represented by one of the following formulae (IIIa) to (IIIf):

**—(CH₂)ₙ₂—(C=A)-**′ (IIIa)

**—(C=A)-(CH₂)ₙ₃—**′ (IIIb)

**—(CH₂)ₙ₂—(C=A)-(CH₂)ₙ₃—**′ (IIIc)

**—(CH₂)ₙ₂—(C=A)-(C(CH₃)₂)—(CH₂)ₙ₃—**′ (IIId)

**—(CH₂)ₙ₂—(C(CH₃)₂)—(C=A)-(CH₂)ₙ₃—**′ (IIIe)

**—(C=A)-(NH)ₙ₄—(CH₂)ₙ₃—(NH)ₙ₅—(C=A)-**′ (IIIf)
wherein
n2, n3 are each independently selected from 0 to 5, preferably 0, 1 or 2, more preferably 0 or 1;
n4, n5 are each selected from 0 or 1;
each A is independently selected from O and S, preferably O;
*** represents covalent attachment to (D); and
**′ represents covalent attachment to (L).
5. The compound of any of items 1 to 3, wherein (X) is represented by one of the following formulae (IVa) to (IVj′):

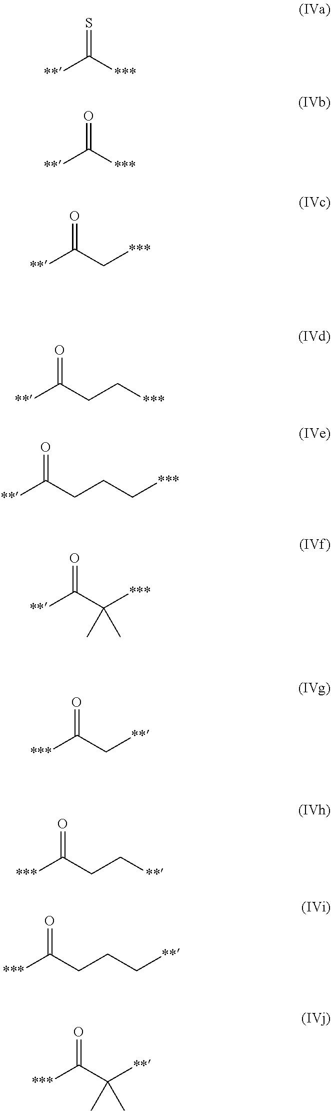

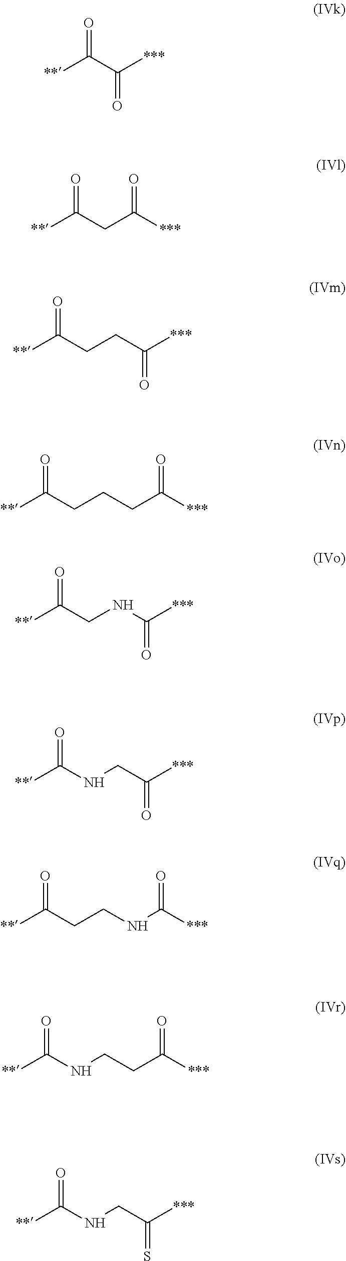

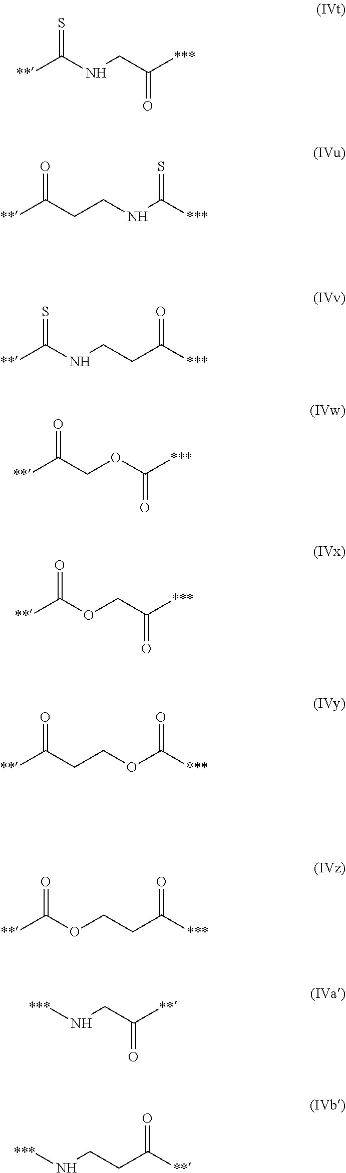

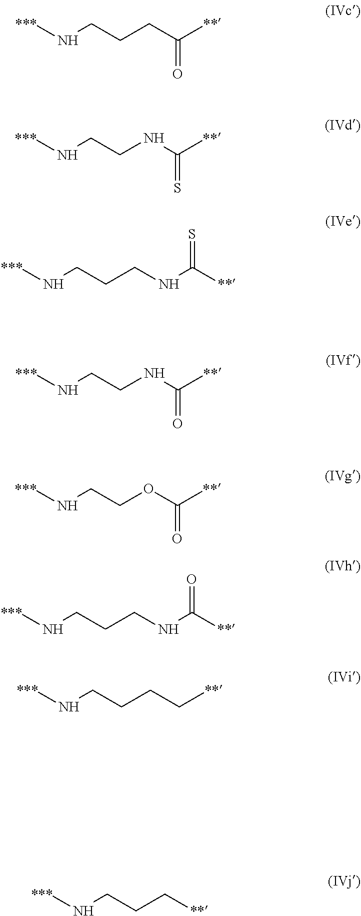

wherein,
*** represents covalent attachment to (D);
**′ represents covalent attachment to (L);
with the proviso that if (X) is represented by formula (IVg), (IVh), (IVi), (IVj), (IVk), (IV/), (IVm), (IVn), (IVp), (IVr), (IVt), (IVv), (IVw), (IVx), (IVy) or (IVz), (D) in formula (I) represents an amino-containing drug; if (X) is represented by formula (IVj), (IVq), (IVs) or (IVu), (D) in formula (I) represents an amino-containing drug or a hydroxyl-containing drug; and if (X) is represented by formula (IVa′), (IVb′), (IVc′), (IVd′), (IVe′), (IVf′), (IVg′), (IVh′), (IVi′) or (IVj′), (D) in formula (I) represents a carboxyl-containing drug.
6. The compound of item 5, wherein (X) is represented by formula (IVb′), (IVc), (IVm), (IVn), (IVo), (IVp), (IVs) or (IVt), preferably by formula (IVc) or (IVm).
7. The compound of any of items 1 to 6, wherein each solubilizing group comprised in (S) is independently selected from the group consisting of:
   moieties comprising one or more ionic or ionizable groups, such as ammonium, guanidinium, sulfate or sulfonate groups, preferably of moieties derived from Arg, (D)-Arg, Dap, (D)-Dap, Dab, (D)-Dab, Orn, (D)-Orn, Lys, D-Lys or carnitine;
   saccharide moieties selected from monosaccharides, disaccharides and linear or branched oligosaccharides, in particular linear or branched oligosaccharides having 3 to 10 monosaccharide units being linked by glycosidic bonds, wherein each of the monosaccharide units in the monosaccharide, disaccharide and oligosaccharide is independently selected from glucose, fructose, mannose, ribose, and galactose; and
   polyalkylene oxide groups, preferably C₂₋₃ polyalkylene oxide groups, more preferably C₂₋₃ polyalkylene oxide groups independently comprising from 6 to 200, preferably from 10 to 150, more preferably from 12 to 80 repeating units.
8. The compound of any of items 1 to 7, wherein (S) is a moiety derived from a compound comprising one or more polyethylene oxide groups, wherein preferably each polyethylene oxide group independently comprises from 6 to 200, more preferably from 10 to 150, most preferably from 12 to 80 repeating units;
   (S) being preferably a moiety represented by the formula (V):

****—X¹—(CH₂CH₂O)ₙ₃—X² (V)
wherein,
n3 is an integer of 6 to 200, preferably 10 to 150, more preferably 12 to 80;
**** indicates covalent attachment to (T);
X¹ is selected from a single covalent bond, —(C═O)—, and —N(R)— in which R represents a hydrogen atom, an alkyl group or a cycloalkyl group;
X² represents an alkyl group having 1 to 6 carbon atoms, a carbonyl-containing group such an acetyl group or a group of formula —(CH₂)ₙ₄—CO₂H, a thiocarbonyl-containing group, a group of formula —(CH₂)ₙ₄OR, a group of formula —(CH₂)ₙ₄—SO₃H, or an amino-containing group such as a group of formula —(CH₂)ₙ₄—(C=A)-N(R)₂ or —(CH₂)ₙ₄—N(R)₂, in which A is O or S, each R is independently selected from a hydrogen atom, an alkyl group and a cycloalkyl group, and n4 is an integer of 1 to 6;
X² being preferably —CH₃, —CH₂CH₂OH, or a group represented by the following formula (VI):

—(CH₂)ₙ₅—(C=A)N(R)—(CH₂)ₙ₆—(C=A)N(H)(R) (VI)
wherein,
each A is independently selected from O and S, preferably O;
each R is independently selected from a hydrogen atom, an alkyl group and a cycloalkyl group; and
n5 and n6 are each independently an integer of 1 to 6, preferably 1 or 2;
X² being most preferably —CH₃; and
if more than one (S) is present, each (S) is preferably a moiety of formula (V) above.
9. The compound of any of items 1 to 8, wherein (T) is represented by the following formula (VII):

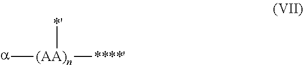

wherein,
each AA independently represents a moiety derived from a trifunctional amino acid such as a diamino-carboxylic acid, an amino dicarboxylic acid, an azido amino acid or an alkyne-containing amino acid, preferably derived from an amino acid selected from N-ε-propargyloxycarbonyl-L-Lysine (Lys(Poc)), Asp, Glu, Orn, Lys, Dab and Dap, more preferably derived from Lys(Poc), Glu, Orn or Lys, most preferably derived from Lys; a indicates covalent attachment to (Y);
if n=1, the side chain originating from the trifunctional amino acid is covalently attached to (L) or (S), the C-terminus is covalently attached to the other moiety (S) or (L), respectively;
if n=2, 3 or 4:
indicates covalent attachment to (L);
****′ indicates covalent attachment to (S); and
n is as defined in item 1.
10. The compound of any of items 1 to 8, wherein (T) is represented by the formula (VIII) or (IX):

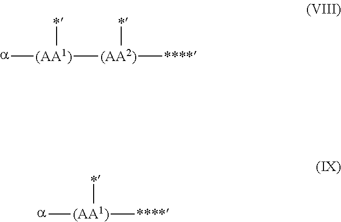

wherein,
each AA¹ and AA² is independently a moiety derived from a trifunctional amino acid, such as a diamino-carboxylic acid, an amino dicarboxylic acid, an azido amino acid or an alkyne-containing amino acid, preferably a moiety derived from an amino acid selected from Lys(Poc), Asp, Glu, Orn, Lys, Dab and Dap, more preferably a moiety derived from Lys(Poc), Glu, Orn or Lys, most preferably a moiety derived from Lys;
   a indicates covalent attachment to (Y);
   in formula (IX), the side chain originating from the trifunctional amino acid is covalently attached to (L) or (S), the C-terminus is covalently attached to the other moiety (S) or (L), respectively;
   in formula (VIII), *′ indicates covalent attachment to (L), and ****′ indicates covalent attachment to (S).
11. The compound of any of items 1 to 10, wherein (D) is a moiety derived from a drug selected from
   (i) an antineoplastic agent such as
      a DNA-alkylating agent, such as duocarmycin,
      a topoisomerase inhibitor, such as doxorubicin,
      an RNA-polymerase II inhibitor, such as alpha-amanitin,
      a DNA cleaving agent, such as calicheamicin,
      an antimitotic agent or microtubule disruptor, such as a taxane, an auristatin or a maytansinoid,
      an anti-metabolite, such as derivatives of gemcitabine,
      a Kinesin spindle protein inhibitor, such as Filanesib,
      a kinase inhibitor, such as ipatasertib or gefitinib,
      nicotinamide phosphoribosyltransferase inhibitor,
      a matrix metallopeptidase 9 inhibitor,
      a phosphatase inhibitor such as mycrocystin-LR,
      (ii) an immunomodulatory agent, such as fluticasone
      (iii) an anti-infectious disease agent, such as rifamycin, clindamycin or reptamulin, and
      (iv) radioisotopes, metabolites, pharmaceutically acceptable salts, and/or prodrugs of any of the foregoing;
   with the proviso that the drug selected from (i) to (iv) is a carboxyl-containing drug, a thiol-containing drug, an amino-containing drug, or a hydroxyl-containing drug; and
   if more than one (D) is present, each (D) is independently selected from the aforementioned moieties (i) to (iv), the moieties (D) being preferably identical to each other.
12. The compound of any of items 1 to 11, wherein (D) is a moiety derived from a drug selected from amanitin, duocarmycin, auristatin, auristatin F (AF), monomethyl auristatin F (MMAF), maytansine, mertansine (DM1), ravtansine (DM4), tubulysin, calicheamicin, camptothecin, SN-38, exatecan, Maaa-1181a, taxol, daunomycin, vinblastine, doxorubicin, methotrexate, pyrrolobenzodiazepine (PBD) and dimers thereof, indilinobenzodiazepine (IBD) and dimers thereof, or radioisotopes and/or pharmaceutically acceptable salts thereof; preferably a moiety derived from a drug selected from auristatin, MMAF, exatecan, maytansine, DM1 and DM4; more preferably a moiety derived from DM1 or DM4.
13. The compound of any one of items 1 to 12, which is represented by the general formula (X) or (X′):

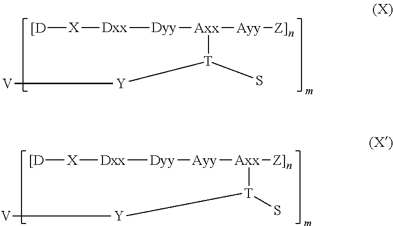

wherein,
Axx in formula (X) and in formula (X′) represents a moiety derived from an amino acid selected from Glu, Apa, Aaa, Dap, Dab, Lys, Orn, Ser, Ama and homo-Lys, preferably a moiety derived from an amino acid selected from Dap, Dab, Lys, Orn and homo-Lys, more preferably a moiety derived from a moiety derived from Lys;
Ayy in formula (X) represents a moiety derived from an amino acid selected from Phe, homo-Phe, Ala, Trp, Phg, Leu, Val, Tyr, homo-Tyr, Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24, preferably a moiety derived from Phe, homo-Phe, Tyr, homo-Tyr, Tyr(OR₁) or homo-Tyr(OR₁), more preferably a moiety derived from Tyr;
Ayy in formula (X′) represents a moiety derived from an amino acid selected from Phe, homo-Phe, Ala, Trp, Phg, Leu, Val, Tyr and Ser, preferably a moiety derived from Phe, home-Phe or Ser, more preferably a moiety derived from Phe or Ser;
D, Dxx, Dyy, X, Y, T, S, V, Z, m and n have the same meanings as specified in any of items 1, 2, 4, 5, 6, 7, 8, 9, 10, 11 and 12; and wherein preferably at least one, e.g., two, three, four, five, six, seven or eight, of D, Dxx, Dyy, X, Y, T, S and Z is/are defined as follows:
(a) D is a moiety derived from a drug selected from auristatin, MMAF, exatecan, maytansine, DM1 and DM4, preferably a moiety derived from DM1 or DM4;
(b) Dxx is a moiety derived from an amino acid selected from Phe, Val, Tyr, homo-Phe and Ala, preferably a moiety derived from Phe or Val;
(c) Dyy is a covalent bond or a moiety derived from an amino acid selected from Arg, Lys, Cit, Orn, Dap and Dab, preferably a covalent bond or a moiety derived from Arg or Cit;
(d) X is a group of formula (III) wherein n2 is 1 or 2, or a group represented by any of formulae (IVa) to (IVz), preferably a group represented by any of formula (IVc), (IVm), (IVn), (IVo), (IVp), (IVs) or (IVt), more preferably by formula (IVc) or (IVm);
(e) Y is a group derived from a compound selected from maleimides, triazoles, hydrazones, carbonyl-containing compounds and derivatives thereof, preferably from maleimides and derivatives thereof such as opened hydrolyzed maleimides, and more preferably from an opened hydrolyzed maleimide;
(f) T is a group of formula (VII), (VIII) or (IX);
(g) S is a moiety of formula (V); and
(h) Z is —OH.
14. The compound of item 13, wherein in formula (X), each Dyy-Dxx-Axx-Ayy is independently selected from Arg-Lys-Phe wherein Dyy is a covalent bond, Arg-Lys-homoPhe wherein Dyy is a covalent bond, Arg-Lys-Tyr wherein Dyy is a covalent bond, Cit-Lys-Phe wherein Dyy is a covalent bond, Cit-Lys-Tyr wherein Dyy is a covalent bond, Arg-Lys-homoTyr wherein Dyy is a covalent bond, Cit-Lys-homoTyr wherein Dyy is a covalent bond, Phe-Cit-Lys-Phe, Phe-Cit-Lys-Tyr, Phe-Arg-Lys-Tyr, Phe-Cit-Lys-homoTyr, Phe-Lys-Lys-Phe, homoPhe-Arg-Lys-Phe, homo-Phe-Cit-Lys-Tyr; and
   in formula (X′), each Dyy-Dxx-Ayy-Axx is independently selected from Arg-Phe-Lys wherein Dyy is a covalent bond, Arg-Ser-Lys wherein Dyy is a covalent bond, Cit-Phe-Lys wherein Dyy is a covalent bond, Cit-Ser-Lys wherein Dyy is a covalent bond, Cit-homoPhe-Lys wherein Dyy is a covalent bond, Phe-Cit-Phe-Lys, homoPhe-Cit-Phe-Lys, and Phe-Arg-Phe-Lys.
15. The compound of any one of items 1 to 14, which is represented by one of the following formulae:

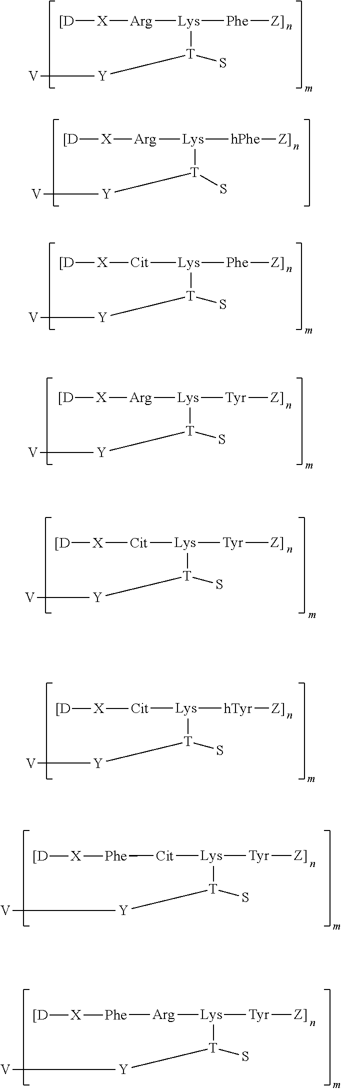

wherein D, X, Y, T, S, V, Z, m and n have the same meanings as specified in item 1, 2, 4, 5, 6, 7, 8, 9, 10, 11 or 12; and wherein preferably at least one, e.g., two, three, four, five or six, of D, X, Y, T, S and Z is/are defined as follows:
(a) D is a moiety derived from a drug selected from auristatin, AF, MMAF, exatecan, maytansine, DM1 and DM4, preferably a moiety derived from DM1 or DM4;
(d) X is a group of formula (III), wherein n2 is 1 or 2, or a group represented by any of formulae (IVa) to (IVz), preferably a group represented by any of formula (IVc), (IVm), (IVn), (IVo), (IVp), (IVs) or (IVt), more preferably by formula (IVc) or (IVm);
(e) Y is a group derived from a compound selected from maleimides, triazoles, hydrazones, carbonyl-containing compounds and derivatives thereof, preferably from maleimides and derivatives thereof such as opened hydrolyzed maleimide derivatives, and more preferably from an opened hydrolyzed maleimide;
(f) T is a group of formula (VII), (VIII) or (IX);
(g) S is a moiety of formula (V); and
(h) Z is —OH.
16. The compound of any of items 1 to 15, which is represented by one of the following formulae:

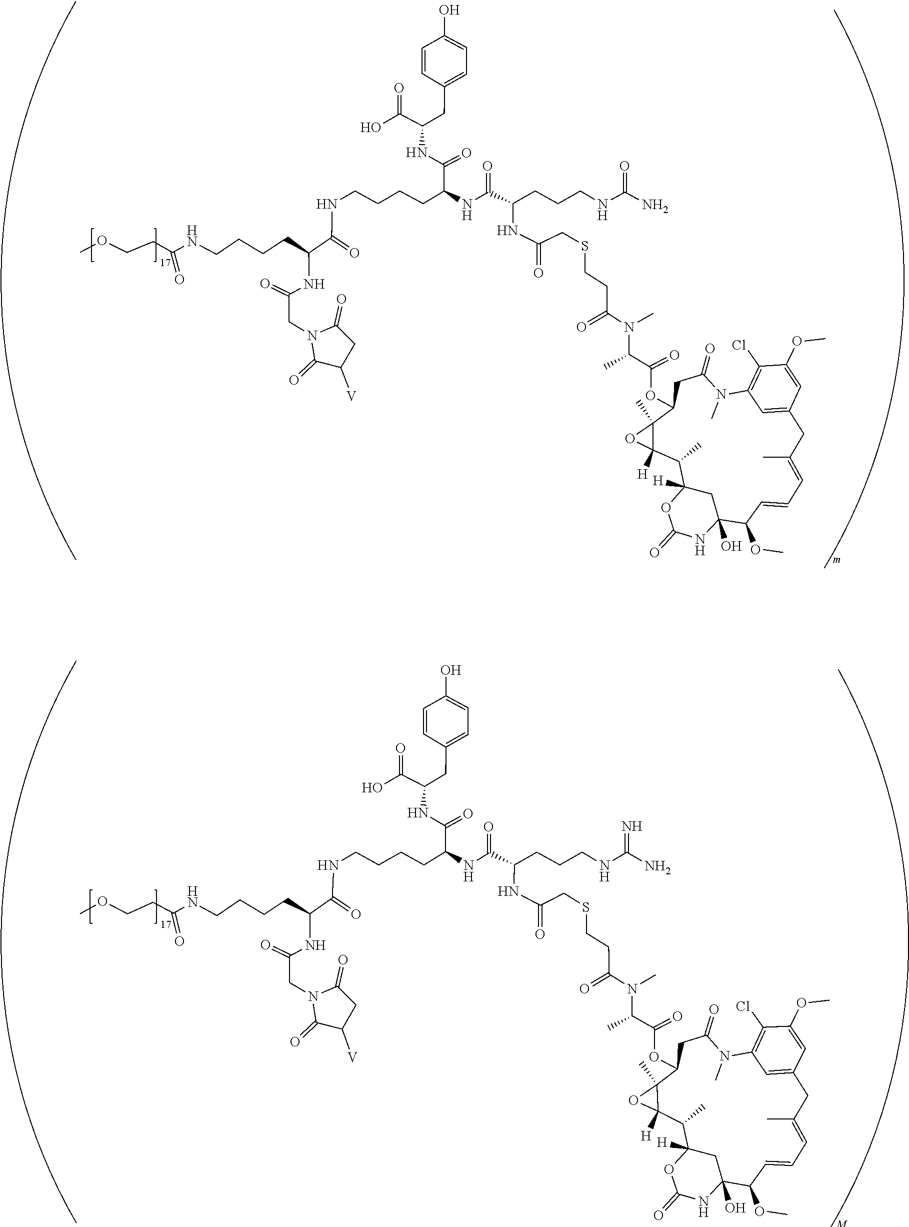

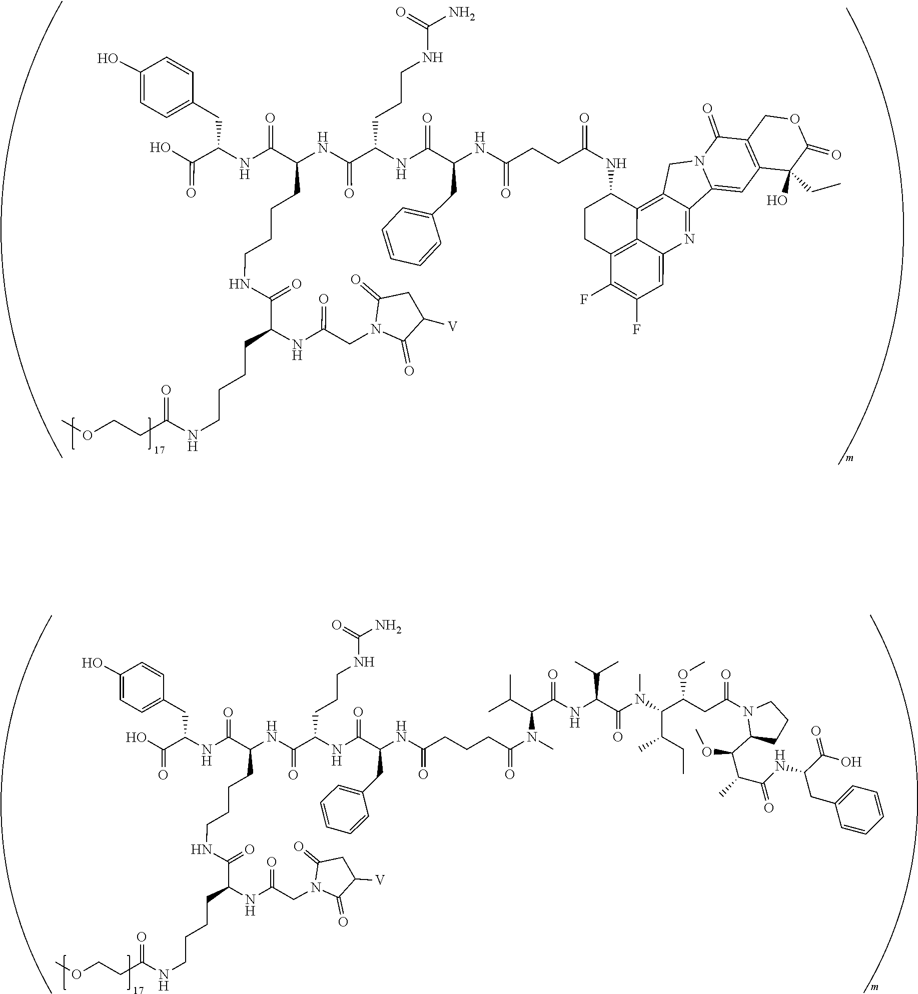

wherein m is an integer of 1 to 12, preferably 2 to 10, more preferably 4 to 8; wherein the number of oxyethylene repeating units (17) may be replaced by 12 to 30, preferably 14 to 25, more preferably 15 to 19 oxyethylene groups; and/or
wherein the maleimide attachment to (V) may be replaced by an opened hydrolyzed maleimide attachment.
17. The compound of any of items 1 to 16, wherein (V) represents a moiety derived from a vector group capable of interacting with a target cell, wherein the target cell is selected from tumor cells, virus infected cells, microorganism infected cells, parasite infected cells, cells involved in autoimmune diseases, activated cells, myeloid cells, lymphoid cells, melanocytes and infectious agents including bacteria, viruses, mycobacteria, fungi;
   preferably the target cell is selected from lymphoma cells, myeloma cells, myeloid cells, lymphoid cells, renal cancer cells, breast cancer cells, prostate cancer cells, ovarian cancer cells, colorectal cancer cells, gastric cancer cells, squamous cancer cells, small-cell lung cancer cells, testicular cancer cells, skin cancer cells, pancreatic cancer cells, liver cancer cells, and any cells growing and dividing at an unregulated and quickened pace to cause cancers.
18. The compound of any of items 1 to 17, wherein (V) represents a moiety derived from a vector group selected from antibodies, antibody fragments, proteins, peptides and non-peptidic molecules,
   preferably a moiety derived from an antibody or an antibody fragment such as a single chain antibody, a monoclonal antibody, a single chain monoclonal antibody, a monoclonal antibody fragment, a chimeric antibody, a chimeric antibody fragment, a domain antibody or fragment thereof, a cytokine, a hormone, a growth factor, a colony stimulating factor, a neurotransmitter or a nutrient-transport molecule.
19. The compound of any of items 1 to 18, wherein (V) represents a moiety derived from:
   a monoclonal antibody, preferably from an antibody selected from the group consisting of adalimumab, aducanumab, alemtuzumab, altumomab pentetate, amivantamab, atezolizumab, anetumab, avelumab, bapineuzumab, basiliximab, bectumomab, belantamab mafadotin, bermekimab, besilesomab, bevacizumab, bezlotoxumab, brentuximab, brentuximab vedotin, brodalumab, catumaxomab, cemiplimab, cetuximab, cinpanemab, clivatuzumab, crenezumab, tetraxetan, daclizumab, daratumumab, denosumab, dinutuximab, dostarlimab, durvalumab, edrecolomab, elotuzumab, emapalumab, enfortumab, enfortumab vedotin, epcoritamab, epratuzumab, epratuzumab-SN-38, etaracizumab, gemtuzumab, gemtuzumab ozogamycin, genmab, glofitamab, girentuximab, gosuranemab, ibritumomab, inebilizumab infliximab, inotuzumab, inotuzumab ozogamicin, ipilimumab, isatuximab ixekizumab, J591 PSMA-antibody, labetuzumab, lecanemab, loncastuximab tesirin, mogamulizumab, mosunetuzumab, necitumumab, nimotuzumab, natalizumab, naratuximab, naxitamab, nivolumab, ocrelizumab, ofatumumab, olaratumab, oregovomab, panitumumab, pembrolizumab, pertuzumab, polatuzumab, polatuzumab vedotin, prasinezumab, racotumomab, ramucirumab, rituximab, sacituzumab, sacituzumab govitecan, semorinemab, siltuximab, solanezumab, tacatuzumab, tafasitamab, teprotumumab, tilavonemab, tocilizumab, tositumomab, trastuzumab, trastuzumab deruxtecan, trastuzumab emtansine, TS23, ustekinumab, vedolizumab, votumumab, zagotenemab, zalutumumab, zanolimumab, fragments and derivatives thereof; more preferably from atezolizumab, durvalumab, pembrolizumab, rituximab or trastuzumab; or
   an antibody fragment incorporated into an Fc-fusion protein, which is preferably selected from belatacept, aflibercept, ziv-aflibercept, dulaglutide, rilonacept, romiplostim, abatacept and alefacept.
20. The compound or salt of any of items 1 to 19, wherein (V) represents a moiety derived from an anti-HER2, anti-CD37, anti-PDL1 or anti-EGFR antibody, preferably from an antibody selected from trastuzumab, pembrolizumab, naratuximab, atezolizumab durvalumab, avelumab, panitumumab, and cetuximab, more preferably from naratuximab, trastuzumab, and cetuximab, most preferably from naratuximab and trastuzumab.
21. The compound of item 20, wherein (D) is a moiety derived from an antineoplastic agent, and preferably a moiety derived from a drug selected from amanitin, duocarmycin, auristatin, auristatin F (AF), monomethyl auristatin F (MMAF), maytansine, mertansine (DM1), ravtansine (DM4), tubulysin, calicheamicin, camptothecin, SN-38, exatecan, Maaa-1181a, taxol, daunomycin, vinblastine, doxorubicin, methotrexate, pyrrolobenzodiazepine (PBD) and dimers thereof, indilinobenzodiazepine (IBD) and dimers thereof, or radioisotopes and/or pharmaceutically acceptable salts thereof.
22. The compound of items 21 wherein (D) represents a moiety derived from a drug selected from auristatin, MMAF, exatecan, maytansine, DM1 and DM4; more preferably a moiety derived from DM1 or DM4.
23. The compound of claim 22, wherein (D) represents a moiety derived from DM1 and wherein the compound is represented by one of the following formulae:

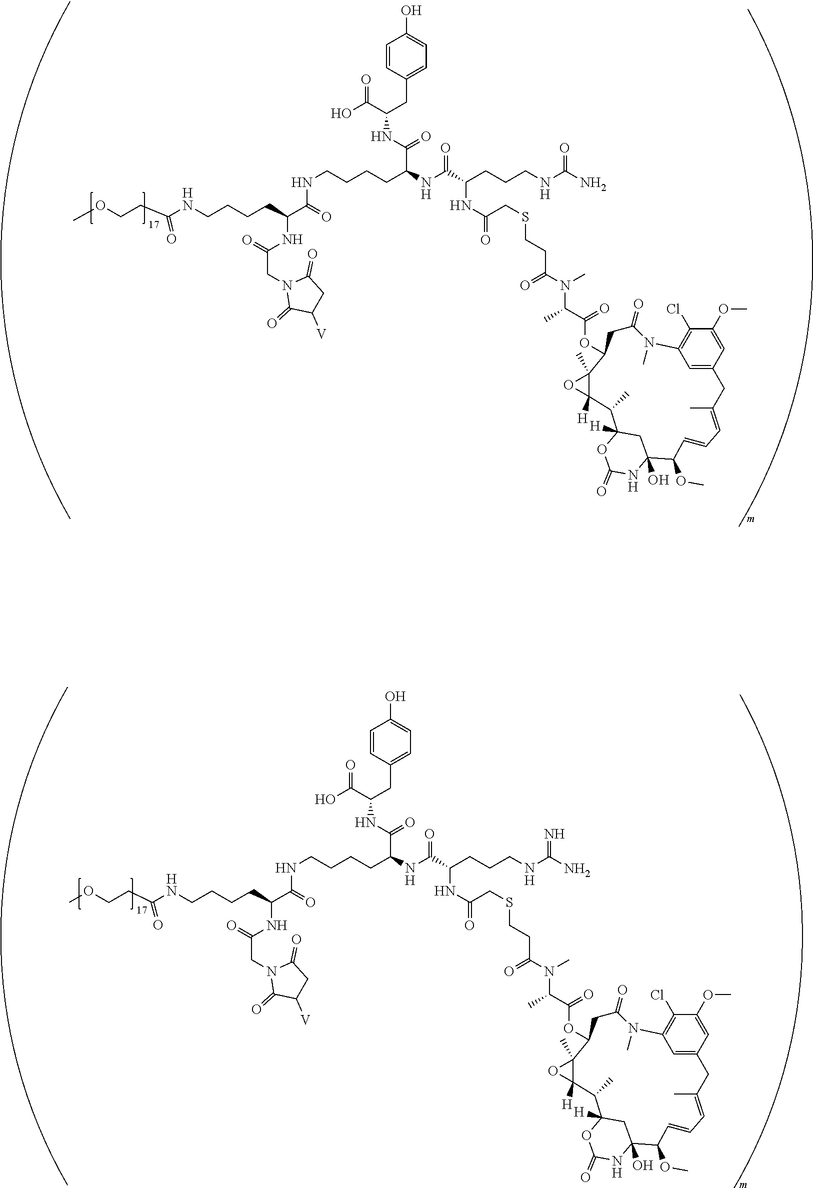

wherein m is an integer of 1 to 12, preferably 2 to 10, more preferably 4 to 8; wherein the number of oxyethylene repeating units (17) may be replaced by 12 to 30, preferably 14 to 25, more preferably 15 to 19 oxyethylene groups; and/or
wherein the maleimide attachment to (V) may be replaced by an opened hydrolyzed maleimide attachment.
24. The compound of claim 23, which is represented by the following formula:

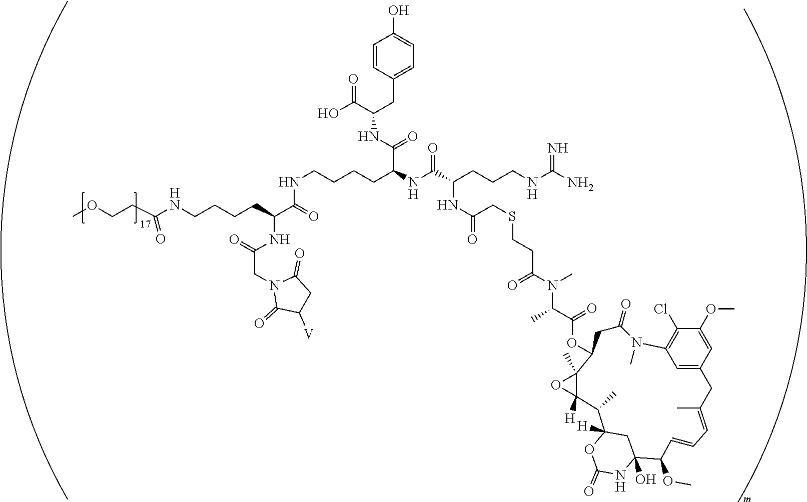

wherein m is an integer of 1 to 12, preferably 2 to 10, more preferably 4 to 8; wherein the number of oxyethylene repeating units (17) may be replaced by 12 to 30, preferably 14 to 25, more preferably 15 to 19 oxyethylene groups; and/or
wherein the maleimide attachment to (V) may be replaced by an opened hydrolyzed maleimide attachment.
25. Composition comprising a therapeutically effective amount of the compound of any of items 1 to 24 or a pharmaceutically acceptable salt thereof, and one or more components selected from a carrier, a diluent and other excipients.
26. The compound or composition of any of items 1 to 25 for use in a method of treating or preventing a cancer, an autoimmune disease and/or an infectious disease.
27. The compound or composition for use of item 26, wherein the method is a method of treating a cancer of the blood and bone marrow, and preferably acute myeloid leukemia (AML).
28. The compound or composition for use of item 26 or 27, wherein the compound is as defined in any of claims 20, 21, 22, 23 and 24, preferably as defined in claim 23 or 24, and most preferably as defined in claim 24.
29. The compound or composition for use of any of items 26 to 28, wherein in the method of treating or preventing a cancer, an autoimmune disease and/or an infectious disease, the compound or composition is administered concurrently with, before or after one or more other therapeutic agents or therapies such as chemotherapeutic agents, radiation therapy, immunotherapy agents, autoimmune disorder agents, anti-infectious agents or other compounds of formula (I).
30. A compound represented by the general formula (XI):

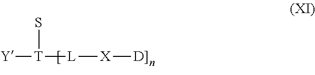

wherein,
D, X, L, T, S and n have the same meanings as specified in item 1, 2, 4, 5, 6, 7, 8, 9, 10, 11 or 12;
Y′ represents a moiety comprising a conjugation group capable of forming a covalent attachment to a molecule capable of interacting with a target cell (V′) such as a monoclonal antibody or an antibody fragment incorporated into an Fc-fusion protein; Y′ being preferably a moiety comprising a conjugation group selected from:
   an optionally substituted maleimide, preferably capable of reacting with one or two thiol groups of (V′),
   an optionally substituted haloacetamide, preferably capable of reacting with a thiol group of (V′),
   an ester, preferably capable of reacting with the side chain of an amino acid of (V′) such as an acyl halide, an N-hydroxy succinimide ester or a phenolic ester
   a carbonate, preferably capable of reacting with the side chain of an amino acid of (V′) such as a haloformate or a carbonate comprising a leaving group such as N-hydroxy succinimide or phenol;
   an isocyanate or isothiocyanate, preferably capable of reacting with the side chain of an amino acid of (V′);
   an azide, preferably capable of reacting with an alkyne group comprised in (V′);
   an alkyne, preferably capable of reacting with an azide group comprised in (V′);
   an amino group, preferably capable of reacting with a molecule (V′) in the presence of an enzyme such as a transglutaminase;
the compound preferably having a structure as shown in item 13 or 15, with the proviso that (Y′) replaces (Y) and m is 1.
31. Kit for the modification of a molecule capable of interacting with a target cell comprising the compound of item 30 and optionally a buffer, said buffer having preferably a pH of from 6.0 to 10, more preferably of from 6.5 to 8.0.
32. Method for the modification of a molecule capable of interacting with a target cell comprising reacting a molecule capable of interacting with a target cell, such as a monoclonal antibody or an antibody fragment incorporated into an Fc-fusion protein, with a compound according to item 30.

### FIGURES

FIG. 1—Exo-Cat B-induced drug release mechanism from a compound of formula (I), wherein L represents a moiety of formula (II). Intracellular exo-Cat B cleavage at the N-terminus of dipeptide Axx-Ayy releases the drug, e.g., moiety D-X-Dxx-Dyy, in the target cell.

FIG. 2—Exo-Cat B-induced drug release mechanism from a compound of formula (I), wherein L represents a moiety of formula (II′). Intracellular exo-Cat B cleavage at the N-terminus of dipeptide Ayy-Axx releases the drug, e.g., moiety D-X-Dxx-Dyy, in the target cell.

FIG. 3—Reverse Phase Liquid Chromatography (RPLC) chromatograms of trastuzumab (Tmab). A: chromatogram with basic gradient; B: chromatogram with gradient dev2; C: chromatogram with gradient dev7; D: chromatogram with gradient dev7/short.

FIG. 4—RPLC chromatograms of naratuximab. A: chromatogram with basic gradient; B: chromatogram with gradient dev7.

FIG. 5—RPLC chromatograms of prepared ADCs (DAR attribution). A: chromatogram of ADC-1 with basic gradient; B: chromatogram of ADC-2 with gradient dev7; C: chromatogram of ADC-3 with gradient dev7/short; D: chromatogram of ADC-4 with gradient dev7.

FIG. 6—RPLC chromatograms of prepared ADCs (DAR attribution). A: chromatogram of ADC-5 with basic gradient; B: chromatogram of ADC-6 with basic gradient; C: chromatogram of ADC-7 with gradient dev7/short; D: chromatogram of ADC-8 with gradient dev7/short; E: chromatogram of ADC-9 with gradient dev2.

FIG. 7—Results of in vitro cytotoxic assay (Example 8.6.1) using trastuzumab and ADC-2 in JIMT-1 (HER2 positive) cells

FIG. 8—Results of in vitro cytotoxic assay (Example 8.6.1) using trastuzumab, ADC-2 and ADC-4 in JIMT-1 cells

FIG. 9—Results of in vitro cytotoxic assay (Example 8.6.1) using trastuzumab, ADC-2, ADC-1, ADC-3 and ADC-9 in JIMT-1 cells

FIG. 10—Results of in vitro cytotoxic assay (Example 8.6.1) using trastuzumab, ADC-2 and ADC-7 in JIMT-1 cells

FIG. 11—Results of in vitro cytotoxic assay (Example 8.6.1) using naratuximab, ADC-5 and ADC-6 in SU-DHL-5 (CD37 positive) cells

FIG. 12—Results of in vivo efficacy assay (tumor volume) using ADC-2 and ADC-4 (Example 8.6.2)

FIG. 13—Results of in vivo efficacy assay (body weight) using ADC-2 and ADC-4 (Example 8.6.2)

FIG. 14—Results of in vivo efficacy assay (tumor volume) using ADC-1, ADC-2, ADC-3 and ADC-9 (Example 8.6.3)

FIG. 15—Results of in vivo efficacy assay (body weight) using ADC-1, ADC-2, ADC-3 and ADC-9 (Example 8.6.3)

FIG. 16—Results of in vivo efficacy assay (tumor volume) using naratuximab and ADC-4 (Example 8.6.4)

FIG. 17—Results of in vivo efficacy assay (survival) using naratuximab, ADC-2, and ADC-4 (Example 8.6.4)

FIGS. 18a and 18b—Results of in vivo efficacy assay (tumor growth and survival) using naratuximab, ADC-2, and ADC-4 (Example 8.6.4)

FIG. 19a, 19b, 20a, and 20b—Results of in vivo efficacy assay (tumor growth and survival) using naratuximab emtansine (Debio 1562) or ADC-4 (0.3 mg/kg, 1 mg/kg or 3 mg/kg) (Example 8.6.4)

FIG. 21a—Results of cellular binding assay—cellular binding of naratuximab antibodies to CD37 expressing AML cell lines MV-4-11, MOLM-13, HL-60 and THP-1 (Example 8.6.5)

FIG. 21b—Results of internalisation assays—internalisation of naratuximab antibodies in CD37 expressing AML cell lines MV-4-11, MOLM-13, HL-60 and THP-1 (Example 8.6.5).

FIG. 21c—Results of cytotoxicity assays—cytotoxicity of ADC-4 for the CD37 expressing AML cell lines MV-4-11, MOLM-13, HL-60 and THP-1 (Example 8.6.5).

FIG. 22a—Results of cytotoxicity assays—comparative cytotoxicity of ADC-4, naratuximab, and Debio 1562 for the CD37 expressing AML cell lines MV-4-11 (Example 8.6.6).

FIG. 22b—Results of cytotoxicity assays—comparative cytotoxicity of ADC-4 and Debio 1562 for the CD37 expressing AML cell lines THP-1 (Example 8.6.6).

FIG. 23—Results of in vivo efficacy assay—in vivo tumor growth in NCG mice (inoculated with MV4; Luc AML cells) after treatment with ADC-4 (1 mg/kg), ADC-4 (5 mg/kg) or venetoclax and azacytidine (Example 8.6.7).

FIG. 24a—Results of in vivo pharmacokinetics assays—total antibody in vivo pharmacokinetic profile (plasma concentrations) following administration of ADC-4 (5 mg/kg) and naratuximab (5 mg/kg) to male Swiss mice (Example 8.6.8).

FIG. 24b—Results of in vivo pharmacokinetics assays—total antibody and total ADC in vivo pharmacokinetic profile (plasma concentrations) following administration of ADC-4 to female CD1 mice (Example 8.6.8).

FIG. 25a—Results of in vitro plasma stability—comparative human and mouse plasma stability (DAR reduction) of ADC-4, Enhertu, and Adcetris (Example 8.6.9).

FIG. 25b—Results of in vivo pharmacokinetics assays—comparative human and mouse plasma stability (DAR reduction) of ADC-2, Enhertu, and Adcetris (Example 8.6.9).

FIG. 26—Results of in vivo efficacy assay—in vivo efficacy (% survival) in female SCID beige mice (inoculated with Farage DLBCL cells) of ADC-4 (1 mg/kg), ADC-4 (1 mg/kg)+rituximab, Debio 1562 (10 mg/kg), and Debio 1562 (10 mg/kg)+rituximab (Example 8.6.11).

FIG. 27—Results of the Cat B cleavage assay—Cat B cleavage of DM1-Ac-Cit-Lys(Ac-Cys-ma-Lys(PEG16))-Tyr-OH to release DM1-Ac-Cit (Example 8.7.3).

FIG. 28—Results of the Cat B cleavage assay—Cat B cleavage of exatecan-Suc-Phe-Cit-Lys(Ac-Cys-ma-Lys(PEG16))-Tyr-OH to release exatecan-Suc-Phe-Cit (Example 8.7.3).

FIG. 29—Scheme of a retro-Michael reaction for an LDC with a (closed) maleimide attachment.

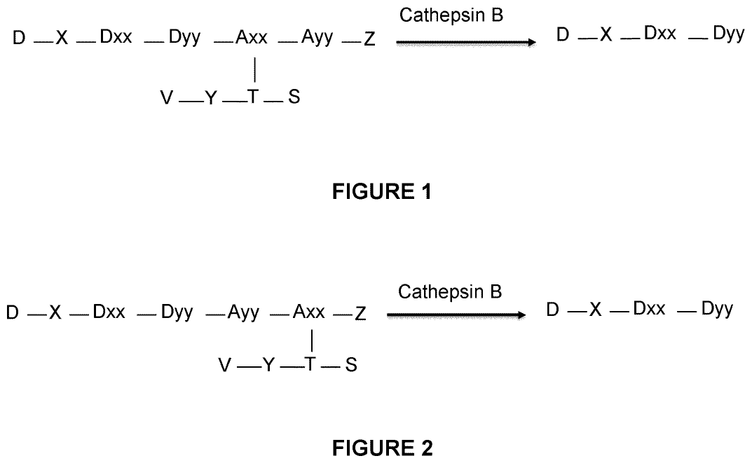

### DETAILED DESCRIPTION OF THE PRESENT INVENTION

### 1. Definitions

The term “drug” as used herein characterizes a substance (e.g., a naturally occurring or synthetic substance) which can inhibit or prevent the function of cells and/or kill cells. In some embodiments, the term “drug” is to be understood as being synonymous with other terms commonly used in the art such as “cytotoxic agent”, “toxin” or “payload” used in the field of cancer therapy. The drug may include a group derivable from a functional group that allows covalent attachment of the drug to the remainder of the compound, e.g., to divalent group (X) in formula (I), such as a carboxylic acid, a thiol group, a primary amine, a secondary amine, a hydroxyl group, or the like.

The expression “moiety derived from a drug” as used herein characterizes a moiety that contains a group, which is identical to a native drug except for the structural modifications necessary for bonding the drug to the remainder of the compound of the present invention. Depending on the functional groups available in the native drug, bonding may be effected using one of the functional groups already present in the native drug or it may be effected by incorporating a new functional group. By consequence, the (native) drug can be used for bonding in unmodified or in a modified form. That is, the drug can be unmodified (in its natural form) except for the replacement of a hydrogen atom by a covalent bond, or it can be chemically modified in order to incorporate one functional group (e.g., a group selected from hydroxyl, carboxyl, amino and thiol groups) allowing covalent attachment(s) to an adjacent group or moiety, e.g., to a divalent group (X) in formula (I). Said adjacent group or moiety will however remain attached to the drug after cleavage by Cathepsin B. The expression “moiety derived from a drug” as used herein thus refers to a moiety that differs from the unmodified (native) or modified (to incorporate one functional group) drug only by virtue of the covalent bond needed for bonding to the remainder of the molecule, and is not meant to encompass any further group, for instance, a group or moiety that remains attached thereto after cleavage by Cathepsin B.

The term “carboxyl-containing drug” characterizes an unmodified (natural) or modified drug that includes a carboxylic acid group allowing covalent attachment(s) to an adjacent group or moiety. The term “thiol-containing drug” characterizes an unmodified (natural) or modified drug that includes a thiol group allowing covalent attachment(s) to an adjacent group or moiety. Non-limiting examples of thiol-containing drugs include mertansine (DM1) and ravtansine (DM4). The term “amino-containing drug” characterizes an unmodified (natural) or modified drug that includes a primary or secondary amino group allowing covalent attachment(s) to an adjacent group or moiety. Non-limiting examples of amino-containing drugs include monomethyl auristatin F (MMAF), exatecan and the indilinobenzodiazepine (IBD) derivative 2554618-79-8 (shown below). The term “hydroxyl-containing drug” characterizes an unmodified (natural) or modified drug that includes a hydroxyl group allowing covalent attachment(s) to an adjacent group or moiety. An example of a hydroxyl-containing drug is the exatecan derivative Maaa-1181a (shown below).

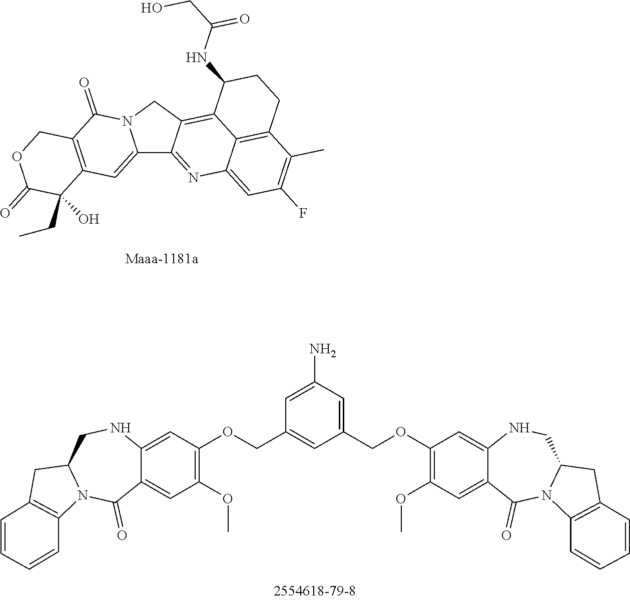 [SMILES: CC[C@@]1(O)C(=O)OCc2c1cc1n(c2=O)Cc2c-1nc1cc(F)c(C)c3c1c2[C@@H](NC(=O)CO)CC3 ; COc1cc2c(cc1OCc1cc(N)cc(COc3cc4c(cc3OC)C(=O)N3c5ccccc5C[C@H]3CN4)c1)NC[C@@H]1Cc3ccccc3N1C2=O]

In an analogous manner, the term “derivative” is used to characterize moieties bonded to adjacent moieties, which moieties differ from the molecules from which they are derived only by the structural elements responsible for bonding to adjacent moieties. This may include covalent bonds formed by existing functional groups or covalent bonds and adjacent functional groups newly introduced for this purpose.

Likewise, unless specified otherwise or unless the context dictates otherwise, the expression “derived from” (such as in “derived from a compound”), when used in connection with other groups or moieties, is meant to describe a group or moiety, which is identical to the referenced compound or the like except for the structural modifications necessary for bonding the group or moiety to the one or more adjacent groups or moieties, typically by replacing a hydrogen atom or atomic group by a covalent bond (e.g. replacement of OH in a carboxyl group by a covalent bond upon amide bond formation with an amino group; further examples are given in the below table at the end of the section “Divalent group (X)”).

The term “native drug” refers to a compound, for which therapeutic efficacy has been established by in vitro and/or in vivo tests. In a preferred embodiment, the native drug is a compound for which therapeutic efficacy has been established by clinical trials. Most preferably, the native drug is a drug that is already commercially available. The type of therapeutic efficacy to be established and suitable tests to be applied depend of course on the type of medical indication to be treated.

When referring to specific classes of drug molecules, such as an antineoplastic agent, a topoisomerase inhibitor, an RNA-polymerase II inhibitor, a DNA cleaving agent, an antimitotic agent or microtubule disruptor, an anti-metabolite, a kinase inhibitor, an immunomodulatory agent, or an anti-infectious disease agent, these terms are intended to have the meaning generally accepted in the field of medicine, as reflected, for instance, in the Mosby's Medical Dictionary, Mosby, Elsevier 10^{th} ed. (2016), or in Oxford Textbook of Oncology, David J. Kerr, OUP Oxford 3^{rd} ed. (2016).

Accordingly, the drug to be used in the ligand-drug-conjugate of the present invention can be a native drug (e.g. a drug naturally containing one or more functional groups allowing covalent attachment to the conjugate), or can be a drug chemically modified to incorporate one functional group (e.g., a group selected from hydroxyl, carboxyl, amino and thiol groups) allowing covalent attachment(s) to an adjacent group or moiety) provided that the modified drug is pharmacologically active. Pharmacological activity in this connection means at least 20%, preferably at least 50%, more preferably at least 80% of the pharmacological activity of the native drug.

Furthermore, in those instances where the drug is released in a modified form insofar as a group or moiety remains attached thereto after cleavage by Cathepsin B, e.g., released as a moiety D-X or D-X-Dxx-Dyy, the modified drug can be referred to as an “intra-drug”. In some instances, the remaining groups X, Dxx and Dyy may subsequently be removed via other mechanisms, e.g., by hydrolysis of an ester linkage that may be present between D and X. For those linkages that are not cleaved by other mechanisms, it is advantageous if the remaining modified drug D-X or D-X-Dxx-Dyy is pharmacologically active. Pharmacological activity in this connection means that the released modified drug, e.g., the moiety D-X or D-X-Dxx-Dyy, retains at least 20%, preferably at least 50%, more preferably at least 80% of the pharmacological activity of the native drug when released intracellularly by the ADC. To ensure realistic conditions, such a test for activity should not be made via cell cytotoxicity comparison of the released modified drug and the native drug because these conditions require entry of the modified drug into the cell, which may introduce a cell permeability bias. Differences in permeability between these two entities are not relevant here due to the intracellular release of the modified drug. It may be possible to compare activities of the modified drug and the native drug in a cell-free binding assay to determine Ki values (binding affinities) for the appropriate target receptor of the drug. If it is not possible to determine the Ki values, one may compare the IC50 of the cytotoxicity in HER2+ cell lines of two trastuzumab ADCs with exact same linker system (e.g. Val-Cit-PAB), one designed to release the modified drug and one to release the native drug.

The term “pharmaceutically acceptable salts” as used herein refers to derivatives of disclosed compounds (including the reactive conjugates) wherein the parent compound is modified by making acid or base salts thereof. The pharmaceutically acceptable salts include the non-toxic salts or the quaternary ammonium salts of the parent compound formed, for example, from non-toxic inorganic or organic acids or bases. Lists of suitable salts can be found in Remington's Pharmaceutical Sciences, 17^{th} ed., Mack Publishing Company, Easton, PA, 1985, page 1418, S. M. Berge, L. M. Bighley, and D. C. Monkhouse, “Pharmaceutical Salts” J. Pharm. Sci. 1977, 66(1), 1-19; P. H. Stahl and C. G. Wermuth, editors, Handbook of Pharmaceutical Salts: Properties, Selection and Use, Weinheim/Zürich, Wiley-VCH, 2008 and in A. K. Bansal et al., Pharmaceutical Technology, 3(32), 2008. The pharmaceutical salts can be synthesized from the parent compound which contains a basic or acidic moiety by conventional chemical methods. For the reactive conjugates, this can be done before or after incorporating the drug moiety into the compound of the present invention. Unless the context dictates otherwise, all references to compounds (conjugates, modified antibodies, etc.) of the invention are to be understood also as references to pharmaceutically acceptable salts of the respective compounds.

The term “backbone” refers to the connected link of atoms that span the length of a molecule. Accordingly, the expression “divalent group comprising X to Y backbone atoms” defines a group in which atoms, e.g., C, N, O, S, are covalently attached to each other to form a connected chain of atoms, said chain being covalently attached at its extremities to adjacent groups or moieties. The divalent group may include pendant atoms or groups that are attached to the backbone portion. Hydrogen atoms that saturate valencies of the backbone atoms are not counted as backbone atoms. If cyclic groups are present, the backbone is identified as the shortest connection between the termini.

The term “functional group” refers to a group that is capable of bonding to another functional group by forming at least one covalent bond without need for breaking any C—C or C—H covalent bonds.

The expression “capable of being cleaved by Cathepsin B” characterizes any compound (or moiety that may be incorporated into a compound), which is cleaved when being contacted with Cathepsin B (Cat B) under suitable conditions e.g., as set out in WO2019096867. In preferred embodiments, said cleavage is (a) fast and/or (b) cleavage is via the exopeptidase activity of Cat B. Said embodiment (b), relating to a compound or moiety that is “capable of being cleaved by the exopeptidase activity of Cat B”, is defined in more detail in the next paragraph. The above-mentioned “fast” cleavage of embodiment (a) typically means for a compound of interest that the corresponding unconjugated compound (i.e. compound not having a vector group V and being quenched at the conjugation group, for example with cysteine being covalently attached to a maleimide conjugation group) has a cleavage rate T_{1/2} of 25 min or less, preferably 20 min or less, more preferably 18 min or less, even more preferably 16 min or less and most preferably 14 min or less. There is no particular lower limit. However, as the solubilizing group (PEG or the like) tends to reduce the cleavage rate, it is realistic to expect cleavage rates T_{1/2} of 1 min or more, typically 2 min or more and even more typically 5 min or more.

The expression “capable of being cleaved by the exopeptidase activity of Cat B” as used herein indicates that the respective moiety of the compound, in particular the linker, e.g., C-terminal peptide unit, can be specifically recognized and cleaved by the exopeptidase (i.e., carboxydipeptidase) of Cathepsin B. Said cleavage gives rise to the rapid release of the drug (or a modified drug having group or moiety that remains attached thereto after cleavage by Cathepsin B, “intra-drug”) into the target cell. The cleavage of a linker, e.g., a C-terminal peptide unit, via the exopeptidase activity of Cat B can be assessed by the in vitro enzymatic cleavage assay using recombinant human Cat B and UHPLC-MS/MS analysis as described further below. Considering that exopeptidase activity of Cat B is typically associated with higher cleavage rates compared to endopeptidase activity of Cat B, in some aspects, the expression “compound capable of being cleaved by the exopeptidase activity of Cat B” may be confirmed by confirming a high Cat B cleavage rate. According to this aspect, a “compound capable of being cleaved by the exopeptidase activity of Cat B” refers to a compound for which the following criterion is fulfilled: the corresponding unconjugated compound (i.e. compound not having a vector group V and being quenched at the conjugation group, for example with cysteine being covalently attached to a maleimide conjugation group) has a cleavage rate T_{1/2} of 25 min or less, preferably 20 min or less, more preferably 18 min or less, even more preferably 16 min or less and most preferably 14 min or less. There is no particular lower limit. However, as the solubilizing group (PEG or the like) tends to reduce the cleavage rate, it is realistic to expect cleavage rates T_{1/2} of 1 min or more, typically 2 min or more and even more typically 5 min or more. In some aspects, the expression “compound capable of being cleaved by the exopeptidase activity of Cat B” can refer to a compound having a cleavage ratio as compared with reference compound A or reference Cys quenched compound B, the structures of which are given below

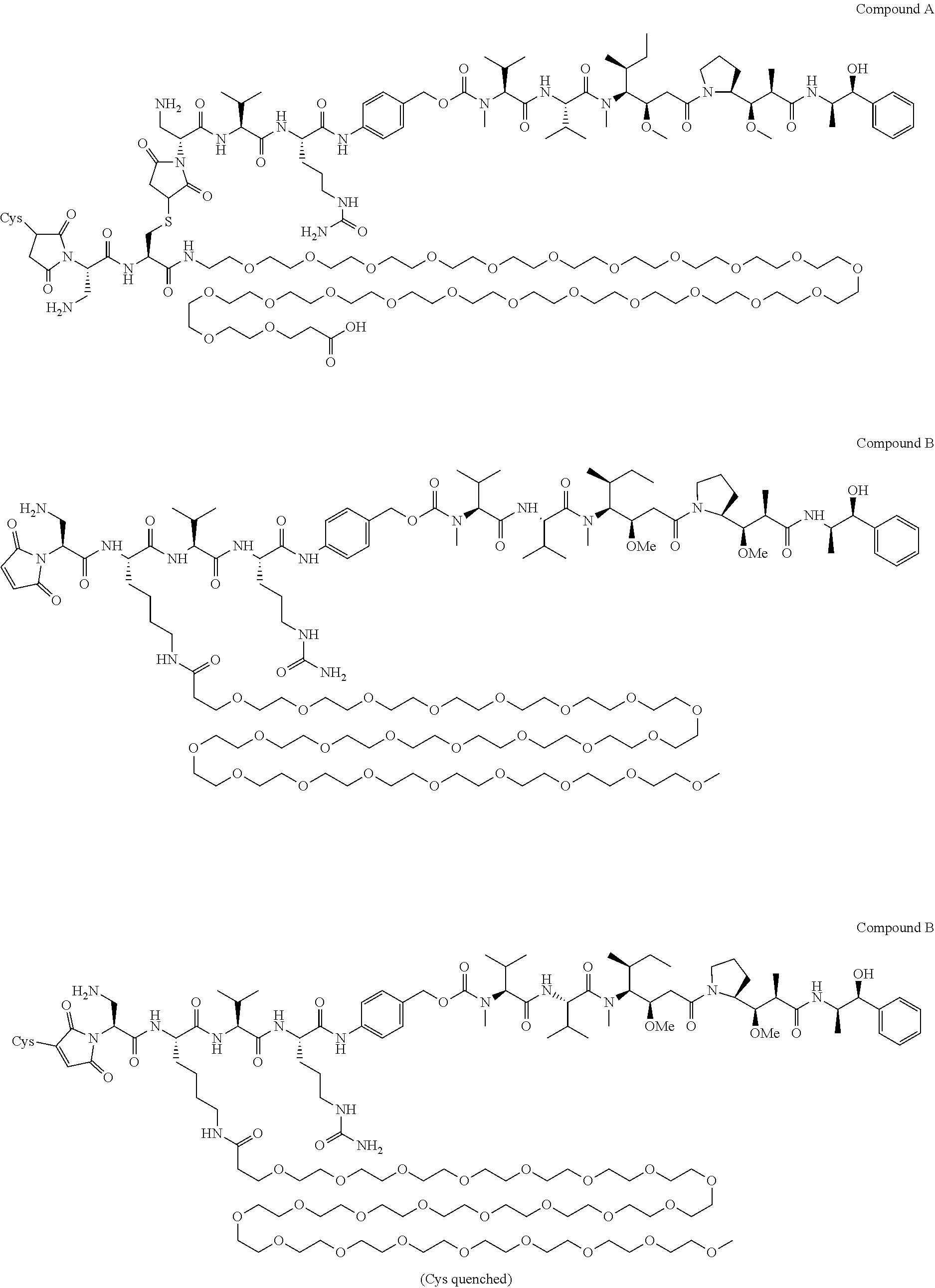

wherein said ratio (T_{1/2 Compound}/T_{1/2 Reference}) is 0.6 or less, preferably 0.4 or less, and more preferably 0.2 or less. Again, there is no particular lower limit for this range, but typical cleavage ratios are 0.001 or more, more typically 0.01 or more and especially 0.05 or more.

The term “solubilizing group” or “solubilizing moiety” as used herein refers to a hydrophilic group or moiety, which can enhance (improve) the water solubility of the moiety or compound to which it is attached. The solubilizing group can be, for example, a polyalkylene oxide group, such as a polyethylene oxide (PEO) or a polypropylene oxide (PPO) group, a saccharide group or a moiety comprising one or more ionic or ionizable groups, i.e., functional groups which are charged (anionic or cationic) at physiological pH (7.4), such as moieties derived from amino acids, e.g., from Lys, Glu, Asp, His, Arg, diaminopropionic acid (Dap), diaminobutyric acid (Dab), 2-aminoadipic acid (Aad), carnitine, Orn. Examples of ionic or ionizable groups include ammonium groups, guanidinium groups, sulfate groups, phosphate groups, phosphonate groups, and sulfonate groups. Examples of saccharide groups include monosaccharides, disaccharides and linear or branched oligosaccharides, in particular linear or branched oligosaccharides having 3 to 10 monosaccharide units being linked by glycosidic bond, wherein each of the monosaccharide units in the monosaccharide, disaccharide and oligosaccharide is independently selected from glucose, fructose, mannose, ribose, and galactose. In the present invention, the term “solubilizing moiety” refers to a moiety derived from a compound comprising one or more, e.g., two, three or four, solubilizing groups. In further embodiments, the solubilizing moiety can consist of one or more solubilizing groups, e.g., amino acids, PEO groups.

The term “polyalkylene oxide” (or “polyalkylene glycol”, “polyoxyalkylene”) as used herein refers to substances of the general structure HO—(X-G)ₙ-H, wherein X represents an akylene group having 2 or 3 carbons atoms, and n indicates the number of repeating units, e.g., 6 to 200, 10 to 150, or 12 to 80 repeating units, such as 16 or 40 repeating units, e.g., 17, 18, 20 or 24 PEO repeating units. Thus, the term “polyalkylene oxide group” is to be understood as a divalent group of formula *—O—(X—O)ₙ—**, wherein X and n are as defined above, and * and ** indicate covalent attachment to adjacent moieties. In some embodiments, the term “polyalkylene oxide” can refer to polyethylene oxide (or polyethylene glycol, C₂-polyalkylene oxide), or polypropylene oxide (or polypropylene glycol, C₃-polyalkylene oxide). It is also possible to provide a polyalkylene oxide group, in which two or more different alkylene groups, as defined above, are arranged in a random or block-wise manner.

The term “peptide” as used herein refers to a compound comprising a continuous sequence of at least two amino acids linked to each other via peptide linkages. The terms “dipeptide”, “tripeptide” and “tetrapeptide” respectively refer to a compound comprising a continuous sequence of two, three and four amino acids linked to each other via peptide linkages. The term “peptide linkage” in this connection is meant to encompass (backbone) amide bonds as well as modified linkages, which can be obtained if non-natural amino acids are introduced in the peptidic sequence. In this case, the modified linkage replaces the (backbone) amide bond which is formed in the continuous peptide sequence by reacting the amino group and the carboxyl group of two amino acid residues. For instance, the modified linkage may be an ester, a thioester, a carbamide, a thiocarbamide or a triazole linkage. Preferably, the amino acids forming the continuous peptide sequence are linked to each other via backbone amide bonds. The peptide may be linear or branched. In preferred aspects, the peptide is a linear di-, tri-, tetra-peptide, more preferably a linear tri- or tetra-peptide.

The term “amino acid” as used herein refers to a compound that contains or is derived from a compound containing at least one amino group and at least one acidic group, preferably a carboxyl group. The distance between amino group and acidic group is not particularly limited. α-, β-, and γ-amino acids are suitable but α-amino acids and especially α-amino carboxylic acids are particularly preferred. The term “amino acid” encompasses both naturally occurring amino acids such as the naturally occurring proteinogenic amino acids, as well as synthetic amino acids that are not found in nature. In the following, a reference to amino acids may be made by means of the 3-letter amino acid code (Arg, Phe, Ala, Cys, Gly, Gln, etc.), or by means of the 1-letter amino acid code (R, F, A, C, G, Q, etc.).

Hereinafter, amino acid sequences are written from the N-terminus to the C-terminus (left to right). Unless specified otherwise or dictated otherwise by the context, all connections between adjacent amino acid groups are formed by peptide (amide) bonds.

The expression “amino acid in the (D) configuration” as used herein refers to the (D)-isomer of any naturally occurring or synthetic amino acid. This applies to α-amino acids as well as to β- and γ-amino acids. The expression “amino acid in the (D) configuration” as used herein is not meant to encompass non-chiral amino acids such as glycine or other non-chiral amino acids such as aminoisobutyric acid.

The expression “side chain of an amino acid” as used herein may refer to a moiety attached to the α-carbon of an amino acid. For example, the side chain of Ala is methyl, the side chain of Phe is phenylmethyl, the side chain of Cys is thiomethyl, the side chain of Tyr is 4-hydroxyphenylmethyl, etc. Both naturally occurring side chains and non-naturally occurring side chains are included by this definition.

The term “trifunctional” as used herein refers to a compound or moiety having three functional groups that can form or have formed three covalent bonds to adjacent moieties. Thus, the term “trifunctional amino acid” refers to a compound that contains or is derived from a compound containing at least an amino group, an acid group (e.g. a carboxyl group) and another functional group such as an amino group or a carboxyl group. Non-limiting examples of trifunctional amino acids include Ser, Cys, Tyr, N-ε-propargyloxycarbonyl-L-Lysine (Lys(Poc)), Asp, Glu, Orn, Lys, Dab and Dap.

The term “C-terminal” as used herein refers to the C-terminal end of the amino acid (peptide) chain. Binding to the “C-terminus” means that a covalent bond is formed between the acid group in the main chain (backbone) of the amino acid residue and the binding partner. For instance, binding of group “X” to the C-terminus of amino acid residue “Axx” yields an ester or amide-type structural element *—C(O)—X, wherein the carbonyl group is derived from the acid group of Axx and (*) indicates attachment to main chain. The term “C-terminal peptide unit” is used herein to characterize a peptide sequence of 2, 3 or 4 amino acids wherein the C-terminal amino acid forms the C-terminus of the peptide sequence.

The term “N-terminal” as used herein refers to the N-terminal end of the amino acid (peptide) chain. Binding to the “N-terminus” means that a covalent bond is formed between the amino group in the main chain (backbone) of the amino acid residue and the binding partner (which replaces one hydrogen atom). For instance, binding of group “X” to the N-terminus of amino acid residue “Axx” yields a structural element X—NH—*, wherein the amino group is derived from Axx and (*) indicates attachment to main chain.

The term “hydrophobic” is used herein to characterize compounds, groups or moieties, which lack affinity for water. For instance, the term “amino acid with hydrophobic side chain” is used to characterize amino acids with a hydrophobic or partially hydrophobic aliphatic side chain or amino acids with aromatic side chain such as Phe, Leu, Ile, Val, Tyr, Trp, Ala. Of course, any other amino acid exhibiting the same or a higher degree of hydrophobicity should also be treated as hydrophobic in the sense of the present invention. A comparison of the degree of hydrophobicity can be done by determining the n-octanol/water partition coefficient (at 25° C. and pH 7): if the ratio of concentrations in n-octanol/water for another amino acid is equal or higher than that of one or more of the amino acids Phe, Leu, Ile, Val, Tyr, Trp, Ala, such other amino acid is to be treated as a hydrophobic amino acid.

The term “amino acid with a basic side chain” is used herein to characterize natural or unnatural amino acids wherein the side chain contains one or more ionizable groups having a pKa value equal to or greater than 6. Examples of natural amino acids with a basic side chain include Arg (guanidino group, pKa=12.5), Lys (amino group, pKa=10.5), His (imidazole group, pKa=6). Examples of unnatural amino acids with a basic side chain include citrulline (Cit), ornithine (Orn), 2,3-diamino-propionic acid (Dap), 2,4-diamino-butyric acid (Dab).

The term “alkyl group” as used herein refers to a linear or branched hydrocarbon group having from 1 to 20 carbon atoms, preferably from 1 to 5 carbon atoms, more preferably a methyl or an ethyl group, or to a cycloalkyl group having from 3 to 20 carbon atoms, preferably from 5 to 8 carbon atoms. The cycloalkyl group may consist of a single ring, but it may also be formed by two or more condensed rings.

The term “divalent maleimide derivative” (or, e.g., “divalent group derived from a compound selected from maleimide . . . ”) as used herein refers to a divalent moiety derived from maleimide (e.g., a succinimide moiety), in which the double bond is hydrogenated and two hydrogen atoms are replaced by two covalent bonds allowing attachment to adjacent moieties. For example, the divalent maleimide derivative may have the following structure (wherein R and R′ represent adjacent moieties to which said maleimide derivative is attached):

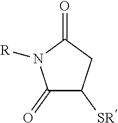

Said moiety contains a chiral carbon atom (i.e. the atom carrying the sulfur atom). Unless specified otherwise, references to a divalent maleimide derivative are to be understood as references to the pure stereoisomers as well as any mixture thereof and especially the racemic mixture thereof.

The term “divalent maleimide derivative” or “divalent group derived from a compound selected from maleimides” is further to be understood as encompassing any derivative of maleimide (as described above) additionally being substituted at other positions than positions 2 and 3, as well as opened hydrolyzed maleimide derivatives.

A divalent maleimide-type disulfide bridge (e.g. a divalent group of formula —S—X²—S—/—S—X³—S— wherein X²/X³ represents a divalent group derived from maleimide) can be obtained by side-chain-to-side-chain cyclization in the presence of e.g. 2,3-dibromomaleimide or another suitable reagent as described by Kuan et al. in Chem. Eur. J. 2016, 22, 17112-17129.

In the context of the invention, an “opened hydrolyzed maleimide derivative” refers to a divalent moiety derived from maleimide (as described above) wherein the maleimide ring has been opened by hydrolysis. For instance, the hydrolysis of the divalent maleimide derivative R—X—S—R′ (wherein X represents an unsubstituted divalent group derived from maleimide and R/R′ represent adjacent groups or moieties; in X the double bond of maleimide is no longer present) leads to an opened hydrolyzed maleimide derivative of formula R—NH—C(═O)—CH(S—R′)—CH₂—COOH, or R—NH—C(═O)—CH₂—CH(S—R′)—COOH, or a mixture thereof. The ring hydrolysis can be performed, for example, under basic conditions. The following conditions are especially suitable: at the end of a cysteine-maleimide conjugation reaction (e.g., after the reaction of a maleimide moiety (Y′) with the side chain of a cysteine residue contained in a molecule capable of interacting with a target cell (V′)), pH is adjusted to pH 8 by adding 10× pH 8 DPPS (0.2 to 0.5 reaction volume) and excess reactive drug linker and reducing agent (TCEP) are removed via gel filtration using suitable columns for gel filtration (e.g., PF column, elution with pH 8 buffer), the eluent is then stirred overnight for 16 h to complete the opening before final buffer exchange with DPBS into an Amicon concentrating unit. Unless specified otherwise, any reference to an “opened hydrolyzed maleimide derivative” is to be understood as a reference to one of these structures alone or any mixture of these structures. Moreover, the carbon carrying the sulfur atom is chiral. Unless specified otherwise, any reference to an “opened hydrolyzed maleimide derivative” is to be understood as a reference to the pure stereoisomers as well as any mixture thereof and especially the racemic mixture thereof.

Furthermore, in the context of the invention, a “maleimide attachment” refers to a divalent moiety derived from maleimide as described above which contains two covalent bonds allowing attachment to adjacent groups or moieties. For example, in the maleimide derivative of the formula R—X—S—R′, where R/R′ represent adjacent groups or moieties, X represents the maleimide attachment (a divalent group derived from maleimide in which the double bond of maleimide is no longer present). Thus, the term “maleimide attachment” is synonymous with “maleimide derivative attachment”.

Similarly, in the context of the invention, an “opened hydrolyzed attachment” refers to a divalent moiety derived from maleimide as described above which contains two covalent bonds allowing attachment to adjacent groups or moieties. For example, in the opened hydrolyzed maleimide derivative of the formula R—X—S—R′, where R/R′ represent adjacent groups or moieties, X represents the opened hydrolyzed maleimide attachment. Thus, the term “opened hydrolyzed attachment” is synonymous with “opened hydrolyzed derivative attachment”.

References to “a divalent group derived from a compound selected from . . . triazoles” are meant to characterize divalent groups resulting from a 3+2 cycloaddition of an alkyne and an azide. Such divalent groups are typically characterized by the following structures:

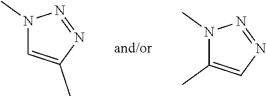

wherein the single bonds characterize attachment to adjacent groups, such that there is no particular restriction as to which adjacent group is bonded to the nitrogen atom and which adjacent group is bonded to the carbon atom. In the context of group Y, the divalent group may be formed by reacting an alkyne group attached to V with an azide group attached to T or vice versa.

References to “a divalent group derived from a compound selected from . . . hydrazones” are meant to characterize divalent groups —CH═N—NH— resulting from condensation of a carbonyl group with a hydrazine group. In the context of group Y, the divalent group may be formed by reacting a carbonyl group attached to V with a hydrazine group attached to T or vice versa.

References to “a divalent group derived from a compound selected from . . . carbonyl-containing compounds” are meant to characterize divalent groups —C(═O)—X—with X representing O, S or NH, resulting from reacting (an activated) carbonyl with a nucleophilic group such as formation of an amide group, ester group, thioester group. In the context of group Y, the divalent group may be formed by reacting a carbonyl-containing group (e.g. —C(═O)—Cl) attached to V with a nucleophilic group (e.g. —NH₂) attached to T, or vice versa.

In the above divalent groups, the term “derivative thereof” means that any hydrogen atom may be replaced by a substituent, as defined hereinbelow, as long as the substitution does not interfere with divalent group formation.

The term “leaving group” as used herein refers to an atom or group (which may be charged or uncharged) that becomes detached from an atom or a molecule in what is considered to be the residual or main part of the molecule taking part in a specific reaction, for instance a nucleophilic substitution reaction (Pure Appl. Chem. 1994, 66, 1134). Examples of leaving groups include thiophenolates, phenolates, carboxylates, sulfonates.

The expression “moiety derived from a vector group” as used herein indicates that the vector group can be in an unmodified or modified form. That is, the vector group can be unmodified (in its natural form) except for the replacement of a hydrogen atom by a covalent bond, or chemically modified so as to introduce one or more functional groups (e.g. a group selected from hydroxyl, carboxyl, thiol and/or amino groups) allowing covalent attachment(s) of the vector group to T provided that such modifications do not interfere to a significant degree with the interaction between vector group and target cell.

The expression “capable of interacting with a target cell” as used herein indicates that the vector group can bind to, complex with, or react with a moiety, e.g. a protein or receptor, that is exposed on the surface of a target cell. Said interaction may give rise to a targeting effect (i.e. to a local increase of the concentration of the vector-carrying compound in the vicinity of the target cell) and/or it may cause internalization of the vector-carrying compound of the present invention into the target cell.

The term “antibody” (also synonymously called “immunoglobulin” (Ig)) as used herein covers monoclonal antibodies, polyclonal antibodies, dimers, multimers, multi-specific antibodies (e.g. bispecific antibodies), veneered antibodies, and small immune proteins. An antibody is a protein generated by the immune system that is capable of recognizing and binding to a specific antigen. A target antigen generally has numerous binding sites, also called epitopes, recognized by complementary-determining regions on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e. a molecule that contains an antigen-binding site that immuno-specifically binds an antigen of a target of interest or part thereof. The antibodies may be IgG e.g. IgG1, IgG2, IgG3, IgG4. Preferably, the antibody is an IgG protein and more preferably an IgG1, IgG2 or IgG4 protein. Most preferably the antibody is an IgG1 protein. The antibody can be human or derived from other species. Preferably the antibody is a human antibody.

The term “monoclonal antibodies” as used herein characterizes antibodies that are identical because they are produced by one type of immune cell and are all clones of a single parent cell.

The term “antibody fragment” as used herein refers to a molecule comprising at least one polypeptide chain derived from an antibody that is not full length.

The term “Fc-fusion protein” as used herein refers to a protein comprising at least an Fc-containing antibody fragment—i.e. an immunoglobulin-derived moiety comprising at least one Fc region—and a moiety derived from a second, non-immunoglobulin protein. The Fc-containing antibody fragment forms part of the Fc-fusion protein and therefore is incorporated into the Fc-fusion protein. The Fc-containing antibody fragment can be derived from an antibody as described hereinabove, in particular from IgG e.g. IgG1, IgG2, IgG3, IgG4. Preferably, the Fc-containing moiety is derived from an IgG1 protein, more preferably from a human IgG1 protein. The non-Ig protein can be a therapeutic protein, for instance a therapeutic protein derived from erythropoietin (EPO), thrombopoietin (THPO) such as THPO-binding peptide, growth hormone, interferon (IFN) such as IFNα, IFNβ or IFNγ, platelet-derived growth factor (PDGF), interleukin (IL) such as IL1a or IL1p, transforming growth factor (TGF) such as TGFα or TGFβ, or tumor necrosis factor (TNF) such as TNFα or TNFβ, or a therapeutic protein derived from a receptor, in particular from a ligand-binding fragment of the extracellular domain of a receptor, for instance derived from cluster of differentiation 2 (CD2), CD4, CD8, CD11, CD14, CD18, CD20, CD22, CD23, CD25, CD33, CD40, CD44, CD52, CD58 (LFA3), CD80, CD86, CD147, CD164, IL2 receptor, IL4 receptor, IL6 receptor, IL12 receptor, epidermal growth factor (EGF) receptor, vascular endothelial growth factor (VEGF) receptor, epithelial cell adhesion molecule (EpCAM), or cytotoxic T-lymphocyte-associated protein 4 (CTLA4). Examples of Fc-fusion proteins include belatacept (Nulojix®), aflibercept (Eyla®), rilonacept (Arcalyst®), romiplostim (NPlate®), abtacept (Orencia®), alefacept (Amevine®), and etanercept (Enbrel®).

The term “cancer” as used herein means the physiological condition in mammals that is characterized by unregulated cell growth. A tumor comprises one or more cancer cells. Examples of cancer include carcinoma, lymphoma, blastoma, sarcoma, and lymphoid malignancies. Further examples of cancer include squamous cell cancer (e.g. epithelial squamous cell cancer), non-Hodgkin lymphoma (NHL) e.g., Diffuse large B cell lymphoma, lung cancer including small-cell lung cancer, non-small cell lung cancer, acute myeloid leukemia (AML), adenocarcinoma of the lung and squamous carcinoma of the lung, cancer of the peritoneum, hepatocellular cancer, gastric or stomach cancer including gastrointestinal cancer, gastrointestinal stromal tumor, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, thyroid cancer and hepatic cancer.

The term “drug-antibody-ratio” (or “DAR”) as used herein refers to the average number of drug molecule(s) attached to one (e.g., antibody) moiety (V). The DAR is sometimes referred to in the art as “drug load”, or “drug loading”. The DAR in the compound of the present invention may be calculated by multiplying n by m in Formula (1). If n=1, then m in Formula (1) represents the DAR. However, it is also understood the DAR will often be an average value when used to describe a sample containing many molecules, due to some degree of inhomogeneity, typically associated with the conjugation step. The average DAR, for instance, may be in the range of about 1 to about 10, and may be about 1, 1.5, 2, 2.5, 3, 3.5, 4, 4.5, 5, 5.5, 6, 6.5, 7, 7.5, 8, 8.5, 9, 9.5 or 10. In some aspects, the DAR may be from about 3 and about 8, and may be typically about 3, 3.5, 4, 4.5, 5, 5.5, 6, 6.5, 7, 7.5, 8, 8.5 or 9. In some aspects, the DAR may be about 4. In some aspects, the DAR may be about 8. In some aspects, a DAR of ‘about n’ means that the measured value for DAR is within ±20% of n (e.g., between 80% of n and 120% of n).

The term “emtansine” as used herein refers to the complex formed by covalent attachment of the non-reducible linker N-succinimidyl-4-(N-maleimidomethyl) cyclohexane-1-carboxylate (“SMCC”, designated as “MCC” after conjugation to e.g. an antibody) to the antimitotic agent mertansine (DM1). Emtansine is used in the known antibody-drug-conjugate trastuzumab emtansine (Kadcyla®).

The term “patient” as used herein refers to a subject to which a compound of the present invention, i.e., a ligand-drug-conjugate, is administered. In the context of the present invention, the patient is a mammal, and preferably a human (male or female).

Where the present description refers to “preferred” embodiments/features, combinations of these “preferred” embodiments/features shall also be deemed as disclosed as long as this combination of “preferred” embodiments/features is technically meaningful.

Hereinafter, in the present description of the invention and the claims, the use of the terms “containing” and “comprising” is to be understood such that additional unmentioned elements may be present in addition to the mentioned elements. However, these terms should also be understood as disclosing, as a more restricted embodiment, the term “consisting of” as well, such that no additional unmentioned elements may be present, as long as this is technically meaningful. For instance, the expression “divalent carbonyl-containing group” includes as a preferred embodiment a divalent group consisting of carbonyl (—CO—). Moreover, the expression “at least one of X and Y” is to be understood broadly as disclosing one or both of X and Y, i.e. as being equivalent to the expression “at least one selected from the group of X and Y”.

Unless specified otherwise or the context dictates otherwise, references to groups being “substituted” or “optionally substituted” are to be understood as references to the presence (or optional presence, as the case may be) of at least one substituent selected from F, Cl. Br, I, CN, NO₂, NH₂, NH—C₁₋₆-alkyl, N(C₁₋₆-alkyl)₂, —X—C₁₋₆-alkyl, —X—C₂₋₆-alkenyl, —X—C₂₋₆-alkynyl, —X—C₆₋₁₄-aryl, —X-(5-14-membered heteroalkyl with 1-3 heteroatoms selected from N, O, S), wherein X represents a single bond, —(CH₂)—, —O—, —S—, —S(O)—, —S(O)₂—, —NH—, —CO—, or any combination thereof including, for instance, —C(O)—NH—, —NH—C(O)—. The number of substituents is not particularly limited and may range from 1 to the maximum number of valences that can be saturated with substituents. It is typically 1, 2 or 3 and usually 1 or 2, most typically 1.

Unless specified otherwise, all valencies of the individual atoms of the compounds or moieties described herein are saturated. In particular, they are saturated by the indicated binding partners. If no binding partner or a too small number of binding partners is indicated, the remaining valencies of the respective atom are saturated by a corresponding number of hydrogen atoms.

Unless specified otherwise, chiral compounds and moieties may be present in the form of a pure stereoisomer or in the form of a mixture of stereoisomers, including the 50:50 racemate. In the context of the present invention, references to specific stereoisomers are to be understood as references to compounds or moieties, wherein the designated stereoisomer is present in at least 90% enantiomeric excess (ee), more preferably at least 95% ee and most preferably 100% ee, wherein % ee is defined as (|R−S|)/(R+S)*100% with R and S representing the amount of moles of the respective enantiomers.

Unless the context dictates otherwise, and/or alternative meanings are explicitly provided herein, all terms are intended to have meanings generally accepted in the art, as reflected by IUPAC Gold Book (status of 1 Aug. 2020), or the Dictionary of Chemistry, Oxford, 6^{th} Ed.

### 2. Overview

The present invention is based on the discovery of a new cleavable linker system comprising a linker, e.g., a C-terminal peptide unit, carrying (1) a drug covalently attached thereto via a specific spacer group and (2) a solubilizing group. The linker, e.g., C-terminal peptide unit, acts as highly specific substrate for the fast cleavage by Cat B and/or cleavage by exopeptidase (i.e., carboxydipeptidase) activity of Cat B, resulting in improved (high rate) cleavage and release of the drug into the target cell. The linker system is not prone to premature cleavage and remains stable in systemic circulation (blood). Thus, the linker system of the present invention can be used in ligand-drug-conjugates (LDCs).

Cat B is a lysosomal cysteine protease of the papain superfamily acting in intracellular protein turnover as well as in a variety of physiological and pathological processes. Extended structural and functional data are presently available, making this protease a versatile tool in the context of intracellular drug delivery.

The papain fold is composed of two domains, referred to the left (L-) and right (R—) domain. The L-domain contains three α-helices, while the R-domain forms a kind of β-barrel as described by Turk et al. (Biochim. Biophys. Acta 2012, 1824(1), 68-88). The two domain interface opens on the top, forming the active-site cleft of the enzyme. In the center of the active-site cleft are the residues Cys25 (at the N-terminus of the central helix, L-domain) and His163 (within the β-barrel residues, R-domain). These two catalytic residues form the thiolate-imidazolium ion pair, essential for the proteolytic activity of the enzyme. The substrate binds along the active-site cleft in an extended conformation as described by Turk et al. (Biochem. Soc. Symp. 2003, 70, 15-30), making alternating contacts with L- and R-domains. Most cysteine cathepsins exhibit predominantly endopeptidase activity (F, L, K, S, V), whereas Cat X and C exhibit only exopeptidase activity. In contrast, Cat B exhibits both endopeptidase and exopeptidase activity. X-ray analysis reveals that exopeptidases/carboxydipeptidases such as Cat B contain additional structural features, i.e. an additional (“occluding”) loop, which modify the active site cleft and serve as rationale for substrate binding in both endopeptidase and exopeptidase activity. In particular, the occluding loop provides the structural base for the dominant exo-versus endo-Cat B activity as shown by Renko et al. (FEBS Journal 2010, 277, 4338-4345).

The Cat B-cleavable linker systems described in the prior art (e.g. the Val-Cit-PABC linker system) are mainly based on the endopeptidase activity of Cat B. On the other hand, the linker system of the present invention is specifically designed to meet the structural requirements for acting as specific substrate for fast cleavage by Cat B and/or the exopeptidase activity of Cat B. Therefore, the linker system can be used in LDCs as highly specific substrate for fast cleavage by Cat B and/or for the exopeptidase activity of Cat B, i.e., in the compound of formula (I) described below, resulting in improved cleavage profiles (e.g., fast intracellular drug release). The linker system also enables the intracellular release of multiple drug molecules, wherein individual drug molecules may be the same or different. If the drug is a cytotoxic agent, the linker system enables the intracellular release of multiple drug molecules, which may be multiple molecules of the same drug or multiple molecules of different drugs (e.g. 2 or more different drugs).

Furthermore, it was surprisingly found that any effect on Cat B binding due to the presence of a sterically demanding solubilizing moiety was sufficiently compensated for by fast cleavage by Cat B, and thus that sterically demanding solubilizing groups may be incorporated into the ADC, to improve ADC PK and manufacturability, without affecting cytotoxicity. Cleavage by Cat B and especially by the exopeptidase activity of Cat B is very fast for many substrates and this high cleavage rate can compensate some slowing down of the cleavage rate resulting from incorporation of the solubilizing moiety, such that the resulting cleavage rate may still be reasonably fast. Without wishing to be bound to any theory, it is believed that the solubilizing moiety is directed towards the outside of the Cat B binding groove, thus leading to superior selectivity and cleavage rate by Cat B, e.g., via the exopeptidase activity of Cat B, while allowing to achieve high DAR.

Even further, it was found that the attachment of the drug to the linker via a specific spacer group and the presence of a solubilizing moiety give rise to a surprising improvement of efficacy and allow to overcome the problems of resistance which may occur with other conjugates. Without wishing to be bound to any theory, it is believed that the spacer group and the solubilizing moiety cooperate in such a manner that, after cleavage by Cat B, the drug can not only engage its molecular target(s) in the cytoplasm but can also escape lysosomal trapping and diffuse to proximal cells to induce bystander effect, e.g., cytotoxic bystander effect.

### 3. Compound of Formula (I)

The present invention relates to a compound, i.e., a ligand-drug-conjugate (LDC), represented by the general formula (I):

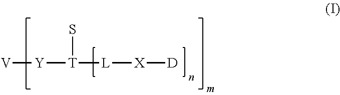

The compound of formula (I) contains a linker (L), e.g., a C-terminal peptide unit, which serves as substrate for specific recognition and cleavage by Cathepsin B and especially fast cleavage and/or cleavage by the exopeptidase activity of Cat B. The linker is covalently attached to branching group (T), as well as to divalent group (X).

Furthermore, in formula (I), D represents a moiety derived from a drug, wherein the said drug is selected from a carboxyl-containing drug such as auristatin F (AF), a thiol-containing drug such as mertansine (DM1) or ravtansine (DM4), an amino-containing drug such as monomethyl auristatin F (MMAF), exatecan or 2554618-79-8, and a hydroxyl-containing drug such as Maaa-1181a. The drug is covalently attached to divalent group (X) via a functional group comprised therein, i.e., a carboxyl, thiol, amino or hydroxyl functional group. Preferably, the drug is selected from a thiol-containing drug, an amino-containing drug and a hydroxyl-containing drug. More preferably, the drug is a thiol-containing drug such as DM1 or DM4.

If more than one (D) is present in a conjugate molecule (n>1 and/or m>1), at least one and preferably each (D) is independently selected from a carboxyl-containing drug, a thiol-containing drug, an amino-containing drug and a hydroxyl-containing drug, preferably from a thiol-containing drug, an amino-containing drug and a hydroxyl-containing drug, the multiple moieties (D) being preferably identical to each other.

X represents a divalent group comprising one to seven, preferably two to six, more preferably two to five, e.g., 2, 3, 4 or 5, backbone atoms independently selected from C, N, O, and S; X being covalently attached to (D) via an atom selected from C, S, N and O derived from the carboxyl, thiol, amino, or hydroxyl functional group comprised in (D).

Y represents a divalent group comprising one or more atoms selected from C, N, O, P and S, preferably a divalent group derived from a compound selected from maleimides, triazoles, hydrazones, carbonyl-containing compounds and derivatives thereof, more preferably a divalent group derived from maleimides and derivatives thereof such as opened hydrolyzed maleimide derivatives, and most preferably a divalent group derived from an opened hydrolyzed maleimide.

T represents a (2+n)-valent branching group.

S represents a moiety derived from a compound comprising one or more, e.g., 2, 3, 4 or 5, solubilizing groups.

V represents a moiety derived from a vector group capable of interacting with a target cell.

n is an integer of 1 to 4, preferably 1 or 2, more preferably 1; and m is an integer of 1 to 12, preferably 2 to 10, more preferably 4 to 8.

In one embodiment, n is 1, and m is an integer of 1 to 12, preferably 2 to 10, more preferably 4 to 8, such as 5 to 8 or 6 to 8.

### 3.1 Cleavable Linker (L)

The compound of formula (I) contains a linker (L), e.g., a C-terminal peptide unit, which serves as substrate for specific recognition and cleavage by Cathepsin B and especially fast cleavage and/or cleavage by the exopeptidase activity of Cat B. The linker enables fast (high rate) release of the drug-containing moiety, e.g., moiety D-X or D-X-Dxx-Dyy, from the compound into the target cell.

In some embodiments of the present invention, the linker (L) is represented by the general formula (II) or (11′):

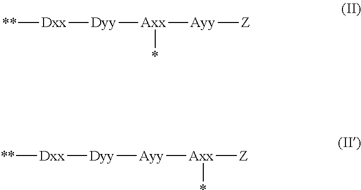

In formulae (II) and (II′), Axx represents a moiety derived from a trifunctional amino acid. Axx can be a moiety derived from any natural or non-natural trifunctional amino acid with the proviso that Axx in formula (II) is not a moiety derived from an amino acid in the (D) configuration. Examples of trifunctional amino acids include amino-dicarboxylic acids and diamino-carboxylic acids, such as α-amino adipic acid (Aaa), diamino propionic acid (Dap), diamino butyric acid (Dab), and amino malonic acid (Ama). Further suitable trifunctional amino acids include Glu, 2-amino pimelic acid (Apa), Lys, Orn, Ser and homo-lysine (homo-Lys).

According to one embodiment, Axx represents a moiety derived from an amino acid selected from Glu, Apa, Aaa, Dap, Dab, Lys, Orn, Ser, Ama, and homolysine (homoLys). According to one preferred embodiment, Axx represents a moiety derived from an amino acid selected from Dap, Dab, Lys, Orn and homoLys. More preferably, Axx represents a moiety derived from Orn or Lys, and most preferably a moiety derived from Lys.

Ayy represents a moiety derived from an amino acid selected from Phe, Ala, Trp, Tyr, Phenylglycine (Phg), Met, Val, His, Lys, Arg, Citrulline (Cit), 2-amino butyric acid (Abu), Orn, Ser, Thr, Leu and Ile; or Ayy in formula (II) represents a moiety derived from an amino acid selected from homo-tyrosine (homo-Tyr), homo-phenylalanine (homo-Phe), beta-phenylalanine (beta-Phe), beta-homo-phenylalanine (beta-homo-Phe), Tyr(OR₁) and homo-Tyr(OR₁) wherein R₁ is a solubilizing group, preferably —(CH₂CH₂O)ₙ₁—R₂, wherein R₂ is a hydrogen atom or a methyl group and n1 is an integer of 2 to 24, e.g. an integer of 5 to 20, or 8 to 16; with the proviso that Ayy in formula (II′) is not an amino acid in the (D) configuration. Without wishing to be bound by theory, it is believed that Ayy provides the compound of the present invention with the structural features for specific recognition and cleavage by the exopeptidase activity of Cat B. As a result, the compound can release the drug at a significantly higher rate as compared to a compound cleaved by the endopeptidase activity of Cat B, e.g., a compound comprising a Val-Cit-PABC linker system.

According to one embodiment, Ayy in formula (II) represents a moiety derived from an amino acid selected from Phe, homo-Phe, Ala, Trp, Leu, Tyr, Phg, Met, Abu, Val, Lys, Cit, Tyr(OR₁) and homo-Tyr(OR₁), preferably a moiety derived from an amino acid selected from Phe, homo-Phe, Ala, Trp, Leu, Val, Tyr, homo-Tyr, Tyr(OR₁) and homo-Tyr(OR₁), more preferably a moiety derived from Phe, homo-Phe, Tyr, homo-Tyr, Tyr(OR₁) or homo-Tyr(OR₁), wherein R₁ is as specified above, in particular a moiety derived from Phe or Tyr, most preferably a moiety derived from Tyr; and Ayy in formula (II′) represents a moiety derived from an amino acid selected from Phe, homo-Phe, Ala, Ser, Thr, Leu, Val, Tyr, Phg, Trp, Ile and Arg, preferably a moiety derived from an amino acid selected from Phe, homo-Phe, Ala, Trp, Phg, Leu, Val, Tyr and Ser, more preferably a moiety derived from Phe, home-Phe or Ser, in particular a moiety derived from Phe or Ser, most preferably a moiety derived from Phe.

In connection with formulae (II) and (II′) depicted above, Dxx represents a single covalent bond or a moiety derived from an amino acid having a hydrophobic side chain, preferably a single covalent bond or a moiety derived from an amino acid selected from Phe, Val, Tyr, homo-Phe and Ala. More preferably, Dxx represents a single covalent bond or a moiety derived from Phe or Val.

Dyy represents a single covalent bond, a moiety derived from Phe or a moiety derived from an amino acid having a basic side chain, preferably a moiety derived from an amino acid selected from Arg, Lys, Cit, Orn, Dap, and Dab; with the proviso that if Dxx is a moiety derived from an amino acid having a hydrophobic side chain, Dyy is a moiety derived from Phe or a moiety derived from an amino acid having a basic side chain, and if Dxx is a single covalent bond, Dyy is a single covalent bond, a moiety derived from Phe or a moiety derived from an amino acid having a basic side chain. More preferably, Dyy represents a moiety derived from Arg or Cit.

Z in formulae (II) and (II′) represents a group covalently bonded to the C-terminus of Ayy or Axx selected from —OH and —N(H)(R) wherein R represents a hydrogen atom, an alkyl group or a cycloalkyl group, preferably —OH.

In formulae (II) and (II′), * indicates covalent attachment to (T) and ** indicates covalent attachment to (X).

### 3.2 Divalent Group (X)

The compound of formula (I) contains a divalent group (X) comprising one to seven, preferably two to six, more preferably two to five, e.g., 2, 3, 4 or 5, backbone atoms independently selected from C, N, O, and S; X being covalently attached to (D) via an atom selected from C, S, N and O derived from the carboxyl, thiol, amino, or hydroxyl functional group comprised in (D).

The divalent group (X) remains covalently attached to (D) after cleavage of the linker (L) by Cat B such that that a modified drug (intra drug), e.g., moiety D-X, is released into the target cell. In the present invention, it was surprisingly found that such a modified drug exhibits improved efficacy, e.g., cytotoxic efficacy, as compared with the same drug not carrying a group (X) attached thereto. Without wishing to be bound to any theory, it is believed that the spacer group (X) has a size which is appropriate, e.g., one-seven backbone atoms, for not detrimentally affecting the pharmacokinetic properties of the conjugate even at high DAR (DAR>4), while contributing to the membrane permeation properties of the released drug moiety, e.g., due to an appropriate level of hydrophobicity. As a result, upon Cat B-induced high rate cleavage, the drug can not only engage its molecular target(s) in the cytoplasm but also diffuse to proximal cells to induce bystander effect, e.g., cytotoxic bystander effect.

In one embodiment, (X) represents a divalent carbonyl- or thiocarbonyl-containing group, preferably a group represented by one of the following formulae (IIIa) to (IIIf):

***—(CH₂)ₙ₂—(C=A)-**′ (IIIa)

***—(C=A)-(CH₂)ₙ₃—**′ (IIIb)

***—(CH₂)ₙ₂—(C=A)-(CH₂)ₙ₃—**′ (IIIc)

***—(CH₂)ₙ₂—(C=A)-(C(CH₃)₂)—(CH₂)ₙ₃—**′ (IIId)

***—(CH₂)ₙ₂—(C(CH₃)₂)—(C=A)-(CH₂)ₙ₃—** (IIIe)

***—(C=A)-(NH)ₙ₄—(CH₂)ₙ₃—(NH)ₙ₅—(C=A)-**′ (IIIf)
wherein
n2, n3 are each independently selected from 0 to 5, preferably 0, 1 or 2, more preferably 0 or 1;
n4, n5 are each selected from 0 or 1, wherein, in one embodiment n4 is 0 and n5 is 1, in another specific embodiment n4 is 1 and n5 is 0, in yet another specific embodiment both n4 and n5 are 1 while in yet another specific embodiment both n4 and n5 are 0;
each A is independently selected from O and S, preferably O;
*** represents covalent attachment to (D); and
**′ represents covalent attachment to (L).

In one embodiment, (X) is represented by one of the following formulae (IVa) to (IVj′):

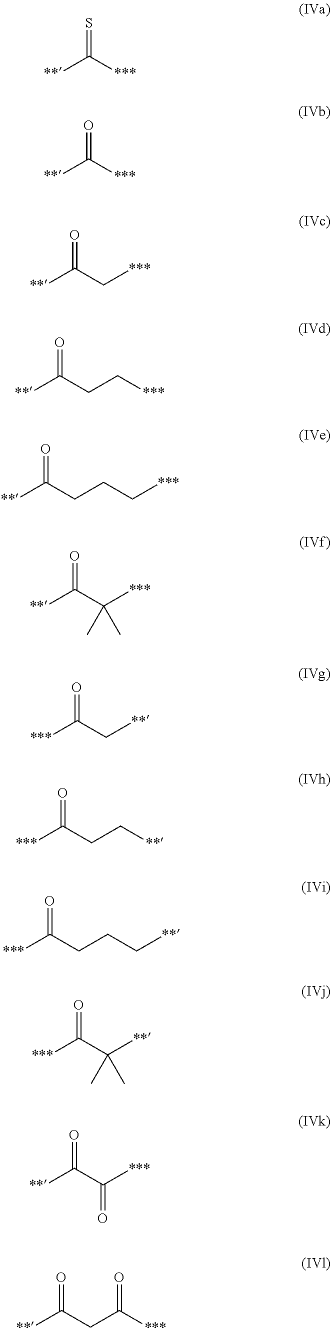

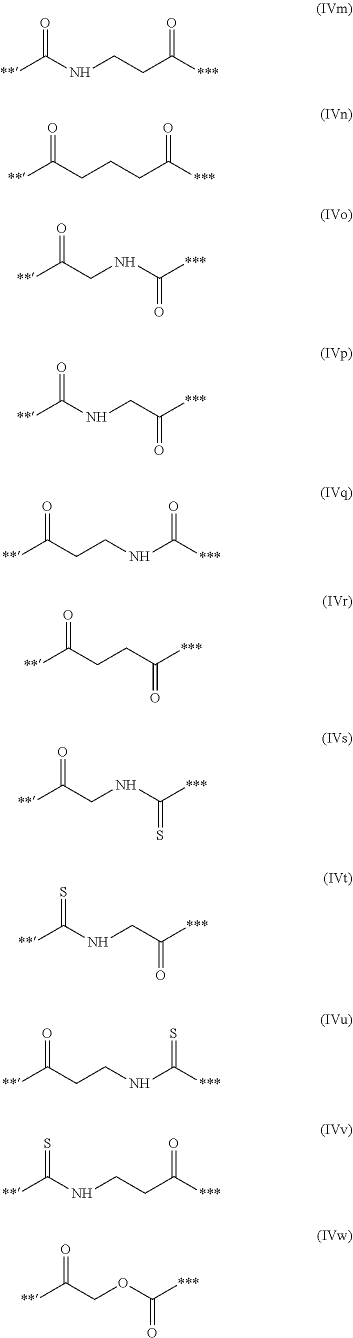

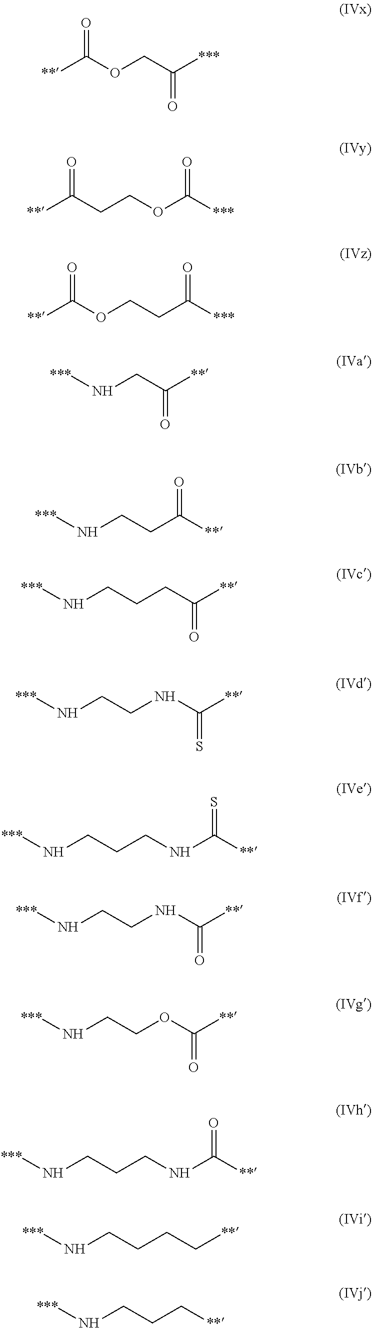

wherein,
** represents covalent attachment to (D);
**′ represents covalent attachment to (L);
with the proviso that if (X) is represented by formula (IVg), (IVh), (i), (IVj), (IVk), (IV/), (IVm), (IVn), (IVp), (IVr), (IVt), (IVv), (IVw), (IVx), (IVy) or (IVz), (D) in formula (I) represents an amino-containing drug; if (X) is represented by formula (IVj), (IVq), (IVs) or (IVu), (D) in formula (I) represents an amino-containing drug or a hydroxyl-containing drug; and if (X) is represented by formula (IVa′), (IVb′), (IVc′), (IVd′), (IVe′), (IVf′), (IVg′), (IVh′), (IVi′) or (IVj′), (D) in formula (I) represents a carboxyl-containing drug.

In one preferred embodiment, (X) is represented by formula (IVc), (IVm), (IVn), (IVo), (IVp), (IVs) or (IVt). More preferably, (X) is represented by formula (IVc) or (IVm).

It is advantageous to select a structure for X which can conveniently bond to the functional group of (D). According to one embodiment, if (D) has a carboxyl group, (X) preferably has an amino group to allow bonding to (D) via the formation of an amide bond (linkage); if (D) has an amino group, (X) preferably has a carbonyl (or thiocarbonyl group) to allow bonding to (D) via the formation of an amide bond; if (D) has a thiol group, (X) preferably has a methylene group to allow bonding to (D) via the formation of a thioether bond; and if (D) has a hydroxyl group, (X) preferably has a methylene group or a carbonyl (or thiocarbonyl group) to allow bonding to (D) respectively via the formation of an ether or an ester (or thioester) bond. Further matches of structural groups and resulting linkages are also conceivable. The following table provides a summary of available options.

| Chemical functionality | Chemical functionality | Chemical functionality |
|---|---|---|
| on starting material associated to X | on starting material associated to D | on D-X linkage |
| R¹—X′ | R²—SH | R¹—S—R² |
| X′ = Cl, Br, I, OTs, OMs | | |
| R′—X | R²—OH | R¹—O—R² |
| X′ = Cl, Br, I, OTs or OMs | | |
| R¹—NCO | R²—OH | 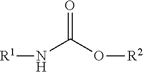 |
| R¹—CO₂H | R²—OH | 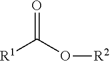 |
| R¹—CO₂H | R²—NH₂ | 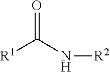 |
| R¹—X′ X′ = Cl, Br, I, OTs or OMs | R²—NH₂ | 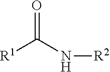 |
| 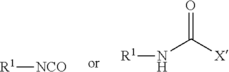 | R²—NH₂ | 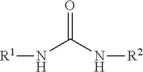 |
| X′ = Cl, OSu, OPFP, OPNP | R²—NH₂ | 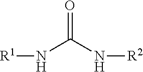 |
| R¹—NCS | R²—NH₂ | 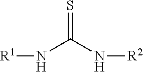 |
| R¹—NH₂ | R²—CO₂H | 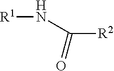 |
| R¹—OH | R²—CO₂H | 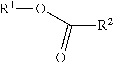 |

In the above table, R¹ characterizes the remainder of the group (X) whereas R² characterizes the remainder of the drug (D).

### 3.3 Solubilizing Moiety (S)

The compound of formula (I) contains a solubilizing moiety (S), which is a moiety derived from a compound comprising one or more, e.g., 2, 3, 4 or 5, solubilizing groups.

The presence of a solubilizing moiety enables to reduce (or prevent) the tendency of the conjugate molecules for aggregation and thus to achieve excellent pharmacokinetic properties, e.g., biodistribution, hepatic clearance, even at high DAR (DAR>4, e.g., DAR=8). In some instances, aggregation of the conjugate molecules can be completely suppressed, even at high DAR. The present inventors have surprisingly found that cleavage by Cat B is possible even in the presence of a sterically demanding solubilizing moiety. Preferably fast cleavage of the linker by Cat B can be accomplished, preferably by the exopeptidase mechanism of Cat B. Without being bond to any theory, it is believed that the solubilizing moiety is directed towards the outside of the Cat B binding groove, thus allowing for superior selectivity and cleavage rate, e.g., via the exopeptidase mechanism. In addition, it was also surprisingly found that the solubilizing moiety is capable to compensate for the potential hydrophobicity of the drug moiety D-X, such that excellent pharmacokinetic properties can be retained even if multiple drug moieties are attached to the linker (e.g., n>1).

In one embodiment, S represents a moiety derived from a compound comprising one or more, e.g., two, three or four, solubilizing groups; wherein each solubilizing group comprised in (S) is independently selected from the group consisting of:
moieties comprising one or more ionic or ionizable groups, such as ammonium, guanidinium, sulfate or sulfonate groups, preferably of moieties derived from Arg, (D)-Arg, Dap, (D)-Dap, Dab, (D)-Dab, Orn, (D)-Orn, Lys, D-Lys or carnitine;
saccharide moieties selected from monosaccharides, disaccharides and linear or branched oligosaccharides, in particular linear or branched oligosaccharides having 3 to 10 monosaccharide units being linked by glycosidic bonds, wherein each of the monosaccharide units in the monosaccharide, disaccharide and oligosaccharide is independently selected from glucose, fructose, mannose, ribose, and galactose; and
polyalkylene oxide groups, preferably C₂₋₃ polyalkylene oxide groups, more preferably C₂₋₃ polyalkylene oxide groups independently comprising from 6 to 200, preferably from 10 to 150, more preferably from 12 to 80 repeating units.

There is no particular limitation as to the general arrangement of the solubilizing groups in the solubilizing moiety. Hence, the solubilizing moiety can have a linear structure, e.g., in which several solubilizing groups are arranged in a random or block-wise manner, a cyclic structure, or a branched structure, e.g., in which several solubilizing groups are attached to a core molecule, such as pentaerythritol or glycerol, in a graft or dendrimeric manner. The solubilizing moiety can also comprise several blocks, each block independently having a linear or branched structure.

In one aspect, the solubilizing moiety comprises one or more solubilizing groups arranged in a linear, block-wise manner. For example, the solubilizing moiety can comprise a structure represented by -(So)_{n′}- as illustrated in more detail by the following formula: -(So¹)-(So²)-[ . . . ](Soⁿ), wherein each So¹ to Soⁿ represents a solubilizing group, such as a polyalkylene oxide group, e.g., a PEO group having from 6 to 200 repeating units, or a moiety comprising one or more ionic or ionizable groups, such as Arg, and n′ is an integer of 1 to 20, e.g., 1 to 10, with the proviso that directly connected polyalkylene oxide groups of the same structure are to be regarded as multiple repeating units of the same solubilizing group (and not as adjacent So groups).

That is, adjacent polyalkylene oxide groups must be of different structure and/or be connected via a functional group like —C(O)—O— or the like in order to be treated as separate So groups.

In another aspect, the solubilizing moiety comprises one or more solubilizing groups attached to a core molecule, such as pentaerythritol or glycerol, in an untethered, graft or dendrimeric manner. For example, the solubilizing moiety can have a graft structure represented by —((—Y′—X′(So_{m′}))_{n′}-H as illustrated in more detail below:

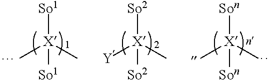

wherein X′ is a (m′+2)-valent, e.g., tri- or tetravalent, group, Y′ is a divalent group, each So is independently selected to be a solubilizing group, such as a polyalkylene oxide group, e.g., a PEO group having from 4 to 600 repeating units, or a moiety comprising one or more ionic groups, m′ is 1, 2, 3, or more and preferably 1 or 2, and n′ is an integer of 1 to 20, e.g., 1 to 10;
   or a tree-like, dendrimeric structure represented by —X′(So)_{n′} as illustrated in more detail below:

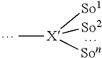

wherein X′ is a n-valent (branching) group, each So¹ to Soⁿ is independently selected to be a solubilizing group as described above, such as a polyalkylene oxide group, e.g., a PEO group having from 4 to 600 repeating units, or a moiety comprising one or more ionic groups, and n′ is an integer of 1 to 20, e.g., 1 to 10.

In one embodiment, (S) is a moiety derived from a compound comprising one or more polyethylene oxide groups, wherein preferably each polyethylene oxide group independently comprises from 6 to 200, more preferably from 10 to 150, most preferably from 12 to 80 repeating units.

In one preferred embodiment, (S) is a moiety represented by the formula (V):

****—X¹—(CH₂CH₂O)ₙ₃—X² (V)
wherein,
n3 is an integer of 6 to 200, preferably 10 to 150, more preferably 12 to 80;
**** indicates covalent attachment to (T);
X¹ is selected from a single covalent bond, —(C═O)—, and —N(R)— in which R represents a hydrogen atom, an alkyl group or a cycloalkyl group;
X² represents an alkyl group having 1 to 6 carbon atoms, a carbonyl-containing group such an acetyl group or a group of formula —(CH₂)ₙ₄—CO₂H, a thiocarbonyl-containing group, a group of formula —(CH₂)ₙ₄OR, a group of formula —(CH₂)ₙ₄—SO₃H, or an amino-containing group such as a group of formula —(CH₂)ₙ₄—(C=A)-N(R)₂ or —(CH₂)ₙ₄—N(R)₂, in which A is O or S, each R is independently selected from a hydrogen atom, an alkyl group and a cycloalkyl group, and n4 is an integer of 1 to 6;
X² being preferably —CH₃, —CH₂CH₂OH, or a group represented by the following formula (VI):

—(CH₂)ₙ₅—(C=A)N(R)—(CH₂)ₙ₆—(C=A)N(H)(R) (VI)
wherein,
each A is independently selected from O and S, preferably O;
each R is independently selected from a hydrogen atom, an alkyl group and a cycloalkyl group; and
n5 and n6 are each independently an integer of 1 to 6, preferably 1 or 2; and
X² being most preferably —CH₃.

If more than one (S) is present, each (S) is preferably a moiety of formula (V) as described above.

### 3.4 Branching Group (T)

T represents a (2+n)-valent, e.g., 3-, 4-, 5-, 6-valent, branching group. The branching group connects the vector group (V), the solubilizing moiety (S) and one or more (n) linker moieties (L) thereby forming a branched structure. Preferably, T is a 3-valent (n=1), or 4-valent (n=2) branching group. More preferably, T is a 3-valent branching group.

In one embodiment, the branching group is a group comprising at least one moiety derived from a trifunctional amino acid, e.g., from Lys. The branching group can comprise further (optional) linkers and/or amino acids in addition to the trifunctional amino acid mentioned above, provided that the said further linkers do not contain a solubilizing group, such as a polyalkylene oxide group, and/or that the said further amino acids are not trifunctional amino acids or moieties comprising one or more ionic or ionizable groups. Such further amino acids can, for example, be selected from homo-Phe (hPHe) and Phe. In some aspects, the branching group consists of a moiety derived from a trifunctional amino acid (i.e., does not include any further linkers and/or amino acids).

In one embodiment, (T) is a moiety represented by the following formula (VII):

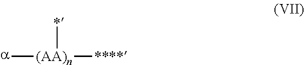

wherein,
each AA independently represents a moiety derived from a trifunctional amino acid such as a diamino-carboxylic acid, an amino dicarboxylic acid, an azido amino acid or an alkyne-containing amino acid, preferably derived from an amino acid selected from N-ε-propargyloxycarbonyl-L-Lysine (Lys(Poc)), Asp, Glu, Orn, Lys, Dab and Dap, more preferably derived from Lys(Poc), Glu, Orn or Lys, most preferably derived from Lys;
α indicates covalent attachment to (Y);
if n=1, the side chain originating from the trifunctional amino acid is covalently attached to (L) or (S), the C-terminus is covalently attached to the other moiety (S) or (L), respectively;
if n=2, 3 or 4:
indicates covalent attachment to (L);
****′ indicates covalent attachment to (S); and
n is as defined in formula (I).

In one further embodiment, (T) is a moiety represented by the formula (VIII) or (IX):

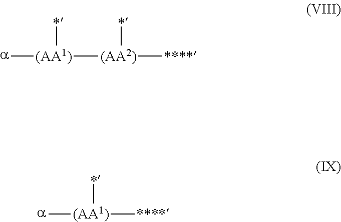

wherein,
each AA¹ and AA² is independently a moiety derived from a trifunctional amino acid, such as a diamino-carboxylic acid, an amino dicarboxylic acid, an azido amino acid or an alkyne-containing amino acid, preferably a moiety derived from an amino acid selected from Lys(Poc), Asp, Glu, Orn, Lys, Dab and Dap, more preferably a moiety derived from Lys(Poc), Glu, Orn or Lys, most preferably a moiety derived from Lys;
α indicates covalent attachment to (Y);
in formula (IX), the side chain originating from the trifunctional amino acid is covalently attached to (L) or (S), the C-terminus is covalently attached to the other moiety (S) or (L), respectively,
in formula (VIII), *′ indicates covalent attachment to (L), and ****′ indicates covalent attachment to (S).

### 3.5 Divalent Group (Y)

The compound of formula (I) contains a divalent group (Y) comprising one or more atoms selected from C, N, O, P and S. The divalent group connects the vector group (V) to the branching group (T). The divalent group is typically attached to the side chain of an amino acid contained in the vector, such as Cys. Preferably, Y is a divalent group derived from a compound selected from maleimides, triazoles, hydrazones, carbonyl-containing compounds, and derivatives thereof. More preferably, Y is a divalent group derived from maleimides and derivatives thereof, such as opened hydrolyzed maleimide derivatives. Most preferably, Y is a divalent group derived from an opened hydrolyzed maleimide.

Hydrolysis of a maleimide attachment to V is typically performed under basic conditions as a final step of the conjugation of the maleimide derivative to V, as in general procedure 1 to 3 set out in the examples herein. The following conditions are especially suitable: at the end of a cysteine maleimide conjugation reaction, pH is adjusted to pH 8 by adding 10×pH 8 DPPS (0.2 to 0.5 reaction volume) and excess reactive drug linker and reducing agent (TCEP) are removed via gel filtration using suitable columns for gel filtration (PF column, elution with pH 8 buffer). The eluent is then stirred overnight for 16 h to complete the opening before final buffer exchange with DPBS into an Amicon concentrating unit.

In some instances, when m in the compound of formula (I) is at least 2, said compound may comprise a mix of (closed) maleimide derivatives (Y) and opened hydrolyzed maleimide derivatives (Y) attached to V. Accordingly, in the compounds described herein in which a group (R) is attached to a vector (V) via a maleimide (as shown below, left hand side), hydrolysis may be carried out such that, when m is at least 2, a compound of the invention may comprise both closed maleimide attachments (A) and opened hydrolyzed maleimide attachments (B) (as shown below, left hand side) to V.

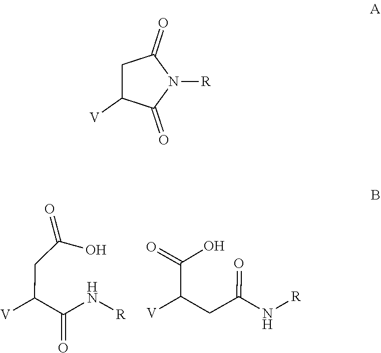

In preferred embodiments, in the compound of formula (I) wherein m is at least 2, at least 50% of the Y attachments to V are opened hydrolyzed maleimide attachments (B), the remaining attachments being closed maleimide attachments (A). In some instances, at least 70%, at least 75%, at least 80%, at least 90%, at least 95%, preferably at least 98% of the Y attachments to V are opened hydrolyzed maleimide attachments (B).

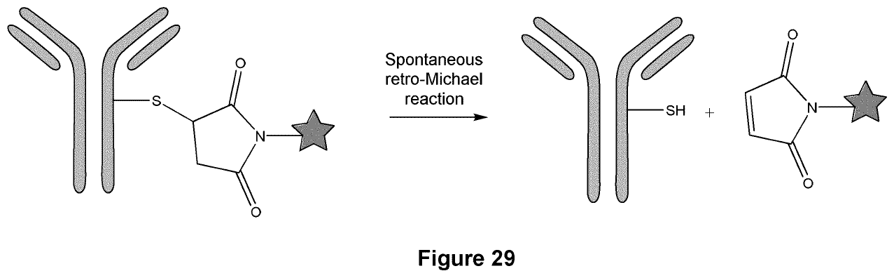

The presence of one or more opened hydrolyzed maleimide attachments (even if present together with one or more closed maleimide attachments) can contribute to the stability and therapeutic efficacy of the compounds of the invention. Without being bound to any theory, it is believed that the opened hydrolyzed maleimide attachment e.g., prevents a retro-Michael reaction (as shown in FIG. 29) that causes the liberation of reactive maleimide in the circulation and ultimately leads to transfer of linker-payload to other thiol-containing molecule in the body, such as albumin. The opened maleimide may also cooperate with the divalent group X and the solubilizing moiety S to achieve improved stability and therapeutic efficacy.

In a preferred embodiment, Y is a divalent group derived from maleimides and derivatives thereof, such as opened hydrolyzed maleimides, preferably a divalent group represented by any of the following formulae (XIIIa) to (XIIIc):

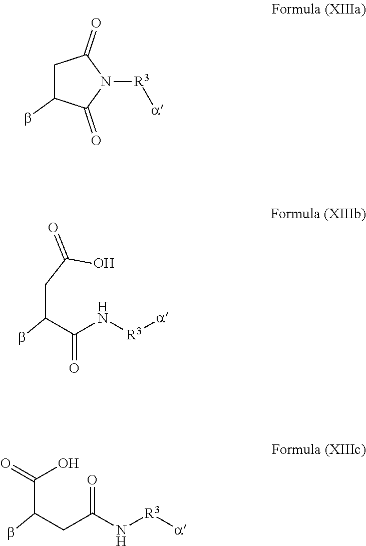

wherein,
R³ represents —(CH₂)ₙ₇—(C=A)ₙ₉- or —(CH₂CH₂O)ₙ₈—(C=A)ₙ₉-, preferably —(CH₂)ₙ₇—(C=A)ₙ₉-, wherein,
   n7 is 1 to 6, preferably 1 or 2, more preferably 1,
   n8 is 1 to 6, preferably 1,
   n9 is 0 or 1, preferably 1, and
   A is O or S, preferably O;
wherein the methylene carbon atom is covalently attached to the nitrogen atom of formulae (XIIIa)-(XIIIc) and the carbonyl or thiocarbonyl-carbon is covalently attached to T;
β indicates covalent attachment to V; and
α′ indicates covalent attachment to T.

In a more preferred embodiment, Y is represented by formula (XIIIb) or (XIIIc), wherein R³ is preferably a group represented by the formula —(CH₂)ₙ₇—(C=A)ₙ₉- in which n7 is 1 or 2, n9 is 1 and A is O. Most preferably, R³ is —CH₂—C═O—.

### 3.6 Moiety (D)

The compound of formula (I) contains a moiety derived from a drug, wherein the drug is selected from a carboxyl-containing drug, a thiol-containing drug, an amino-containing drug, and a hydroxyl-containing drug. Preferably, the drug is selected from a thiol-containing drug, an amino-containing drug and a hydroxyl-containing drug. More preferably, (D) is moiety derived from a thiol-containing drug, such as DM1 or DM4.

If more than one (D) is present in the compound of formula (I) (n>1 and/or m>1), each (D) is independently selected from a carboxyl-containing drug, a thiol-containing drug, an amino-containing drug and a hydroxyl-containing drug. Nonetheless, it is preferred that the multiple moieties (D) are identical to each other.

The drug can be unmodified (in its natural form except for the replacement of a hydrogen atom by a covalent bond), or can be chemically modified in order to incorporate one or more functional groups (e.g. one or more groups selected from hydroxyl, carboxyl, amino and thiol groups) allowing covalent attachment(s) to the divalent group (X), the drug moiety, e.g., moiety D-X or moiety D-X-Dxx-Dyy, being preferably pharmacologically active once it is released from the conjugate. According to one embodiment, the drug moiety that is released from the conjugate, e.g., moiety D-X or moiety D-X-Dxx-Dyy, is pharmacologically active in such a sense that it retains at least 20%, preferably at least 35%, more preferably at least 50%, and even more preferably at least 70% of the pharmacological activity of the corresponding unmodified (native) drug.

In some aspects of the present invention, each moiety derived from a drug independently represents a prodrug-group which is not pharmacologically active in the conjugated form, e.g., when found in the compound of formula (I), but which becomes pharmacologically active either once released from the conjugate.

According to one embodiment, each moiety derived from a drug is independently selected from:
(i) an antineoplastic agent such as
   a DNA-alkylating agent, such as duocarmycin,
   a topoisomerase inhibitor, such as doxorubicin,
   an RNA-polymerase II inhibitor, such as alpha-amanitin,
   a DNA cleaving agent, such as calicheamicin,
   an antimitotic agent or microtubule disruptor, such as a taxane, an
   auristatin or a maytansinoid,
   an anti-metabolite, such as derivatives of gemcitabine,
   a Kinesin spindle protein inhibitor, such as Filanesib,
   a kinase inhibitor, such as ipatasertib or gefitinib,
   nicotinamide phosphoribosyltransferase inhibitor,
   a matrix metallopeptidase 9 inhibitor,
   a phosphatase inhibitor such as mycrocystin-LR,
(ii) an immunomodulatory agent, such as fluticasone
(iii) an anti-infectious disease agent, such as rifamycin, clindamycin or reptamulin, and
(iv) radioisotopes, metabolites, pharmaceutically acceptable salts, and/or prodrugs of any of the foregoing;
with the proviso that the drug selected from (i) to (iv) above is a carboxyl-containing drug, a thiol-containing drug, an amino-containing drug, or a hydroxyl-containing drug.

If more than one (D) is present in the compound of formula (I), each (D) can be independently selected from (i) to (iv) above, with the proviso that each drug selected from (i) to (iv) is a carboxyl-containing drug, a thiol-containing drug, an amino-containing drug or a hydroxyl-containing drug.

Below are exemplary drugs that may be used in a ligand-drug-conjugate of the present invention, with the proviso that the drug is a carboxyl-containing drug, a thiol-containing drug, an amino-containing drug, or a hydroxyl-containing drug.
(i) Antineoplastic agents include:
   (a) Alkylating agents such as nitrogen mustard analogues (e.g. cyclophosphamide chlorambucil, melphalan, chlormethine, ifosfamide, trofosfamide, prednimustine, bendamustine, chlornaphazine, estramustine, mechlorethamine, mechlorethamine oxide hydrochloride, mannomustine, mitolactol, novembichin, phenesterine, uracil mustard); alkyl sulphonates (e.g. busulfan, treosulfan, mannosulfan, improsulfan and piposulfan); ethylene imines (e.g. thiotepa, triaziquone, carboquone); nitrosoureas (e.g. carmustine, lomustine, semustine, streptozocin, chlorozotocin, fotemustine, nimustine, ranimustine); epoxides (e.g. etoglucid); other alkylating agents (e.g. mitobronitol, pipobroman, temozolomide, dacarbazine);
   (b) Alkaloids such as vinca alkaloids (e.g. vincristine, vinblastine, vindesine, vinorelbine, navelbin, vinflunide, vintafolide); taxanes (e.g. paclitaxel, docetaxel, paclitaxel polyglumex, cabazitaxel) and their analogs, maytansinoids (e.g. DM1, DM2, DM3, DM4, maytansine and ansamitocins) and their analogs, cryptophycins (e.g. cryptophycin 1 and cryptophycin 8); epothilones, eleutherobin, discodermolide, bryostatins, dolostatins, auristatins (e.g. monomethyl auristatin E, monomethyl auristatin F), tubulysins, cephalostatins; pancratistatin; sarcodictyin; spongistatin; demecolcine; epipodophyllins (e.g. 9-aminocamptothecin, camptothecin, crisnatol, daunomycin, etoposide, etoposide phosphate, irinotecan and metabolites thereof such as SN-38, mitoxantrone, novantrone, retinoic acids (retinols), teniposide, topotecan, 9-nitrocamptothecin (RFS 2000)); mitomycins (e.g. mitomycin C);
   (c) Anti-metabolites such as DHFR inhibitors (e.g. methotrexate, trimetrexate, denopterin, pteropterin, aminopterin (4-aminopteroic acid) or other folic acid analogues such as raltitrexed, pemetrexed, pralatrexate); IMP dehydrogenase inhibitors (e.g. mycophenolic acid, tiazofurin, ribavirin, EICAR); ribonucleotide reductase inhibitors (e.g. hydroxyurea, deferoxamine); pyrimidine analogs (e.g. cytarabine, fluorouracil, 5-fluorouracil and metabolites thereof, tegafur, carmofur, gemcitabine, capecitabine, azacitidine, decitabine, fluorouracil combinations, tegafur combinations, trifluridine combinations, cytosine arabinoside, ancitabine, floxuridine, doxifluridine), uracil analogs (e.g. 6-azauridine, deoxyuridine); cytosine analogs (e.g. enocitabine); purine analogs (e.g. azathioprine, fludarabine, mercaptopurine, thiamiprine, thioguanine, cladribine, clofarabine, nelarabine); folic acid replenisher such as folinic acid;
   (d) Endocrine therapies used specifically in the treatment of neoplastic diseases, such as estrogens, progestogens, gonadotropin releasing hormone analogues, anti-estrogens, anti-androgens, aromatase inhibitors;
   (e) Kinase inhibitors such as BIBW 2992 (anti-EGFR/Erb2), imatinib, gefitinib, pegaptanib, sorafenib, dasatinib, sunitinib, erlotinib, nilotinib, lapatinib, axitinib, pazopanib, vandetanib, afatinib, vemurafenib, crizotinib, regorafenib, masitinib, dabrafenib, trametinib, ibrutinib, ceritinib, lenvatinib, nintedanib, cediranib, palbocidib, osimertinib, alectinib, alectinib, rociletinib, cobimetinib, midostaurin, olmutinib, E7080 (anti-VEGFR2), mubritinib, ponatinib (AP24534), bafetinib (INNO-406), bosutinib (SKI-606), cabozantinib, vismodegib, iniparib, ruxolitinib, CYT387, tivozanib, ispinesib, temsirolimus, everolimus, ridaforolimus;
   (f) Others such as duocarmycin (including synthetic analogues: adozelesin, carzelesin, bizelesin, KW-2189 and CBI-TMI); benzodiazepine dimers (dimers of pyrrolobenzodiazepine or tomaymycin, indolinobenzodiazepines, imidazobenzothiadiazepines, or oxazolidinobenzodiazepines); platinum containing compounds (e.g. carboplatin, cisplatin, oxaliplatin, satraplatin, polyplattilen); aziridines such as benzodopa, meturedopa, and uredopa; methylamelamines including altretamine, triethylenemelamine, trietylenephosphoramide, triethylenethiophosphaoramide and trimethylolomelamine; dynemicin, esperamicin, kedarcidin, maduropeptin, aclacinomysins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; polyketides (e.g. acetogenins); gemcitabine, epoxomicins (e.g. carfilzomib).
(ii) Immunomodulatory agents include immunostimulants, immunosuppressants, cyclosporine, cyclosporine A, aminocaproic acid, azathioprine, bromocriptine, chlorambucil, chloroquine, cyclophosphamide, corticosteroids (e.g. amcinonide, betamethasone, budesonide, hydrocortisone, flunisolide, fluticasone propionate, fluocortolone danazol, dexamethasone, prednisone, triamcinolone acetonide, beclometasone dipropionate), DHEA, hydroxychloroquine, meloxicam, methotrexate, mofetil, mycophenylate, sirolimus, tacrolimus, everolimus, fingolimod, ibrutinib.
(iii) Anti-infectious disease agents include antibacterial drugs, antimitotic drugs, antimycobacterial drugs and antiviral drugs. A non-limiting example of antibiotic used in an antibiotic-antibody drug conjugate is rifalogue, a rafamycin derivative.

The drugs used herein also include radioisotopes thereof. Examples of radioisotopes (radionuclides) are for instance ³H, ^{U}C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹⁷⁷Lu, ¹⁸⁶Re, ¹⁸⁸Re, ²¹¹At, ²¹²Bi, ²¹³Bi or ²²⁵Ac. Radioisotope labeled drugs can be used in targeted imaging experiments, or in targeted treatments (Wu et al Nat. Biotech. 2005, 23, 1137-1146).

The drugs used herein also include pharmaceutically acceptable salts, acids or derivatives thereof.

According to a preferred embodiment, each moiety derived from a drug is independently derived from a drug selected from amanitin, duocarmycin, auristatin, auristatin F (AF), monomethyl auristatin F (MMAF), maytansine, mertansine (DM1), ravtansine (DM4), tubulysin, calicheamicin, camptothecin, SN-38, exatecan, Maaa-1181a, taxol, daunomycin, vinblastine, doxorubicin, methotrexate, pyrrolobenzodiazepine (PBD) and dimers thereof, indilinobenzodiazepine (IBD) and dimers thereof, or radioisotopes and/or pharmaceutically acceptable salts thereof. It is more preferred that each moiety derived from a drug is independently derived from a drug selected from auristatin, MMAF, exatecan, maytansine, DM1 and DM4; and even more preferred that it is derived from a drug selected from DM1 and DM4.

### 3.7 Compound of Formula (I) Including a Linker of Formula (II)/(II′)

The compound of formula (I) may, in some instances, include the linker of formula (II) or (II′) as described above. Accordingly, the compound of the present invention may be represented by the following general formula (X) or (X′):

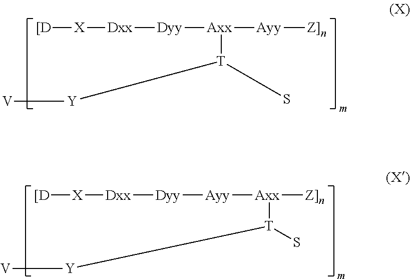

Axx in formula (X) and in formula (X′) represents a moiety derived from an amino acid selected from Glu, Apa, Aaa, Dap, Dab, Lys, Orn, Ser, Ama and homo-Lys, preferably a moiety derived from an amino acid selected from Dap, Dab, Lys, Orn and homo-Lys, more preferably a moiety derived from a moiety derived from Lys;
Ayy in formula (X) represents a moiety derived from an amino acid selected from Phe, homo-Phe, Ala, Trp, Phg, Leu, Val, Tyr, homo-Tyr, Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24, preferably a moiety derived from Phe, homo-Phe, Tyr, homo-Tyr, Tyr(OR₁) or homo-Tyr(OR₁), more preferably a moiety derived from Tyr;
Ayy in formula (X′) represents a moiety derived from an amino acid selected from Phe, homo-Phe, Ala, Trp, Phg, Leu, Val, Tyr and Ser, preferably a moiety derived from Phe, home-Phe or Ser, more preferably a moiety derived from Phe or Ser;
D, Dxx, Dyy, X, T, S, V, Z, m and n in formulae (X) and (X′) have the same meanings as described above.

According to one preferred embodiment, at least one, e.g., two, three, four, five, six, seven or eight, of D, Dxx, Dyy, X, Y, T, S and Z is/are defined as follows:
(a) D is a moiety derived from a drug selected from auristatin, MMAF, exatecan, maytansine, DM1 and DM4, preferably a moiety derived from DM1 or DM4;
(b) Dxx is a moiety derived from an amino acid selected from Phe, Val, Tyr, homo-Phe and Ala, preferably from Phe or Val;
(c) Dyy is a covalent bond or a moiety derived from an amino acid selected from Arg, Lys, Cit, Orn, Dap and Dab, preferably a covalent bond or a moiety derived from Arg or Cit;
(d) X is a group of formula (III) wherein n2 is 1 or 2, or a group represented by any of formulae (IVa) to (IVz), preferably a group represented by any of formula (IVc), (IVm), (IVn), (IVo), (IVp), (IVs) or (IVt), more preferably by formula (IVc) or (IVm);
(e) Y is a group derived from a compound selected from maleimides, triazoles, hydrazones, carbonyl-containing compounds and derivatives thereof, preferably a group derived from maleimides and derivatives thereof such as opened hydrolyzed maleimide derivatives, and more preferably from an opened hydrolyzed maleimide;
(f) T is a group of formula (VII), (VIII) or (IX);
(g) S is a moiety of formula (V); and
(h) Z is —OH.

According to one preferred embodiment, at least one, e.g., two, three, four, five, six, seven, eight or nine, of D, Dxx, Dyy, X, Y, T, S, V and Z is/are defined as follows:
(a) D is a moiety derived from a drug selected from auristatin, MMAF, exatecan, maytansine, DM1 and DM4, preferably a moiety derived from DM1 or DM4;
(b) Dxx is a moiety derived from an amino acid selected from Phe, Val, Tyr, homo-Phe and Ala, preferably from Phe or Val;
(c) Dyy is a covalent bond or a moiety derived from an amino acid selected from Arg, Lys, Cit, Orn, Dap and Dab, preferably a covalent bond or a moiety derived from Arg or Cit;
(d) X is a group of formula (III) wherein n2 is 1 or 2, or a group represented by any of formulae (IVa) to (IVz), preferably a group represented by any of formula (IVc), (IVm), (IVn), (IVo), (IVp), (IVs) or (IVt), more preferably by formula (IVc) or (IVm);
(e) Y is a group derived from a compound selected from maleimides, triazoles, hydrazones, carbonyl-containing compounds and derivatives thereof, preferably a group derived from maleimides and derivatives thereof such as opened hydrolyzed maleimide derivatives, and more preferably from an opened hydrolyzed maleimide;
(f) T is a group of formula (VII), (VIII) or (IX);
(g) S is a moiety of formula (V);
(h) V is a moiety derived from naratuximab; and
(i) Z is —OH.

According to a further preferred embodiment, each Dyy-Dxx-Axx-Ayy in formula (X) is independently selected from Arg-Lys-Phe wherein Dyy is a covalent bond, Arg-Lys-homoPhe wherein Dyy is a covalent bond, Arg-Lys-Tyr wherein Dyy is a covalent bond, Cit-Lys-Phe wherein Dyy is a covalent bond, Cit-Lys-Tyr wherein Dyy is a covalent bond, Arg-Lys-homoTyr wherein Dyy is a covalent bond, Cit-Lys-homoTyr wherein Dyy is a covalent bond, Phe-Cit-Lys-Phe, Phe-Cit-Lys-Tyr, Phe-Arg-Lys-Tyr, Phe-Cit-Lys-homoTyr, Phe-Lys-Lys-Phe, homoPhe-Arg-Lys-Phe, homo-Phe-Cit-Lys-Tyr; and each Dyy-Dxx-Ayy-Axx in formula (X′) is independently selected from Arg-Phe-Lys wherein Dyy is a covalent bond, Arg-Ser-Lys wherein Dyy is a covalent bond, Cit-Phe-Lys wherein Dyy is a covalent bond, Cit-Ser-Lys wherein Dyy is a covalent bond, Cit-homoPhe-Lys wherein Dyy is a covalent bond, Phe-Cit-Phe-Lys, homoPhe-Cit-Phe-Lys, and Phe-Arg-Phe-Lys.

In one embodiment, the compound of the present invention is represented by one of the following formulae:

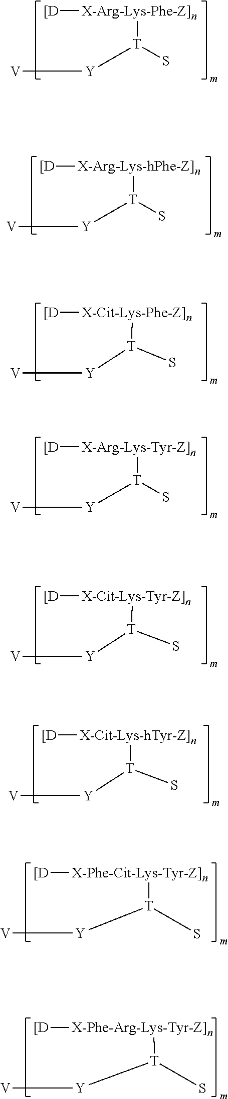

wherein D, X, Y, T, S, V, Z, m and n have the same meanings as described above.

According to one preferred embodiment, at least one, e.g., two, three, four, five or six, of D, X, Y, T, S and Z is/are defined as follows:
(a) D is a moiety derived from a drug selected from auristatin, AF, MMAF, exatecan, maytansine, DM1 and DM4, preferably a moiety derived from DM1 or DM4;
(d) X is a group of formula (III), wherein n2 is 1 or 2, or a group represented by any of formulae (IVa) to (IVz), preferably a group represented by any of formula (IVc), (IVm), (IVn), (IVo), (IVp), (IVs) or (IVt), more preferably by formula (IVc) or (IVm);
(e) Y is a group derived from a compound selected from maleimides, triazoles, hydrazones, carbonyl-containing compounds and derivatives thereof, preferably from maleimides and derivatives thereof such as opened hydrolyzed maleimide derivatives, and more preferably from an opened hydrolyzed maleimide;
(f) T is a group of formula (VII), (VIII) or (IX);
(g) S is a moiety of formula (V); and
(h) Z is —OH.

According to one preferred embodiment, at least one, e.g., two, three, four, five, six or seven, of D, X, Y, T, S, V and Z is/are defined as follows:
(a) D is a moiety derived from a drug selected from auristatin, AF, MMAF, exatecan, maytansine, DM1 and DM4, preferably a moiety derived from DM1 or DM4;
(d) X is a group of formula (III), wherein n2 is 1 or 2, or a group represented by any of formulae (IVa) to (IVz), preferably a group represented by any of formula (IVc), (IVm), (IVn), (IVo), (IVp), (IVs) or (IVt), more preferably by formula (IVc) or (IVm);
(e) Y is a group derived from a compound selected from maleimides, triazoles, hydrazones, carbonyl-containing compounds and derivatives thereof, preferably from maleimides and derivatives thereof such as opened hydrolyzed maleimide derivatives, and more preferably from an opened hydrolyzed maleimide;
(f) T is a group of formula (VII), (VIII) or (IX);
(g) S is a moiety of formula (V);
(h) V is a moiety derived from naratuximab; and
(h) Z is —OH.

In one embodiment, the compound of the present invention is represented by one of the following formulae (as is evident from formula (I), in the following formulae, the vector group (V) is to be understood as being outside the parentheses so that indicia m does not apply to (V) whereas all other depicted structural elements are to be understood as being within the parentheses and thus present in the molecule m times):

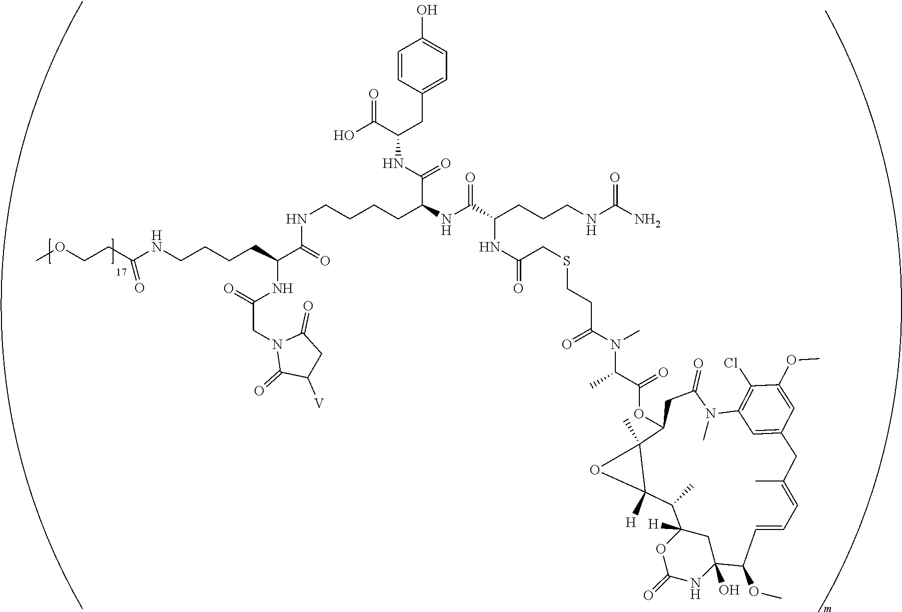

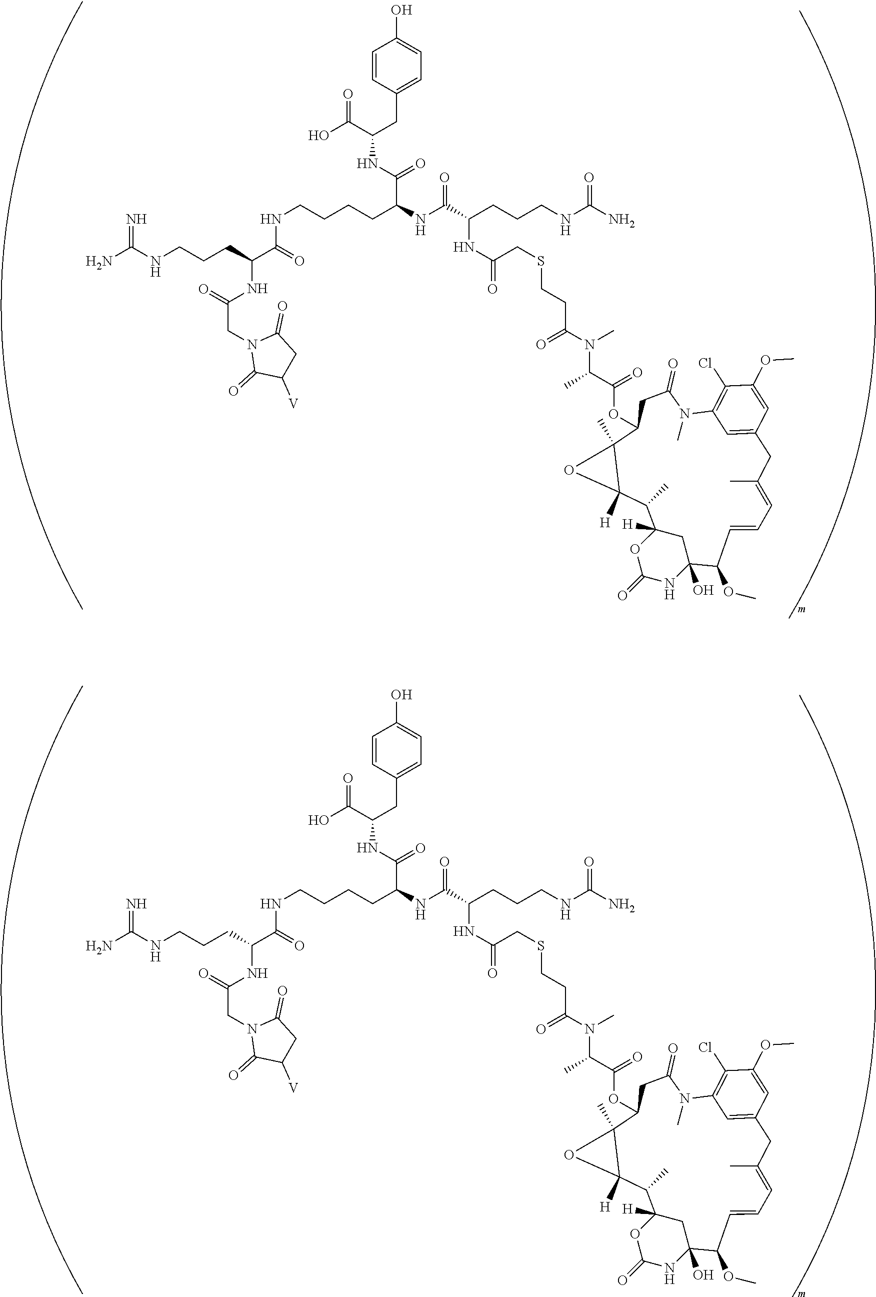

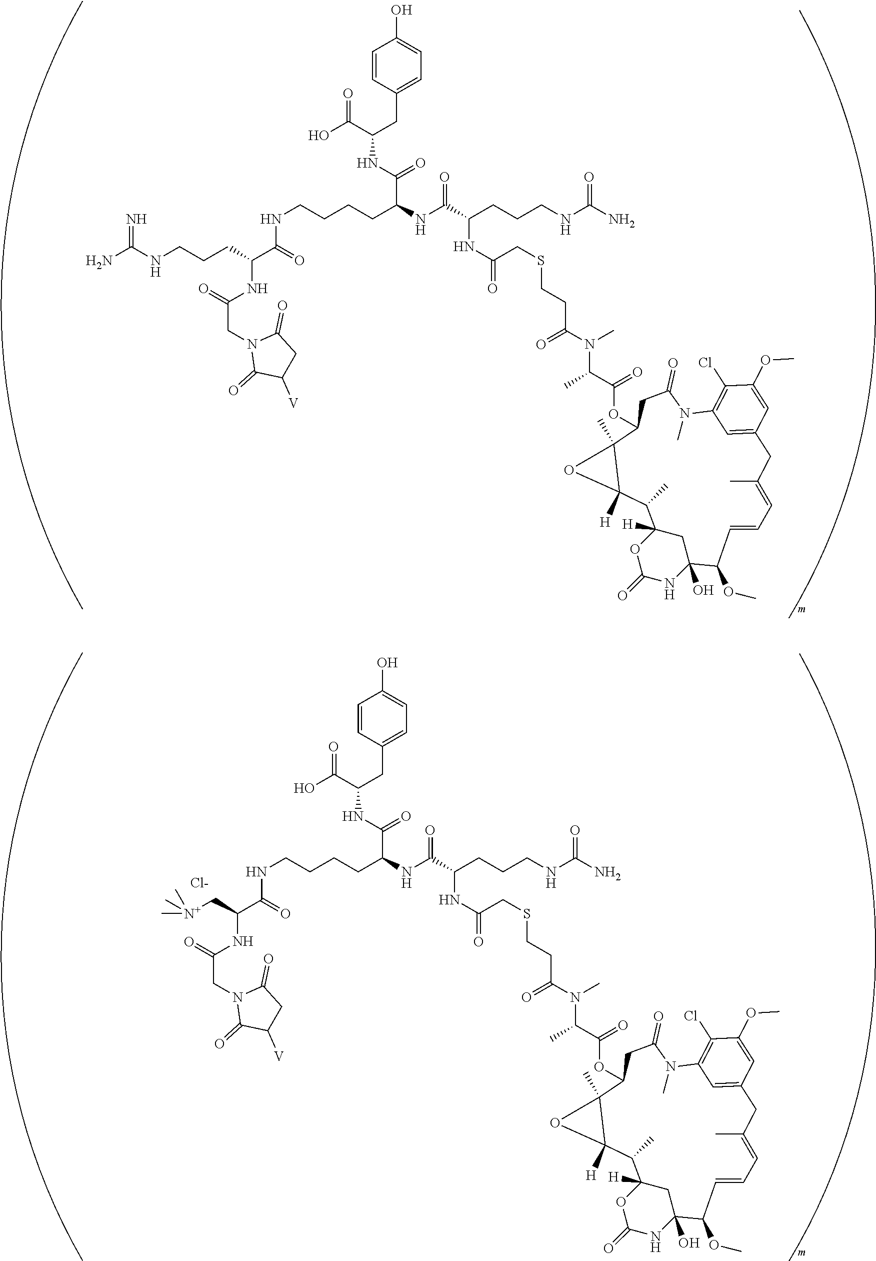

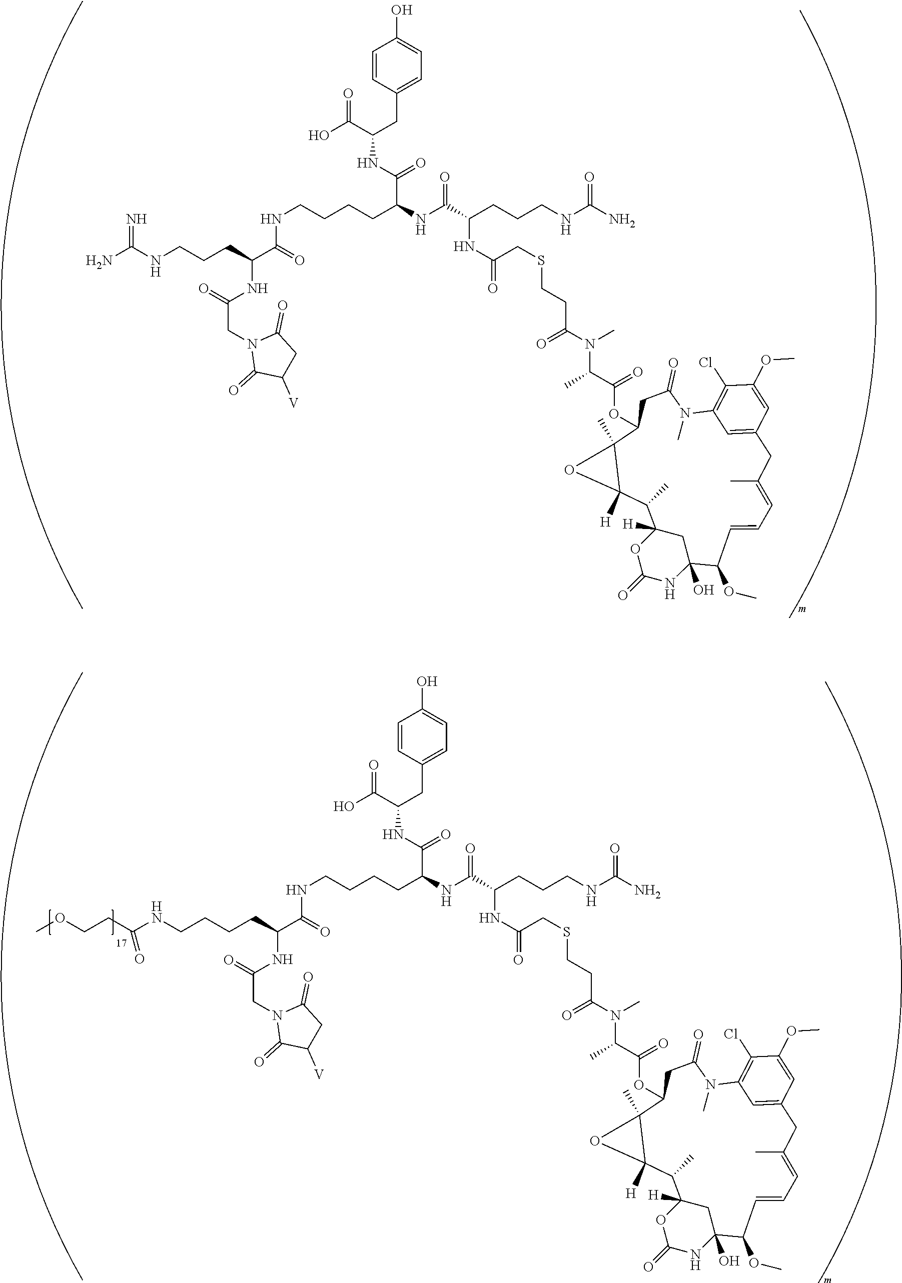

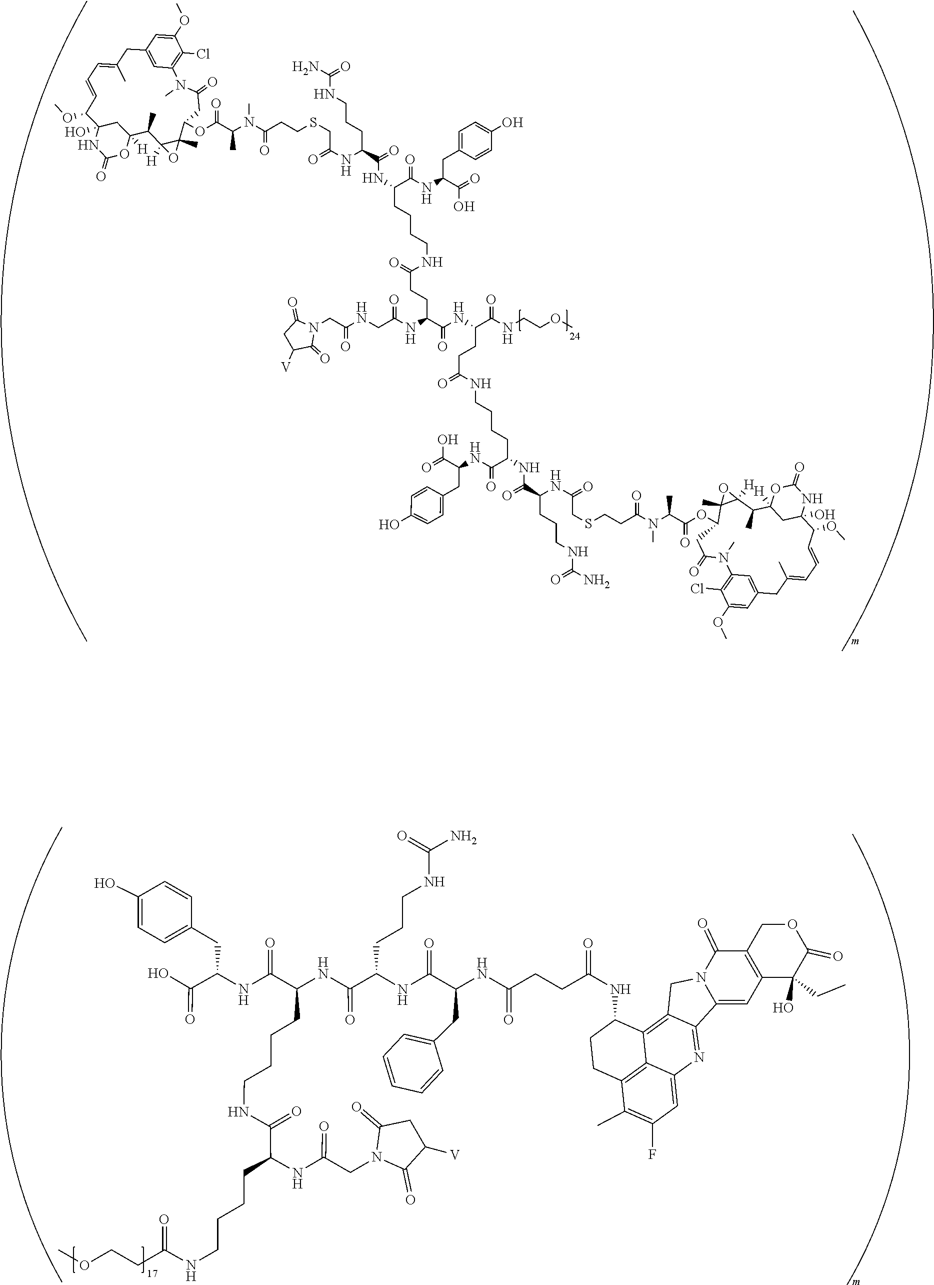

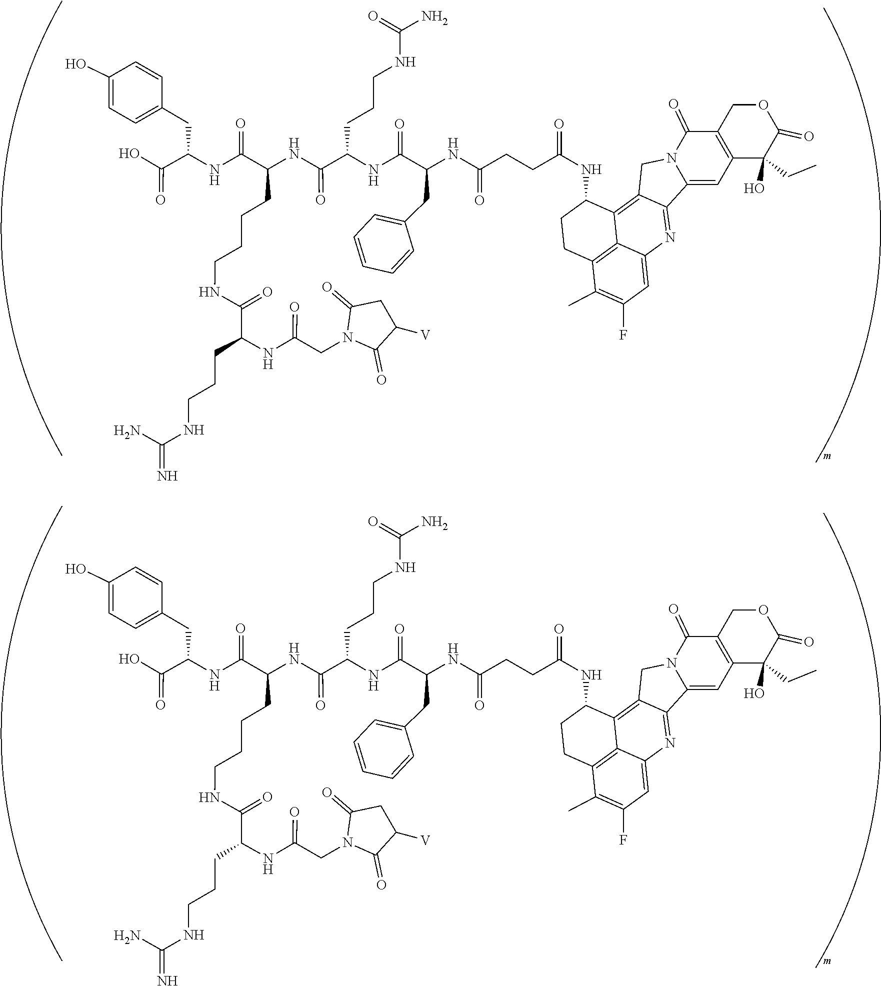

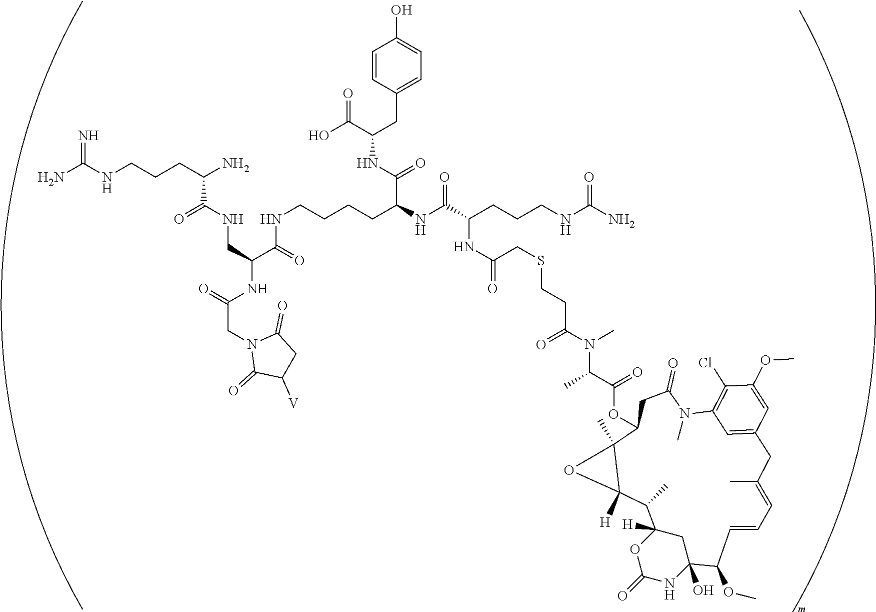

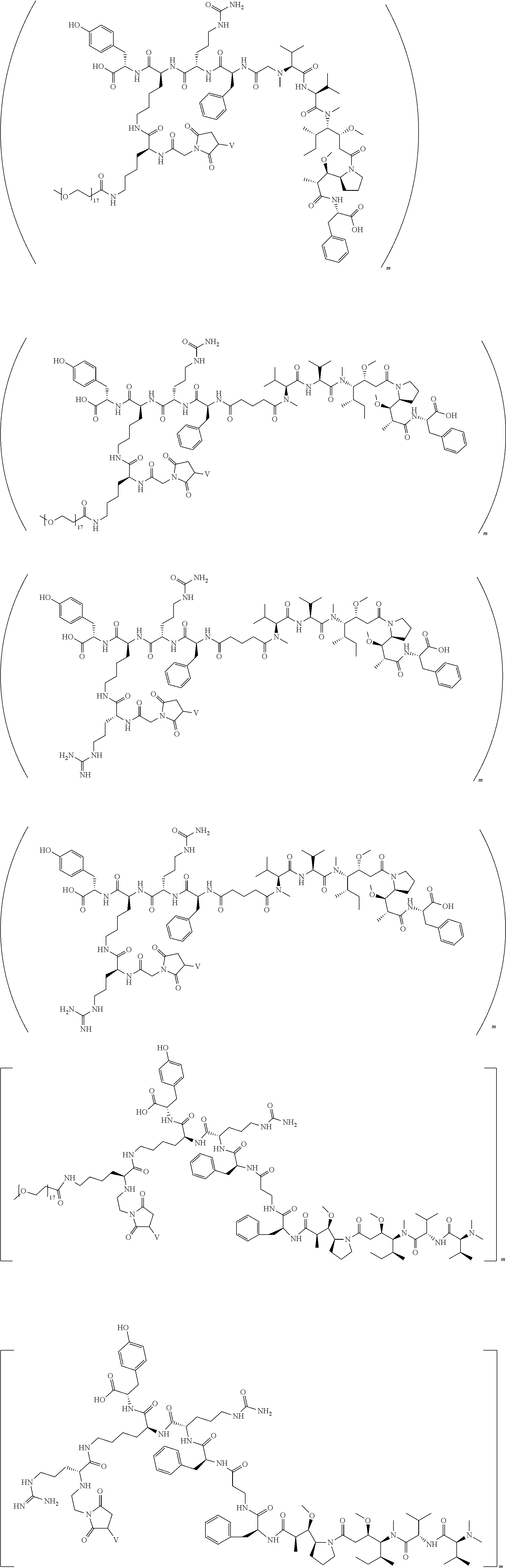

wherein m is an integer of 1 to 12, preferably 2 to 10, more preferably 4 to 8.

According to one embodiment, in the above compounds wherein the solubilizing moiety (S) comprises a C₂ polyoxyalkylene oxide group, the number of oxyethylene repeating units (17) may be replaced by 12 to 22, preferably 15 to 19 oxyethylene groups.

According to one embodiment, in the above compounds the maleimide attachment to (V) may be replaced by an opened hydrolyzed maleimide attachment. In some instances, wherein m is at least 2, said compound may comprise a mix of (closed) maleimide derivatives and opened hydrolyzed maleimide derivatives attached to V, preferably at least 50% of the attachments to V are opened hydrolyzed maleimide attachments.

In one preferred embodiment, the compound (LDC) is represented by one of the following formulae:

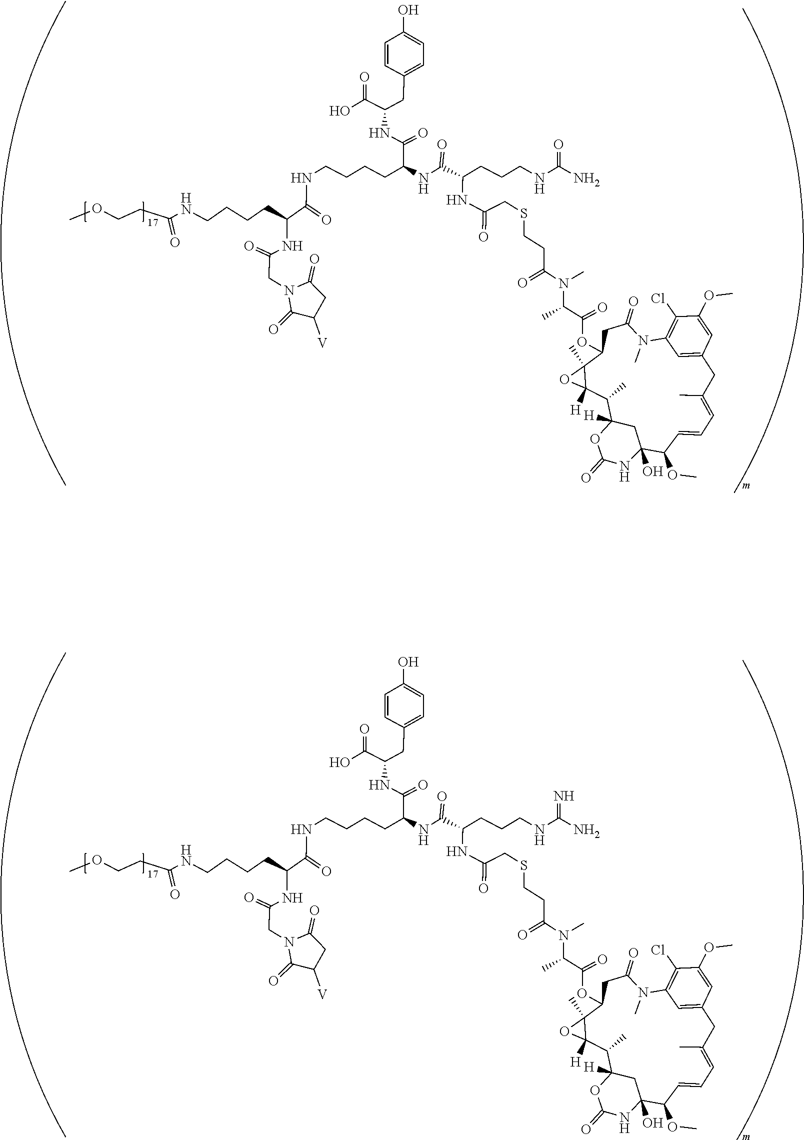

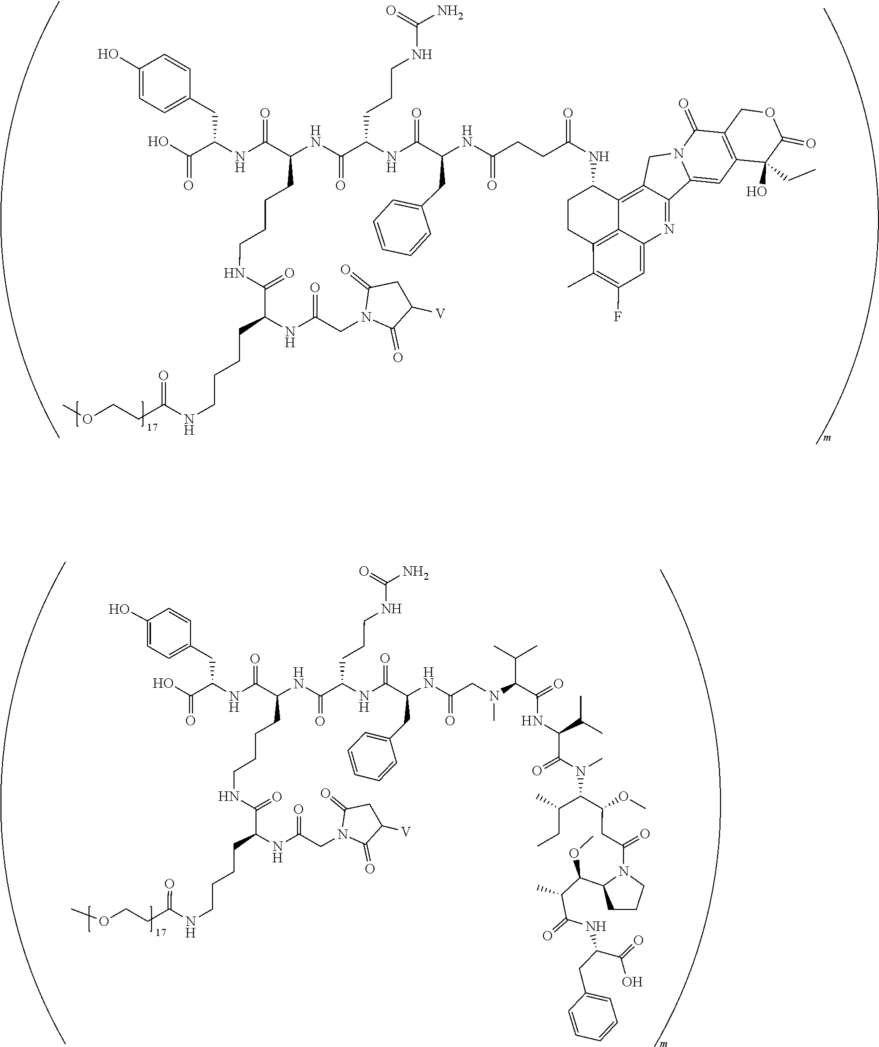

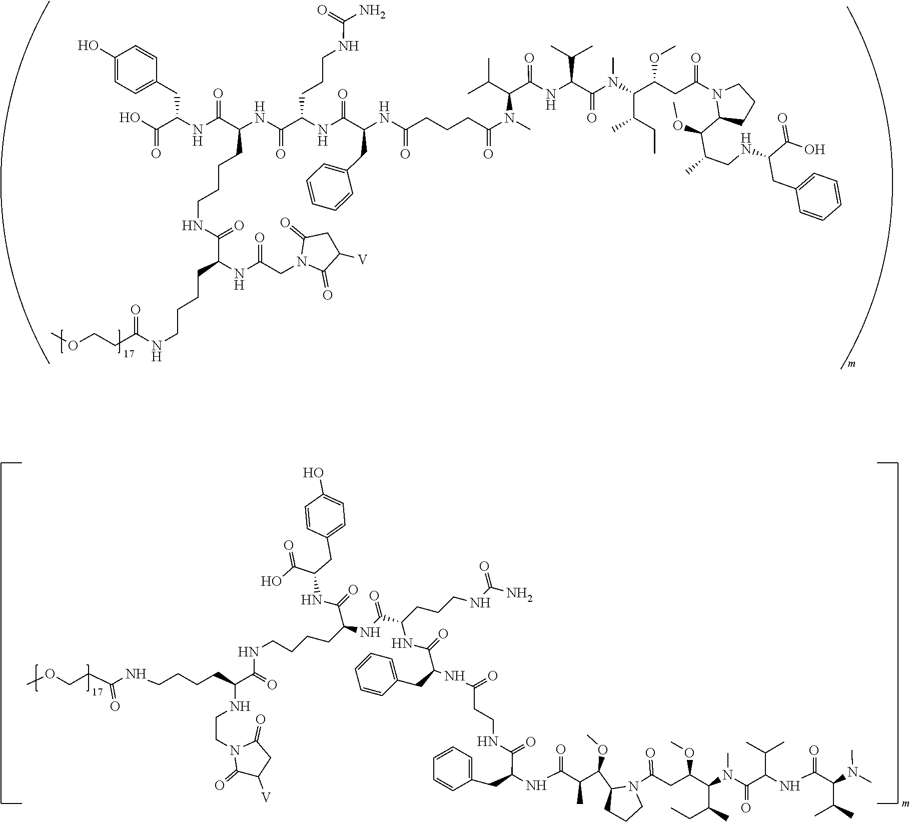

wherein m is an integer of 1 to 12, preferably 2 to 10, more preferably 4 to 8.

As is evident from formula (I), in the above compounds, the vector group (V) is to be understood as being outside the parentheses so that indicia m does not apply to (V) whereas all other depicted structural elements are to be understood as being within the parentheses and thus present in the molecule m times.

According to one embodiment, in the above compounds wherein the solubilizing moiety (S) comprises a C₂ polyoxyalkylene oxide group, the number of oxyethylene repeating units (17 or 24) may be replaced by 12 to 30, preferably 14 to 25, more preferably 15 to 19 oxyethylene groups.

According to one embodiment, in the above compounds the maleimide attachment to (V) may be replaced by an opened hydrolyzed maleimide attachment. In some instances, wherein m is at least 2, said compound may comprise a mix of (closed) maleimide derivatives and opened hydrolyzed maleimide derivatives attached to V, preferably at least 50% of the attachments to V are opened hydrolyzed maleimide attachments.

In one preferred embodiment, the compound (LDC) is represented by one of the following formulae:

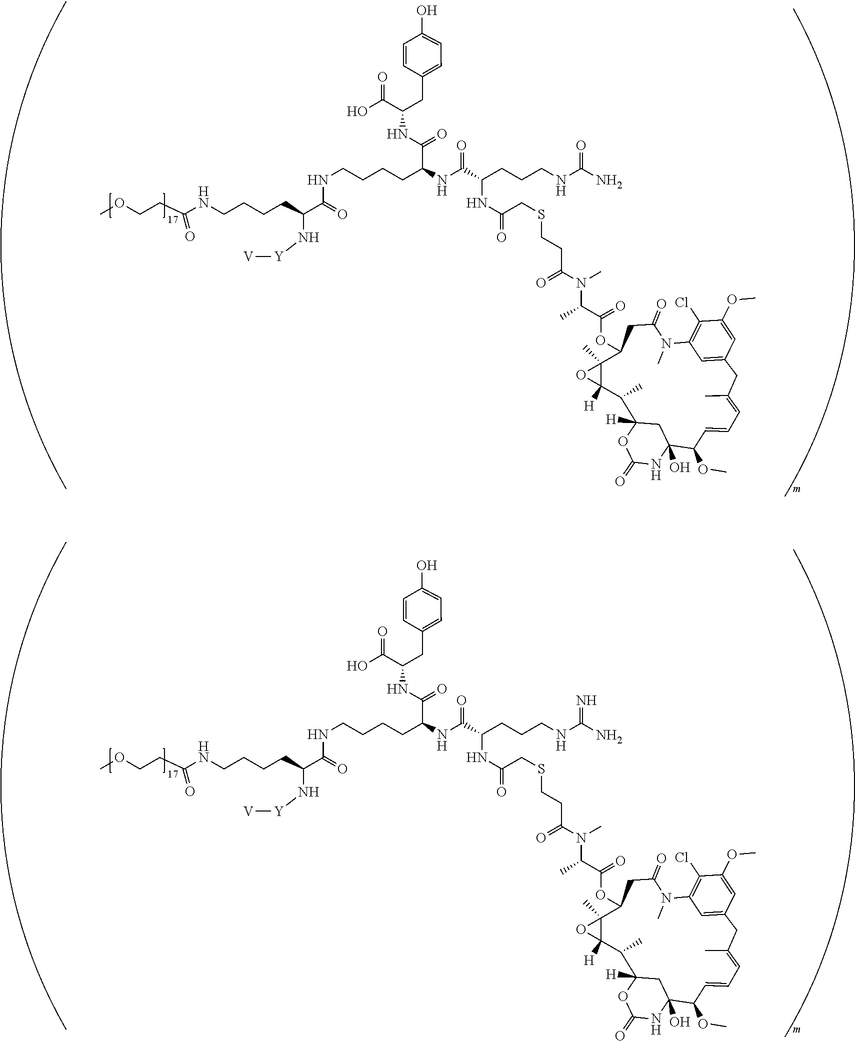

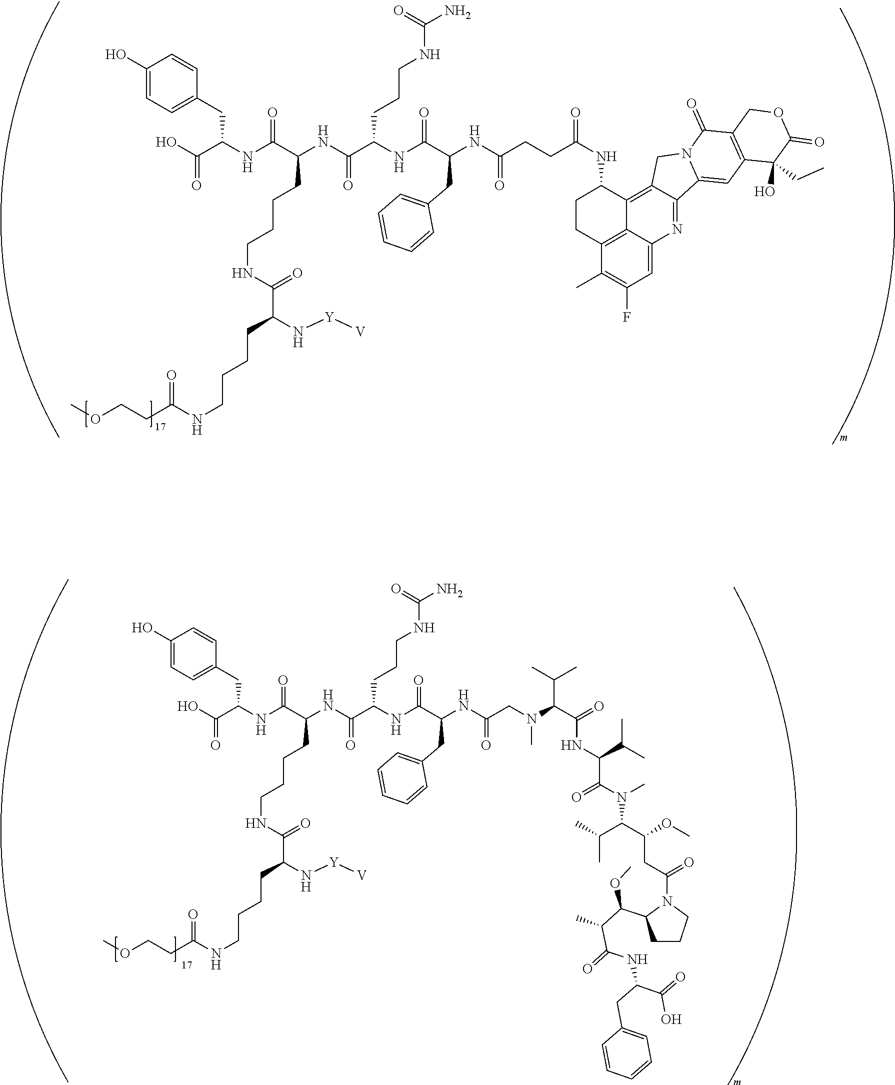

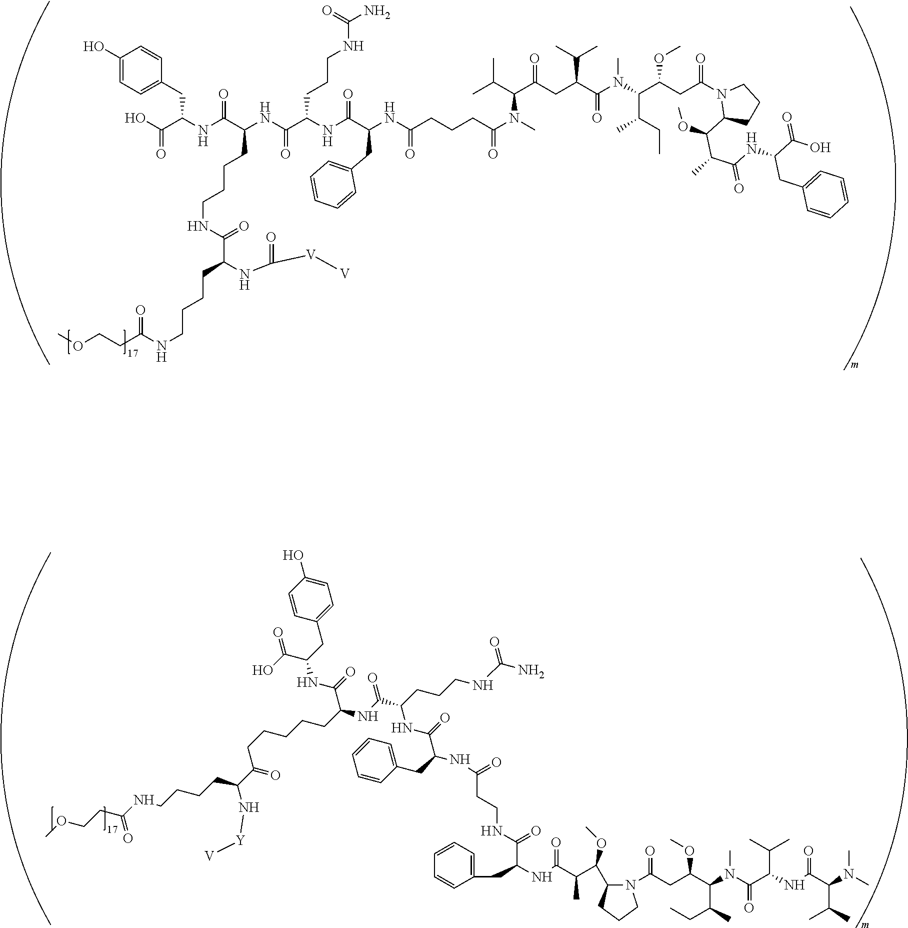

wherein,
V is as defined in formula (I);
Y is a group derived from maleimides and derivatives thereof such as opened hydrolyzed maleimide derivatives, preferably from an opened hydrolyzed maleimide; and
m is an integer of 1 to 12, preferably 2 to 10, more preferably 4 to 8.

As is evident from formula (I), in the above compounds, the vector group (V) is to be understood as being outside the parentheses so that indicia m does not apply to (V) whereas all other depicted structural elements are to be understood as being within the parentheses and thus present in the molecule m times.

According to one embodiment, in the above compounds wherein the solubilizing moiety (S) comprises a C₂ polyoxyalkylene oxide group, the number of oxyethylene repeating units (17 or 24) may be replaced by 12 to 30, preferably 14 to 25, more preferably 15 to 19 oxyethylene groups.

According to a preferred embodiment, in the above compounds, Y is represented by any of the following formulae (XIIIa) to (XIIIc):

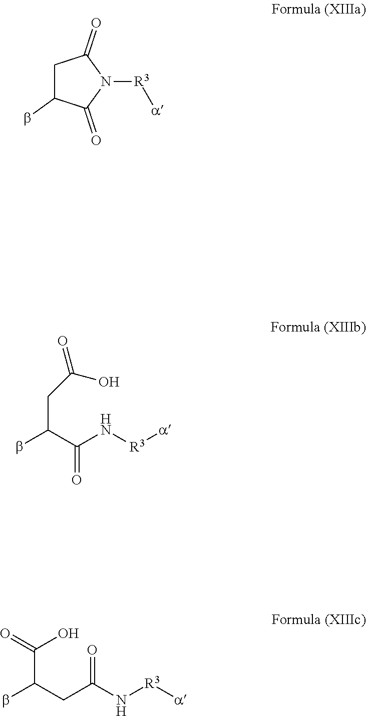

wherein,
R³ represents —(CH₂)ₙ₇—(C=A)ₙ₉- or —(CH₂CH₂O)ₙ₈—(C=A)ₙ₉-, preferably —(CH₂)ₙ₇—(C=A)ₙ₉-, wherein,
   n7 is 1 to 6, preferably 1 or 2, more preferably 1,
   n8 is 1 to 6, preferably 1,
   n9 is 0 or 1, preferably 1, and
   A is O or S, preferably O;
wherein the methylene carbon atom is covalently attached to the nitrogen atom of formulae (XIIIa)-(XIIIc) and the carbonyl or thiocarbonyl-carbon is covalently attached to the nitrogen atom of T (to the amino group derived from the Lys residue of T);
β indicates covalent attachment to V; and
α′ indicates covalent attachment to T (to the amino group derived from the Lys residue of T).

In a more preferred embodiment, Y is represented by formula (XIIIb) or (XIIIc), wherein R³ is preferably a group represented by the formula —(CH₂)ₙ₇—(C=A)ₙ₉- in which n7 is 1 or 2, n9 is 1 and A is O. Most preferably, R³ is —CH₂—C═O—.

### 3.8 Vector Group (V)

The vector group (V) in formulae (I), (X) and (X′) represents a moiety derived from a vector group capable of interacting with a target cell. The expression “capable of interacting with a target cell” as used herein indicates that the vector group can bind to, complex with, or react with a moiety, e.g., an antigen or a receptor, on the surface of a target cell. Such an interaction with the target cell can be experimentally verified by methods known in the art, for instance by providing a compound of formula (I), which carries a label (such as a fluorescence marker), by contacting said compound with tissue containing target cells and by detecting the distribution of the fluorescence marker within the tissue (e.g., by fluorescence microscopy). An increase of fluorescence intensity at the target cells indicates an interaction with the target cell in accordance with the present invention. In some preferred embodiments, the vector group is also capable of causing or contributing to internalization of the targeted-drug-conjugate, i.e., the compound of formula (I), into the target cell.

In one embodiment, (V) represents a moiety derived from a vector group selected from antibodies, antibody fragments, proteins, peptides, and non-peptidic molecules.

In one embodiment, (V) represents a moiety derived from an antibody or an antibody fragment such as a single chain antibody, a monoclonal antibody, a single chain monoclonal antibody, a monoclonal antibody fragment, a chimeric antibody, a chimeric antibody fragment, a domain antibody or fragment thereof, a cytokine, a hormone, a growth factor, a colony stimulating factor, a neurotransmitter or a nutrient-transport molecule.

In one preferred embodiment, (V) represents a moiety derived from:
a monoclonal antibody, preferably a moiety derived from an antibody selected from the group consisting of adalimumab, aducanumab, alemtuzumab, altumomab pentetate, amivantamab, atezolizumab, anetumab, avelumab, bapineuzumab, basiliximab, bectumomab, belantamab mafadotin, bermekimab, besilesomab, bevacizumab, bezlotoxumab, brentuximab, brentuximab vedotin, brodalumab, catumaxomab, cemiplimab, cetuximab, cinpanemab, clivatuzumab, crenezumab, tetraxetan, daclizumab, daratumumab, denosumab, dinutuximab, dostarlimab, durvalumab, edrecolomab, elotuzumab, emapalumab, enfortumab, enfortumab vedotin, epcoritamab, epratuzumab, epratuzumab-SN-38, etaracizumab, gemtuzumab, gemtuzumab ozogamycin, genmab, glofitamab, girentuximab, gosuranemab, ibritumomab, inebilizumab infliximab, inotuzumab, inotuzumab ozogamicin, ipilimumab, isatuximab ixekizumab, J591 PSMA-antibody, labetuzumab, lecanemab, loncastuximab tesirin, mogamulizumab, mosunetuzumab, necitumumab, nimotuzumab, natalizumab, naratuximab, naxitamab, nivolumab, ocrelizumab, ofatumumab, olaratumab, oregovomab, panitumumab, pembrolizumab, pertuzumab, polatuzumab, polatuzumab vedotin, prasinezumab, racotumomab, ramucirumab, rituximab, sacituzumab, sacituzumab govitecan, semorinemab, siltuximab, solanezumab, tacatuzumab, tafasitamab, teprotumumab, tilavonemab, tocilizumab, tositumomab, trastuzumab, trastuzumab deruxtecan, trastuzumab emtansine, TS23, ustekinumab, vedolizumab, votumumab, zagotenemab, zalutumumab, zanolimumab, fragments and derivatives thereof; more preferably from atezolizumab, durvalumab, pembrolizumab, rituximab or trastuzumab; or
an antibody fragment incorporated into an Fc-fusion protein, said antibody fragment being preferably selected from belatacept, aflibercept, ziv-aflibercept, dulaglutide, rilonacept, romiplostim, abatacept and alefacept.

In a preferred embodiment, (V) represents a moiety derived from an anti-HER2, anti-CD37, anti-PDL1 or anti-EGFR antibody, preferably from an antibody selected from trastuzumab, pembrolizumab, naratuximab, atezolizumab, durvalumab, panitumumab, avelumab and cetuximab, more preferably from naratuximab, trastuzumab, and cetuximab, and most preferably from naratuximab or trastuzumab.

As used herein, the term “naratuximab” (also referred to as K7153A herein) refers to the antibody huCD37-3 (Version 1.0) described in WO 2019/229677 (incorporated herein by reference). In another aspect, naratuximab is characterized as the monoclonal antibody huCD37-3v1.0 in WO2011/112978, which specifically describes its heavy chain (SEQ ID NO:90) and light chain (SEQ ID 107). In yet further aspects, said antibody comprises the CDRs represented by SEQ ID NOs:2-7 in tables 1 and 2, the VH of SEQ ID NO:8 in table 3 and the VL of SEQ ID NO:10 in table 4 of WO 2019/229677.

In particular aspects, naratuximab may comprise
the full-length light chain of Seq No 11 in table 5 of WO 2019/229677,
the full-length heavy chain of Seq No. 12 in table 6 of WO 2019/229677,
the full-length light chain of Seq No 11 in table 5 and the full-length heavy chain of Seq No. 12 in table 6 of WO 2019/229677,
a light chain or light chain variable region having the same amino acid sequence as the amino acid sequence encoded by the recombinant plasmid DNA phuCD37-3LC (ATCC Deposit Designation PTA-10722, deposited with the ATCC on Mar. 18, 2010),
a heavy chain or heavy chain variable region comprising the same amino acid sequence as the amino acid sequence encoded by the recombinant plasmid DNA phuCD37-3HCv.1.0 (ATCC Deposit Designation PTA-10723, deposited with the ATCC on Mar. 18, 2010),
a light chain or light chain variable region comprising the same amino acid sequence as the amino acid sequence encoded by the recombinant plasmid DNA phuCD37-3LC (PTA-10722) and a heavy chain or heavy chain variable region comprising the same amino acid sequence as the amino acid sequence encoded by the recombinant plasmid DNA phuCD37-3HCv.1.0 (PTA-10723),
comprise (i) VL-CDRs comprising the same amino acid sequences as the VL-CDRs encoded by the recombinant plasmid DNA phuCD37-3LC (PTA-10722) and (ii) VH-CDRs comprising the same amino acid sequences as the VH-CDRs encoded by the recombinant plasmid DNA phuCD37-3HCv.1.0 (PTA-10723).

In one embodiment, (V) represents a moiety derived from an anti-CD37 antibody, preferably naratuximab or an anti-HER2 antibody, preferably trastuzumab.

In another embodiment, (V) represents a moiety derived from an anti-CD37 antibody, preferably naratuximab, or an anti-HER2 antibody, preferably trastuzumab, and (D) represents a moiety derived from an antineoplastic agent, and preferably a moiety derived from a drug selected from amanitin, duocarmycin, auristatin, auristatin F (AF), monomethyl auristatin F (MMAF), maytansine, mertansine (DM1), ravtansine (DM4), tubulysin, calicheamicin, camptothecin, SN-38, exatecan, Maaa1181a, taxol, daunomycin, vinblastine, doxorubicin, methotrexate, pyrrolobenzodiazepine (PBD) and dimers thereof, indilinobenzodiazepine (IBD) and dimers thereof, or radioisotopes and/or pharmaceutically acceptable salts thereof.

In a preferred embodiment (V) represents a moiety derived from an anti-CD37 antibody, preferably naratuximab, or an anti-HER2 antibody, preferably trastuzumab, and (D) represents a moiety derived from a drug selected from auristatin, MMAF, exatecan, maytansine, DM1 and DM4. More preferably, (V) represents a moiety derived from an anti-CD37 antibody, preferably naratuximab, or an anti-HER2 antibody, preferably trastuzumab, and (D) represents a moiety derived from DM1 or DM4.

In another preferred embodiment, (V) represents a moiety derived from an anti-CD37 antibody, preferably naratuximab, or an anti-HER2 antibody, preferably trastuzumab, and (D) represents a moiety derived from DM1, and the compound is represented by one of the following formulae:

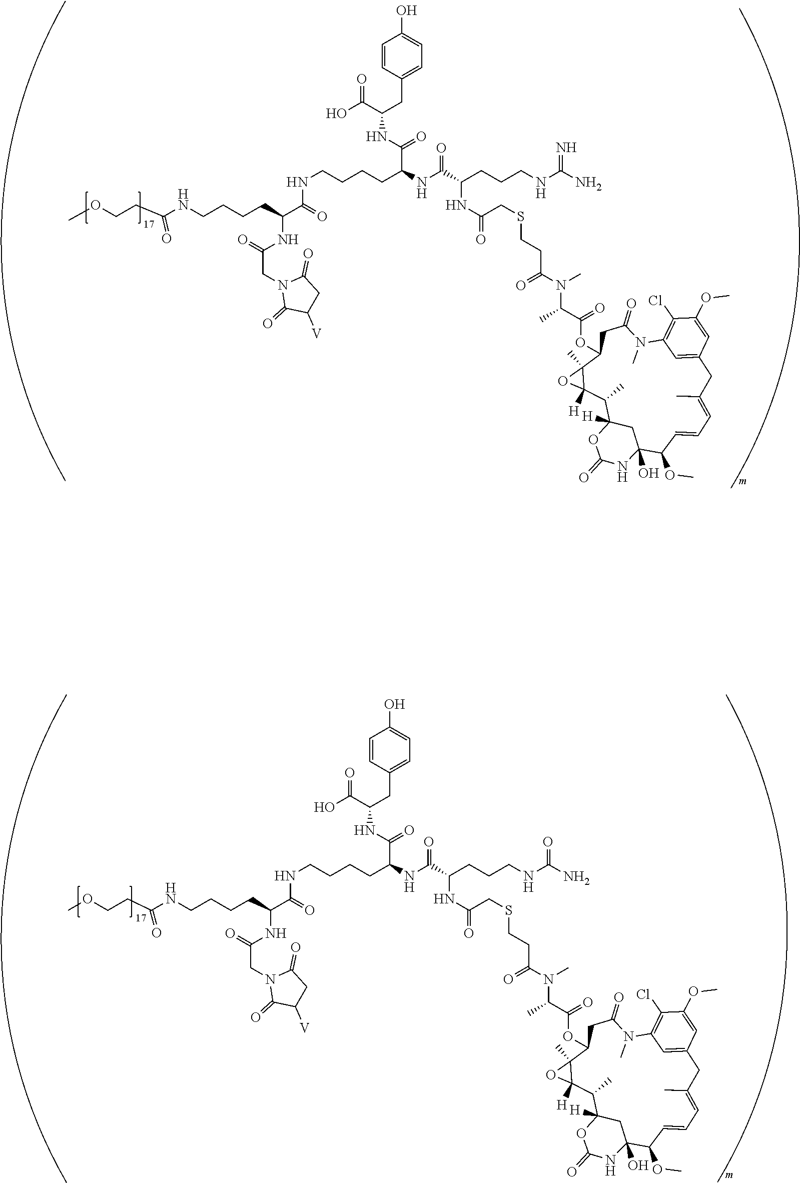

wherein m is an integer of 1 to 12, preferably 2 to 10, more preferably 4 to 8; wherein the number of oxyethylene repeating units (17) may be replaced by 12 to 30, preferably 14 to 25, more preferably 15 to 19 oxyethylene groups; and/or wherein the maleimide attachment to (V) may be replaced by an opened hydrolyzed maleimide.

As is evident from formula (I), in the above compounds, the vector group (V) is to be understood as being outside the parentheses so that indicia m does not apply to (V) whereas all other depicted structural elements are to be understood as being within the parentheses and thus present in the molecule m times.

According to one embodiment, in the above compounds the maleimide attachment to (V) may be replaced by an opened hydrolyzed maleimide attachment. In some instances, wherein m is at least 2, said compound may comprise a mix of (closed) maleimide derivatives and opened hydrolyzed maleimide derivatives attached to V, preferably at least 50% of the attachments to V are opened hydrolyzed maleimide attachments.

In yet another preferred embodiment, (V) represents a moiety derived from an anti-CD37 antibody, preferably naratuximab, or an anti-HER2 antibody, preferably trastuzumab, and (D) represents a moiety derived from DM1, and the compound is represented by one of the following formulae:

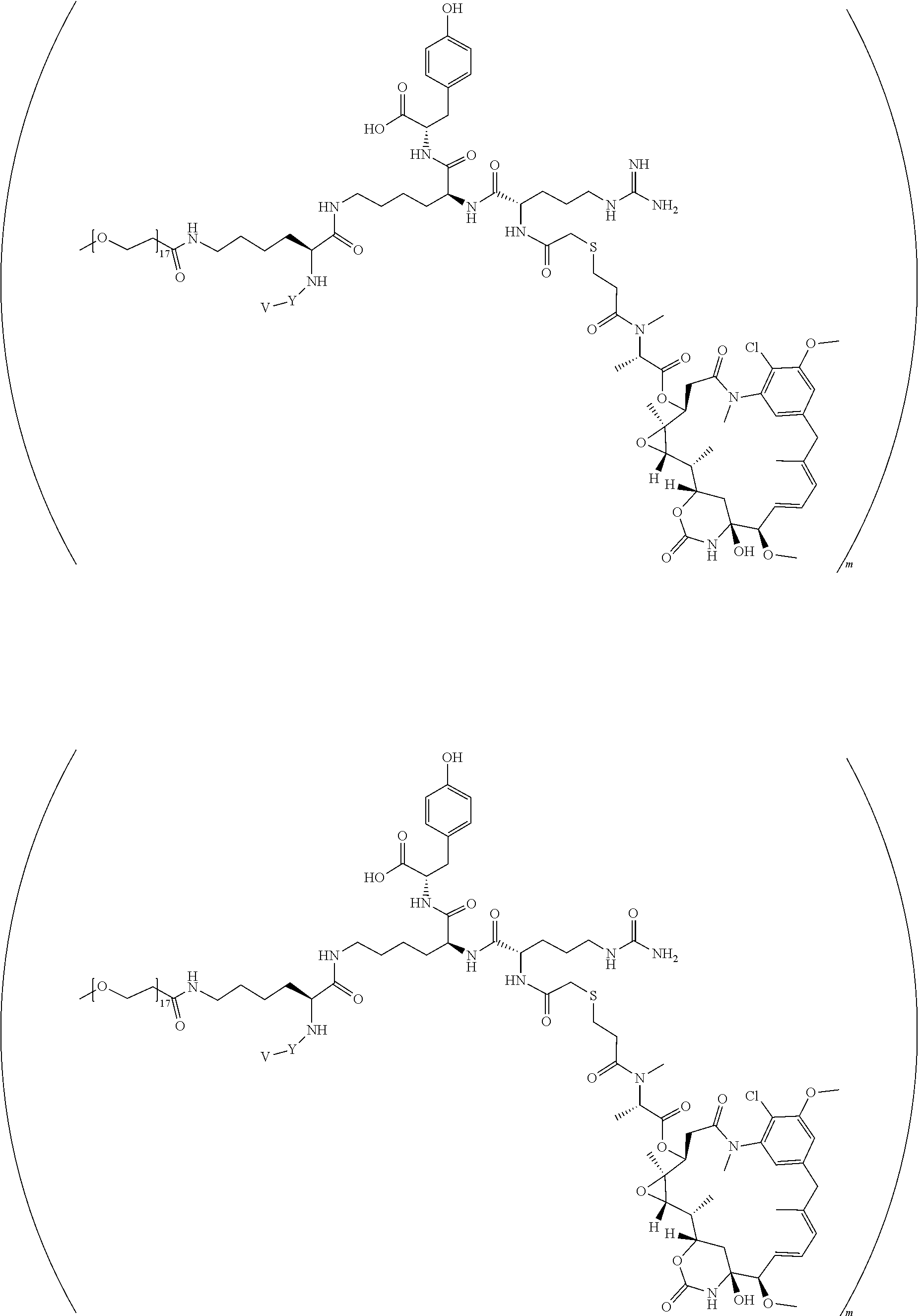

wherein m is an integer of 1 to 12, preferably 2 to 10, more preferably 4 to 8; wherein Y is represented by any of the formulae (XIIIa) to (XIIIc), preferably by formula (XIIIb) or (XIIIc); and/or wherein the number of oxyethylene repeating units (17) may be replaced by 12 to 30, preferably 14 to 25, more preferably 15 to 19 oxyethylene groups.

In a more preferred embodiment, (V) represents a moiety derived from an anti-CD37 antibody, preferably naratuximab, or an anti-HER2 antibody, preferably trastuzumab, and (D) represents a moiety derived from DM1, and the compound is represented by the following formula:

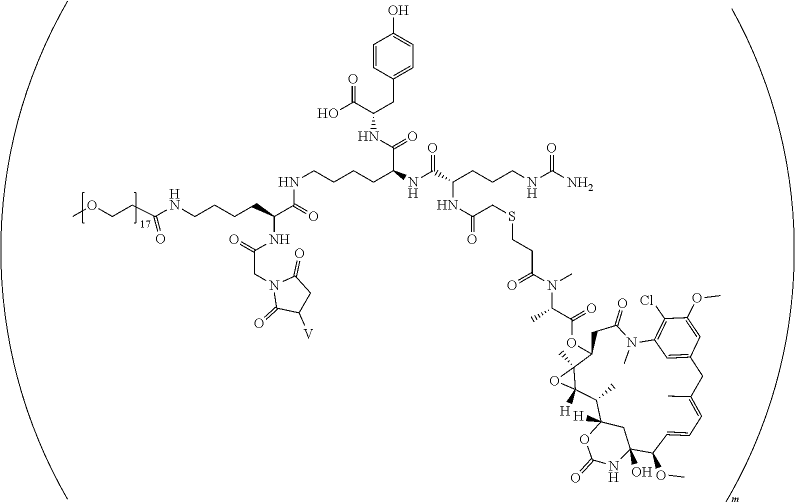

wherein m is an integer of 1 to 12, preferably 2 to 10, more preferably 4 to 8; wherein the number of oxyethylene repeating units (17) may be replaced by 12 to 30, preferably 14 to 25, more preferably 15 to 19 oxyethylene groups; and/or wherein the maleimide attachment to (V) may be replaced by an opened hydrolyzed maleimide.

In an even more preferred embodiment, (V) represents a moiety derived from an anti-CD37 antibody, preferably naratuximab, or an anti-HER2 antibody, preferably trastuzumab, and (D) represents a moiety derived from DM1, and the compound is represented by the following formula:

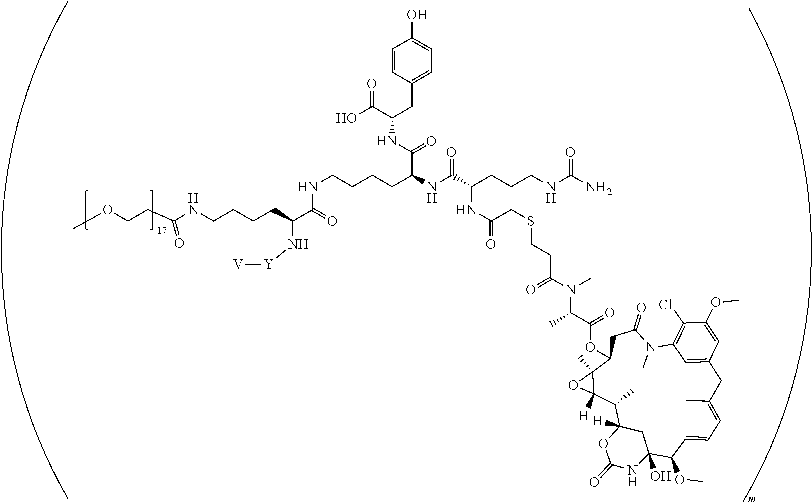

wherein m is an integer of 1 to 12, preferably 2 to 10, more preferably 4 to 8; wherein Y is represented by any of the formulae (XIIIa) to (XIIIc), preferably by formula (XIIIb) or (XIIIc); and/or wherein the number of oxyethylene repeating units (17) may be replaced by 12 to 30, preferably 14 to 25, more preferably 15 to 19 oxyethylene groups.

As is evident from formula (I), in the above compounds, the vector group (V) is to be understood as being outside the parentheses so that indicia m does not apply to (V) whereas all other depicted structural elements are to be understood as being within the parentheses and thus present in the molecule m times.

According to one embodiment, in the above compounds the maleimide attachment to (V) may be replaced by an opened hydrolyzed maleimide attachment. In some instances, wherein m is at least 2, said compound may comprise a mix of (closed) maleimide derivatives and opened hydrolyzed maleimide derivatives attached to V, preferably at least 50% of the attachments to V are opened hydrolyzed maleimide attachments.

In another preferred embodiment, (V) represents a moiety derived from a peptide capable of interacting with a target of interest. Non-limiting examples of peptides include somatostatin or analogues thereof, such as octreotide, Angiopep-2, Gastrin-releasing peptide, transferrin-derived peptide, derivative of the Neuropeptide Y, RGD peptides, alpha-melanocyte stimulating hormone peptide analogs, vasoactive intestinal peptide, neurotensin and luteinizing hormone-releasing hormone (LHRH) analogs.

According to yet another preferred embodiment, (V) represents a moiety derived from a non-peptidic molecule such as folic acid, hyaluronic acid, a Neurotensin Receptor 1 (NRT1) antagonist such as SR 142948A derivatives and a ligand of the prostate specific membrane antigen (PSMA) such as PSMA-617 and PSMA-11.

According to one embodiment, the target cell is selected from tumor cells, virus infected cells, microorganism infected cells, parasite infected cells, cells involved in autoimmune diseases, activated cells, myeloid cells, lymphoid cells, melanocytes, and infectious agents including bacteria, viruses, mycobacteria, fungi.

According to one preferred embodiment, the target cell is any tumor cell from a solid or liquid tumor, including but not limited to lymphoma cells, myeloma cells, myeloid cells, lymphoid cells, renal cancer cells, breast cancer cells, prostate cancer cells, ovarian cancer cells, colorectal cancer cells, gastric cancer cells, squamous cancer cells, small-cell lung cancer cells, testicular cancer cells, or any cells growing and dividing at an unregulated and quickened pace to cause cancers.

### 4. Pharmaceutical Compositions

The compounds of the present invention can be provided in the form of pharmaceutical compositions for human or animal usage in human and veterinary medicine. Such compositions typically comprise a therapeutically effective amount of LDC according to the present invention or a pharmaceutically acceptable salt thereof, and one or more components selected from a carrier, a diluent and other excipients.

In one embodiment, the pharmaceutical composition comprises a mixture of LDCs according to the present invention.

In a more specific embodiment, the pharmaceutical composition comprises a mixture of LDCs according to the present invention wherein said LDCs (compounds of formula I) comprise (closed) maleimide and/or open hydrolysed maleimide attachments to V (as described above).

The proportions of (closed) maleimide derivatives (A) and opened hydrolyzed maleimide derivatives (B) attached to V (in terms of total maleimide attachments in the composition) may be A:B/0-50:50-100%, preferably A:B/10-40:60-90%, more preferably A:B/15-35:65-85%, and most preferably about A:B/30:70%.

The respective proportions of closed maleimide derivatives and opened hydrolyzed maleimide derivatives (and thereby closed (A) and open (B) maleimide attachments) in a composition can be determined by MS techniques such as Tof or Orbitrap analysis of the reduced LDC (Compounds of formula (1)). An example of detailed protocol is available in chapter 6.d. of Chem. Eur. J. 2019, 25, 8208-8213.

Suitable carriers, diluents and other excipients for use in pharmaceutical compositions are well known in the art, and are for instance described in Remington's Pharmaceutical Sciences, Mack Publishing Co. (Gennaro AR, 1985). The carrier, diluent and/or other excipient can be selected with regard to the intended route of administration and pharmaceutical practice. The pharmaceutical compositions can comprise as the carrier, diluents and/or other excipients, or in addition to, any suitable binder(s), lubricant(s), suspending agent(s), coating agent(s), solubilizing agent(s).

The therapeutically effective amount can be determined by a physician on a routine basis. The specific dose level and frequency of dosage for any particular subject/patient can vary and depends on a variety of factors including the activity of the specific drug compound employed, the metabolic stability and length of action of that compound, the patient's age, body weight, general health, sex, diet, mode and time of administration, rate of excretion, drug combination, the severity of the particular condition, and the individual undergoing therapy. These factors are taken into account by the physician when determining the therapeutically affective dose.

### 5. Use of LDCs or Compositions Thereof in Methods of Preventing or Treating Diseases

The compounds of the present invention can be used to treat disease. The treatment can be a therapeutic and/or prophylactic treatment, with the aim being to prevent, reduce or stop an undesired physiological change or disorder. In some aspects, the treatment can prolong survival of a subject as compared to expected survival if not receiving the treatment.

The disease that is treated by the LDC can be any disease that benefits from the treatment, including chronic and acute disorders or diseases and also those pathological conditions which predispose to the disorder. In some aspects, the disease is a neoplastic disease such as cancer that can be treated via the targeted destruction of tumor cells. Non-limiting examples of cancers that may be treated include benign and malignant tumors, either solid or liquid; lymphoid malignancies e.g., Non-Hodgkin's lymphoma (NHL) e.g., Diffuse large B cell lymphoma (DLBCL), as well as breast, ovarian, stomach, endometrial, salivary gland, lung, kidney, colon, thyroid, pancreatic, prostate or bladder cancer, and cancers of the bone and blood marrow e.g., acute myeloid leukemia (AML). The disease may be a neuronal, glial, astrocytal, hypothalamic or other glandular, macrophagal, epithelial, stromal and blastocoelic disease; or inflammatory, angiogenic or an immunologic disease. An exemplary disease is a solid, malignant tumor, another exemplary disease is a liquid, malignant tumor.

According to one embodiment, the compound of the present invention or composition thereof is used in a method of treating or preventing a cancer, an autoimmune disease or inflammatory disease and/or an infectious disease, for instance by administering a therapeutically effective amount of the compound of the present invention or composition thereof to a patient in need thereof.

Non limiting examples of autoimmune, inflammatory, and/or infectious diseases include: rheumatoid arthritis, multiple sclerosis, type I diabetes mellitus, idiopathic inflammatory myopathy, systemic lupus erythematosus (SLE), myasthenia gravis, Grave's disease, dermatomyositis, polymyositis, Crohn's disease, ulcerative colitis, gastritis, Hashimoto's thyroiditis, asthma, psoriasis, psoriatic arthritis, dertmatitis, systemic scleredema and sclerosis, inflammatory bowel disease (IBD), respiratory distress syndrome, meningitis, encephalitis, uveitis, glomerulonephritis, eczema, atherosclerosis, leukocyte adhesion deficiency, Raynaud's syndrome, Sjogen's syndrome, Reiter's disease, Beheet's diasease, immune complex nephritis, IgA enphropathy, IgM polyneuropathies, immune-mediated thrombocytopenias, acute idiopathic thrombocytopenic purpura, chronic idiopathic thembocytopenic purpura, hemolytic anemia, myasthenia gravis, lupus nephritis, atopic dermatitis, pemphigus vulgaris, opsoclonus-myoclonus syndrome, pure red cell aplasia, mixed cryoglobulinermia, ankylosing spondylitis, hepatitis C-associated cryoglobulinemic vasculitis, chronic focal encephalitis, bullous pemphigoid, hemophilia A, membranoproliferative glomerulonephritis, adult and juvenile dermatomyositis, adult polymyositis, chronic urticaria, primary biliary cirrhosis, neuromyelitis optica, Graves' dysthyroid disease, bullous pemphigoid, membranoproliferative glomerulonephritis, Churg-Strauss syndrome, juvenile onset diabetes, hemolytic anemia, atopic dermatitis, systemic sclerosis, Sjogen's syndrome and glomerulonephritis, dermatomyositis, ANCA, aplastic anemia, autoimmune hemolytic anemia (AIHA), factor VIII deficiency, hemophilia A, autoimmune neutropenia, Castleman's syndrome, Goodpasture's syndrome, solid organ transplant rejection, graft versus host disease (GVHD), autoimmune hepatitis, lymphoid interstitial pneumonitis, bronchiolitis obliterans (non-transplant), Guillain-Barre Syndrome, large vessel vasculitis, giant cell (Takayasu's) arteritis, medium vessel vasculitis, Kawasaki's Disease, polyarteritis nodosa. Wegener's granulomatosis, microscopic polyangiitis (MPA), Omenn's syndrome, chronic renal failure, acute infectious mononucleosis, HIV, hepatitis, herpes, and bacterial infections. Reference is furthermore made to the medical indications mentioned in WO 2012/135740 A, e.g., in paragraphs [0004] and [0039] of this document.

In one specific embodiment, the compound of the present invention or composition thereof is used in a method of treating or preventing a cancer of the bone and blood marrow, preferably acute myeloid leukemia (AML). Preferably, said compound is a compound of the invention, wherein (V) represents a moiety derived from an anti-CD37 antibody and preferably naratuximab.

In another specific embodiment, the compound of the present invention or composition thereof is used in a method of treating or preventing a lymphoma, preferably NHL, more preferably DLBCL. Preferably, said compound is a compound of the invention, wherein (V) represents a moiety derived from an anti-CD37 antibody and preferably naratuximab.

In a more specific embodiment, the compound of the present invention or composition thereof is used in a method of treating or preventing a cancer of the bone and blood marrow, preferably acute myeloid leukemia (AML), said compound being a compound of the invention wherein (V) represents a moiety derived from an anti-CD37 antibody, preferably naratuximab, and wherein (D) is a moiety derived from an antineoplastic agent, and preferably a moiety derived from a drug selected from amanitin, duocarmycin, auristatin, auristatin F (AF), monomethyl auristatin F (MMAF), maytansine, mertansine (DM1), ravtansine (DM4), tubulysin, calicheamicin, camptothecin, SN-38, exatecan, Maaa-1181a, taxol, daunomycin, vinblastine, doxorubicin, methotrexate, pyrrolobenzodiazepine (PBD) and dimers thereof, indilinobenzodiazepine (IBD) and dimers thereof, or radioisotopes and/or pharmaceutically acceptable salts thereof.

In an even more specific embodiment, the compound of the present invention or composition thereof is used in a method of treating or preventing a cancer of the bone and blood marrow, preferably acute myeloid leukemia (AML), said compound being a compound of the invention wherein (V) represents a moiety derived from an anti-CD37 antibody, preferably naratuximab, and wherein (D) is a moiety derived from a drug selected from auristatin, MMAF, exatecan, maytansine, DM1 and DM4; more preferably a moiety derived from DM1 or DM4.

In a preferred embodiment, the compound of the present invention or composition thereof is used in a method of treating or preventing a cancer of the bone and blood marrow, preferably acute myeloid leukemia (AML), said compound being a compound of the invention wherein (V) represents a moiety derived from an anti-CD37 antibody, preferably naratuximab, (D) represents a moiety derived from DM1, and the compound is represented by one of the following formulae:

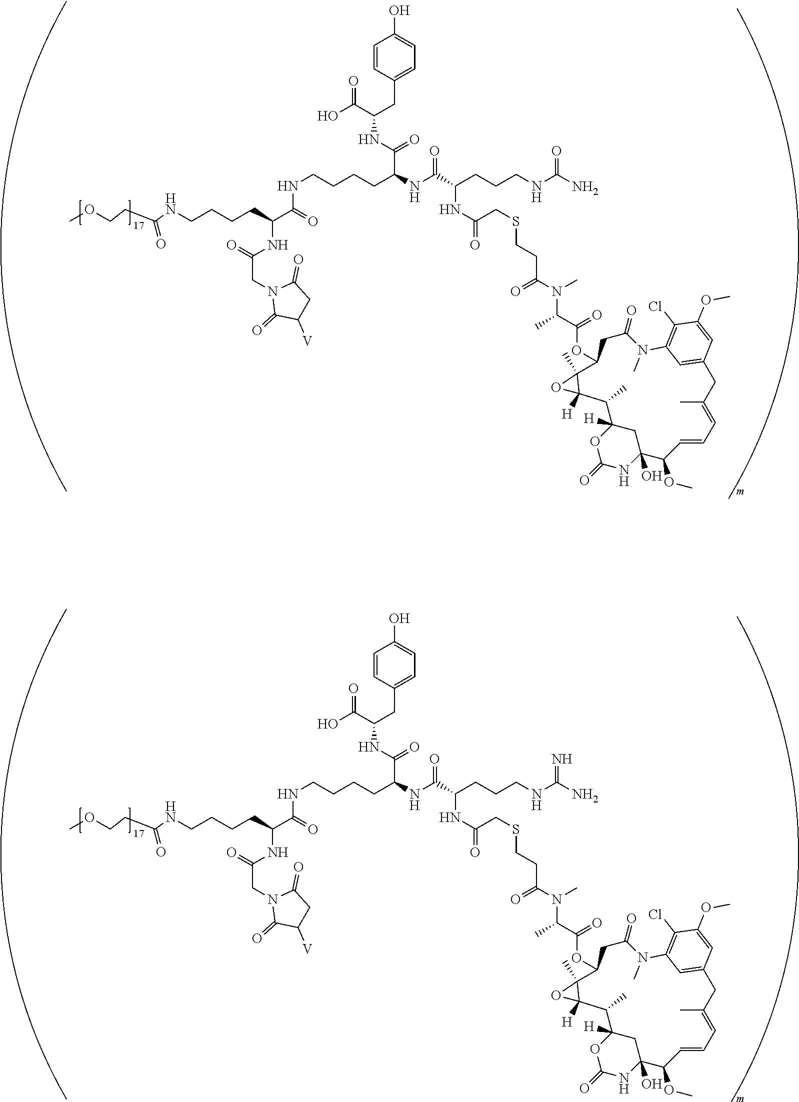

wherein m is an integer of 1 to 12, preferably 2 to 10, more preferably 4 to 8; wherein the number of oxyethylene repeating units (17) may be replaced by 12 to 30, preferably 14 to 25, more preferably 15 to 19 oxyethylene groups; and/or wherein the maleimide attachment to (V) may be replaced by an opened hydrolyzed maleimide,
and wherein preferably the compound is represented by the following formula:

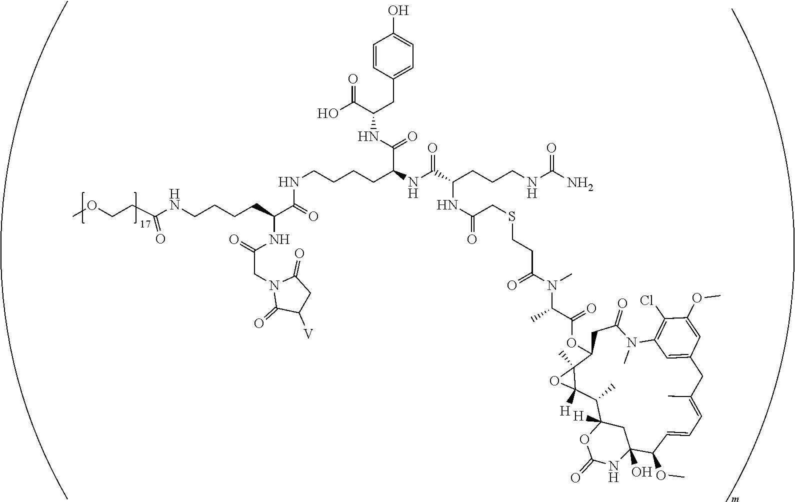

wherein m is an integer of 1 to 12, preferably 2 to 10, more preferably 4 to 8; wherein the number of oxyethylene repeating units (17) may be replaced by 12 to 30, preferably 14 to 25, more preferably 15 to 19 oxyethylene groups; and/or wherein the maleimide attachment to (V) may be replaced by an opened hydrolyzed maleimide.

As is evident from formula (I), in the above compounds, the vector group (V) is to be understood as being outside the parentheses so that indicia m does not apply to (V) whereas all other depicted structural elements are to be understood as being within the parentheses and thus present in the molecule m times.

In a more preferred embodiment, the compound of the present invention or composition thereof is used in a method of treating or preventing a cancer of the bone and blood marrow, preferably acute myeloid leukemia (AML), said compound being a compound of the invention wherein (V) represents a moiety derived from an anti-CD37 antibody, preferably naratuximab, (D) represents a moiety derived from DM1, and the compound is represented by the following formula:

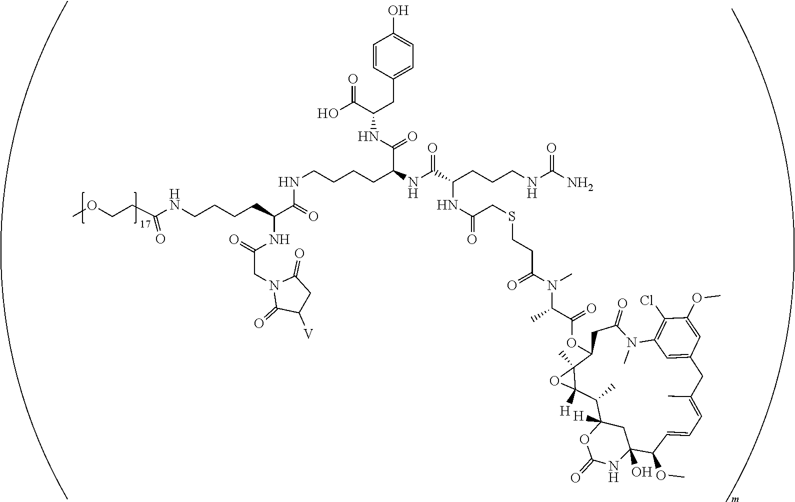

wherein m is an integer of 1 to 12, preferably 2 to 10, more preferably 4 to 8; and wherein the number of oxyethylene repeating units (17) may be replaced by 12 to 30, preferably 14 to 25, more preferably 15 to 19 oxyethylene groups.

As evident from formula (I), in the above compounds, the vector group (V) is to be understood as being outside the parentheses so that indicia m does not apply to (V) whereas all other depicted structural elements are to be understood as being within the parentheses and thus present in the molecule m times.

According to one embodiment, in the above compounds the maleimide attachment to (V) may be replaced by an opened hydrolyzed maleimide attachment. In some instances, wherein m is at least 2, said compound may comprise a mix of (closed) maleimide derivatives and opened hydrolyzed maleimide derivatives attached to V, preferably at least 50% of the attachments to V are opened hydrolyzed maleimide attachments.

In a preferred embodiment, the compound of the present invention or composition thereof is used in a method of treating or preventing a cancer of the bone and blood marrow, preferably acute myeloid leukemia (AML), said compound being a compound of the invention wherein (V) represents a moiety derived from an anti-CD37 antibody, preferably naratuximab, (D) represents a moiety derived from DM1, and the compound is represented by one of the following formulae:

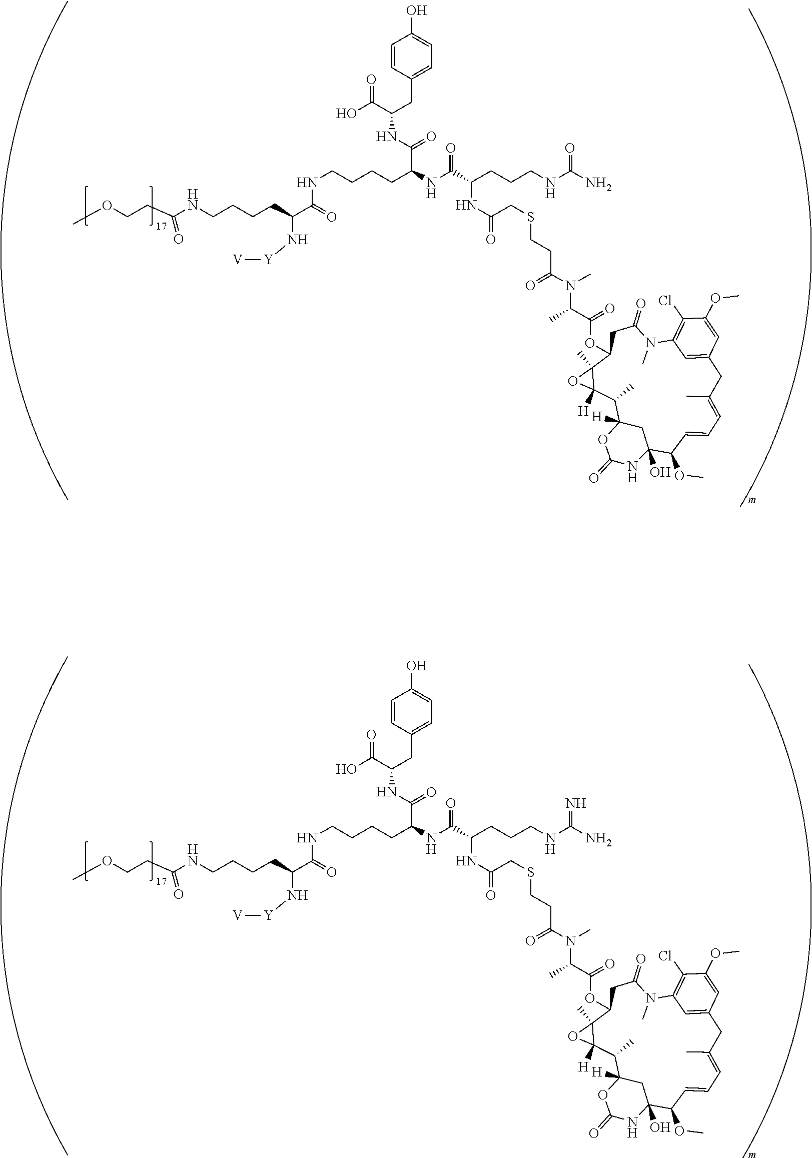

wherein m is an integer of 1 to 12, preferably 2 to 10, more preferably 4 to 8; wherein Y is represented by any of the formulae (XIIIa) to (XIIIc), preferably by formula (XIIIb) or (XIIIc); and/or wherein the number of oxyethylene repeating units (17) may be replaced by 12 to 30, preferably 14 to 25, more preferably 15 to 19 oxyethylene groups.

In a more preferred embodiment, the compound of the present invention or composition thereof is used in a method of treating or preventing a cancer of the bone and blood marrow, preferably acute myeloid leukemia (AML), said compound being a compound of the invention wherein (V) represents a moiety derived from an anti-CD37 antibody, preferably naratuximab, (D) represents a moiety derived from DM1, and the compound is represented by the following formula:

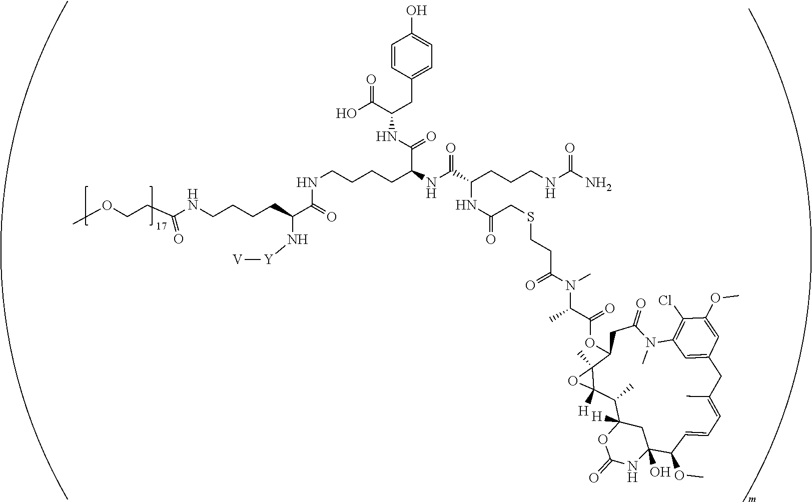

wherein m is an integer of 1 to 12, preferably 2 to 10, more preferably 4 to 8; wherein Y is represented by any of the formulae (XIIIa) to (XIIIc), preferably by formula (XIIIb) or (XIIIc); and/or wherein the number of oxyethylene repeating units (17) may be replaced by 12 to 30, preferably 14 to 25, more preferably 15 to 19 oxyethylene groups.

As evident from formula (I), in the above compounds, the vector group (V) is to be understood as being outside the parentheses so that indicia m does not apply to (V) whereas all other depicted structural elements are to be understood as being within the parentheses and thus present in the molecule m times.

The compound/molecule of the invention may be administered to a subject (e.g., a patient) in any therapeutically effective dose. Said therapeutically effective dose may depend on the characteristics of the patient in question e.g., size and weight. The molecule can be administered to a subject (e.g. a patient) at one time or over a series of treatments. Depending on the type and severity of the disease, between about 0.1 μg/kg to 1 mg/kg of drug may be used as an initial candidate dosage for first administration in a first-in-human trial, e.g. by one or more separate administrations, or by continuous infusion. A typical daily, once weekly (QW), once every two weeks (Q2W), once every 3 weeks (Q3W) or monthly dosage can range from about 0.1 mg/kg to 50 mg/kg or more, or from about 0.5 to about 25 mg/kg of patient weight. The subject to whom the compound/molecule is administered may be a patient in need thereof i.e., a patient in need of treatment e.g., a patient suffering from a disease such as cancer e.g., AML, an automimmune disease or an infectious disease.

When treating cancer, the therapeutically effect that is observed can be a reduction in the number of cancer cells; a reduction in tumor size; inhibition or retardation of cancer cell infiltration into peripheral organs; inhibition of tumor growth; and/or relief of one or more of the symptoms associated with cancer.

The routes for administration (delivery) include one or more of oral (e.g. tablet, capsule, ingestible solution), topical, mucosal (e.g. nasal spray, aerosol for inhalation), nasal, parenteral (e.g. an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual. According to a preferred embodiment, the compound of the present invention is administered by injection, such as parenterally, intravenously, subcutaneously, intramuscularly, transdermally.

According to one further embodiment, the compound of the present invention is used in a method of treating or preventing a cancer, an autoimmune disease or inflammatory disease and/or an infectious disease, and is administered concurrently with one or more other therapeutic agents such as chemotherapeutic agents, radiation therapy, immunotherapy agents, autoimmune disorder agents, anti-infectious agents, or one or more other compounds of formula (I). It is also possible to administer the other therapeutic agent before or after the compound of the present invention.

In a more specific embodiment, the compound of the present invention is used in a method of treating or preventing a cancer, preferably a lymphoma or a cancer of the blood and bone marrow, wherein said compound of the invention is administered concurrently with an anti CD20 antibody, preferably rituximab, and wherein said compound is a compound of the invention wherein (V) represents a moiety derived from an anti-CD37 antibody, preferably naratuximab, (D) represents a moiety derived from DM1, and the compound is represented by the following formula:

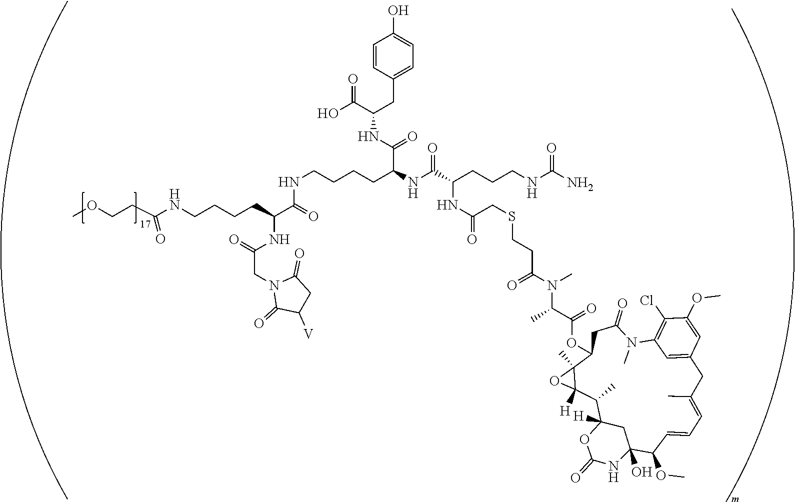

wherein m is an integer of 1 to 12, preferably 2 to 10, more preferably 4 to 8; and wherein the number of oxyethylene repeating units (17) may be replaced by 12 to 30, preferably 14 to 25, more preferably 15 to 19 oxyethylene groups; and wherein the maleimide attachment to V may be replaced by an opened hydrolyzed maleimide attachment; and
wherein preferably the lymphoma is DLBCL and the cancer of the bone and blood marrow is AML.

In a preferred embodiment, the compound of the present invention is used in a method of treating or preventing a cancer, preferably a lymphoma or a cancer of the blood and bone marrow, wherein said compound of the invention is administered concurrently with an anti CD20 antibody, preferably rituximab, and wherein said compound is a compound of the invention wherein (V) represents a moiety derived from an anti-CD37 antibody, preferably naratuximab, (D) represents a moiety derived from DM1, and the compound is represented by the following formula:

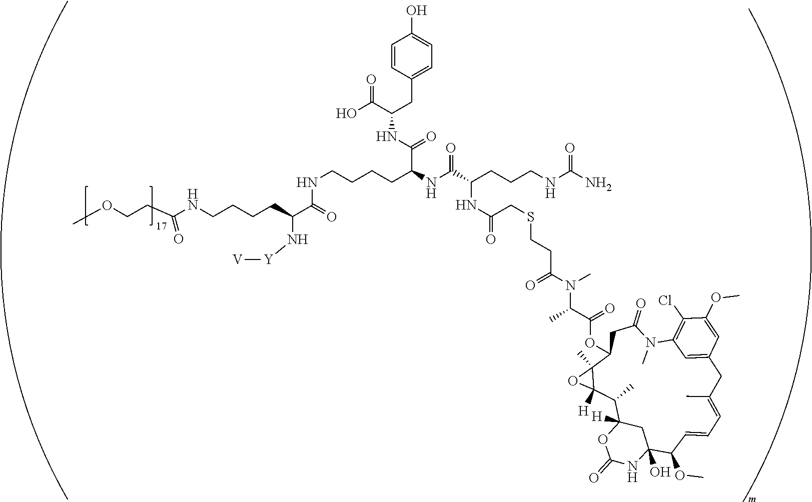

wherein m is an integer of 1 to 12, preferably 2 to 10, more preferably 4 to 8; wherein Y is represented by any of the formulae (XIIIa) to (XIIIc), preferably by formula (XIIIb) or (XIIIc); and/or wherein the number of oxyethylene repeating units (17) may be replaced by 12 to 30, preferably 14 to 25, more preferably 15 to 19 oxyethylene groups; and wherein preferably the lymphoma is DLBCL and the cancer of the bone and blood marrow is AML.

As evident from formula (I), in the above compounds, the vector group (V) is to be understood as being outside the parentheses so that indicia m does not apply to (V) whereas all other depicted structural elements are to be understood as being within the parentheses and thus present in the molecule m times.

The term “anti-CD20 antibody” or “an antibody that binds to CD20” as used herein refers to an antibody that is capable of binding CD20 with sufficient affinity such that the antibody is useful as a diagnostic and/or therapeutic agent in targeting CD20. The extent of binding of an anti-CD20 antibody to an unrelated, non-CD20 protein can be less than about 10% of the binding of the antibody to CD20 as measured, e.g., by a radioimmunoassay (RIA). In certain embodiments, an antibody that binds to CD20 has a dissociation constant (Kd) of ≤1 μM, ≤1100 nM, ≤110 nM, ≤11 nM, or ≤1.1 nM.

Similarly, as used herein, the terms “anti-CD37 antibody”, “anti-HER2 antibody”, “anti-PDL1 antibody” and “anti-EGFR antibody” (or “an antibody that binds to CD37”, “an antibody that binds to HER2”, “an antibody that binds to PDL1” and “an antibody that binds to EGFR”) refer to an antibody capable of binding CD37, HER2, PDL1 or EGFR, respectively, with sufficient affinity such that the antibody is useful as a diagnostic and/or therapeutic agent in targeting the respective antigen CD37, HER2, PDL1 or EGFR. The extent of binding of an anti-CD37, anti-HER2, anti-PDL1 or anti-EGFR antibody to a respective unrelated, non-CD37, non-HER2, non-PDL1 or non-EGFR protein can be less than about 10% of the binding of the antibody to CD37, HER2, PDL1 or EGFR as measured, e.g., by a radioimmunoassay (RIA). In certain embodiments, an antibody that binds to any of CD37, HER2, PDL1 and EGFR has a dissociation constant (Kd) of ≤1 μM, ≤1100 nM, ≤110 nM, ≤11 nM, or ≤1.1 nM.

According to one embodiment, in the above compounds the maleimide attachment to (V) may be replaced by an opened hydrolyzed maleimide attachment. In some instances, wherein m is at least 2, said compound may comprise a mix of (closed) maleimide derivatives and opened hydrolyzed maleimide derivatives attached to V, preferably at least 50% of the attachments to V are opened hydrolyzed maleimide attachments.

The rituximab may be administered before, after, or simultaneously to the compound of the present invention.

### 6. Compound of Formula (XI), Kit for the Modification of Molecules Capable of Interacting with Target Cells, and Method for the Modification of Molecules Capable of Interacting with Target Cells

In some aspects, the present invention relates to a compound, which can be used for the modification of vector molecules capable of interacting with target cells as described above, e.g., antibodies. The present invention thus relates to a compound represented by the general formula (XI):

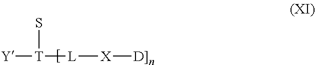

wherein,
D, X, L, T, S and n have the same meanings as described above with respect to the compound of formula (I); and
Y′ represents a moiety comprising a conjugation group capable of forming a covalent attachment to a molecule capable of interacting with a target cell (V′) such as a monoclonal antibody or an antibody fragment optionally incorporated into an Fc-fusion protein.

According to one embodiment, Y′ in formula (XI) is a moiety comprising a conjugation group selected from:
an optionally substituted maleimide, preferably capable of reacting with one or two thiol groups of (V′), wherein said thiol groups of (V′) may each independently and optionally be substituted or protected, e.g., with a monomethoxytrityl group,
an optionally substituted haloacetamide, preferably capable of reacting with a thiol group of (V′),
an ester, preferably capable of reacting with the side chain of an amino acid of (V′) (e.g., Lys) such as an acyl halide, an N-hydroxy succinimide ester (structure shown below, left hand side, α indicates covalent attachment to the remaining of the compound of formula (XI)) or a phenolic ester (structure shown below, right hand side, α indicates covalent attachment to the remaining of the compound of formula (XI), each R being individually selected from H, F, N02 and CN),

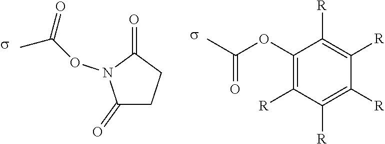

a carbonate, preferably capable of reacting with the side chain of an amino acid of (V′) (e.g., Lys) such as a haloformate or a carbonate comprising a leaving group such as a N-hydroxy succinimide or phenol derivative,
an isocyanate or isothiocyanate, preferably capable of reacting with the side chain of an amino acid (e.g., Lys) of (V′),
an azide, preferably capable of reacting with an alkyne group comprised in a molecule (V′);
an alkyne, preferably capable of reacting with an azide group comprised in a molecule (V′); and
an amino group, e.g., a primary or secondary amino group, preferably capable of reacting with a molecule (V′), e.g., an antibody, the presence of an enzyme such as a transglutaminase.

According to one preferred embodiment, the compound has a structure represented by the general formula (XII) or (XII′):

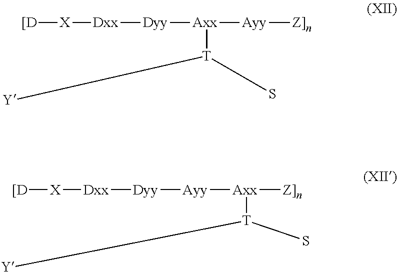

wherein,
Axx in formulae (XII) and (XII′) has the same meanings as in formulae (X) and (X′) described above; Ayy in formula (XII) has the same meanings as in formula (X) described above; Ayy in formula (XII′) has the same meanings as in formula (X′) described above, Y′ has the same meanings as described above with regard to formula (XI); and
D, Dxx, Dyy, X, T, S, Z and n have the same meanings as described above with regard to the ligand-drug-conjugate.

According to one preferred embodiment, at least one, e.g., two, three, four, five, six or seven, of D, Dxx, Dyy, X, T, S and Z in formulae (XII) and (XII′) is/are defined as follows:
(a) D is a moiety derived from a drug selected from auristatin, MMAF, exatecan, maytansine, DM1 and DM4, preferably a moiety derived from DM1 or DM4;
(b) Dxx is a moiety derived from an amino acid selected from Phe, Val, Tyr, homo-Phe and Ala, preferably from Phe or Val;
(c) Dyy is a covalent bond or a moiety derived from an amino acid selected from Arg, Lys, Cit, Orn, Dap and Dab, preferably from Arg or Cit;
(d) X is a group of formula (III) wherein n2 is 1 or 2, or a group represented by any of formulae (IVa) to (IVz), preferably a group represented by any of formula (IVc), (IVm), (IVn), (IVo), (IVp), (IVs) or (IVt), more preferably by formula (IVc) or (IVm);
(e) T is a group of formula (VII), (VIII) or (IX);
(f) S is a moiety of formula (V); and
(g) Z is —OH.

According to a further preferred embodiment, each Dyy-Dxx-Axx-Ayy in formula (XII) is independently selected from Arg-Lys-Phe wherein Dyy is a covalent bond, Arg-Lys-homoPhe wherein Dyy is a covalent bond, Arg-Lys-Tyr wherein Dyy is a covalent bond, Cit-Lys-Phe wherein Dyy is a covalent bond, Cit-Lys-Tyr wherein Dyy is a covalent bond, Arg-Lys-homoTyr wherein Dyy is a covalent bond, Cit-Lys-homoTyr wherein Dyy is a covalent bond, Phe-Cit-Lys-Phe, Phe-Cit-Lys-Tyr, Phe-Arg-Lys-Tyr, Phe-Cit-Lys-homoTyr, Phe-Lys-Lys-Phe, homoPhe-Arg-Lys-Phe, homo-Phe-Cit-Lys-Tyr; and each Dyy-Dxx-Ayy-Axx in formula (XII′) is independently selected from Arg-Phe-Lys wherein Dyy is a covalent bond, Arg-Ser-Lys wherein Dyy is a covalent bond, Cit-Phe-Lys wherein Dyy is a covalent bond, Cit-Ser-Lys wherein Dyy is a covalent bond, Cit-homoPhe-Lys wherein Dyy is a covalent bond, Phe-Cit-Phe-Lys, homoPhe-Cit-Phe-Lys, and Phe-Arg-Phe-Lys.

According to yet another preferred embodiment, the compound has a structure represented by one of the following formulae:

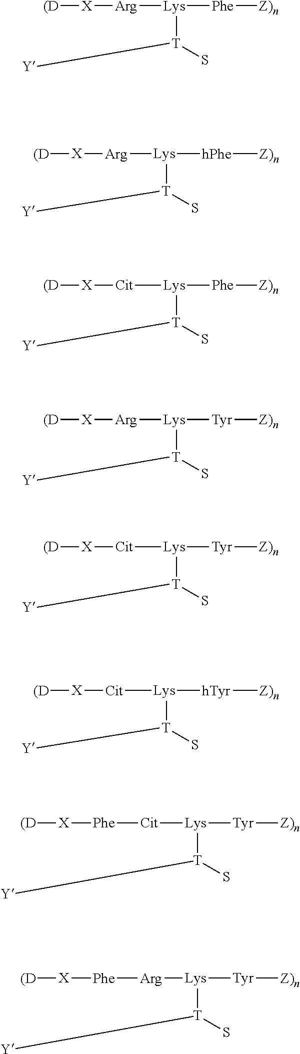

wherein D, X, T, S, Y′, Z and n have the same meanings as described above.

According to one preferred embodiment, at least one, e.g., two, three, four or five, of D, X, T, S and Z in the above formulae is/are defined as follows:
(a) D is a moiety derived from a drug selected from auristatin, AF, MMAF, exatecan, maytansine, DM1 and DM4, preferably a moiety derived from DM1 or DM4;
(d) X is a group of formula (III), wherein n2 is 1 or 2, or a group represented by any of formulae (IVa) to (IVz), preferably a group represented by any of formula (IVc), (IVm), (IVn), (IVo), (IVp), (IVs) or (IVt), more preferably by formula (IVc) or (IVm);
(e) T is a group of formula (VII), (VIII) or (IX);
(f) S is a moiety of formula (V); and
(g) Z is —OH.

In one embodiment, the compound is represented by one of the following formulae:

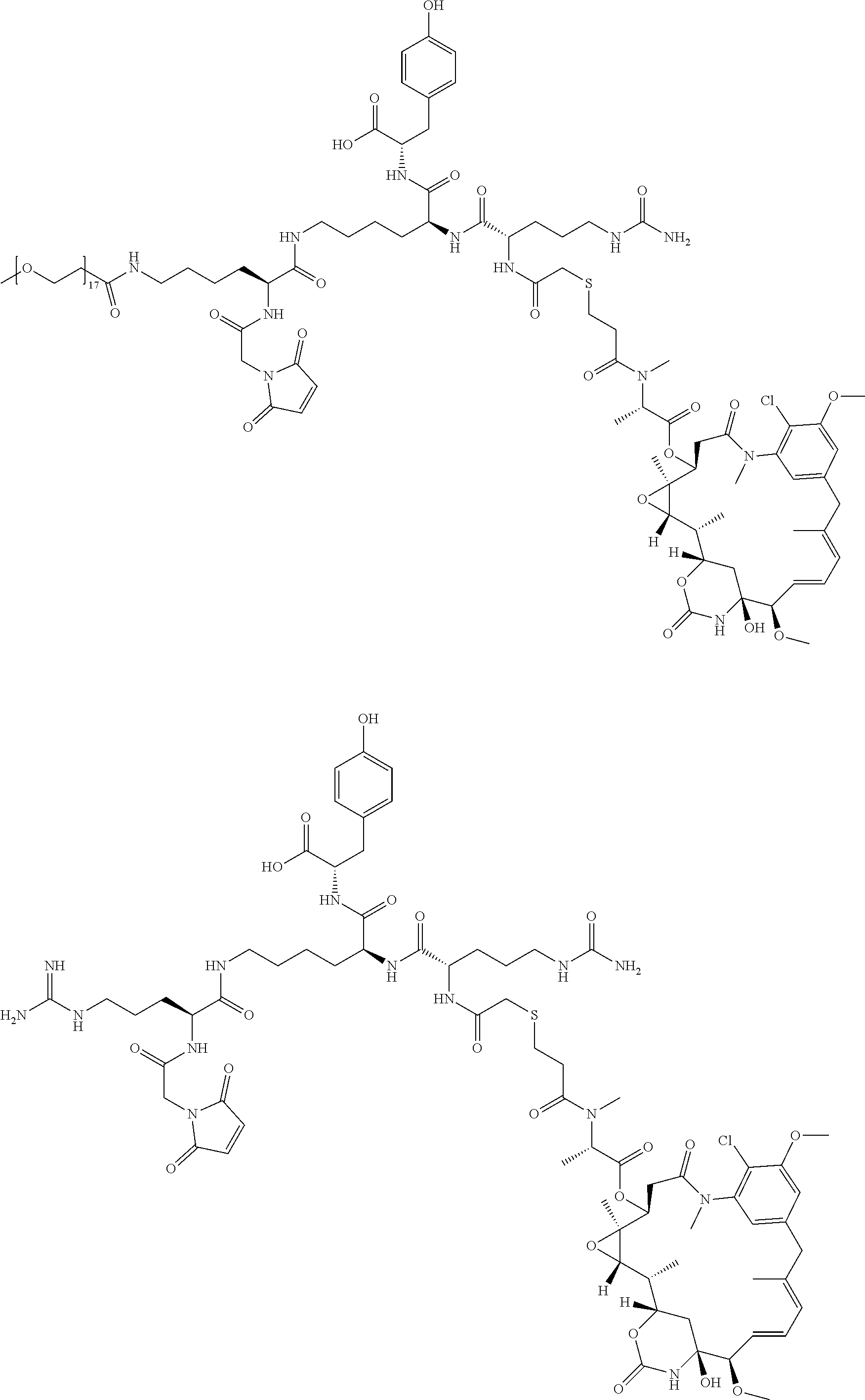

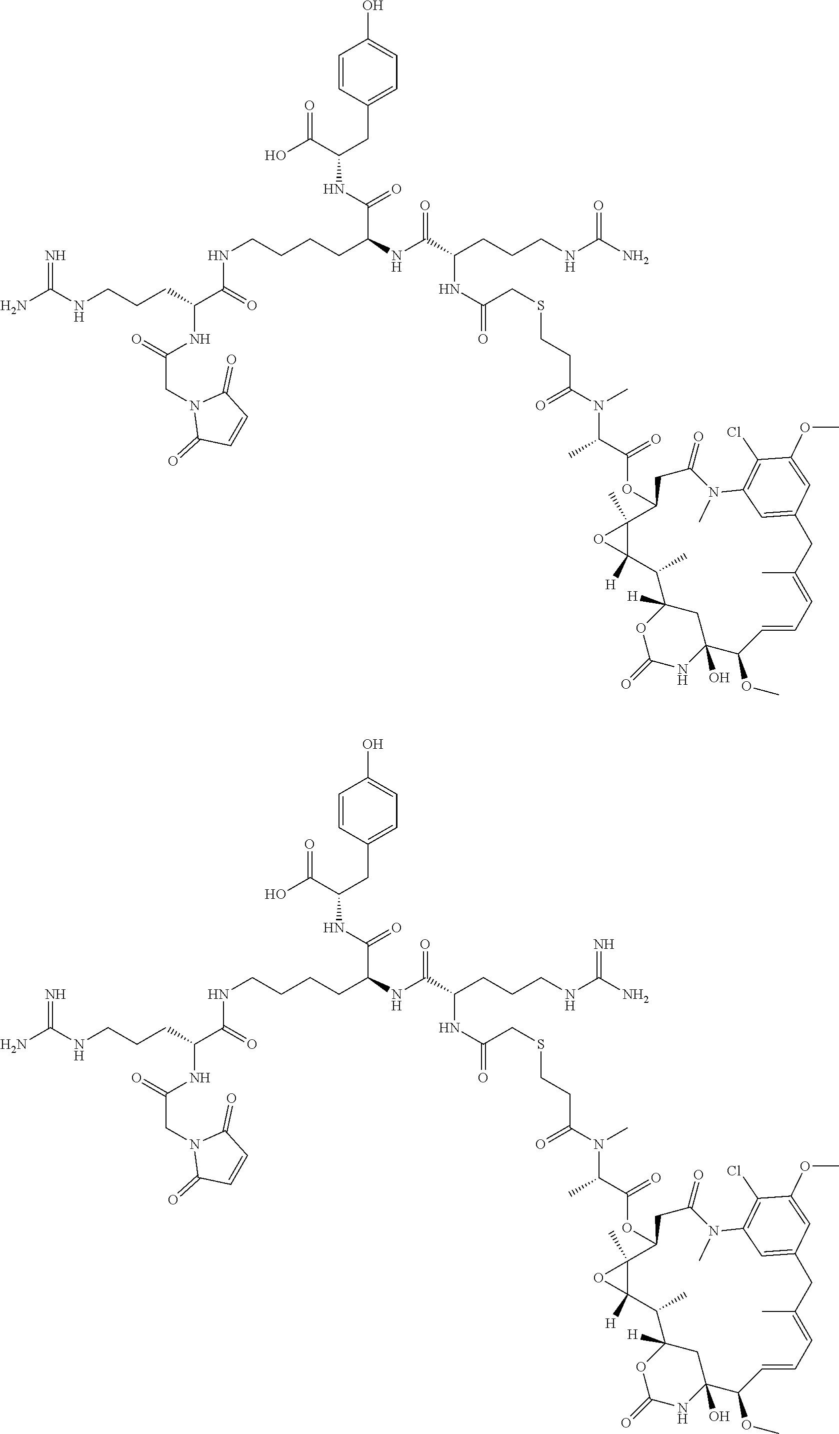

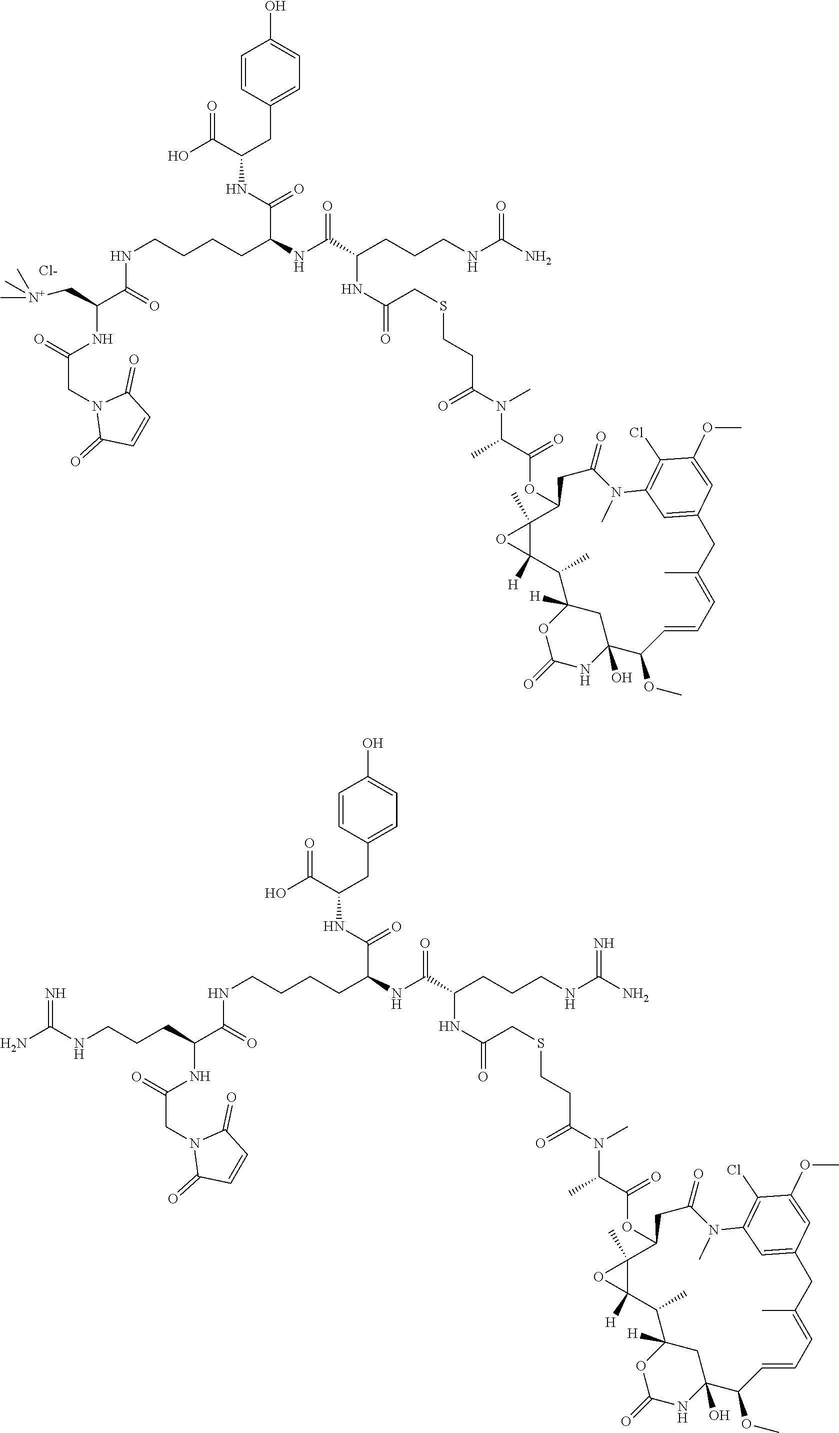

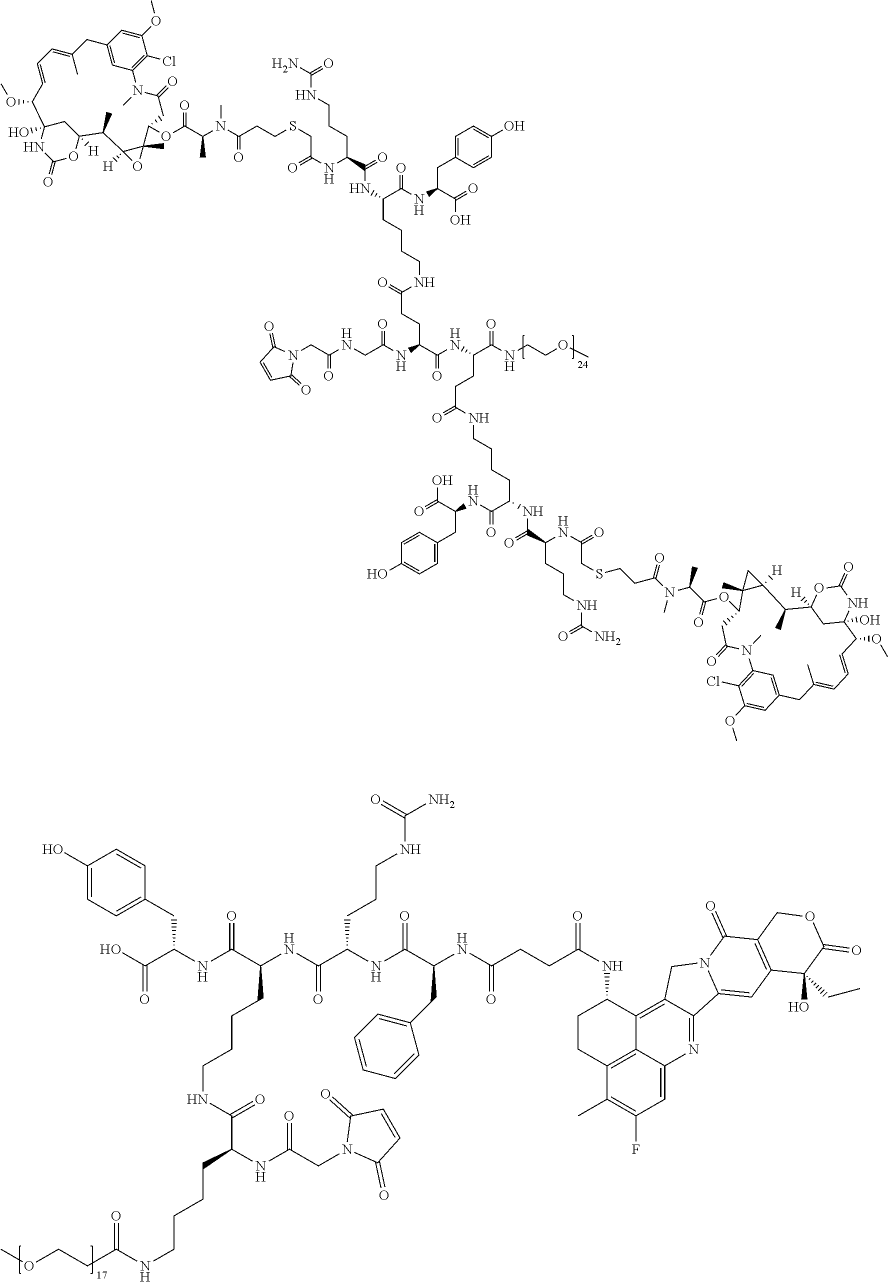

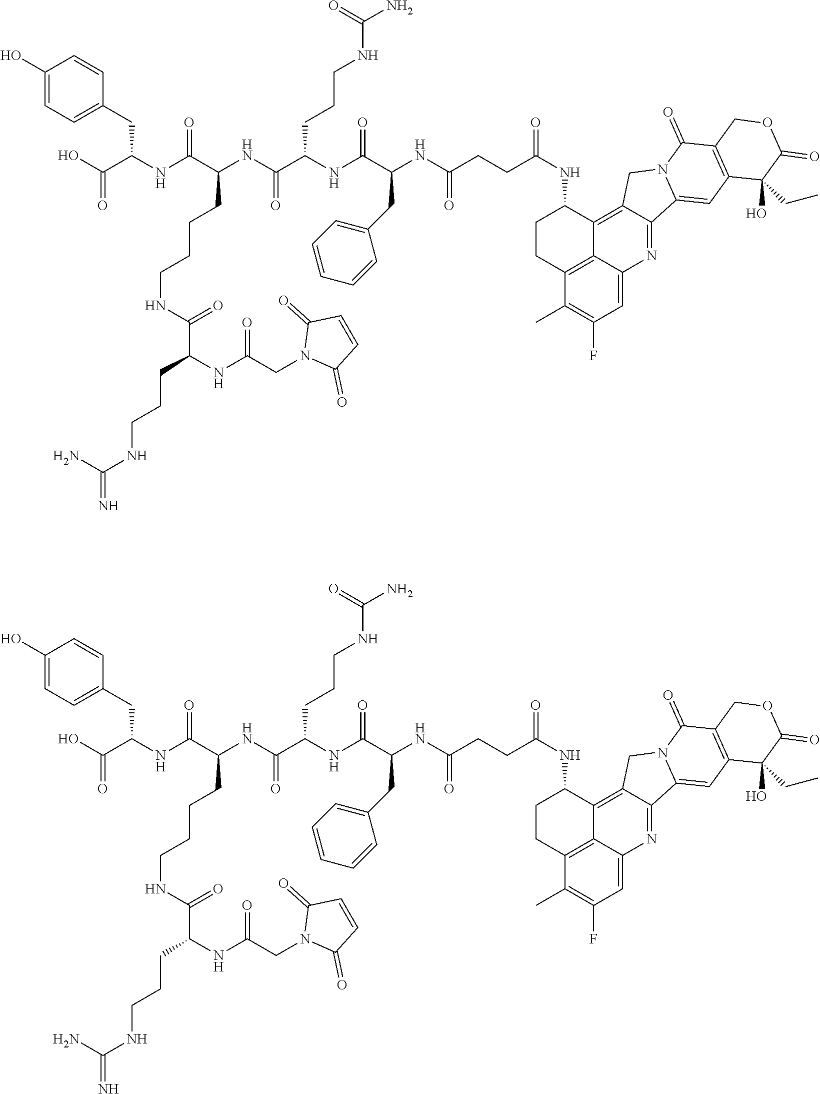 [SMILES: CC[C@@]1(O)C(=O)OCc2c1cc1n(c2=O)Cc2c-1nc1cc(F)c(C)c3c1c2[C@@H](NC(=O)CCC(=O)N[C@@H](Cc1ccccc1)C(=O)N[C@@H](CCCNC(N)=O)C(=O)N[C@@H](CCCCNC(=O)[C@H](CCCNC(=N)N)NC(=O)CN1C(=O)C=CC1=O)C(=O)N[C@@H](Cc1ccc(O)cc1)C(=O)O)CC3 ; CC[C@@]1(O)C(=O)OCc2c1cc1n(c2=O)Cc2c-1nc1cc(F)c(C)c3c1c2[C@@H](NC(=O)CCC(=O)N[C@@H](Cc1ccccc1)C(=O)N[C@@H](CCCNC(N)=O)C(=O)N[C@@H](CCCCNC(=O)[C@@H](CCCNC(=N)N)NC(=O)CN1C(=O)C=CC1=O)C(=O)N[C@@H](Cc1ccc(O)cc1)C(=O)O)CC3]

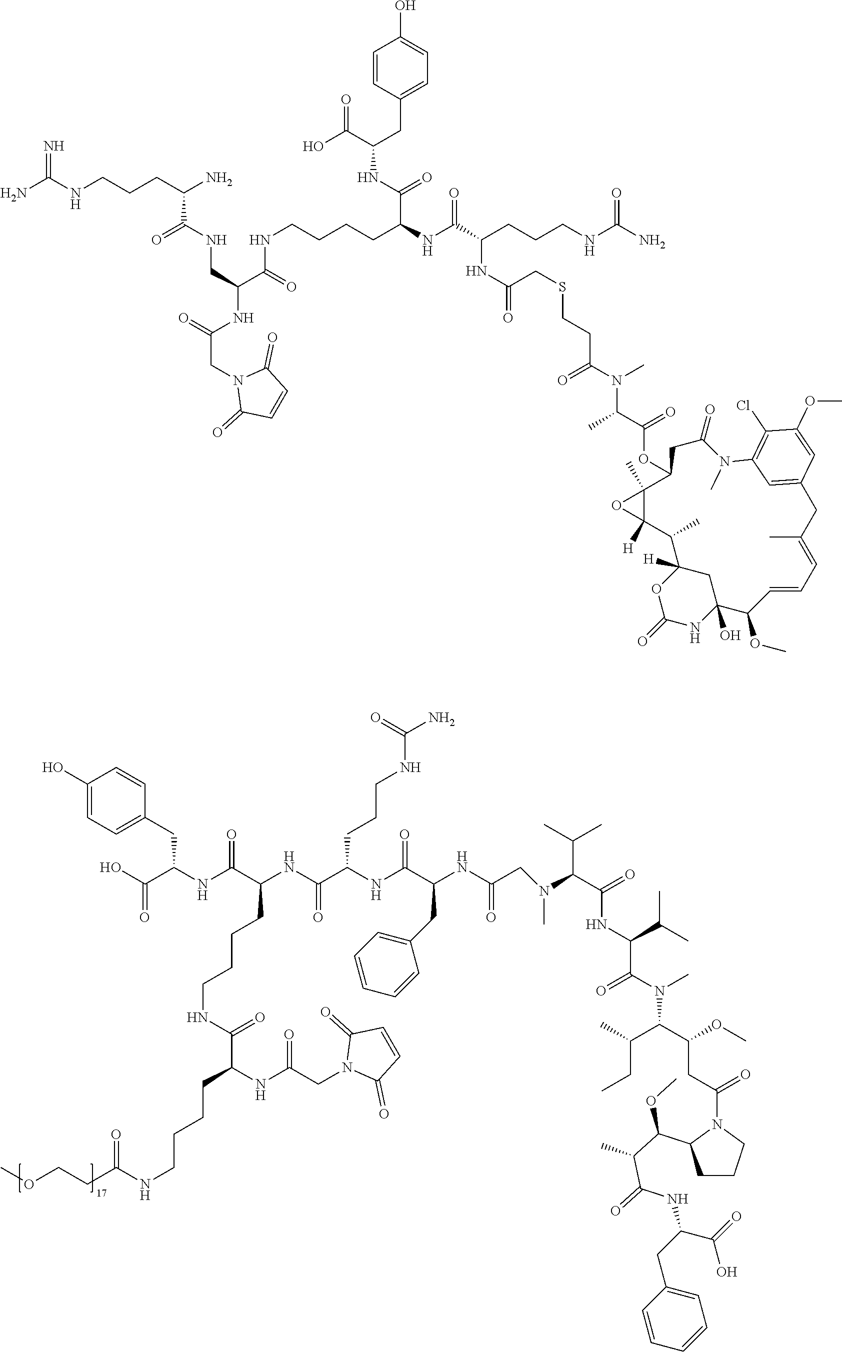

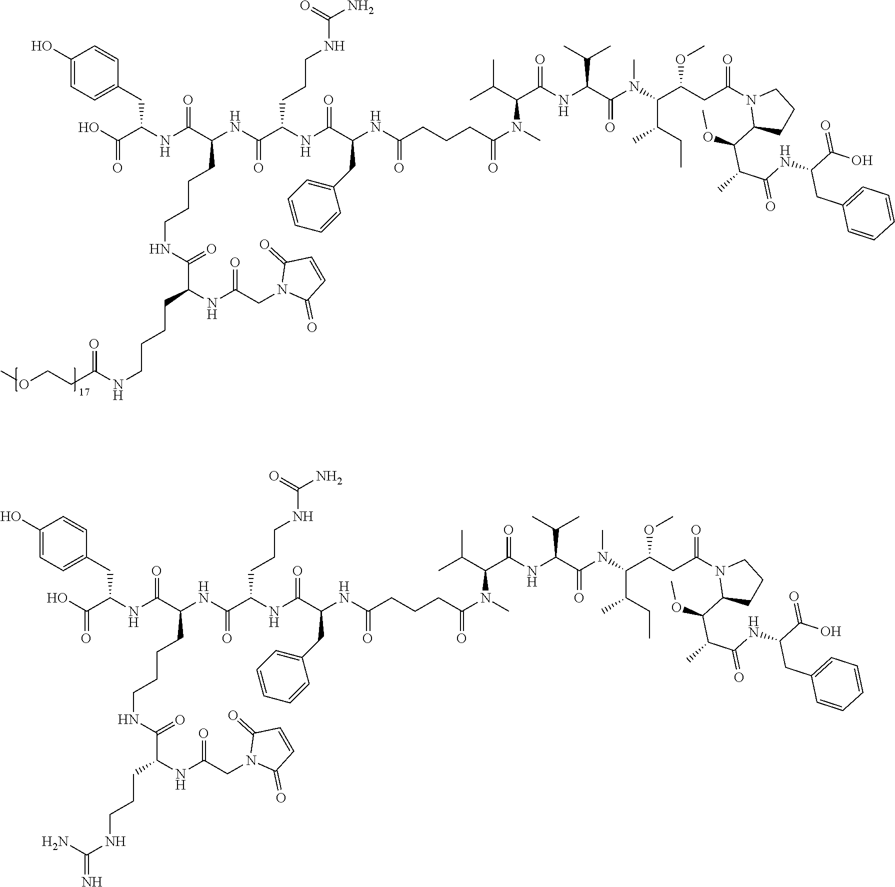

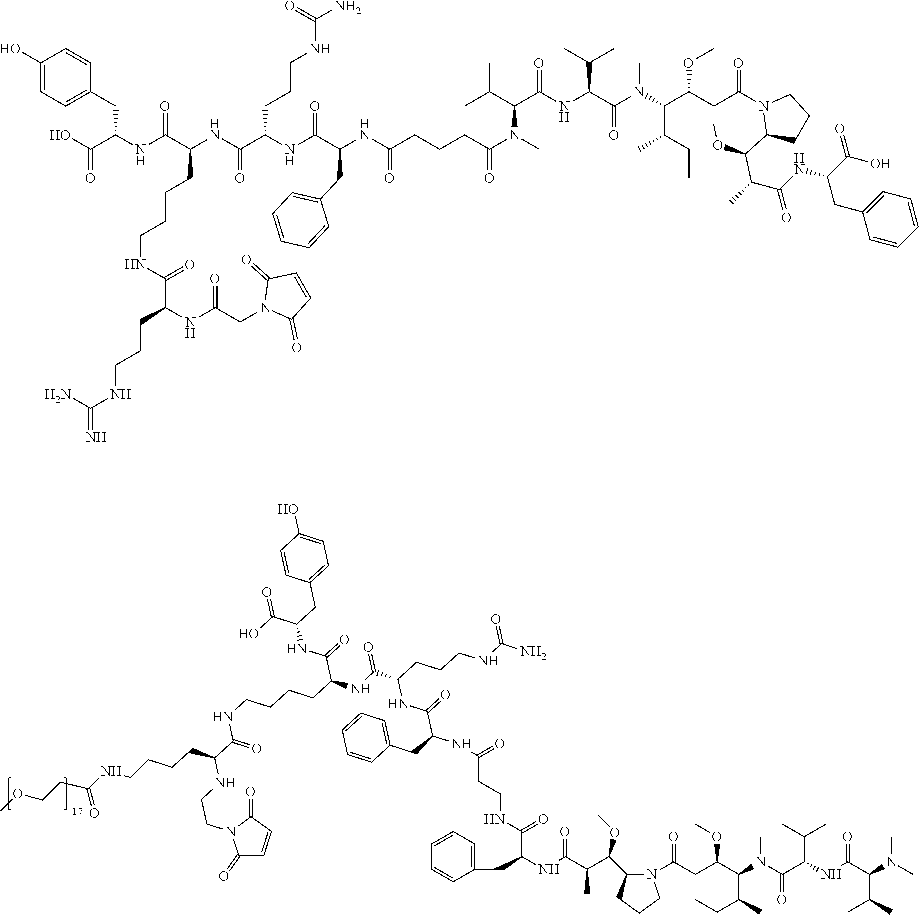

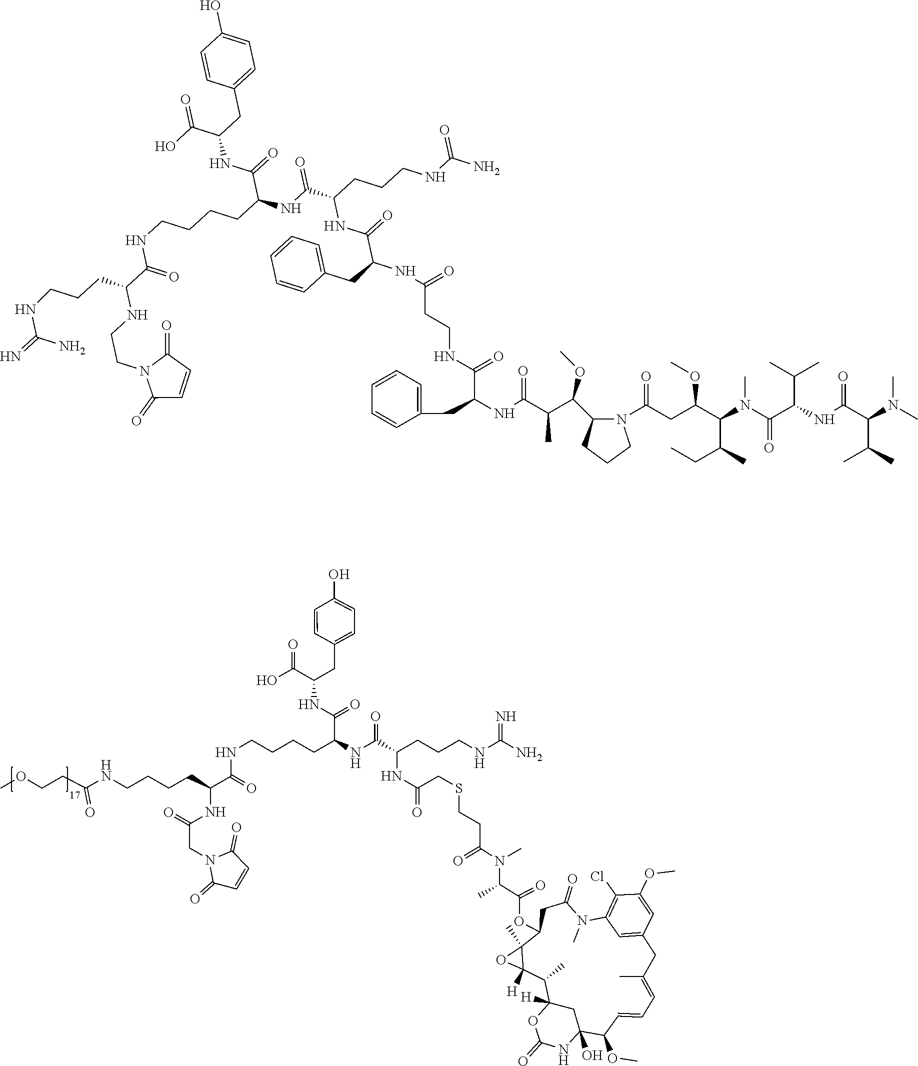

In some aspects, the present invention relates to a kit comprising the compound of described hereinbefore (i.e., the compound according to formula (XI) (XII) or (XII′)) and a buffer, which can be used for the modification of vector molecules capable of interacting with target cells, e.g., antibodies or fragments thereof, said antibody fragments being optionally incorporated into Fc-fusion proteins, in particular for the modification of therapeutic antibodies.

The compound and the buffer (together forming the kit) can be presented individually, e.g. in separate primary containers (which may be shipped to the customer in a single box), which can be stored for a prolonged period, without degradation. The compound and buffer can be formulated and proportioned for a given amount of antibody or fragment thereof to be modified. In some aspects, the compound of the present invention is presented as a solid (e.g. as a lyophilized powder, or non-covalently adsorbed or covalently bound to a solid phase matrix as described further below), or as a solution in a suitable solvent, such as a water-miscible, polar aprotic solvent (e.g. DMF, DMSO), which can be mixed with the buffer shortly prior to antibody or antibody fragment modification.

The buffer to be used in the kit of the present invention is not particularly limited. Preferably, the buffer has a pH of 6.0 to 10, more preferably of 6.5 to 8.0. The buffer can be selected from e.g. 2-bis(2-hydroxyethyl)amino acetic acid (Bicine), carbonate-bicarbonate, tris(hydroxymethyl)methylamino propane sulfonic acid (TAPS), 4-(2-hydroxyethyl)-1-piperazineethane sulfonic acid (HEPES).

In some aspects, the compound of the present invention can be used in a method for the modification of vector molecules capable of interacting with target cells, e.g., antibodies. The method thus produces a ligand-drug-conjugate of formula (I) as described above.

In one embodiment, the method comprises the step of reacting (contacting) a molecule capable of interacting with a target cell, such as a monoclonal antibody or an antibody fragment optionally incorporated into an Fc fusion protein. The reaction mixture can be purified by techniques known in the art, such as diafiltration techniques or gel permeation chromatography using a suitable solvent. Examples of suitable stationary phases for isolating the clean conjugate include polyacrylamide gels, such as Bio-Gel® P-30 and crosslinked dextrans such as Sorbadex®, Zetadex® or Sephadex®.

The method can be applied to any molecule capable of interacting with a target cell. Preferably, the method is applied to a molecule selected from antibodies, antibody fragments, proteins, peptides and non-peptidic molecules. In one embodiment, the method is applied to a monoclonal antibody (mAb) or an antibody fragment incorporated into an Fc fusion protein, preferably to an antibody selected from the group consisting of adalimumab, aducanumab, alemtuzumab, altumomab pentetate, amivantamab, atezolizumab, anetumab, avelumab, bapineuzumab, basiliximab, bectumomab, belantamab mafadotin, bermekimab, besilesomab, bevacizumab, bezlotoxumab, brentuximab, brentuximab vedotin, brodalumab, catumaxomab, cemiplimab, cetuximab, cinpanemab, clivatuzumab, crenezumab, tetraxetan, daclizumab, daratumumab, denosumab, dinutuximab, dostarlimab, durvalumab, edrecolomab, elotuzumab, emapalumab, enfortumab, enfortumab vedotin, epcoritamab, epratuzumab, epratuzumab-SN-38, etaracizumab, gemtuzumab, gemtuzumab ozogamycin, genmab, girentuximab, glofitamab, gosuranemab, ibritumomab, inebilizumab infliximab, inotuzumab, inotuzumab ozogamicin, ipilimumab, isatuximab ixekizumab, J591 PSMA-antibody, labetuzumab, lecanemab, loncastuximab tesirin, mogamulizumab, mosunetuzumab, necitumumab, nimotuzumab, natalizumab, naratuximab, naxitamab, nivolumab, ocrelizumab, ofatumumab, olaratumab, oregovomab, panitumumab, pembrolizumab, pertuzumab, polatuzumab, polatuzumab vedotin, prasinezumab, racotumomab, ramucirumab, rituximab, sacituzumab, sacituzumab govitecan, semorinemab, siltuximab, solanezumab, tafasitamab, tacatuzumab, teprotumumab, tilavonemab, tocilizumab, tositumomab, trastuzumab, trastuzumab deruxtecan, trastuzumab emtansine, TS23, ustekinumab, vedolizumab, votumumab, zagotenemab, zalutumumab, zanolimumab, fragments and derivatives thereof; more preferably from atezolizumab, durvalumab, pembrolizumab, rituximab or trastuzumab; or to an antibody fragment incorporated into an Fc-fusion protein, which is preferably selected from belatacept, aflibercept, ziv-aflibercept, dulaglutide, rilonacept, romiplostim, abatacept and alefacept.

In one embodiment, the method is applied to an anti-HER2, anti-CD37, anti-PDL1 or anti-EGFR antibody, preferably to an antibody selected from trastuzumab, pembrolizumab, naratuximab, atezolizumab, durvalumab, avelumab, panitumumab and cetuximab, more preferably to an antibody selected from naratuximab, trastuzumab, and cetuximab, and most preferably to naratuximab or trastuzumab.

### 7. Preparation of the Compounds of the Invention

In the following, methods are provided for the preparation of linkers, drug-linkers and ligand-drug-conjugates. The compounds of the invention can be synthesized relying on standard organic chemistry reactions or Fmoc-based solid-phase peptide synthesis (SPPS), including in solution and on-resin peptide coupling and convergent strategies.

The introduction of various maleimido-derivatives and subsequent chemoselective ligation to moieties derived from a vector group is also exemplified below. The general strategies and methodology which can be used for preparing the compounds of the present invention are well-known to the person skilled in the art.

### 8. Examples

### 8.1 List of Abbreviations Used in the Examples

Ac: Acetyl
ADC: Antibody-Drug Conjugate
ACN: acetonitrile
AMAS: N-α-maleimidoacet-oxysuccinimide ester
AML: Acute myeloid leukemia
Arg: Arginine
Bn: benzyl
Boc: tert-butyloxycarbonyl
Bu: butyl
Cit: Citrulline
DAR: Drug to Antibody Ratio
DBU: 1,8-Diazabicyclo[5.4.0]undec-7-ene
DCM: dichloromethane
DIEA: diisopropylethylamine
DLBCL: Diffuse large B cell lymphoma
DM1: Mertansine
DMF: dimethyl formamide
DMSO: dimethyl sulfoxide
DOC: degree of conjugation
DPBS: Dulbecco's phosphate-buffered saline
EDC: 1-Ethyl-3-(3-dimethylaminopropyl)carbodiimide
Et: ethyl
eq.: equivalent
FA: formic acid
Fmoc: 9-Fluorenylmethoxycarbonyl
g: gram
Gly: Glycine
Glu: Glutamic acid
h: hour
HATU: 1-[Bis(dimethylamino)methylene]-1H-1,2,3-triazolo[4,5-b]pyridinium 3-oxide
HPLC: high-performance liquid chromatography
HRMS: high resolution mass spectrometry
IR: Infrared
L: litre
Lys: Lysine
m: milli
ma: maleimidoacetic acid
mAb: monoclonal antibody
Me: methyl
min: minute
Mtt: 4-methyltrityl
mol: molar
MS: mass spectroscopy
m/z: ratio mass over charge
NHS: N-hydroxysuccinimide
Nmab: Naratuximab
Pbf: 2,2,4,6,7-pentamethyldihydrobenzofuran-5-sulfonyl
PBS: phosphate-buffered saline
PEG: polyethylene glycol
PEG16 or Peg16: CO—CH₂CH₂O—(CH₂CH₂O)₁₆—CH₃
PF: CentriPure PF filtration columns
PFP: pentafluorophenyl
pH: potential for hydrogen
Phe: Phenylalanine
quant.: quantitative
rt: room temperature
Rt: retention time
SMCC: Succinimidyl 4-(N-maleimidomethyl)cyclohexane-1-carboxylate
Suc: succinic
TCEP: tris(2-carboxyethyl)phosphine
TFA: trifluoroacetic acid
Tmab: Trastuzumab
Tyr: Tyrosine
UPLC: ultra-performance liquid chromatography
UV: ultraviolet
μ: micro

### 8.2 Starting Materials and Chemicals:

The main starting materials and chemicals used in the following examples are listed below:
Solvents for synthesis and deprotection reagents from Merck or Fischer Scientific AG (Switzerland);
TFA, DIEA, N-hydroxysuccinimide, DBU, DPBS, 10×DPBS and H-Glu(OtBu)-OBn from Sigma-Aldrich (Switzerland);
Fmoc-Cit-OPFP, H-Lys(Boc)-OH, Fmoc-Glu(OtBu)-OH, H-Tyr(OtBu)-OtBu, Fmoc-Lys(Mtt)-OH, Fmoc-Cit-OH, Fmoc-Phe-OH and Boc-Cit-OH from Bachem (Switzerland);
AMAS from Astatech Inc (USA);
Solvents and chemicals for high-performance liquid chromatography (HPLC) and ultra-performance liquid chromatography mass spectrometry (UPLC-MS) from Biosolve (France);
PEG24-amine from BroadPharm (USA);
HATU from Combi-Blocks (USA);
Exatecan mesylate from Angel Pharmatech, Ltd. (China);
LiOH·H₂O from VWR (Switzerland);
DM1-SMCC from Enovation Chemicals (USA);
Boc-Gly-OH and dihydrofuran-2,5-dione from Fluka (USA);
EDC·HCl from Apollo Chemical (USA);
DM1 from Angel Pharmatech, Ltd. (USA);
TCEP·HCl, Boc-Lys-OH from Fluorochem (UK);
BrCH₂ COOH, H-Tyr-OMe and Pd/C from Acros Organics (Belgium);
H-Cit-Lys(PEG5-ma)-Tyr-OH from Ambiopharm (USA)
Herceptin® (trastuzumab) from Roche (Switzerland);
Naratuximab—antibody huCD37-3 (version 1.0) described in WO2019/229677;
mPEG16-NHS ester from PurePEG (mPEG₁₆-OCH₂CH₂COO—NHS ester, m=methyl), LLC (USA);
N-succinimidyl-4-(maleimidomethyl)-cyclohexanecarboxylate (SMCC) from Sigma Aldrich Fine Chemicals.

### 8.3 Methods:

The following methods were used to evaluate the compounds and conjugates of the present invention:

### 8.3.1 Purity Determination

The purity of the compounds was determined on UPLC-MS systems:
Method 1: Waters Acquity UPLC System coupled to a Waters SQD mass spectrometer with a CSH C18 column (130 Å, 1.7 μm, 2.1 mm×50 mm) heated at 40° C. using solvent systems A (water+0.1% FA) and B (ACN+0.1% FA) at a flow rate of 0.9 mL/min and a 5-100% gradient of B over 2.7 min.
Method 2: Waters Acquity UPLC System coupled to a Waters SQD mass spectrometer with a CSH Fluoro-phenyl column (130 Å, 1.7 μm, 2.1 mm×50 mm) heated at 40° C. using solvent system A (water+0.1% FA) and B (ACN+0.1% FA) at a flow rate of 0.9 mL/min and a 5-100% gradient of B over 2.9 min.
Method 3: Waters Acquity UPLC System coupled to a Waters SQD mass spectrometer, BEH C18 1.7 μm 50×2.1 mm column heated at 40° C. and fitted with 2 μm insert filter pre-columns (available from Waters), and solvent systems A1 (water+0.1% FA) and B1 (ACN+0.1% FA) at a flow rate of 0.9 mL/min and a 5-100% gradient of B1 over 2.9 min.
Method 4: Waters Acquity UPLC System coupled to a Waters SQD mass spectrometer with a CSH C18 column (130 Å, 1.7 μm, 2.1 mm×50 mm) heated at 40° C. using solvent system A (water+0.1% FA) and B (ACN+0.1% FA) at a flow rate of 0.6 mL/min and a 5-85% gradient of B over 5 min.
Method 5: Equipment: Shimadzu LCMS 2020 Mass Spectrometer. Column: HALO C18 2.7 μm, 3.0 mm×30 mm. Mobile Phase: ACN (0.05% TFA)—water (0.05% TFA). Gradient: MeCN from 5% to 95% over 1.4 min, hold 0.6 min, total run time is 2.5 min, flow rate: 1.8 mL/min. Column temperature: 50° C. Wavelength: 214 and 254 nm PDA.
Method 6: Equipment: Shimadzu LCMS 2020 Mass Spectrometer. Column: Kinetex C18 2.6 μm, 4.6 mm×50 mm. Mobile Phase: ACN (0.05% TFA)—water (0.05% TFA); Gradient: MeCN from 5% to 95% over 1.8 min, hold 0.7 min, total run time is 3.0 min, flow rate: 1.8 mL/min. Column temperature: 50° C.

### Wavelength: 214 and 254 nm PDA.

### 8.3.2 Aggregates: Size Exclusion Chromatography (SEC)

The aggregate content of the conjugates was determined using the following method:

| Equipment 1 | UPLC Waters Acquity | |
|---|---|---|
| Equipment 2 | UPLC Waters Acquity H-Class plus Bio | |
| Detector | Tunable UV detector (TUV) | |
| Detector cell | Titanium | |
| Pre-column | Agilent AdvanceBio SEC 300 Å 2.7 μm 4.6*50 mm | |
| Column | Agilent AdvanceBio SEC 300 Å 2.7 μm 4.6*150 mm | |
| Mobile phase | Phosphate de potassium 50 mM pH 6.8/250 mM KCl | |
| Wavelength | 280 | nm |
| Injection volume | 10 | μL |
| Column temperature | Ambient | |
| Sample manager temperature | 25° | C. |
| Run time | 10 | min |
| Flow | 0.35 | mL/min |

| | |
|---|---|
| Isocratique mode | |
| Weak wash | H₂O/ACN (90/10 v/V) |
| Strong wash | H₂O/ACN (10/90 v/v) |

Mobile Phase preparation: Potassium phosphate 50 mM pH 6.8/250 mM KCl

Weight 3.61 g KH₂PO₄ and 4.09 g K₂HPO₄ into a 1 L volumetric flask. Complete with milliQ water. If necessary, adjust pH with HCl or NaOH 1 mol/L. Add 18.64 g of KCl.

Sample preparation: Prepare an ADC solution between 1 and 2.5 mg/mL in milliQ water.

### 8.3.3 DAR: Reverse Phase Liquid Chromatography (RPLC)

| Equipment | UPLC Waters Acquity | |
|---|---|---|
| Detector | Tunable UV detector (TUV) | |
| Detector cell | Titanium | |
| Column | Waters Bioresolve 2.1*150 2.7 um Part No. 186008946 | |
| Mobile phase A | Water + TFA 0.1% (v/v) | |
| Mobile phase B | ACN + TFA 0.1% (v/v) | |
| Wavelength | 280 | nm |
| Injection volume | 10 | μL |
| Column temperature | 90° | C. |
| Sample manager temperature | 25° | C. |
| Run time | 10 | min |
| Flow | 0.35 | mL/min |

| | |
|---|---|
| Weak wash | H₂O/ACN (90/10 v/v) |
| Strong wash | H₂O/ACN (10/90 v/v) |

### Gradient_Basic:

| t (min) | Flow (mL/min) | % A | % B |
|---|---|---|---|
| 0.0 | 0.6 | 73 | 27 |
| 13.0 | 0.6 | 60 | 40 |
| 13.1 | 0.6 | 73 | 27 |
| 16.0 | 0.6 | 73 | 27 |

### Gradient Dev2:

| t (min) | Flow (mL/min) | % A | % B |
|---|---|---|---|
| 0 | 0.6 | 73 | 27 |
| 16 | 0.6 | 50 | 50 |
| 16.1 | 0.6 | 40 | 60 |
| 17 | 0.6 | 40 | 60 |
| 17.1 | 0.6 | 73 | 27 |
| 19 | 0.6 | 73 | 27 |

### Gradient Dev7:

| t (min) | Flow (mL/min) | % A | % B |
|---|---|---|---|
| 0 | 0.6 | 70 | 30 |
| 40 | 0.6 | 40 | 60 |
| 42 | 0.6 | 40 | 60 |
| 42.1 | 0.6 | 70 | 30 |
| 44 | 0.6 | 70 | 30 |

### Gradient Dev7_Short:

| t (min) | Flow (mL/min) | % A | % B |
|---|---|---|---|
| 0 | 0.6 | 70 | 30 |
| 20 | 0.6 | 55 | 45 |
| 20.1 | 0.6 | 5 | 95 |
| 23 | 0.6 | 5 | 95 |
| 23.1 | 0.6 | 70 | 30 |
| 25 | 0.6 | 70 | 30 |

### Mobile Phase Preparation:

ACN+0.1% TFA (VN): Measure 1000 mL of ACN in a graduated cylinder and pour into a 1 L glass bottle. Add 1 mL of TFA with a 1 mL pipette and shake vigorously.
WATER+0.1% TFA (VN): Measure 1000 mL of ultrapure water in a graduated cylinder and pour into a 1 L glass bottle. Add 1 mL of TFA with a 1 mL pipette and shake vigorously.

### Sample Preparation:

mAb: Prepare a 2.5 mg/mL mAb solution in milliQ water. In a vial dispense 45 μL of this solution and 5 μL of a 1 mol/L DTT solution. Incubate 30 minutes at 45° C.
ADC: Prepare a 2.5 mg/mL ADC solution in milliQ water. In a vial dispense 45 μL of this solution and 5 μL of a 0.1 mol/L DTT solution. Incubate 1 hour at 30° C.

### 8.3.4 DAR: MS Method

### Sample Preparation:

Samples were diluted twice with 50 mM ammonium acetate and 25 ug were injected for each sample.

### UPLC:

The separation was performed using the MAbPac™ SEC-1 column (Thermo Scientific) and 50 mM ammonium acetate, pH 7, at 0.3 mL/min as mobile phase.

### MS:

MS was performed using a QExactive HF Orbitrap operated in the high mass range. MS spectra were acquired in the 1800-8000 m/z at a resolution set to 15 k, SID 50 eV. The mass spectra were deconvoluted using Protein Deconvolution (Thermo Scientific).

### 8.3.5 Concentration: UV Method

### Equipment:

UV spectrophotometer BioTek Synergy HT

### Buffer Preparation:

PBS pH 7.4: Weight 8.0 g NaCl, 0.2 g KCl, 1.44 g Na2HPO_{4.2}H₂O and 0.24 g KH₂PO₄ into a 1 L volumetric flask. Add 900 mL milliQ water. Mix it, when all salts are soluble, adjust pH between 7.35 and 7.44 with HCl 1 mol/L. Complete to 1 L with milliQ water.

### Sample Preparation:

Prepare a 1 mg/mL ADC solution in PBS pH 7.4. Using a UV reader determine the absorbance at 252 and 280 nm.

### Calculation Formula:

$${C}^{mAb} = ( \frac{{A}_{280} \times {ε}_{252}^{DM 1} - {A}_{252} \times {ε}_{280}^{DM 1}}{{ε}_{280}^{mAb} \times {ε}_{252}^{DM 1} - {ε}_{252}^{mAb} \times {ε}_{280}^{DM 1}} ) / l$$

A=absorbance
DM1 (drug)=relevant payload
L=path length [cm]
C=concentration [mol/L]
E=extinction coefficient [L·mol-1·cm-1]
ε mg/mL=c mol/L*MW ADC
MW ADC=MW mAb+(MW payload*DAR moyen)

### 8.3.6 Purification

When prepared, the compounds were purified by Preparative Reverse Phase-HPLC on a Buchi C835 using a Waters column (XSelect CSH130 C18 5 μm 19×150 mm OBD or XBridge Prep C18 5 μm OBD 19×150 mm or XSELECT CSH Prep Fluoro-Phenyl 5 μm 19×150 mm) with the indicated solvent system at a flow rate of 25 mL/min. The elution was monitored by UV at a wavelength of 220 nm and by ELSD. Alternatively, the compounds were purified on Biotage Slekt System using Sfar C18 columns. The purity of the peptides was determined with the UPLC methods described previously (see 9.3.1).

### 8.3.7 pH 8 DPBS and pH8 10×DPBS

pH 8 DPBS and pH8 10×DPBS were obtained by addition of 1M NaOH to DPBS and 10×DPBS, respectively.

### 8.3.8 Naratuximab

Naratuximab (also referred to as K7153A herein) refers to the antibody huCD37-3 (Version 1.0) described in WO 2019/229677 (incorporated herein by reference).

### 8.4 Preparation of Linker Payloads:

The linker-payloads were prepared using standard chemistry methods and convergent strategies. The linker-payloads prepared in Example 8.4.1-8.4.9 are shown in Table 1 below.

TABLE 1
linker-payloads

| Linker-payload | Structure |
|---|---|
| DM1-MCC-Cit- Lys(ma- PEG5)-Tyr-OH | 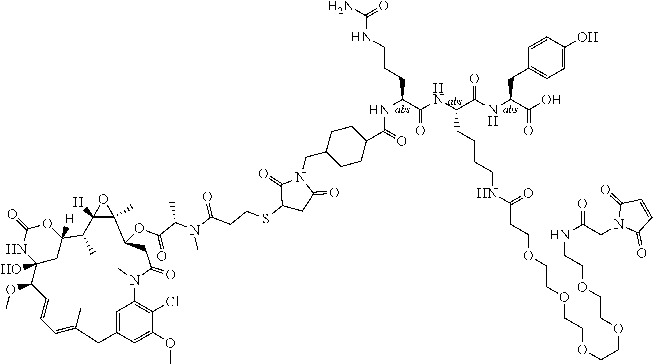 [SMILES: COc1cc2cc(c1Cl)N(C)C(=O)C[C@H](OC(=O)[C@H](C)N(C)C(=O)CCSC1CC(=O)N(CC3CCC(C(=O)N[C@@H](CCCNC(N)=O)C(=O)N[C@@H](CCCCNC(=O)CCOCCOCCOCCOCCOCCNC(=O)CN4C(=O)C=CC4=O)C(=O)N[C@@H](Cc4ccc(O)cc4)C(=O)O)CC3)C1=O)[C@]1(C)O[C@H]1[C@H](C)[C@@H]1C[C@@](O)(NC(=O)O1)[C@H](OC)/C=C/C=C(\C)C2] |
| DM1-Ac-Cit- Lys(ma- Lys(PEG16))- Tyr-OH | 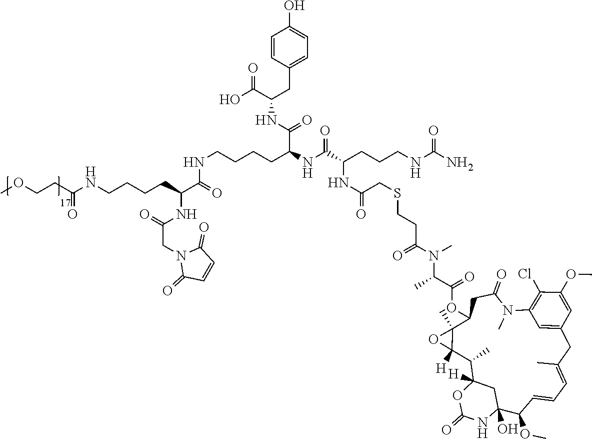 |
| DM1-Ac-Cit- Lys(ma- PEG5)-Tyr-OH | 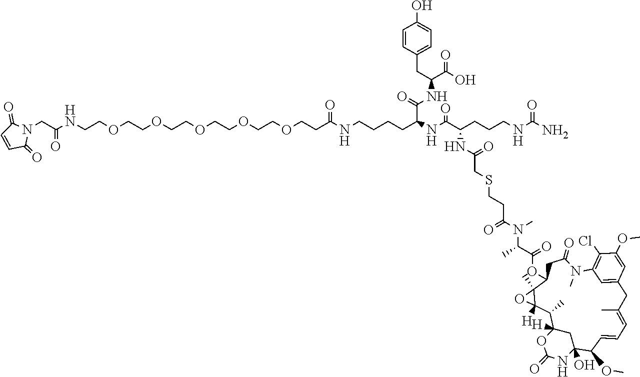 [SMILES: COc1cc2cc(c1Cl)N(C)C(=O)C[C@H](OC(=O)[C@H](C)N(C)C(=O)CCSCC(=O)N[C@@H](CCCNC(N)=O)C(=O)N[C@@H](CCCCNC(=O)CCOCCOCCOCCOCCOCCNC(=O)CN1C(=O)C=CC1=O)C(=O)N[C@@H](Cc1ccc(O)cc1)C(=O)O)[C@]1(C)O[C@H]1[C@H](C)[C@@H]1C[C@@](O)(NC(=O)O1)[C@H](OC)/C=C/C=C(\C)C2] |
| Exatecan-Suc- Phe-cit- Lys(ma- Lys(PEG16))- Tyr-OH | 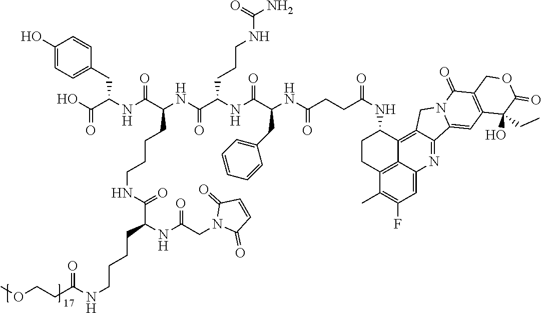 |
| ma-Gly- Glu(DM1-Ac- Cit-Lys-Tyr- OH)-Glu(DM1- Ac-Cit-Lys- Tyr-OH)- PEG24 | 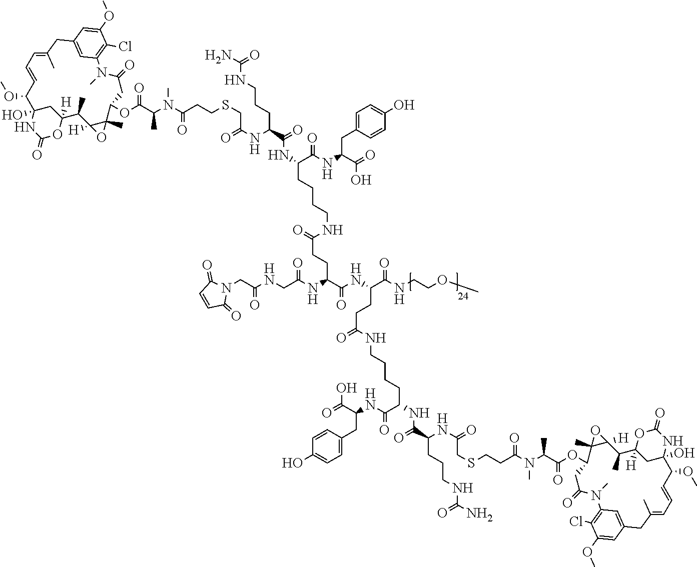 |
| DM1-MCC-Cit- Lys(ma- Lys(PEG16))- Tyr-OH | 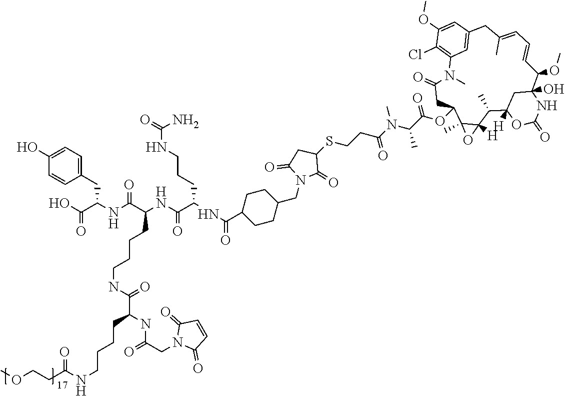 |
| DM1-Ac-Cit- Lys(D-Arg- ma)-Tyr-OH | 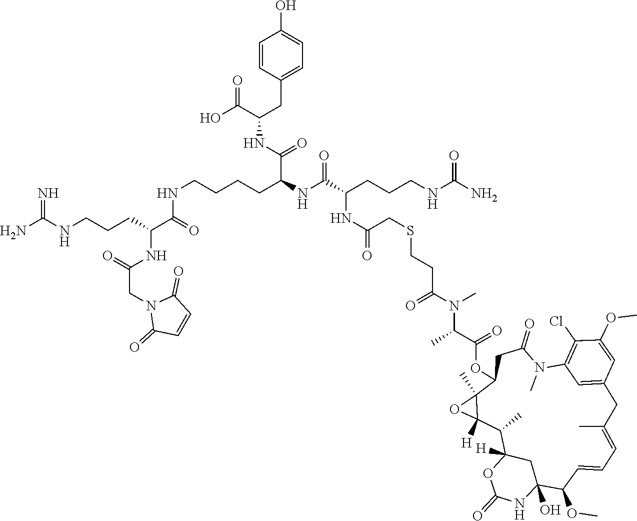 [SMILES: COc1cc2cc(c1Cl)N(C)C(=O)C[C@H](OC(=O)[C@H](C)N(C)C(=O)CCSCC(=O)N[C@@H](CCCNC(N)=O)C(=O)N[C@@H](CCCCNC(=O)[C@@H](CCCNC(=N)N)NC(=O)CN1C(=O)C=CC1=O)C(=O)N[C@@H](Cc1ccc(O)cc1)C(=O)O)[C@]1(C)O[C@H]1[C@H](C)[C@@H]1C[C@@](O)(NC(=O)O1)[C@H](OC)/C=C/C=C(\C)C2] |

### Example 8.4.1: Preparation of DM1-MCC-Cit-Lys(ma-PEG5)-Tyr-OH

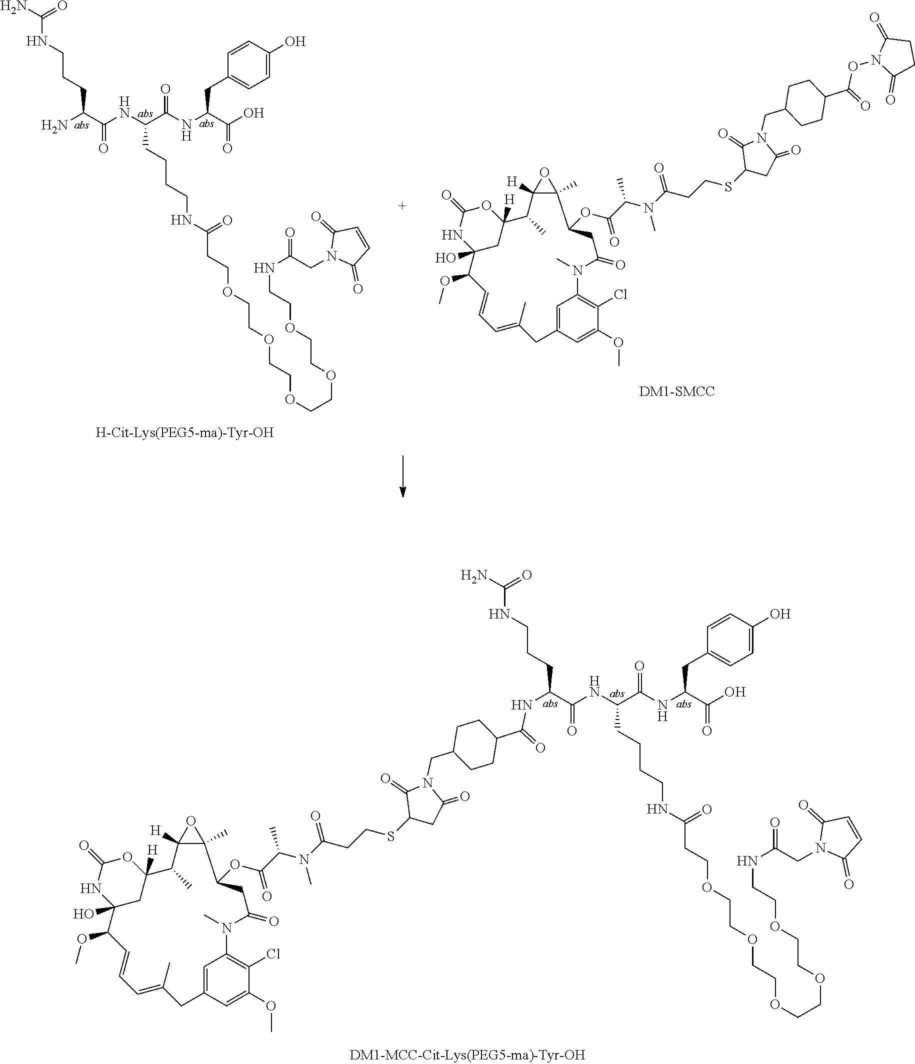

H-Cit-Lys(PEG5-ma)-Tyr-OH was purchased from Ambiopharm and prepared according to the following general procedure:

Peptide synthesis was performed on 2-CTC resin according to the general Fmoc/tBu strategy of solid phase peptide synthesis, with carboxyl group activation carried out by diisopropyl carbodiimide/HOBT. Sequentially, each amino acid was coupled as an active ester to the peptide chain, starting with the C-terminal amino acid. The final amino acid in the sequence was coupled with an N-terminally protected Boc group. The Lys derivative was incorporated with the side chain amino group protected by ivDde which was removed with 2% hydrazine in DMF. Following ivDde removal, the side chain was derivatized with maleimido-PEG₅-OH using the activated ester. Subsequently, the peptide was treated with a TFA-based acidolytic cocktail which resulted in its cleavage from the resin and deprotection of the side chain groups. The peptide was then purified by liquid chromatography (RP-HPLC). The purified peptide TFA salt was lyophilized and obtained as a white to off-white powder.

DIEA (20 μL, 0.11 mmol, 1.0 eq.) was added to a solution of H-Cit-Lys(PEG5-ma)-Tyr-OH (100 mg, 0.11 mmol, 1.0 eq.) and DM1-SMCC (131.8 mg, 0.12 mmol, 1.1 eq.) in DMF (1 mL) at rt. After stirring at rt for 4 h, TFA was added until acidic pH was reached. Purification by preparative HPLC (20 to 80% of ACN+0.1% TFA in water+0.1% TFA) afforded DM1-MCC-Cit-Lys(ma-PEG5)-Tyr-OH (138.5 mg, 74.8 μmol, 100% UV purity, 67% yield) as a white powder after freeze-drying. UPLC-MS (method 4): Rt=2.58 and 2.63 min, m/z=1854 [M+H]⁻, 1852 [M−H]⁻.

### Example 8.4.2: Preparation of DM1-Ac-Cit-Lys(ma-Lys(PEG16))-Tyr-OH

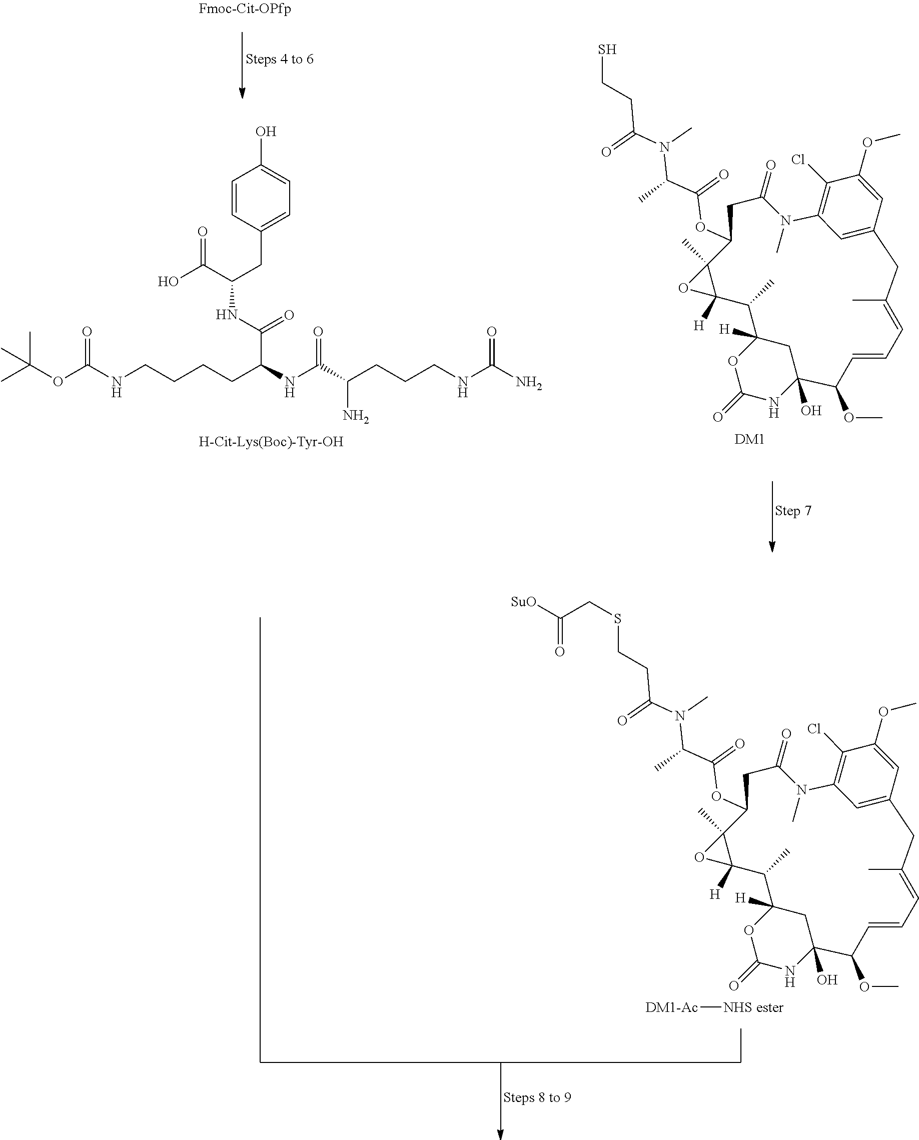

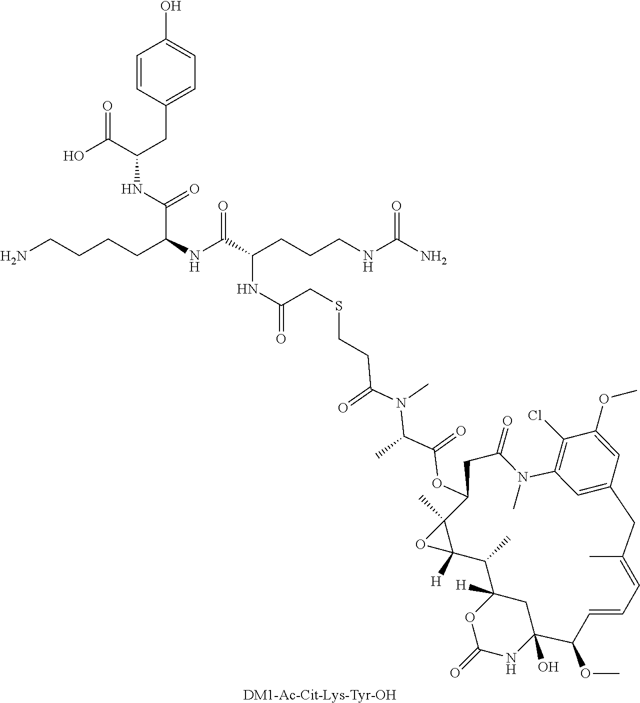 [SMILES: COc1cc2cc(c1Cl)N(C)C(=O)C[C@H](OC(=O)[C@H](C)N(C)C(=O)CCSCC(=O)N[C@@H](CCCNC(N)=O)C(=O)N[C@@H](CCCCN)C(=O)N[C@@H](Cc1ccc(O)cc1)C(=O)O)[C@]1(C)O[C@H]1[C@H](C)[C@@H]1C[C@@](O)(NC(=O)O1)[C@H](OC)/C=C/C=C(\C)C2 ; "DM1-Ac-Cit-Lys-Tyr-OH"]

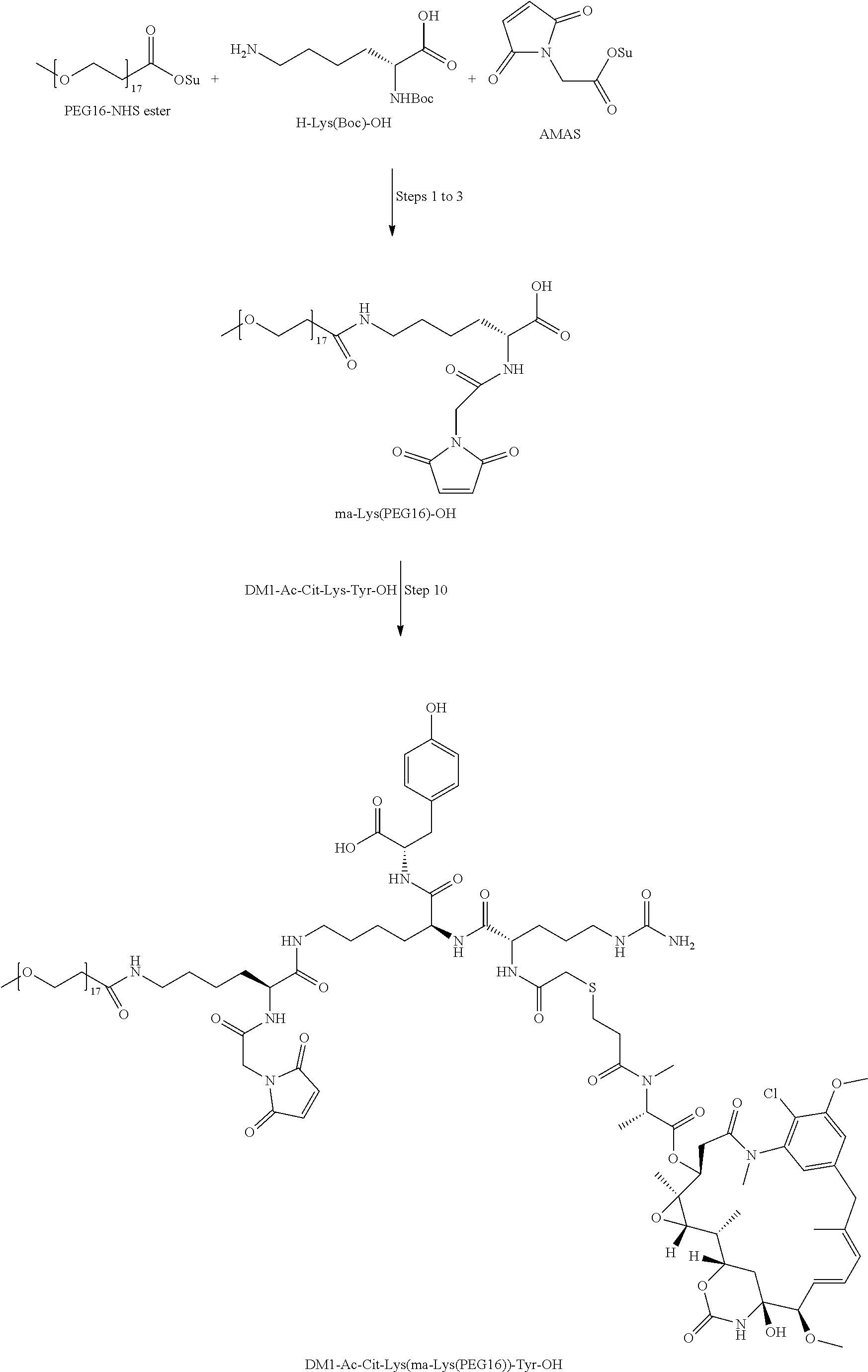

Step 1. DIEA (0.84 mL, 4.82 mmol, 5.0 eq.) was added to a mixture of mPEG16-NHS ester (900 mg, 0.96 mmol, 1.0 eq.) and Boc-Lys-OH (300 mg, 1.16 mmol, 1.2 eq.) in DMF (12 mL) at rt. After stirring at rt for 16 h, TFA was added until acidic pH was reached. Purification by preparative HPLC (20 to 80% of ACN+0.1% TFA in water+0.1% TFA) afforded Boc-Lys(PEG16)-OH (990 mg, 0.95 mmol, 100% UV purity, 99% yield) as a colourless oil after freeze-drying. UPLC-MS (method 1): Rt=1.16 min, m/z=1038 [M+H]⁺.

Step 2. TFA (6 mL) was added to a solution of Boc-Lys(PEG16)-OH (990 mg, 0.95 mmol, 1.0 eq.) in DCM (6 mL) at rt. After stirring at rt for 30 min, the reaction mixture was concentrated in vacuo. The residue was dissolved in a mixture of ACN/water (1:1, 16 mL) then freeze-dried to afford H-Lys(PEG16)-OH·TFA (1.00 g, 0.95 mmol, 100% UV purity, quant.) as a colourless oil. UPLC-MS (method 1): Rt=0.89 min, m/z=938 [M+H]⁺, 936 [M−H]⁻.

Step 3. DIEA (0.15 mL, 0.85 mmol, 4.0 eq.) was added to a mixture of H-Lys(PEG16)-OH (200 mg, 0.21 mmol, 1.0 eq.) and AMAS (56.5 mg, 0.22 mmol, 1.05 eq.) in DMF (2.5 mL) at rt. After stirring at rt for 1 h, TFA was added until acidic pH was reached. Purification by preparative HPLC (10 to 40% ACN+0.1% TFA in water+0.1% TFA) afforded ma-Lys(PEG16)-OH (220 mg, 0.20 mmol, 100% UV purity, 96% yield) as a colourless oil after freeze-drying. UPLC-MS (method 4): Rt=1.74 min, m/z=1075 [M+H]⁺, 1073 [M−H]⁻.

Step 4. DIEA (0.6 mL, 3.44 mmol, 3.7 eq.) was added to a mixture of Fmoc-Cit-OPFP (520.7 mg, 0.92 mmol, 1.0 eq.) and H-Lys(Boc)-OH (218.1 mg, 0.89 mmol, 0.96 eq.) in a mixture of DMF (6 mL) and water (3 mL) at rt. After stirring at rt for 1 h, TFA was added dropwise until acidic pH was reached. Purification by C18 flash column chromatography (20 to 80% of ACN+0.1% TFA in water+0.1% TFA) afforded Fmoc-Cit-Lys(Boc)-OH (593.3 mg, 0.92 mmol, 97% UV purity, quant.) as a white powder after freeze-drying. UPLC-MS (method 3): Rt=1.80 min, m/z=626 [M+H]⁺, 526 [M-Boc+H]⁺, 624 [M−H]⁻.

Step 5. H-Tyr-OMe (139.2 mg, 0.71 mmol, 1.1 eq.) followed by HATU (334.2 mg, 0.88 mmol, 1.4 eq.) and DIEA (0.5 mL, 2.87 mmol, 4.5 eq.) were added to a solution of Fmoc-Cit-Lys(Boc)-OH (400 mg, 0.64 mmol, 1.0 eq.) in DMF (10 mL) at rt. After stirring at rt for 2 h, TFA was added until acidic pH was reached. Purification by C18 flash column chromatography (20 to 80% ACN+0.1% TFA in water+0.1% TFA) gave H-Cit-Lys(Boc)-Tyr-H (513.3 mg, 0.57 mmol, 89% UV purity, 89% yield) as a white powder after freeze-drying. UPLC-MS (method 3): Rt=1.83 min, m/z=803 [M+H]⁺, 703 [M−Boc+H]⁺, 847 [M+FA-H]⁻.

Step 6. Lithium hydroxide monohydrate (158.5 mg, 3.78 mmol, 4.2 eq.) was added to a solution of Fmoc-Cit-Lys(Boc)-Tyr-OMe (730.6 mg, 0.91 mmol, 1.0 eq.) in THF (8 mL) and water (5 mL) at rt. After stirring at rt for 40 min, TFA was added until slightly acidic pH was reached then the reaction mixture was concentrated in vacuo. Purification by preparative HPLC (15 to 40% of ACN+0.1% TFA in water+0.1% TFA) afforded H-Cit-Lys(Boc)-Tyr-OH (510 mg, 0.90 mmol, 100% UV purity, 99% yield) as a white powder after freeze-drying. UPLC-MS (method 1): Rt=0.80 min, m/z=567 [M+H]⁺, 565 [M−H]⁻.

Step 7. DIEA (0.28 mL, 1.63 mmol, 6.0 eq.) was added to a solution of bromoacetic acid (64.8 mg, 0.47 mmol, 1.7 eq.) and DM1 (200 mg, 0.27 mmol, 1.0 eq.) in DMF (2 mL) at rt. After stirring at rt for 1 h, a mixture of HATU (113.3 mg, 0.30 mmol, 1.1 eq.) and 1-hydroxypyrrolidine-2,5-dione (34.3 mg, 0.30 mmol, 1.1 eq.). After 30 min stirring at rt, TFA was added until acidic pH was reached. Purification on preparative HPLC (30 to 60% ACN+0.1% TFA in water+0.1% TFA) afforded DM1-Ac-NHS ester (134.7 mg, 0.12 mmol, 80% UV purity, 45% yield) as a white powder after freeze-drying. UPLC-MS (method 2): Rt=1.57 min, m/z=891 [M−H]⁻.

Step 8. DIEA (0.22 mL, 1.24 mmol, 4.0 eq.) was added to a mixture of DM1-Ac—NHS ester (347 mg, 0.31 mmol, 1.0 eq.) and H-Cit-Lys(Boc)-Tyr-OH (222.1 mg, 0.33 mmol, 1.05 eq.) in DMF (4 mL) at rt. After stirring at rt for 1 h, TFA was added until acidic pH was reached. Purification by preparative HPLC (30 to 60% of ACN+0.1% TFA in water+0.1% TFA) afforded DM1-Ac-Cit-Lys(Boc)-Tyr-OH (417 mg, 0.31 mmol, 100% UV purity, quant.) as a white powder after freeze-drying. UPLC-MS (method 3): Rt=1.67 min, m/z=1344 [M−H]⁻.

Step 9. A mixture of TFA (0.9 mL) and DCM (3.6 mL) was added to a mixture of DM1-Ac-Cit-Lys(Boc)-Tyr-OH (112.0 mg, 83.3 μmol, 1.0 eq.) at rt. After stirring at rt for 5 min, a mixture of ACN/water (1:1, 8 mL) was added to the reaction mixture. The DCM was removed by concentration under vacuo. Purification by preparative HPLC (2 to 40% of ACN+0.1% TFA in water+0.1% TFA) afforded DM1-Ac-Cit-Lys-Tyr-OH (80 mg, 64.3 μmol, 100% UV purity, 77% yield) as a white powder after freeze-drying. UPLC-MS (method 2): Rt=1.03 min, m/z=1242 [M+H]⁺, 1240 [M−H]⁻.

Step 10. 1-hydroxypyrrolidine-2,5-dione (2.14 mg, 18.6 μmol, 1.0 eq.) and EDC·HCl (3.57 mg, 18.6 μmol, 1.0 eq.) were added to a solution of ma-Lys(PEG16)-OH (20.0 mg, 18.6 μmol, 1.0 eq.) in DCM (2 mL) at rt. After stirring at rt for 2.5 h, DM1-Ac-Cit-Lys-Tyr-OH (37.1 mg, 22.3 μmol, 1.2 eq.) and DIEA (13 μL, 74.5 μmol, 4.0 eq.) were added to the reaction mixture at rt. After stirring at rt for 10 min, TFA was added until acidic pH was reached. Purification by preparative HPLC (5 to 100% of ACN+0.1% FA in water+0.1% FA) afforded DM1-Ac-Cit-Lys(ma-Lys(PEG16))-Tyr-OH (17.2 mg, 7.50 μmol, 100% UV purity, 40% yield) as a white powder after freeze drying. UPLC-MS (method 4): Rt=2.59 min, m/z=1173 [M+FA-H]⁻.

### Example 8.4.3: Preparation of DM1-Ac-Cit-Lys(ma-PEG5)-Tyr-OH

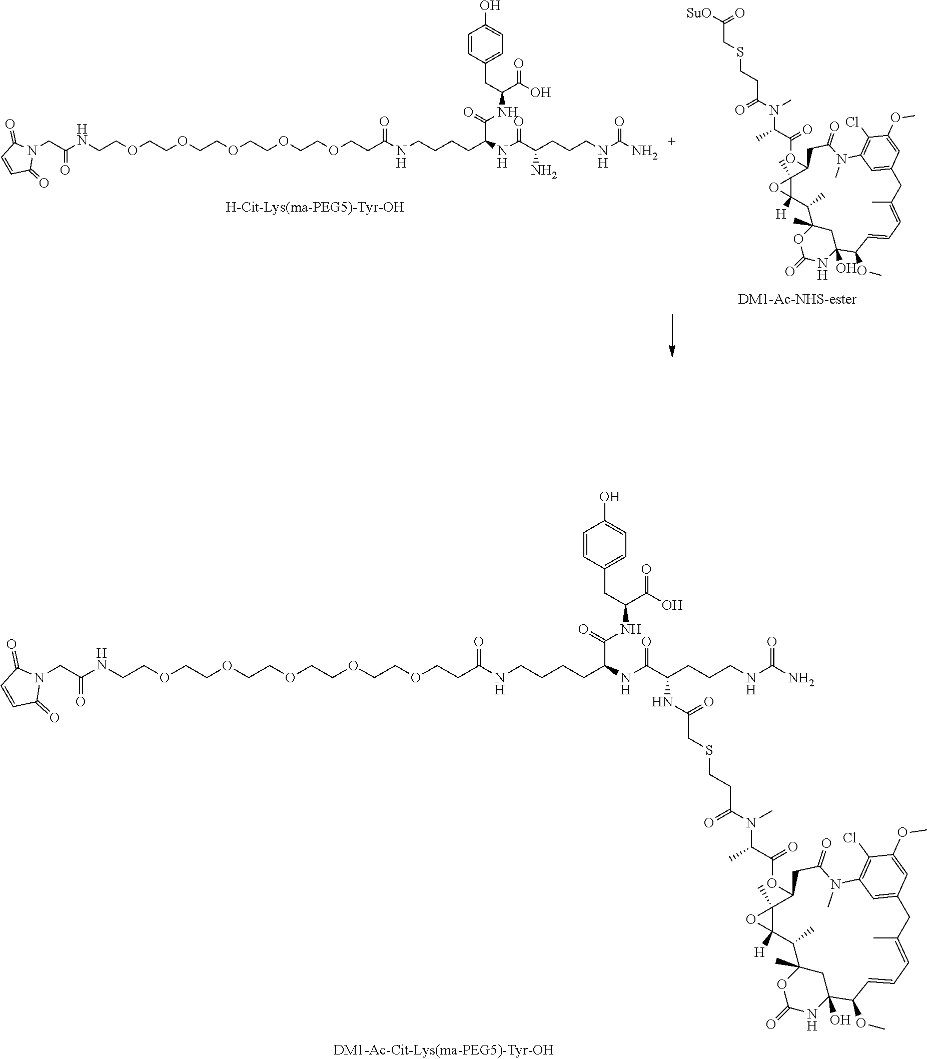

DIA (31 μL, 0.18 mmol, 4.0 eq.) was added to a mixture of DM1-Ac—NHS ester (50.0 mg, 44.8 μmol, 1.0 eq.) and H-Cit-Lys(ma-PEG5)-Tyr-OH·TFA (45.2 mg, 44.8 μmol, 1.0 eq.) in DMF (1 mL) at rt. After stirring at rt for 30 min TFA was added until acidic pH was reached. Purification by preparative HPLC (20 to 50% of ACN+0.1% TFA in water+0.1% TFA) afforded DM1-Ac-Cit-Lys(ma-PEG5)-Tyr-OH (74.9 mg, 44.8 μmol, 100% UV purity, quant.) as a white powder after freeze-drying. UPLC-MS (method 1): Rt=1.31 min, m/z=1672 [M−H]⁻

### Example 8.4.4: Preparation of Exatecan-Suc-Phe-Cit-Lys(ma-Lys(PEG16))-Tyr-OH

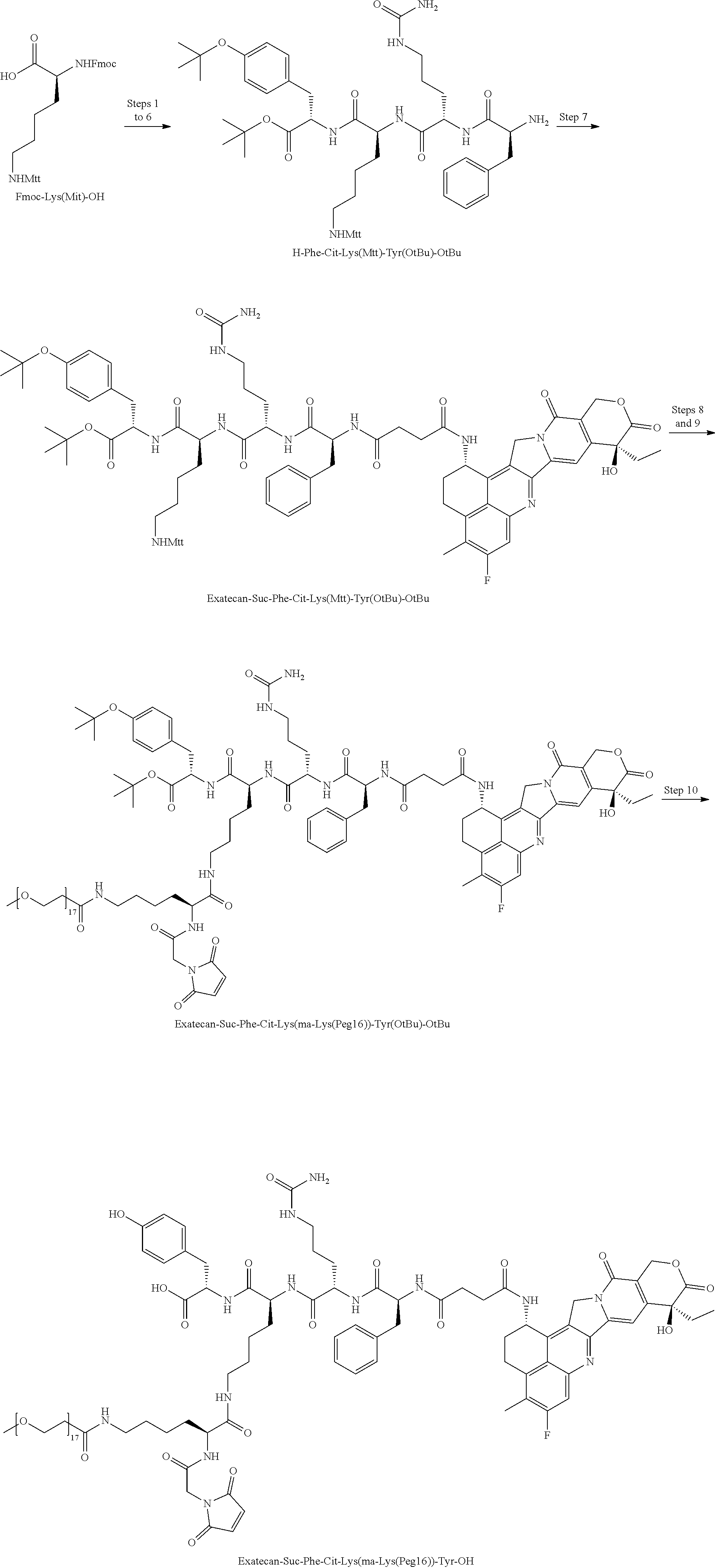

Step 1. DIEA (2.0 mL, 11.8 mmol, 3.0 eq.) was added to a mixture of H-Tyr(OtBu)-OtBu (2.59 g, 7.85 mmol, 1.0 eq.), Fmoc-Lys(Mtt)-OH (2.51 g, 3.94 mmol, 1.0 eq.) and HATU (1.95 g, 5.12 mmol, 1.3 eq.) in DMF (40 mL) at 0-5° C. After stirring at rt for 50 min, the reaction mixture was diluted with EtOAc (300 mL) and washed with half saturated brine (2×200 mL). The organic layer was dried over MgSO₄, filtered and concentrated under reduced pressure to afford Fmoc-Lys(Mtt)-Tyr(OtBu)-OtBu (7.07 g, 7.69 mmol, 98% UV purity, 98% yield) as a light brown oil. UPLC-MS (method 3): Rt=2.42 min, m/z=900 [M+H]⁺, 944 [M+FA-H]⁻.

Step 2. A mixture of DMF/piperidine (9:1, 40 mL) was added to Fmoc-Lys(Mtt)-Tyr(OtBu)-OtBu (7.07 g, 7.69 mmol, 1.0 eq.) at rt. After stirring at rt for 10 min, the reaction mixture was concentrated to dryness. The residue was purified by flash chromatography (n-heptane, then DCM and finally DCM:MeOH 95:5 to 90:10) to afford H-Lys(Mtt)-Tyr(OtBu)-OtBu (5.10 g, 7.15 mmol, 95% UV purity, 93% yield) as an orange oil after concentration in vacuo. UPLC-MS (method 3): Rt=1.58 min, m/z=678 [M+H]⁺, 722 [M+FA-H]⁻.

Step 3. DIEA (1.73 mL, 9.96 mmol, 3.0 eq.) was added to a mixture of H-Lys(Mtt)-Tyr(OtBu)-OtBu (2.50 g, 3.32 mmol, 1.0 eq.), Fmoc-Cit-OH (1.48 g, 3.65 mmol, 1.1 eq.) and HATU (1.70 g, 4.31 mmol, 1.3 eq.) in DMF (20 mL) at 0° C. After stirring at rt for 30 min, the reaction mixture was poured into 5% NaHCO₃ aqueous solution (200 mL) and extracted with EtOAc (2×100 mL). The combined organic layer was washed with brine (100 mL), dried over MgSO₄, filtered and concentrated under reduced pressure. Purification by flash chromatography (heptane:EtOAc 80:20 to plain EtOAc, then 5-10% EtOAc:MeOH 95:5 to 90:10) afforded Fmoc-Cit-Lys(Mtt)-Tyr(OtBu)-OtBu (3.40 g, 3.05 mmol, 95% UV purity, 92% yield) as a beige gum after concentration in vacuo. UPLC-MS (method 3): Rt=2.15 min, m/z=1058 [M+H]⁺, 1102 [M+FA-H]⁻.

Step 4. A mixture of DMF/piperidine (20 mL, 9:1 V/V) was added to Fmoc-Cit-Lys(Mtt)-Tyr(OtBu)-OtBu (3.40 g, 3.22 mmol, 1.0 eq.) at rt. After stirring at rt for 10 min, the reaction mixture was concentrated to dryness. Purification by flash chromatography (heptane:EtOAc 80:20 to plain EtOAc, then 5-10% EtOAc:MeOH 95:5 to 90:10, then 5-15% of aqueous 25% NH₃ in ACN) afforded H-Cit-Lys(Mtt)-Tyr(OtBu)-OtBu (2.55 g, 2.96 mmol, 97% UV purity, 92% yield) as a colourless oil after concentration in vacuo. UPLC-MS (method 3): Rt=1.54 min, m/z=836 [M+H]⁺, 880 [M+FA-H]⁻.

Step 5. DIEA (0.61 mL, 3.48 mmol, 3.0 eq.) was added to a mixture of H-Cit-Lys(Mtt)-Tyr(OtBu)-OtBu (1.00 g, 1.16 mmol, 1.0 eq.), Fmoc-Phe-OH (539 mg, 1.39 mmol, 1.2 eq.) and HATU (594 mg, 1.51 mmol, 1.3 eq.) in DMF (15 mL) at 0° C. After stirring at rt for 15 min, the reaction mixture was poured into 5% NaHCO₃ aqueous solution (50 mL) and extracted with EtOAc (2×70 mL). The combined organic layer was washed with brine (50 mL), dried over Na2SO₄, filtered and partially concentrated under reduced pressure. Precipitation with ether afforded Fmoc-Phe-Cit-Lys(Mtt)-Tyr(OtBu)-OtBu (1.33 g, 1.06 mmol, 96% UV purity, 92% yield) as a white solid. UPLC-MS (method 3): Rt=2.29 min, m/z=1205 [M+H]⁺, 1249 [M+FA-H]⁻.

Step 6. A mixture of DMF/piperidine (10 mL, 9:1 V/V) was added to Fmoc-Phe-Cit-Lys(Mtt)-Tyr(OtBu)-OtBu (1.33 g, 1.06 mmol, 1.0 eq.) at rt. After stirring at rt for 10 min, the reaction mixture was concentrated to dryness. Purification by flash chromatography (heptane:EtOAc 80:20 to plain EtOAc, then 5-10% EtOAc:MeOH 90:10, then 10-20% of aqueous 25% NH₃ in ACN) afforded H-Phe-Cit-Lys(Mtt)-Tyr(OtBu)-OtBu (0.93 g, 0.90 mmol, 95% UV purity, 81% yield) as a white powder after concentration in vacuo. UPLC-MS (method 3): Rt=1.57 min, m/z=983 [M+H]⁺, 1027 [M+FA-H]⁻.

Step 7. Dihydrofuran-2,5-dione (1.16 mg, 11.5 μmol, 1.0 eq.) was added to a mixture of Exatecan mesylate (5.0 mg, 11.5 μmol, 1.0 eq.) and DIEA (0.02 mL, 0.14 mmol, 12.0 eq.) in DMF (0.5 mL) at rt. After stirring at rt for 15 min, H-Phe-Cit-Lys(Mtt)-Tyr(OtBu)-OtBu (11.28 mg, 11.5 μmol, 1.0 eq.) was added followed by HATU (4.37 mg, 11.5 μmol, 1.0 eq.). After stirring at rt for 1 h, TFA was added until acidic pH was reached. Purification by preparative HPLC (10 to 100% of ACN+0.1% TFA in water+0.1% TFA) afforded Exatecan-Suc-Phe-Cit-Lys(Mtt)-Tyr(OtBu)-OtBu (8.1 mg, 5.30 mmol, 98% UV purity, 46% yield) as a yellow powder after freeze-drying. UPLC-MS (method 4): Rt=3.70 min, m/z=1501 [M+H]⁺, 1545 [M+FA-H]⁻.

Step 8. A mixture of DCM (5 mL) and TFA (0.1 mL) was added to Exatecan-Suc-Phe-Cit-Lys(Mtt)-Tyr(OtBu)-OtBu (61.0 mg, 40.7 μmol, 1.0 eq.) at rt. The reaction mixture was stirred at rt for 1 h then concentrated in vacuo. Purification by preparative HPLC (20 to 40% of ACN+0.1% TFA in water+0.1% TFA) afforded Exatecan-Suc-Phe-Cit-Lys-Tyr(OtBu)-OtBu (6.95 mg, 5.4 μmol, 97% UV purity, 13% yield) as a yellow powder after freeze-drying. UPLC-MS (method 4): Rt=2.82 min, m/z=1244 [M+H]⁺, 1288 [M+FA-H]⁻.

Step 9. 1-hydroxypyrrolidine-2,5-dione (9.0 mg, 78 μmol, 2.0 eq.) and EDC·HCl (15.0 mg, 78 μmol, 2.0 eq.) were added to a solution of ma-Lys(PEG16)-OH (41.9 mg, 39 μmol, 1.0 eq.) in DCM (2.0 mL) at rt. After stirring at rt for 1 h, Exatecan-Suc-Phe-Cit-Lys-Tyr(OtBu)-OtBu (50.0 mg, 39 μmol, 1 eq.) and DIEA (27 μL, 0.16 mmol, 4.0 eq.) were added to the reaction mixture at rt. After stirring at rt for 20 min, TFA was added until acidic pH was reached. Purification by preparative HPLC (30 to 80% of ACN+0.1% TFA in water+0.1% TFA) afforded Exatecan-Suc-Phe-Cit-Lys(ma-Lys(Peg16))-Tyr(OtBu)-OtBu (10.0 mg, 4.30 mmol, 100% UV purity, 11% yield) as a white powder after freeze-drying. UPLC-MS (method 4): Rt=3.15 min, m/z=1151 [M+H]⁺.

Step 10. TFA (0.11 mL) was added to a solution of Exatecan-Suc-Phe-Cit-Lys(ma-Lys(Peg16))-Tyr(OtBu)-OtBu (10.0 mg, 4.30 μmol, 1.0 eq.) in DCM (0.11 mL) at rt. After stirring at rt for 3 h, the reaction mixture was concentrated in vacuo. Purification by preparative HPLC (20 to 60% of ACN+0.1% TFA in water+0.1% TFA) afforded Exatecan-Suc-Phe-Cit-Lys(ma-Lys(Peg16))-Tyr-OH (5.3 mg, 2.40 μmol, 100% UV purity, 56% yield) as a white powder after freeze-drying. UPLC-MS (method 4): Rt=2.44 min, m/z=1094 [M+H]⁺, 1092 [M+FA-H]⁻.

### Example 8.4.5: Preparation of ma-Gly-Glu(DM1-Ac-Cit-Lys-Tyr-OH)-Glu(DM1-Ac-Cit-Lys-Tyr-OH)-PEG24

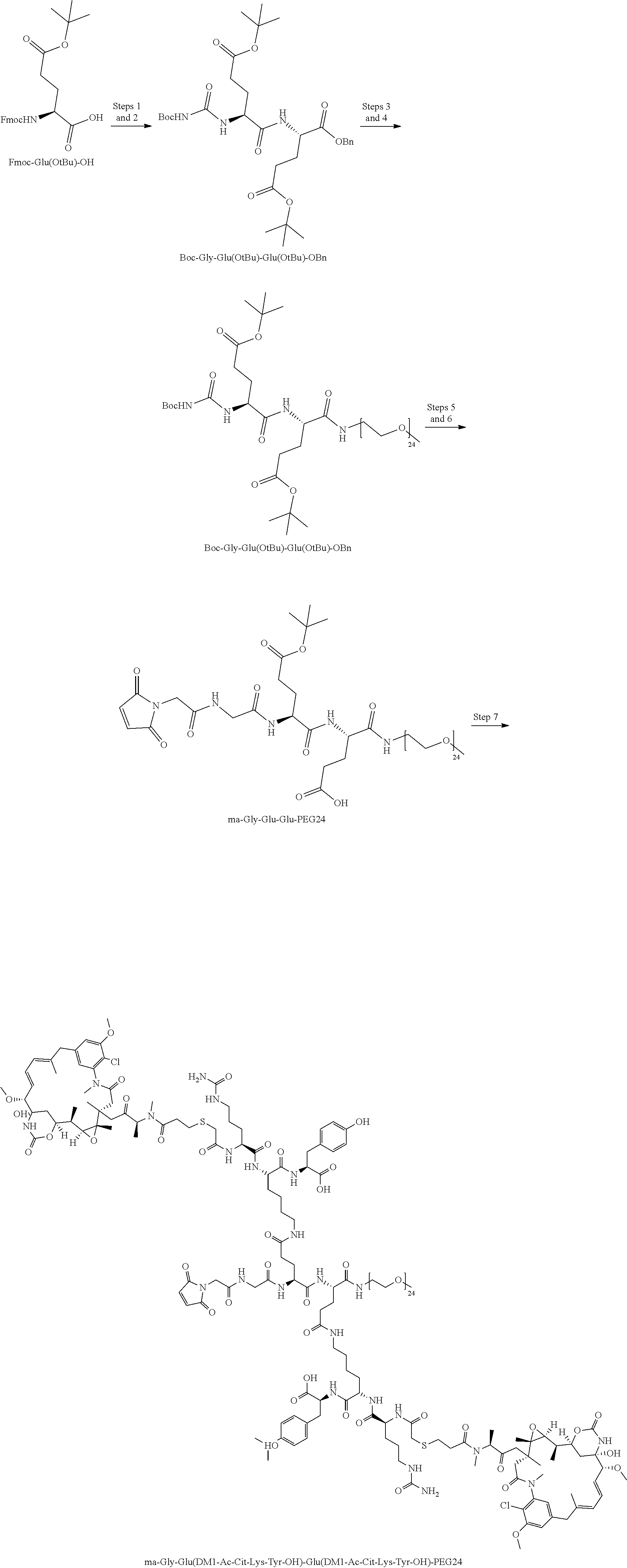

Step 1. DIEA (1.17 mL, 6.66 mmol, 3.0 eq.) was added to a mixture of Fmoc-Glu(OtBu)-OH·H₂O (1.00 g, 2.22 mmol, 1.0 eq.), H-Glu(OtBu)-OBn·HCl (750 mg, 2.22 mmol, 1.0 eq.) and HATU (1.14 g, 2.99 mmol, 1.35 eq.) in DMF (15 mL) at 0° C. After stirring at 0° C. for 15 min and at rt for 1 h, the reaction mixture was concentrated to dryness. The resulting brown oil was diluted with EtOAC and washed with a 5% aqueous NaHCO₃ solution. The organic phase was washed with brine, dried over MgSO₄, filtered and concentrated to dryness. Flash chromatography (n-heptane:EtOAc 80:20 to 0:100) afforded Fmoc-Glu(OtBu)-Glu(OtBu)-OBn (1.46 g, 2.09 mmol, 100% UV purity, 94% yield) as a white foam. UPLC-MS (method 3): Rt=2.62 min, m/z=701 [M+H]⁺, 745 [M+FA-H]⁻.

Step 2. DBU (0.3 mL, 2.00 mmol, 1.2 eq.) was added to a solution of Fmoc-Glu(OtBu)-Glu(OtBu)-OBn (1.17 g, 1.67 mmol, 1.0 eq.) in DMF (5 mL) at rt. After stirring at rt for 5 min, the reaction mixture was added dropwise over 3 min to a mixture of Boc-Gly-OH (351 mg, 2.00 mmol, 1.2 eq.), HATU (855.3 mg, 2.17 mmol, 1.3 eq.) and DIEA (0.87 mL, 5.01 mmol, 3.0 eq.) in DMF (12 mL) at rt. After stirring at rt for 30 min, the reaction mixture was concentrated to dryness. The residue was diluted with EtOAc then washed with citric acid 10%. The organic layer was dried over MgSO₄ and concentrated to dryness. Flash chromatography (DCM:EtOAc 90:10 to 0:100) afforded Boc-Gly-Glu(OtBu)-Glu(OtBu)-OBn (982 mg, 1.47 mmol, 95% UV purity, 88% yield) as a white solid. UPLC-MS (method 3): Rt=2.22 min, m/z=636 [M+H]⁺, 634 [M−H]⁻.

Step 3. Palladium (164.4 mg, 0.15 mmol, 0.1 eq.) was added under nitrogen atmosphere into a 25 mL flask for hydrogenation. A solution of Boc-Gly-Glu(OtBu)-Glu(OtBu)-OBn (982 mg, 1.54 mmol, 1.0 eq.) in ethanol (9.8 mL) was added. The resulting suspension was stirred under 1 bar of hydrogen at rt for 30 min then filtered through a pad of Celite®. The filtrate was concentrated under reduced pressure to afford Boc-Gly-Glu(OtBu)-Glu(OtBu)-OH (898 mg, 1.65 mmol, 94% UV purity, quant.) as a white foam. UPLC-MS (method 3): Rt=1.73 min, m/z=546 [M+H]⁺, 544 [M−H]⁻.

Step 4. DIEA (48 mL, 0.27 mmol, 3.0 eq.) was added to a mixture of Boc-Gly-Glu(OtBu)-Glu(OtBu)-OH (50.0 mg, 91.6 mmol, 1.0 eq.), PEG24-amine (120.5 mg, 0.11 mmol, 1.2 eq.) and HATU (59.0 mg, 0.15 mmol, 1.6 eq.) in DMF (0.5 mL) at rt. After stirring at rt for 10 min, the reaction mixture was acidified with TFA to pH 4-5. Purification by preparative HPLC (10-100% of ACN+0.1% TFA in water+0.1% TFA) afforded Boc-Gly-Glu(OtBu)-Glu(OtBu)-PEG24 (100 mg, 61.9 mmol, 100% UV purity, 68% yield) as a white solid after freeze-drying. UPLC-MS (method 3): Rt=1.82 min, m/z=1614 [M−H]⁻, 1660 [M+FA-H]⁻.

Step 5. TFA (0.5 mL) was added to a solution of Boc-Gly-Glu(OtBu)-Glu(OtBu)-PEG24 (100 mg, 61.9 mmol, 1.0 eq.) in DCM (0.5 mL) at rt. After stirring at rt for 30 min, the reaction mixture was concentrated to dryness to afford H-Gly-Glu-Glu-PEG24.TFA (93 mg, 61.3 mmol, 99% yield) as a yellow oil. UPLC-MS (method 1): Rt=0.94 min, m/z=1404 [M+H]⁺, 1402 [M−H]⁻.

Step 6. DIEA (53 μL, 0.31 mmol, 5.0 eq.) was added to a mixture of H-Gly-Glu-Glu-PEG24.TFA (93.0 mg, 61.3 μmol, 1.0 eq.) and AMAS (21.2 mg, 79.7 μmol, 1.3 eq.) in DMF (0.3 mL) at rt. After stirring at rt for 1 h, TFA was added (24 μL). Purification by preparative HPLC (20-100% of ACN+0.1% TFA in water+0.1% TFA) afforded ma-Gly-Glu-Glu-PEG24 (94.0 mg, 58.0 mmol, 95% UV purity, 95% yield) as colourless oil. UPLC-MS (method 3): Rt=1.20 min, m/z=1539 [M−H]⁻.

Step 7. DIEA (27 μL, 0.16 mmol, 8.0 eq.) was added to a mixture of ma-Gly-Glu-Glu-PEG24 (30.0 mg, 19.5 μmol, 1.0 eq.) and HATU (14.8 mg, 38.9 μmol, 2.0 eq.) in DMF (3 mL) at rt. After stirring at rt for 7 min, the reaction mixture was added to DM1-Ac-Cit-Lys-Tyr-OH (52.9 mg, 38.9 μmol, 2.0 eq.) at rt. After stirring at rt for 15 min, TFA was added until acidic pH was reached. Purification by preparative HPLC (30 to 70% of ACN+0.1% TFA in water+0.1% TFA) afforded ma-Gly-Glu(DM1-Ac-Cit-Lys-Tyr-OH)-Glu(DM1-Ac-Cit-Lys-Tyr-OH)-PEG24 (15.1 mg, 3.40 μmol, 89% UV purity, 17% yield) as a white powder after freeze-drying. UPLC-MS (method 3): Rt=1.70 min, m/z=1996 [M-2H]²⁻.

### Example 8.4.6: Preparation of DM1-MCC-Cit-Lys(ma-Lys(PEG16))-Tyr-OH

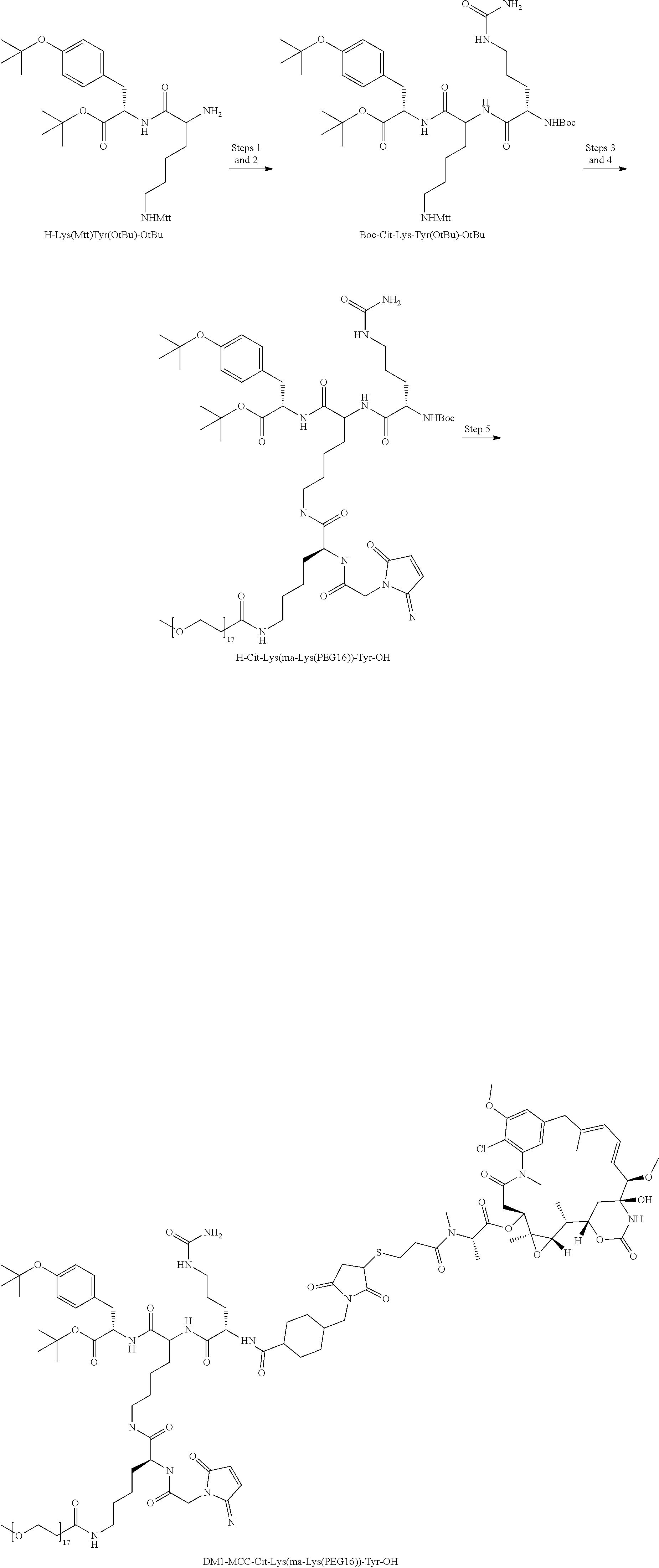

Step 1. DIEA (0.7 mL, 3.98 mmol, 3.0 eq.) was added to a mixture of H-Lys(Mtt)-Tyr(OtBu)-OtBu (1.00 g, 1.33 mmol, 1.0 eq.), HATU (680 mg, 1.73 mmol, 1.3 eq.) and Boc-Cit-OH (373 mg, 1.33 mmol, 1.0 eq.) in DMF (8 mL) at 0-5° C. After stirring at rt for 30 min, the reaction mixture was poured into EtOAc (200 mL), washed with brine (100 mL) then half saturated brine (50 mL). The resulting emulsion was first evaporated under reduced pressure then freeze-dried to afford Boc-Cit-Lys(Mtt)-Tyr(OtBu)-OtBu (2.10 g, 1.80 mmol, 60% purity, quant.) as a beige solid. UPLC-MS (method 3): Rt=2.02 mi, m/z=936 [M+H]⁺, 980 [M+FA-H]⁻.

Step 2. A mixture of DCM (65 mL) and TFA (2 mL) was added to Boc-Cit-Lys(Mtt)-Tyr(OtBu)-OtBu (1.24 g, 1.33 mmol, 1.0 eq.) at rt. After stirring at rt for 10 min, the reaction mixture was partially concentrated then added slowly into cold ether. The oily product obtained by centrifugation was purified by preparative HPLC (25 to 80% of ACN+0.1% TFA in water+0.1% TFA) to afford Boc-Cit-Lys-Tyr(OtBu)-OtBu (602 mg, 0.76 mmol, 100% UV purity, 57% yield) as a white powder after freeze-drying. UPLC-MS (method 3): Rt=1.46 min, m/z=680 [M+H]⁺, 723 [M+FA-H]⁻.

Step 3. DIEA (0.06 mL, 0.37 mmol, 4.0 eq.) was added to a mixture of ma-Lys(PEG16)-OH (100 mg, 93.1 μmol, 1.0 eq.), Boc-Cit-Lys-Tyr(OtBu)-OtBu (81.2 mg, 102.4 μmol, 1.1 eq.) and HATU (47.7 mg, 121 μmol, 1.3 eq.) in DMF (1.5 mL) at 5° C. After stirring at 5° C. for 10 min, TFA was added until acidic pH was reached. Purification by preparative HPLC (20 to 70% of ACN+0.1% TFA in water+0.1% TFA) afforded Boc-Cit-Lys(ma-Lys(PEG16))-Tyr(OtBu)-OtBu (158 mg, mmol, 96% UV purity, 94% yield) as a white powder after freeze-drying. UPLC-MS (method 3): Rt=1.81 min, m/z=1736 [M+-H]⁺, 1780 [M+FA-H]⁻.

Step 4. A mixture of TFA (2.5 mL) and DCM (2.5 mL) was added to Boc-Cit-Lys(ma-Lys(PEG16))-Tyr(OtBu)-OtBu (158 mg, 87.2 μmol, 1.0 eq.) at rt. After stirring at 4° C. for 16 h, the reaction mixture was concentrated in vacuo. Purification by preparative HPLC (10 to 50% of ACN+0.1% TFA in water+0.1% TFA) afforded H-Cit-Lys(ma-Lys(PEG16))-Tyr-OH (132 mg, 79.6 μmol, 99% UV purity, 91% yield) as a colourless oil after freeze-drying. UPLC-MS (method 3): Rt=1.04 min, m/z=1523 [M+H]⁺, 1521 [M−H]⁻.

Step 5. DIEA (41 μL, 0.25 mmol, 4.0 eq.) was added to a mixture of DM1-SMCC (68.6 mg, 62.0 μmol, 1.0 eq.) and H-Cit-Lys(ma-Lys(PEG16))-Tyr-OH (103 mg, 62.0 μmol, 1.0 eq.) in DMF (1.5 mL) at rt. After stirring at rt for 1.5 h, TFA was added until acidic pH was reached. Purification by preparative HPLC (20 to 90% of ACN+0.1% TFA in water+0.1% TFA) afforded DM1-MCC-Cit-Lys(ma-Lys(PEG16))-Tyr-OH (73.4 mg, 29.4 μmol, 99% UV purity, 47% yield) as a white powder after freeze-drying. UPLC-MS (method 3): Rt=1.60 and 1.62 min, m/z=827 [M+3H]³+, 870 [M+FA-3H]³⁻

### Example 8.4.7: Preparation of DM1-Ac-Cit-Lys(Ac-Cys-ma-Lys(PEG16))-Tyr-OH

### Step 1.

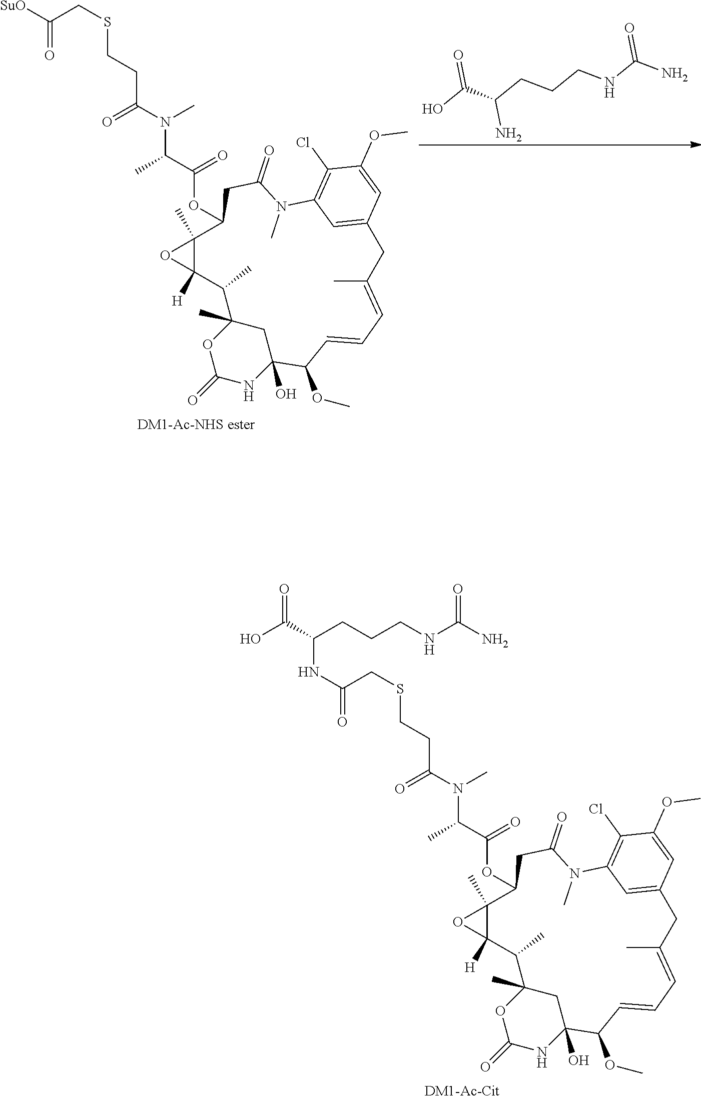

DIEA (0.14 mL, 0.83 mmol, 4.0 eq.) was added to a mixture of DM1-Ac—NHS ester (185 mg, 0.21 mmol, 1.0 eq.) and Citrulline (72.6 mg, 0.41 mmol, 2.0 eq.) in DMSO (3.7 mL) at rt. After stirring at rt for 18 h, TFA was added until acidic pH was reached. Purification by preparative HPLC (10 to 40% of can+0.1% TFA in water+0.1% TFA) afforded DM1-Ac-Cit (130 mg, 0.14 mmol, 100% UV purity, 66% yield) as a white powder after freeze-drying. UPLC-MS (method 1): Rt=1.33 min, m/z=952 [M−H]⁻.

### Step 2.

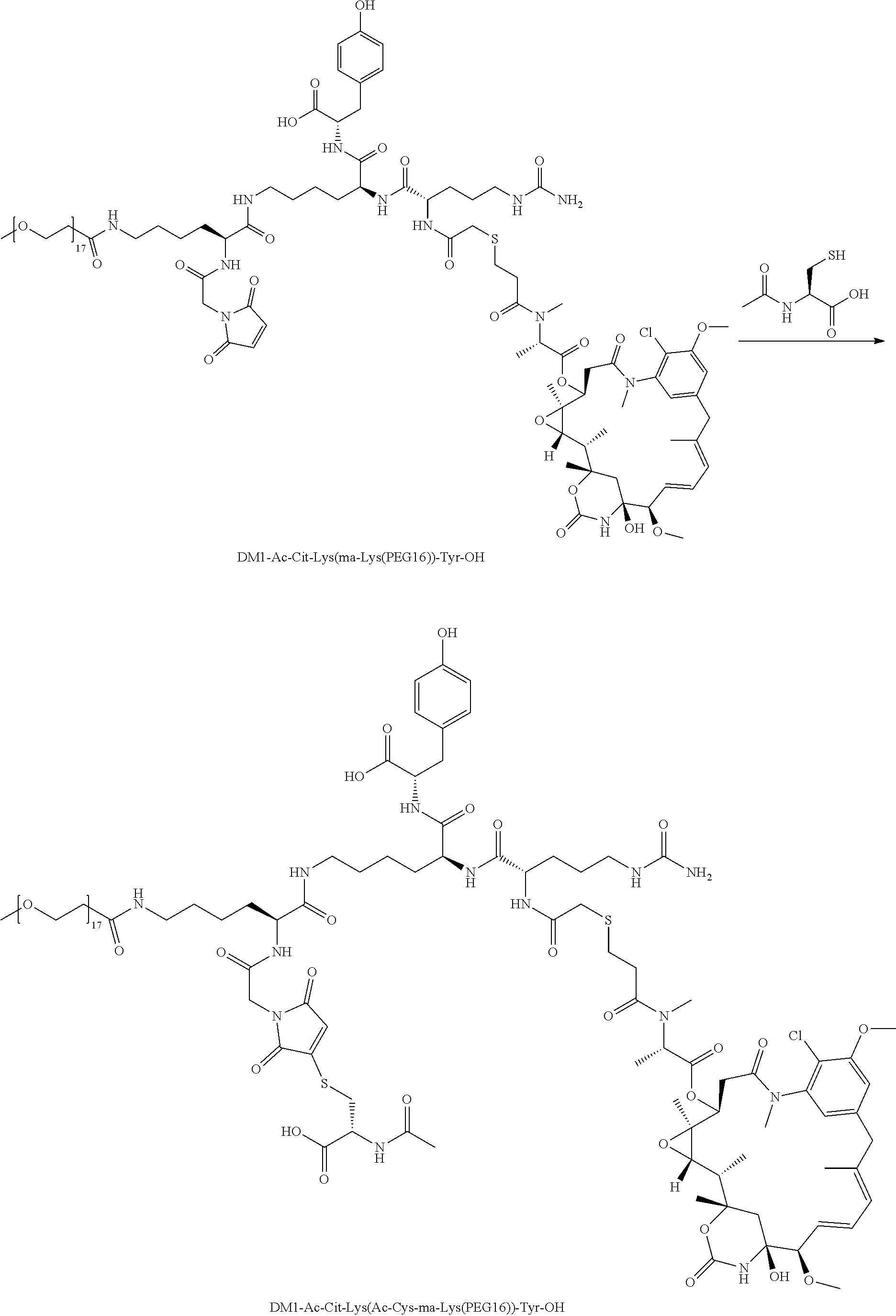

Acetyl-L-cysteine (0.32 mg, 2.17 μmol, 1.0 eq.) was added to a solution of DM1-Ac-Cit-Lys(ma-Lys(PEG16))-Tyr-OH (5.0 mg, 2.17 μmol, 1.0 eq.) in DMF (1 mL) at rt. After stirring at rt for 40 min, TFA was added until acidic pH was reached. Purification by preparative HPLC (5 to 100% can+0.1% TFA in water+0.1% TFA) afforded DM1-Ac-Cit-Lys(Ac-Cys-ma-Lys(PEG16))-Tyr-OH (4.59 mg, 1.86 μmol, 99% UV purity, 94% yield) as a white powder after freeze-drying. UPLC-MS (method 4): Rt=2.41 min, m/z=1231 [M-2H]²⁻.

### Example 8.4.8: Preparation of Exatecan-Suc-Phe-Cit-Lys(Ac-Cys-ma-Lys(Peg16))-Tyr-OH

### Step 1.

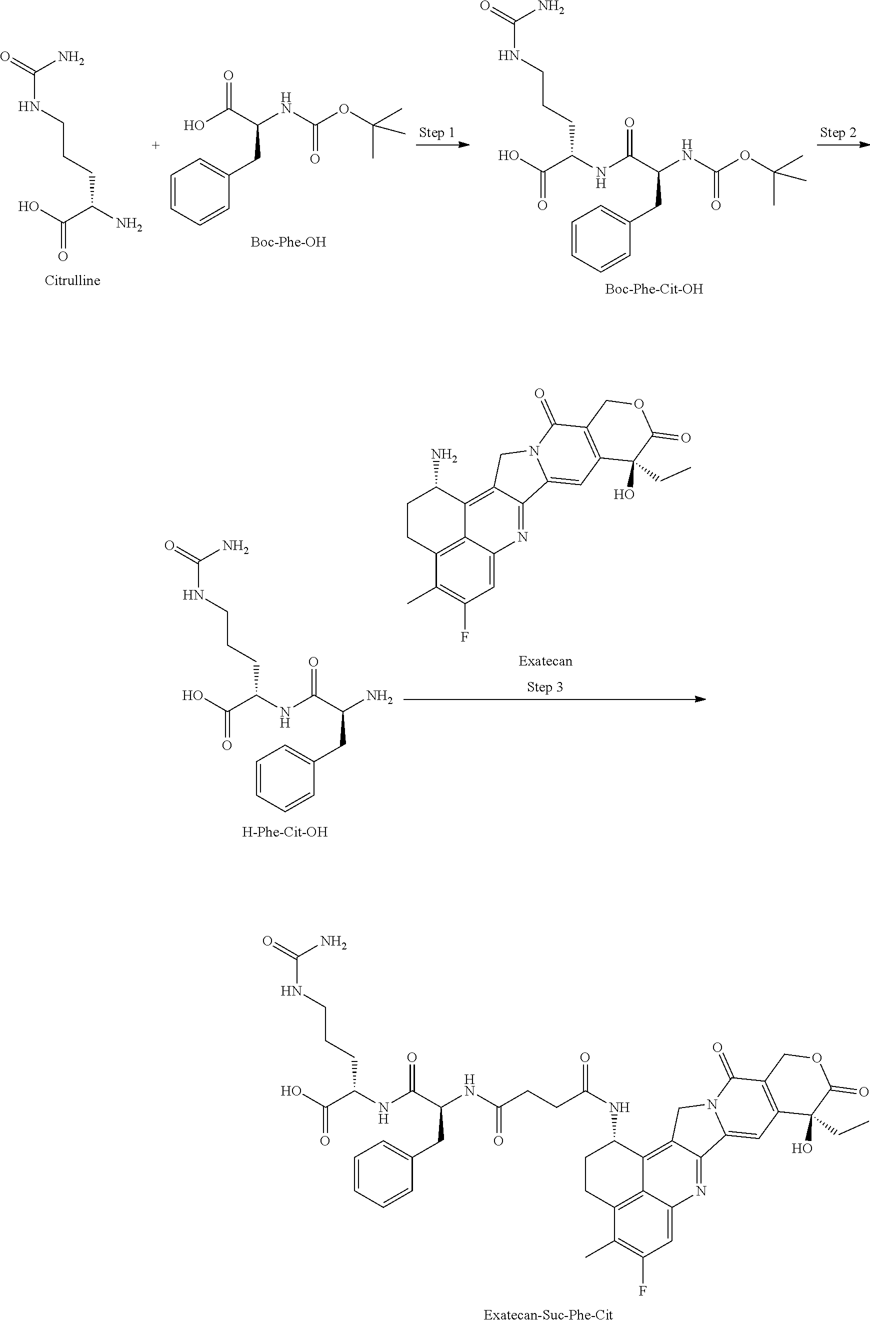

Step 1. DIEA (0.78 mL, 4.48 mmol, 4.0 eq.) was added to a mixture of Citrulline (200 mg, 1.12 mmol, 1.0 eq.) and Boc-Phe-OSu (405 mg, 1.12 mmol, 1.0 eq.) in DMF (10 mL) at rt. After stirring at rt for 16 h, the reaction mixture was filtered then concentrated under reduced pressure. A mixture of water/CAN/TFA (1:1:0.5%) was added. The mixture was filtered then freeze-dried. Purification by preparative HPLC (10 to 60% of ACN+0.1% TFA in water+0.1% TFA) afforded Boc-Phe-Cit-OH (86 mg, 0.20 mmol, 100% UV purity, 18% yield) as a white powder after freeze-drying. UPLC-MS (method 3): Rt=1.27 min, m/z=423 [M+H]⁺, 421 [M−H]⁻.

Step 2. A mixture of DCM (0.9 mL) and TFA (0.9 mL) was added to Boc-Phe-Cit-OH (60 mg, 0.14 mmol, 1.0 eq.) at rt. After stirring at rt for 20 min, the reaction mixture was concentrated. Water (10 mL) and ACN (10 mL) were added and the mixture was freeze-dried to afford H-Phe-Cit-OH (30 mg, 83.8 μmol, 90% UV purity, 59% yield) as a white powder. UPLC-MS (method 3): Rt=0.35 min, m/z=323 [M+H]⁺, 321 [M−H]⁻.

Step 3. Dihydrofuran-2,5-dione (8.4 mg, 83.5 μmol, 1.0 eq.) was added to a mixture of Exatecan (36 mg, 83.5 μmol, 1.0 eq.) and DIEA (0.17 mL, 1.00 mmol, 12 eq.) in DMF (0.8 mL) at rt. After stirring at rt for 10 min, 1-hydroxypyrrolidine-2,5-dione (9.6 mg, 83.5 μmol, 1.0 eq.) was added followed by HATU (31.7 mg, 83.5 μmol, 1.0 eq.). After stirring at rt for 15 min, H-Phe-Cit-OH (29.9 mg, 83.5 μmol, 1.0 eq.) was added to the reaction mixture. After stirring at rt for 1 h, H-Phe-Cit-OH (29.9 mg, 83.5 μmol, 1.0 eq.) was added to the reaction mixture. After stirring at rt for 1 h, TFA was added until acidic pH was reached. Purification by preparative HPLC (20 to 60% of ACN+0.1% TFA in water+0.1% TFA) afforded Exatecan-Suc-Phe-Cit (26.4 mg, 30.8 mmol, 98% UV purity, 37% yield) as a yellow powder after freeze-drying. UPLC-MS (method 2): Rt=1.39 min, m/z=840 [M+H]⁺, 838 [M−H]⁻.

### Step 4

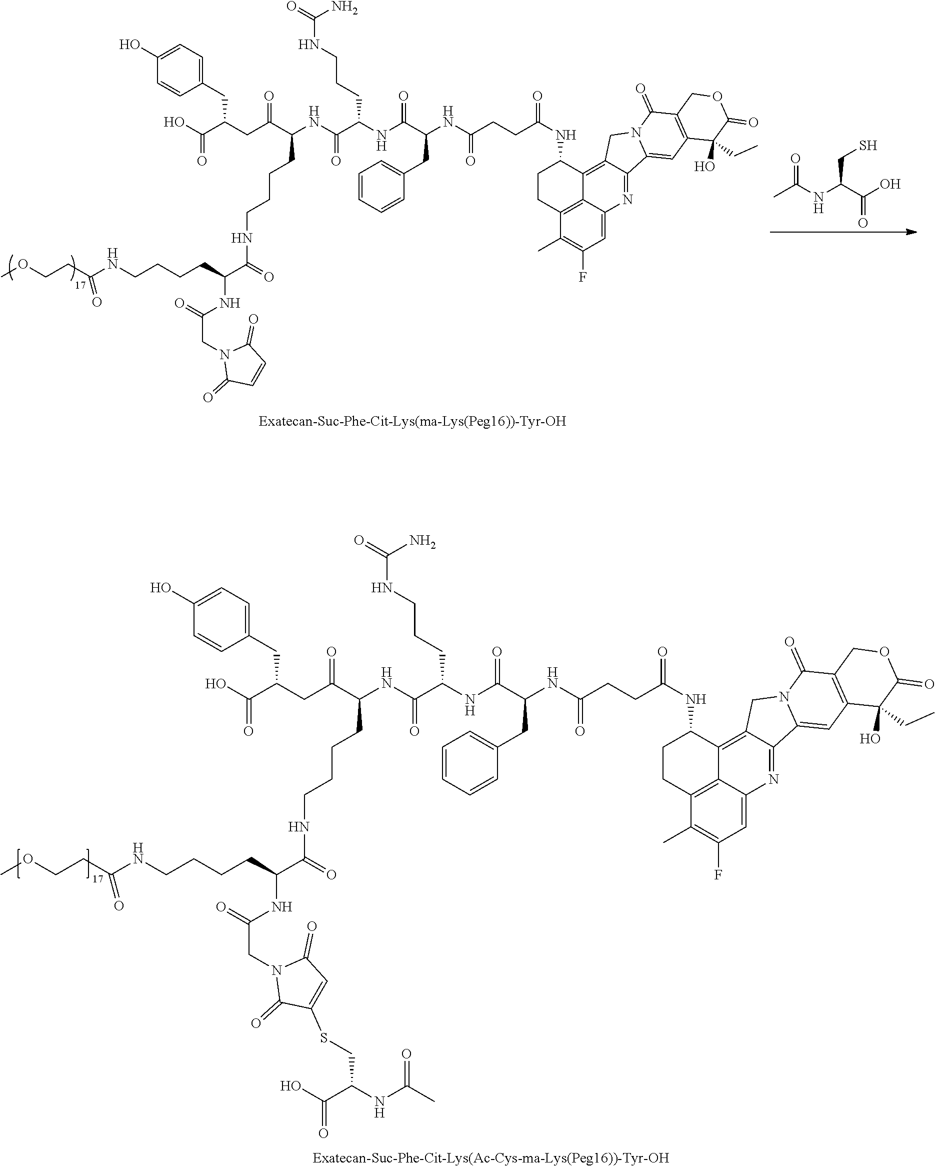

DIEA (1.2 μL, 6.8 μmol, 4.0 eq.) was added to a mixture of Exatecan-Suc-Phe-Cit-Lys(ma-C1-Lys(Peg16))-Tyr-OH (3.71 mg, 1.7 mmol, 1.0 eq.) and acetyl-L-cysteine (0.28 mg, 1.7 μmol, 1.0 eq.) in DMF (0.6 mL) at rt. After stirring at rt for 1 h, TFA was added until acidic pH was reached. Purification by preparative HPLC (20 to 60% of ACN+0.1% TFA in water+0.1% TFA) afforded Exatecan-Suc-Phe-Cit-Lys(Ac-Cys-ma-Lys(Peg16))-Tyr-OH (2.87 mg, 1.2 μmol, 100% UV purity, 72% yield) as a white powder after freeze-drying. UPLC-MS (method 4): Rt=2.37 min, m/z=1176 [M+2H]²+, 1174 [M-2H]²⁻.

### Example 8.4.9: Preparation of DM1-Ac-Cit-Lys(D-Arg-ma)-Tyr-OH

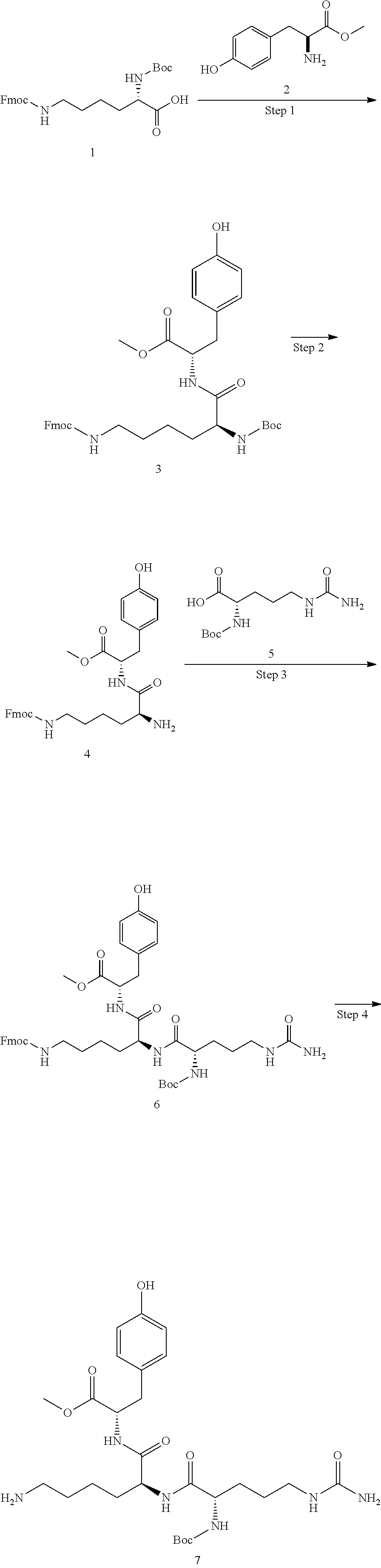

Step 1. DIEA (22.0 g, 0.17 mol, 4.0 eq) was added to a mixture of Boc-Lys(Fmoc)—OH (20.0 g, 43.0 mmol, 1.0 eq), H-Tyr-OMe (8.30 g, 43.0 mmol, 1.0 eq) and HATU (21.0 g, 55.0 mmol, 1.3 eq) in DMF (150 mL) at rt. After stirring at rt for 16 h, the reaction mixture was poured into water (400 mL) and extracted with EtOAc (3×80 mL). The combined organic phases were dried over sodium sulfate, concentrated and purified by silica gel column chromatography (eluting with MeOH/DCM, 0% to 5%) to afford Boc-Lys(Fmoc)-Tyr-OMe (26.0 g, 32.6 mmol, 85% UV purity, 76% yield) as a white solid. LC-MS (method 5): Rt=0.71 min, m/z=668.3 [M+Na]⁺.

Step 2. TFA (0.23 g, 2.00 mmol, 2.0 eq) was added to a solution of Boc-Lys(Fmoc)-Tyr-OMe (1.00 g, 1.00 mmol, 1.0 eq) in DCM (10 mL) at 0° C. After stirring at rt for 1 h, the reaction mixture was concentrated to give H-Lys(Fmoc)-Tyr-OMe (800 mg, 0.70 mmol, 85% UV purity, 70% yield) which was used directly in next step without further purification. LC-MS (method 5): Rt=1.11 min, m/z=546.3 [M+H]⁺.

Step 3. DIEA (11.3 g, 87.8 mmol, 2.4 eq) was added to a mixture of H-Lys(Fmoc)-Tyr-OMe (20.0 g, 36.6 mmol, 1.0 eq), Boc-Cit-OH (10.1 g, 36.6 mmol, 1.0 eq) and HATU (15.3 g, 40.2 mol, 1.1 eq) in DMF (100 mL) at rt. After stirring at rt for 16 h, the reaction mixture was poured into water (400 mL) and extracted with EtOAc (3×80 mL). The combined organic phases were dried over sodium sulfate, concentrated and purified by silica gel column chromatography (eluting with MeOH/DCM, 0% to 5%) to afford Boc-Cit-Lys(Fmoc)-Tyr-OMe (12.6 g, 15.4 mmol, 90% UV purity, 42% yield) as a white solid. LC-MS (method 5): Rt=1.25 min, m/z=803.4 [M+H]⁺.

Step 4. Diethylamine (1.36 g, 18.6 mmol, 3.0 eq) was added to a solution of Boc-Cit-Lys(Fmoc)-Tyr-OMe (5.00 g, 6.20 mmol, 1.0 eq) in DCM (10 mL) at rt. After stirring at rt for 10 h, the reaction mixture was concentrated, washed with Et20 and triturated in Et20 to afford Boc-Cit-Lys-Tyr-OMe (3.00 g, 4.59 mmol, 90% UV purity, 74% yield) which was used directly in next step without further purification. LC-MS (method 5): Rt=1.26 min, m/z=581.4 [M+H]⁺.

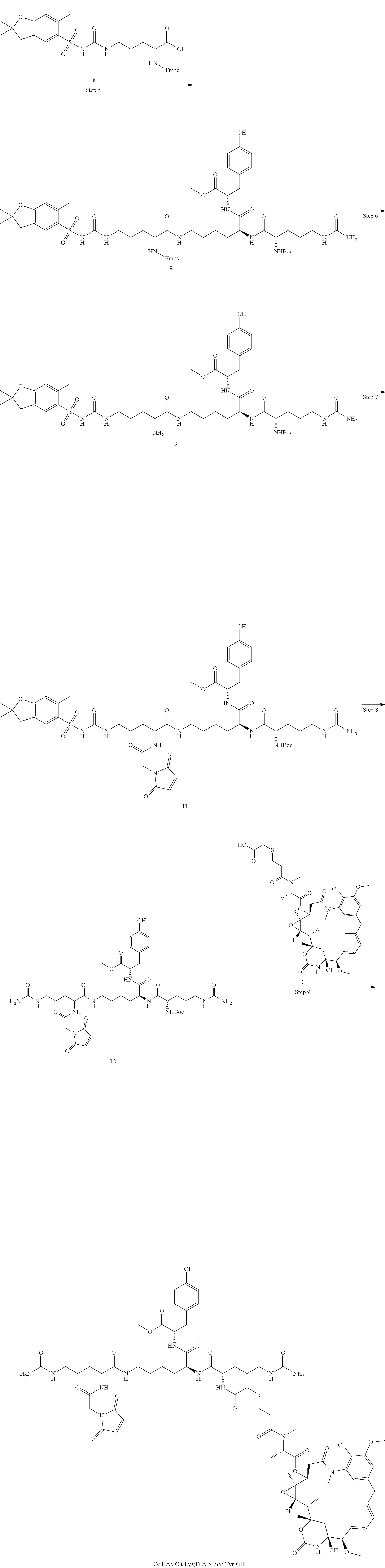

Step 5. DIEA (0.80 g, 6.20 mol, 2.0 eq) was added to a mixture of Boc-Cit-Lys-Tyr-OMe (1.98 g, 3.40 mmol, 1.1 eq), Fmoc-D-Arg (Pbf)-OH (2.00 g, 3.10 mmol, 1.0 eq) and HATU (1.30 g, 3.40 mmol, 1.1 eq) in DMF (30 mL) at rt. After stirring at rt for 16 h, the reaction mixture was poured into water (300 mL) and extracted with EtOAc (3×80 mL). The combined organic phases were dried over sodium sulfate, concentrated, and purified by silica gel column chromatography (eluting with MeOH/DCM, 0% to 5%) to afford Boc-Cit-Lys(Fmoc-D-Arg(Pbf))-Tyr-OMe (2.10 g, 2.26 mmol, 95% UV purity, 73% yield) as a white solid. LC-MS (method 6): Rt=2.08 min, m/z=1211.7 [M+H]⁺.

Step 6. LiOH (50 mg, 1.20 mmol, 1.5 eq) was added to a solution of Boc-Cit-Lys(Fmoc-D-Arg(Pbf))-Tyr-OMe (1.00 g, 0.80 mmol, 1.0 eq) in THF/water (1:1, 10 mL) at rt. After stirring at rt for 10 h, the organic solvent was concentrated then the aqueous solution was adjusted to pH 2-3 with citric acid. The precipitate was collected by filtration, washed with water, and purified on a Biotage Isolera One (C18 column, eluting with 5% to 95% ACN/water containing 0.1% FA) to provide Boc-Cit-Lys(D-Arg(Pbf))-Tyr-OH (0.40 g, 0.37 mmol, 90% UV purity, 46% yield) as a white solid. LC-MS (method 5): Rt=0.98 min, m/z=975.4 [M+H]⁺.

Step 7. Triethylamine (15 mg, 0.15 mmol, 1.5 eq) was added to a mixture of Boc-Cit-Lys(D-Arg(Pbf))-Tyr-OH (100 mg, 0.10 mmol, 1.0 eq), AMAS (28 mg, 0.11 mmol, 1.1 eq) in DMF (1 mL) at rt. After stirring at rt for 30 min, TFA was added until pH 5-6 was reached. Purification on a Biotage Isolera One (C18 column, eluting with 5% to 95% ACN/water containing 0.1% TFA) afforded Boc-Cit-Lys(D-Arg(Pbf)-ma)-Tyr-OH (38 mg, 29 μmol, 88% UV purity, 29% yield) as a white solid. LC-MS (method 6): Rt=1.70 min, m/z=1112.6 [M+H]⁺.

Step 8. Boc-Cit-Lys(D-Arg(Pbf)-ma)-Tyr-OH (10 mg, 9.0 μmol, 1.0 eq) was solubilized in a mixture of TFA/DCM (1:1, 1 mL) at rt. After stirring at rt for 30 min, the mixture was concentrated, washed with Et20 and triturated in Et20 to provide H-Cit-Lys(D-Arg-ma)-Tyr-OH (6.0 mg, 6.2 μmol, 90% UV purity, 69% yield) which was used directly in next step without further purification. LC-MS (method 6): Rt=0.97 min, m/z=758.4 [M+H]⁺.

Step 9. HOSu (1.6 mg, 13.8 μmol, 1.1 eq) was added to a mixture of DM1-Ac (10 mg, 12.6 μmol, 1.0 eq) and EDCl (2.6 mg, 13.8 μmol, 1.1 eq) in DMF (1 mL) at rt. After stirring at rt for 1 h, the reaction mixture was poured into water (40 mL) and extracted with EA (3×20 mL). The combined organic phases were dried over sodium sulfate and concentrated under vacuum. The residue was dissolved in DMF (1 mL). H-Cit-Lys(D-Arg-ma)-Tyr-OH (11.4 mg, 12.6 μmol, 1.0 eq) and DIEA (2.44 mg, 18.9 μmol, 1.5 eq) were added to the later solution at rt. After stirring at rt for 1 h, TFA was added until pH 5-6 was reached. Purification on a Biotage Isolera One (C18 column, eluting with 5% to 75% ACN/water containing 0.1% TFA) afforded DM1-Ac-Cit-Lys(D-Arg-ma)-Tyr-OH (3.0 mg, 2.27 μmol, 88% UV purity, 18% yield) as a white solid. LC-MS (method 5): Rt=1.01 min, m/z=1537.5 [M+H]⁺.

### 8.5 Preparation and Characterisation of Antibody-Drug Conjugates (ADCs):

### 8.5.1 Preparation of ADCs: General Procedures

All linker-payloads prepared in part 8.4 were conjugated to Herceptin® (Trastuzumab). Linker-payloads DM1-Ac-Cit-Lys(ma-Lys(PEG16))-Tyr-OH and Exatecan-Suc-Phe-Cit-Lys(ma-Lys(PEG16))-Tyr-OH were also conjugated to Naratuximab.

### General Procedure 1: For DOC 4 Tmab Conjugation:

A solution of TCEP·HCl (0.32 mg, 1.09 μmol, 2.3 eq.) in DPBS (0.32 mL) was added to a solution of Trastuzumab (70 mg, 0.45 μmol, 1.0 eq.) in water (3.33 mL) and DPBS (3.33 mL) at rt. The reaction mixture was purged with nitrogen then stirred at 40° C. After stirring at 40° C. for 70 min, a solution of linker-payload (3.55 μmol, 7.4 eq.) in DMSO (0.7 mL) was added. The reaction mixture was stirred at rt for 70 min then diluted to V=10 mL with 10×pH8 DPBS. Purification using a PF100 column and pH8 DPBS as eluent afforded a fraction containing the desired ADC (14 mL). This fraction was stirred at rt for 16 h then centrifugated (10 min, 4000 rpm) and finally the supernatant was transferred to an Amicon concentrating cell (15 mL, 50 kDa). The mixture was concentrated by centrifugation (4500 rpm, 3800 G) to V=1 mL, DPBS buffer was added (14 mL) and the mixture was concentrated again (4500 rpm, 3800 G) to V=1 mL. DPBS buffer was added (14 mL) and the mixture were concentrated again (4500 rpm, 3800 G) to V=1 mL. The final volume was adjusted to V=7.0 mL with DPBS buffer. The solution was filtered using a 25 mm PES 0.22 μm Millex filter then aliquoted and stored at −80° C.

### General Procedure 2: For DOC 8 Tmab Conjugation:

A solution of TCEP·HCl (1.10 mg, 3.80 μmol, 8.0 eq.) in DPBS (0.11 mL) was added to a solution of Trastuzumab (70 mg, 0.45 μmol, 1.0 eq.) in water (3.33 mL) and DPBS (3.33 mL) at rt. The reaction mixture was purged with nitrogen then stirred at 40° C. After stirring at 40° C. for 70 min, a solution of linker-payload (9.60 μmol, 20.0 eq.) in DMSO (0.7 mL) was added. The reaction mixture was stirred at rt for 70 min then diluted to V=10 mL with 10×pH8 DPBS. Purification using a PF100 column and pH8 DPBS as eluent afforded a fraction containing the desired ADC (14 mL). This fraction was stirred at rt for 16 h then centrifugated (10 min, 4000 rpm) and finally the supernatant was transferred to an Amicon concentrating cell (15 mL, 50 kDa). The mixture was concentrated by centrifugation (4500 rpm, 3800 G) to V=1 mL, DPBS buffer was added (14 mL) and the mixture was concentrated again (4500 rpm, 3800 G) to V=1 mL. DPBS buffer was added (14 mL) and the mixture were concentrated again (4500 rpm. 3800 G) to V=1 mL. The final volume was adjusted to V=7.0 mL with DPBS buffer. The solution was filtered using a 25 mm PES 0.22 μm Millex filter then aliquoted and stored at −80° C.

### General Procedure 3: For DOC 8 Naratuximab Conjugation:

A solution of TCEP·HCl (1.10 mg, 3.80 μmol, 8.0 eq.) in DPBS (0.11 mL) was added to a solution of naratuximab (70 mg, 0.48 μmol, 1.0 eq.) in buffer (7.00 mL, 50 mM potassium phosphate, 50 mM potassium chloride, 2 mM EDTA, pH 6.5) at rt. The reaction mixture was purged with nitrogen then stirred at 40° C. After stirring at 40° C. for 70 min, a solution of linker-payload (9.60 μmol, 20.0 eq.) in DMSO (0.7 mL) was added. The reaction mixture was stirred at rt for 70 min then diluted to V=10 mL with 10×pH8 DPBS. Purification using a PF100 column and pH8 DPBS as eluent afforded a fraction containing the desired ADC (14 mL). This fraction was stirred at rt for 16 h then centrifugated (10 min, 4000 rpm) and finally the supernatant was transferred to an Amicon concentrating cell (15 mL, 50 kDa). The mixture was concentrated by centrifugation (4500 rpm, 3800 G) to V=1 mL, DPBS buffer was added (14 mL) and the mixture was concentrated again (4500 rpm, 3800 G) to V=1 mL. DPBS buffer was added (14 mL) and the mixture were concentrated again (4500 rpm. 3800 G) to V=1 mL. The final volume was adjusted to V=7.0 mL with DPBS buffer. The solution was filtered using a 25 mm PES 0.22 μm Millex filter then aliquoted and stored at −80° C.

### 8.5.2 Summary of Characterisation of Prepared ADCs

TABLE 2
Summary of prepared ADCs

| | | | | General |
|---|---|---|---|---|
| ADC | | | Targeted | procedure |
| number | mAb | Linker name | DOC/DAR | number |
| ADC-1 | Tmab | DM1-MCC-Cit-Lys(ma- | 8/8 | 2 |
| | | PEG5)-Tyr-OH | | |
| ADC-2 | Tmab | DM1-Ac-Cit-Lys(ma- | 8/8 | 2 |
| | | Lys(PEG16))Tyr-OH | | |
| ADC-3 | Tmab | DM1-Ac-Cit-Lys(ma- | 8/8 | 2 |
| | | PEG5)-Tyr-OH | | |
| ADC-4 | Nmab | DM1-Ac-Cit-Lys(ma- | 8/8 | 3 |
| | | Lys(PEG16))Tyr-OH | | |
| ADC-5 | Tmab | Exatecan-Suc-Phe-Cit- | 8/8 | 2 |
| | | Lys(ma-Lys(Peg16))- | | |
| | | Tyr | | |
| ADC-6 | Nmab | Exatecan-Suc-Phe-Cit- | 8/8 | 3 |
| | | Lys(ma-Lys(Peg16))- | | |
| | | Tyr | | |
| ADC-7 | Tmab | ma-Gly-Glu(DM1-Ac- | 8/16 | 2 |
| | | Cit-Lys-Tyr-OH)- | | |
| | | Glu(DM1-Ac-Cit-Lys- | | |
| | | Tyr-OH)-PEG24 | | |
| ADC-8 | Tmab | ma-Gly-Glu(DM1-Ac- | 4/8 | 1 |
| | | Cit-Lys-Tyr-OH)- | | |
| | | Glu(DM1-Ac-Cit-Lys- | | |
| | | Tyr-OH)-PEG24 | | |
| ADC-9 | Tmab | DM1-MCC-Cit-Lys(ma- | 8/8 | 2 |
| | | Lys(PEG16)-Tyr-OH | | |
| ADC-10 | Tmab | DM1-Ac-Cit-Lys(D-Arg- | 8/8 | 2 |
| | | ma)-Tyr-OH | | |

All characterisations are given in Table 3 below.

TABLE 3
Characterisations of prepared ADC

| | | | | | Aggregation |
|---|---|---|---|---|---|
| | | | | | (%)_{by SEC} |
| | | | | | _{after a} |
| ADC | | Concentration | DAR by | DAR by | _{freeze/thaw} |
| number | Yield (%) | (mg/mL) | RPLC | MS | _{cycle} |
| ADC-1 | 18.1 | 1.91 | 7.30 | — | 2.04 |
| ADC-2 | 79.9 | 8.16 | 8.15 | — | 1.50 |
| ADC-3 | 22.3 | 2.37 | 6.78 | — | 3.18 |
| ADC-4 | 89.1 | 9.11 | 7.66 | — | 1.61 |
| ADC-5 | 65.6 | 3.28 | 7.58 | — | 4.74 |
| ADC-6 | 78.8 | 3.94 | 7.90 | — | 3.16 |
| ADC-7 | 52.8 | 7.30 | 15.29 | — | 1.46 |
| ADC-8 | 74.5 | 7.74 | 10.35 | — | 1.33 |
| ADC-9 | 75.6 | 7.56 | 8.13 | — | 0.71 |
| ADC-10 | 15.5 | 1.37 | — | 8.27 | 1.65 |

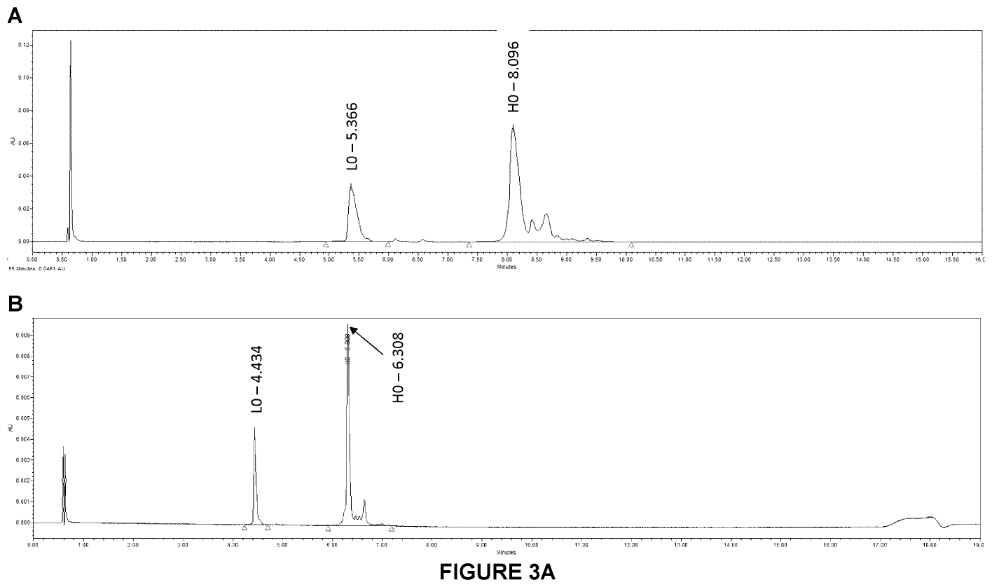

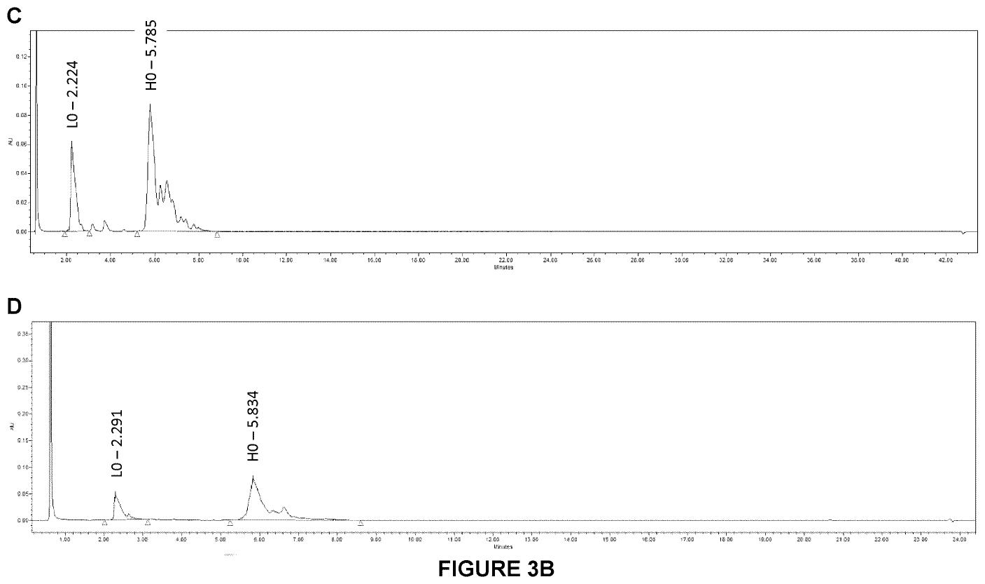

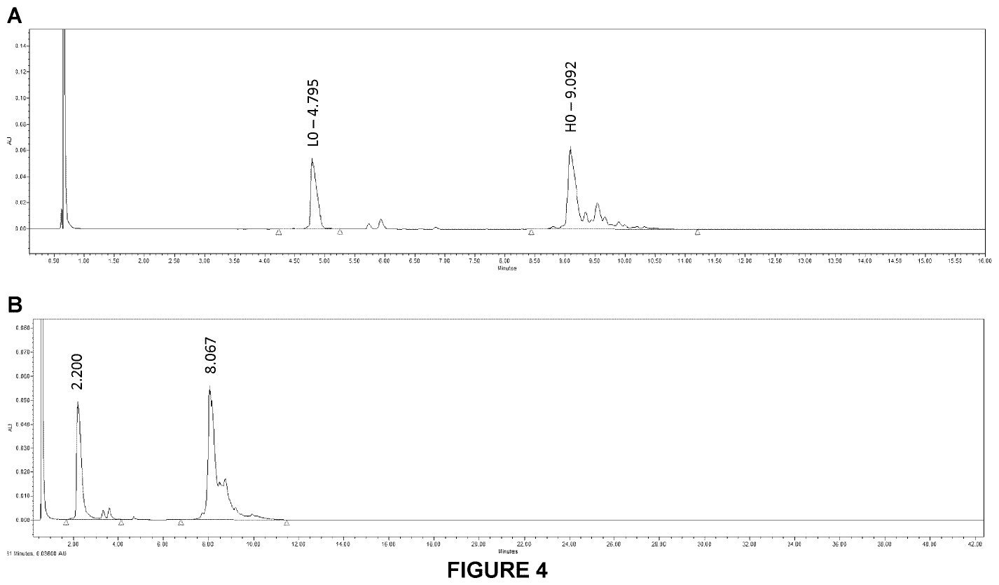

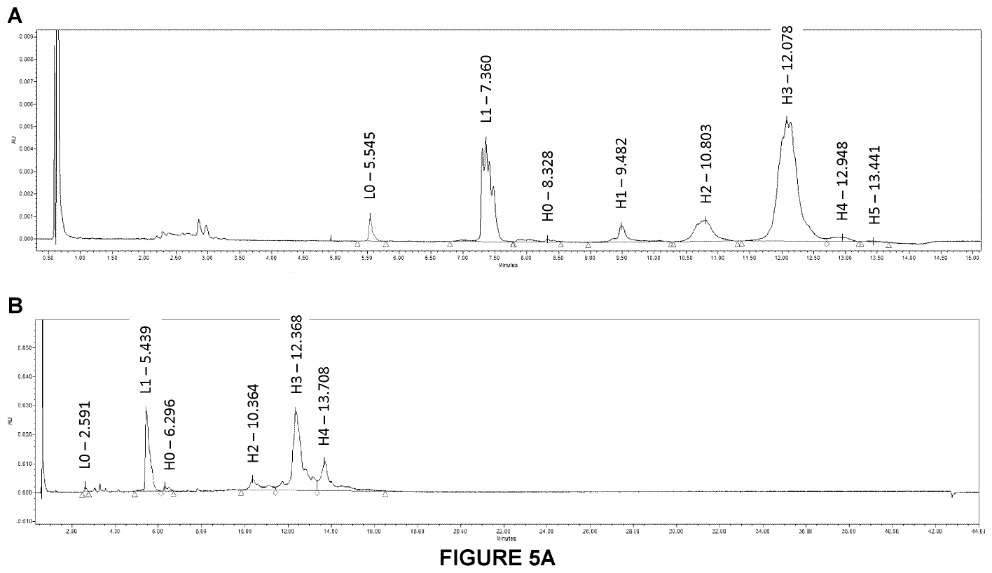

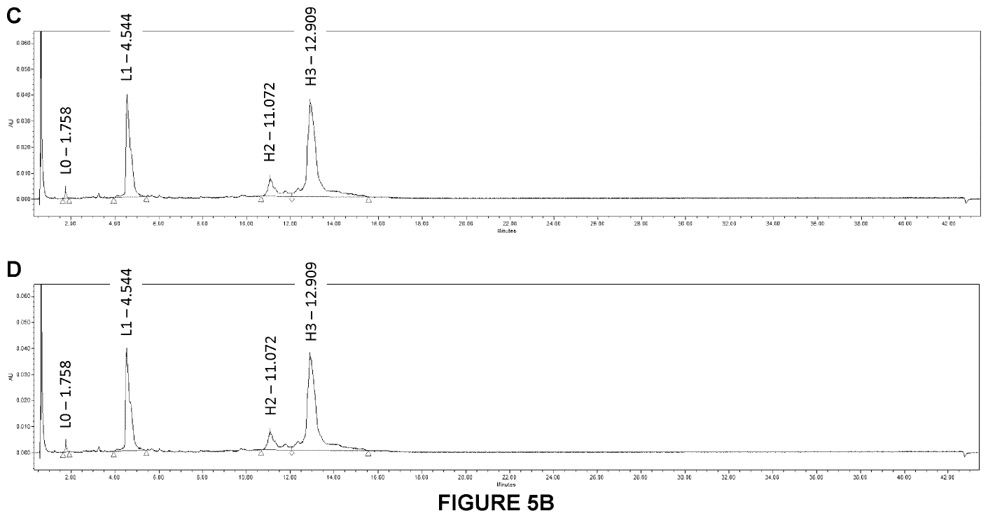

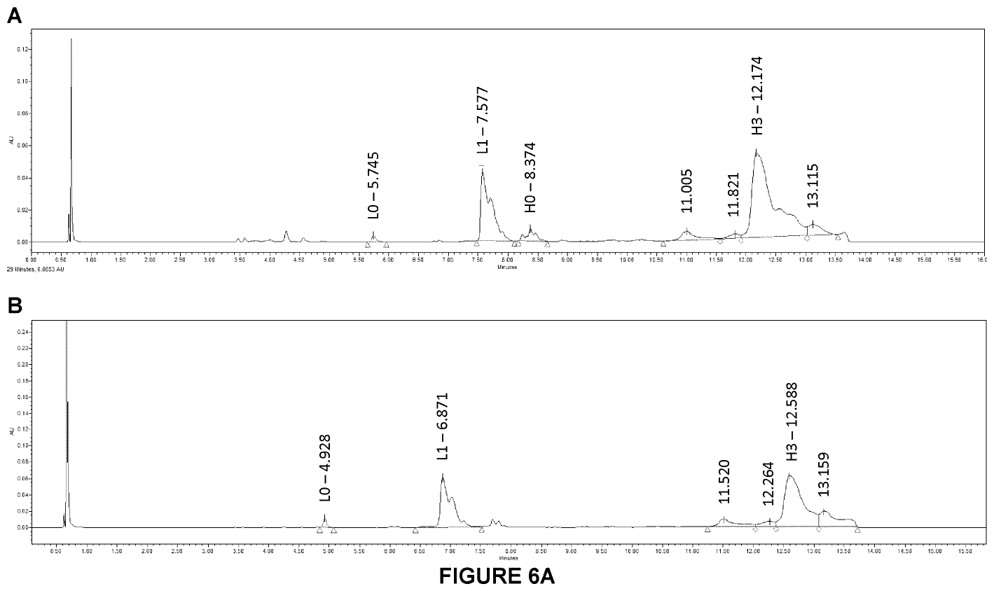

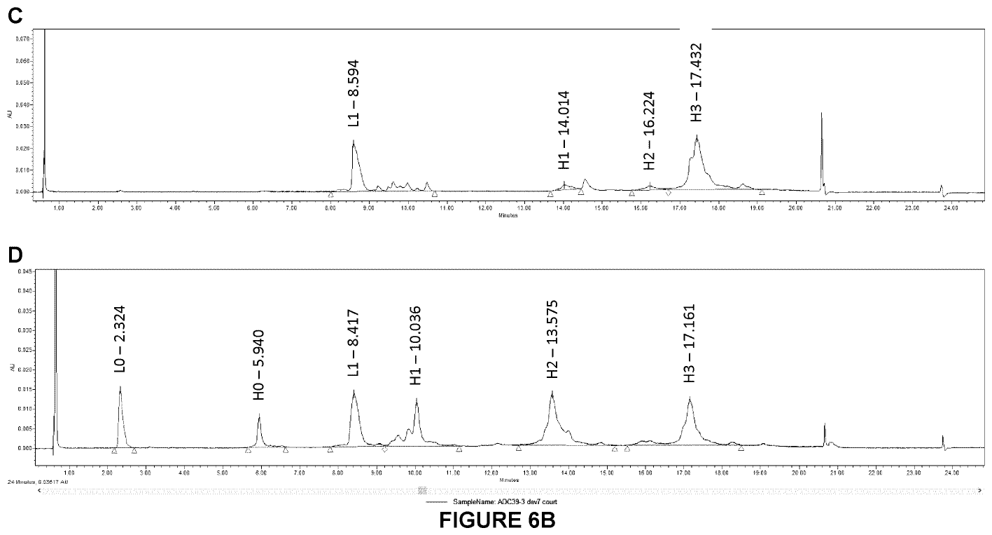

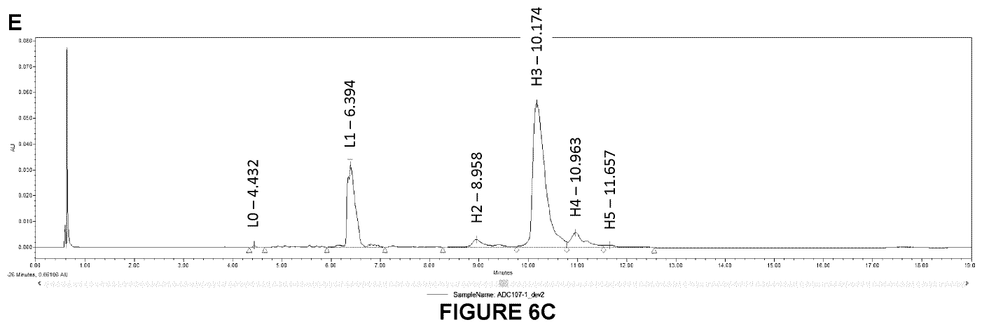

The RPLC chromatograms of the prepared ADCs are shown in FIGS. 3 to 6.

### 8.5.3 Preparation of Naratuximab Emtansine (Debio1562) (Comparative Example)

Naratuximab was reacted with the heterobifunctional crosslinking reagent SMCC and the maytansinoid DM1 using the one step process described in WO2012135517 Å2.

The DAR was 3.5 (measured by UV at 2 different wavelengths 280 nm and 252 nm).

### 8.6 in Vitro and in Vivo Studies

### 8.6.1 in Vitro Cytotoxicity Data

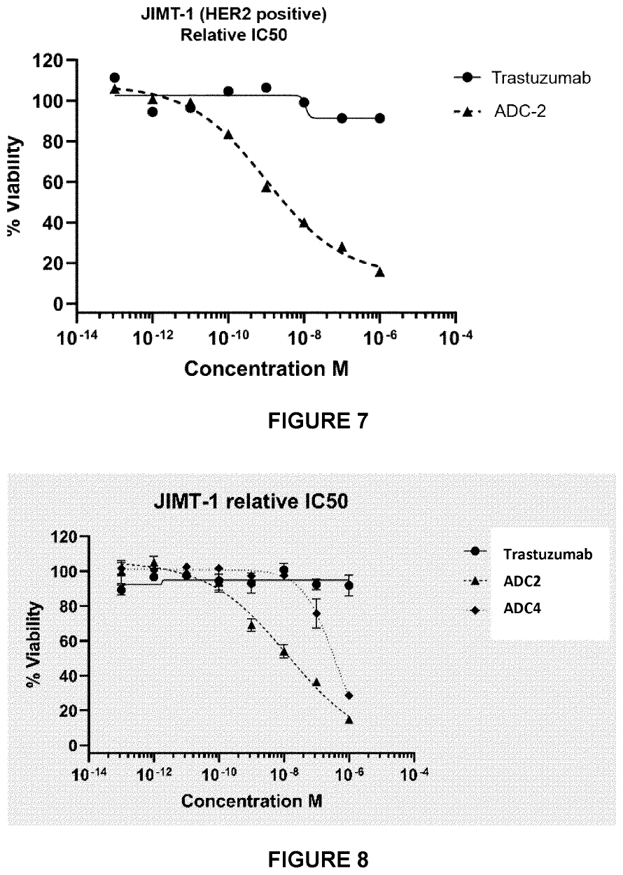

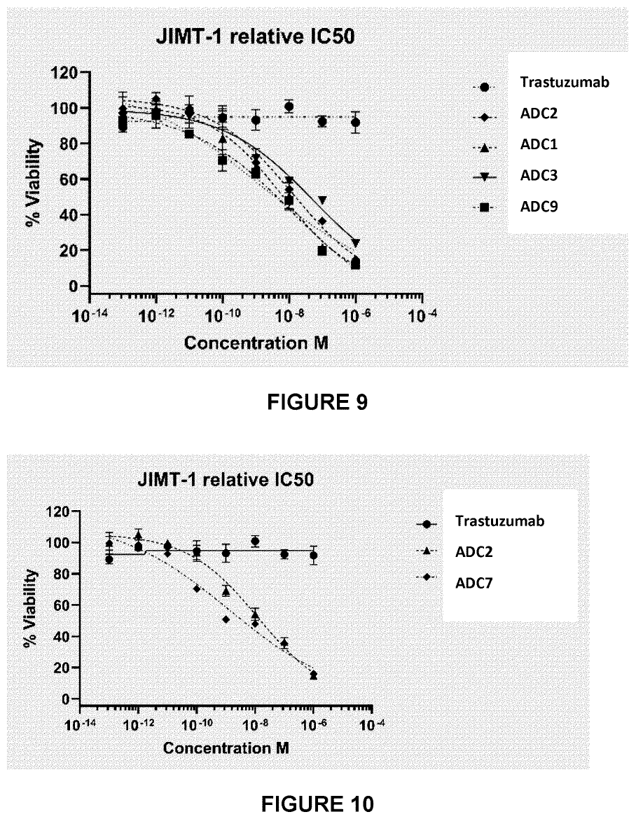

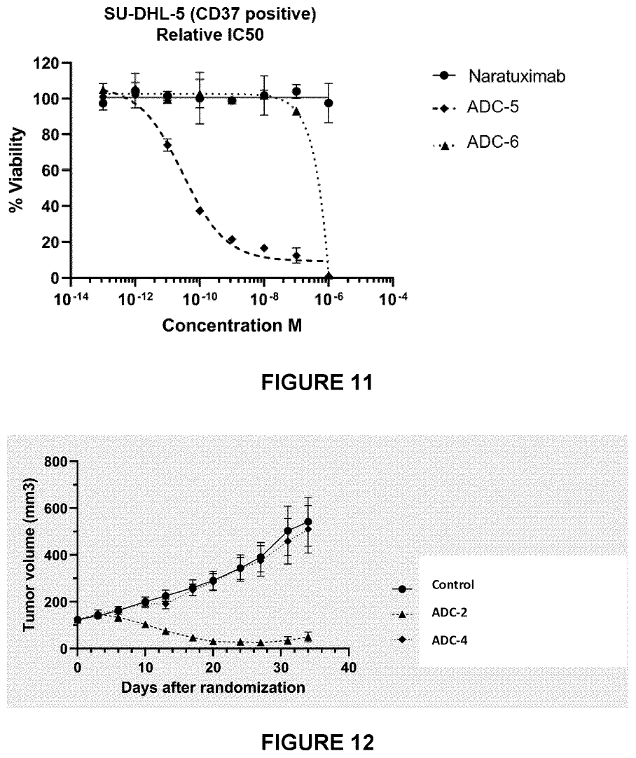

In vitro cytotoxic assay: JIMT-1 (HER2 positive) cells were plated and after overnight resting (12 hours), serial dilutions of drugs (Trastuzumab, Naratuximab, ADC-1, ADC-2, ADC-3, ADC-4, ADC-5, ADC-6, ADC-7, or ADC-9) were added to the cells. After 72 hours of incubation, CellTiter-Glo 2.0 (Promega kit (according to manufacturer's instructions) were added to each well before reading of luminescence from a luminometer. Relative IC50 were calculated using GraphPad Prism. The results of the in vitro cytotoxic assay are shown in FIGS. 7 to 11.

### 8.6.2 in Vivo Efficacy Data

A) Briefly, NMRI mice were inoculated with Jimt-1 breast cancer cells (5×10⁶ cells per mouse). When tumors reached an average size of 150 mm3, mice were randomized into 3 groups of 8 mice (day 0) and treatment started the day after (Day 1). In the non-control group mice two (100 ul) intravenous injections of 5 mg/kg of ADC-2 or ADC-4 were performed 1 week apart. The vehicle was phosphate buffer saline (PBS). The control group mice received two (100 ul) intravenous injections of vehicle.

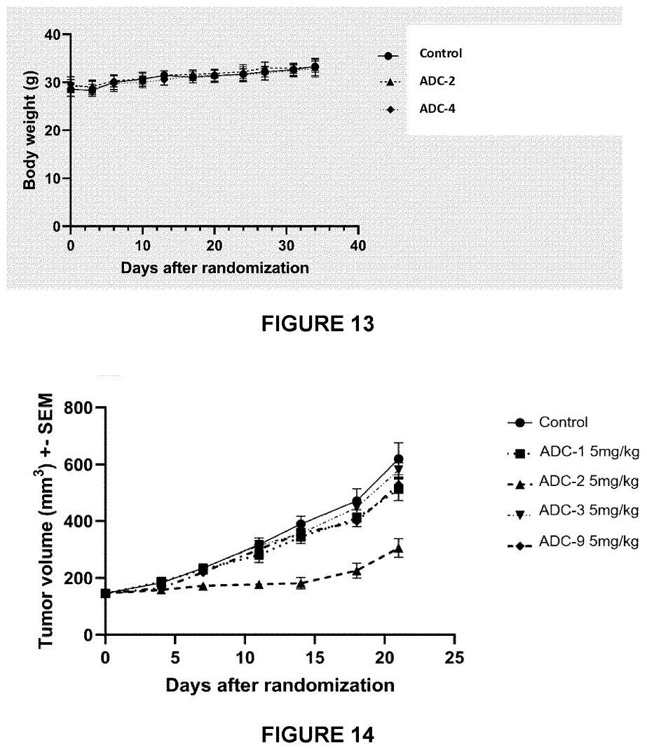

After tumor cells inoculation, the animals were routinely monitored as set out below. Mean tumor volume and body weight results from the study are shown in FIGS. 12 and 13.

### 8.6.3 Routine Monitoring

After tumor cells inoculation, the animals were checked daily for morbidity and mortality. During routine monitoring, the animals were checked for any effects of tumor growth and treatments on behavior such as mobility, food and water consumption, body weight gain/loss (body weights were measured 2 times a week after randomization), eye/hair matting and any other abnormalities. Mortality and observed clinical signs were recorded for individual animals in detail.

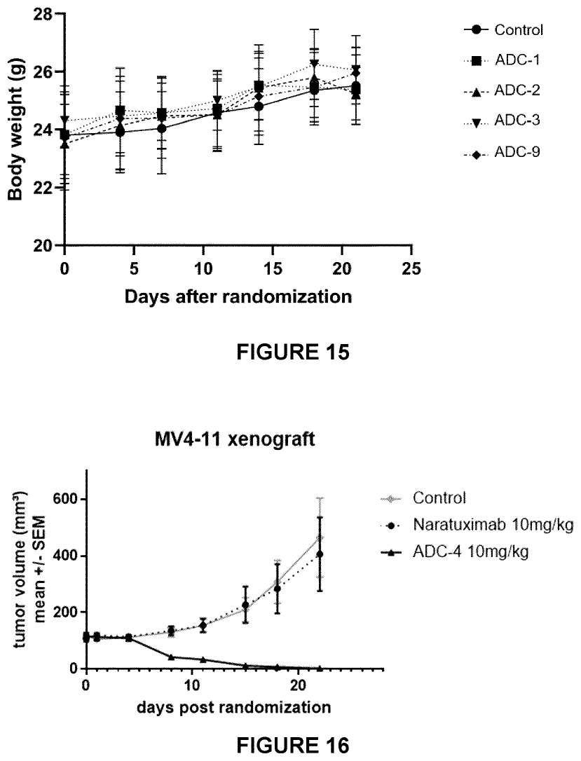

Tumor volumes were measured 2 times a week in two dimensions using a caliper, and the volume is expressed in mm3 using the formula: “V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet.
B) Briefly, NOD/SCID mice were inoculated with Jimt-1 breast cancer cells (5×10⁶ cells per mouse). When tumors reached an average size of 150 mm3, mice were randomized into 5 groups of 8 mice and treatment started the same day (Day 0). In the non-control group mice two intravenous injections of 5 mg/kg of ADC-1, ADC-2, ADC-3, or ADC-9 were performed 1 week apart. The vehicle was PBS. The control group mice received two (100 ul) intravenous injections of vehicle. After tumor cells inoculation, the animals were routinely monitored as set out above. Mean tumor volume and body weight results from the study are shown in FIGS. 14 and 15.

### 8.6.4 in Vivo Efficacy Data

NMRI nude mice were inoculated with MV4;11 Acute Myeloid Leukemia cells (5×10⁶ cells per mouse). When tumors reached an average size of 115 mm³, mice were randomized in groups of 8 mice for vehicle (group 1) or 6 mice for treated conditions (group 2 and 3) (DO), and treatment started the day after (D1). In the non-control group mice, one intravenous injection of 10 mg/kg of naratuximab or ADC-4 was performed. The Vehicle was PBS. The control group mice received one intravenous injection of the vehicle (5 mL/kg). After tumor cells inoculation, the animals were routinely monitored as set out above. The mean tumor volume results are shown in FIG. 16.

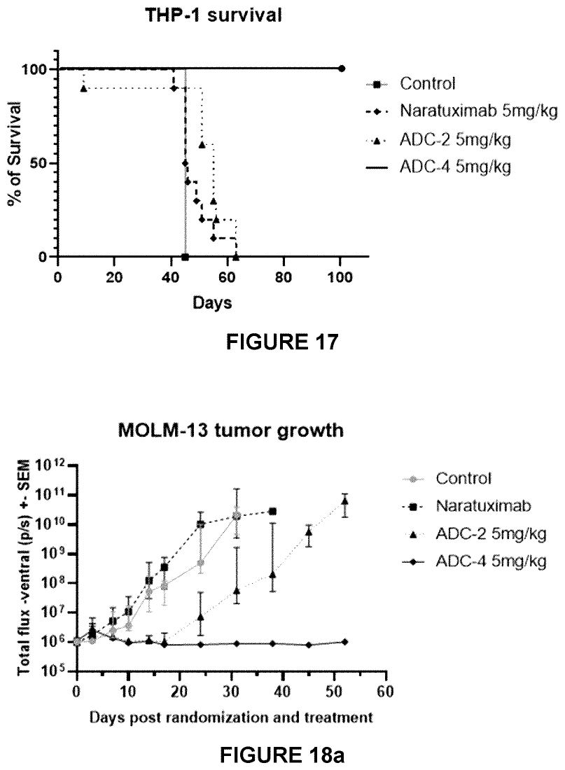

NOD/SCID mice were irradiated with 200 rad Co60 24 hours before being inoculated with THP-1 Acute Myeloid Leukemia cells in tails to allow tumor dissemination (1×10⁷ cells per mouse). Mice were randomized (Randomization was performed based on “Matched distribution” method/“Stratified” method (StudyDirector™ software, version 3.1.399.19)/randomized block design) into 4 groups of 10 mice (DO), and treatment started seven days after (D7). In the non control group mice, one intravenous injection of 5 mg/kg of naratuximab, ADC-2, or ADC-4 was performed. The Vehicle was PBS. The control group mice received an injection of the vehicle (5 mL/kg). After tumor cells inoculation, the animals were routinely monitored as set out above. The % survival results from the study are shown in FIG. 17.

NOD/SCID mice were inoculated with MOLM-13 Luciferase Acute Myeloid Leukemia cells in tails to allow tumor dissemination (2×10⁶ cells per mouse). Mice were randomized based on total flux value 3 days after inoculation in 4 groups of 8 mice (DO) and treatment started 7 days after inoculation (D4). In the non-control group mice, one intravenous injection of 5 mg/kg of naratuximab, ADC-2 or ADC-4 was performed.

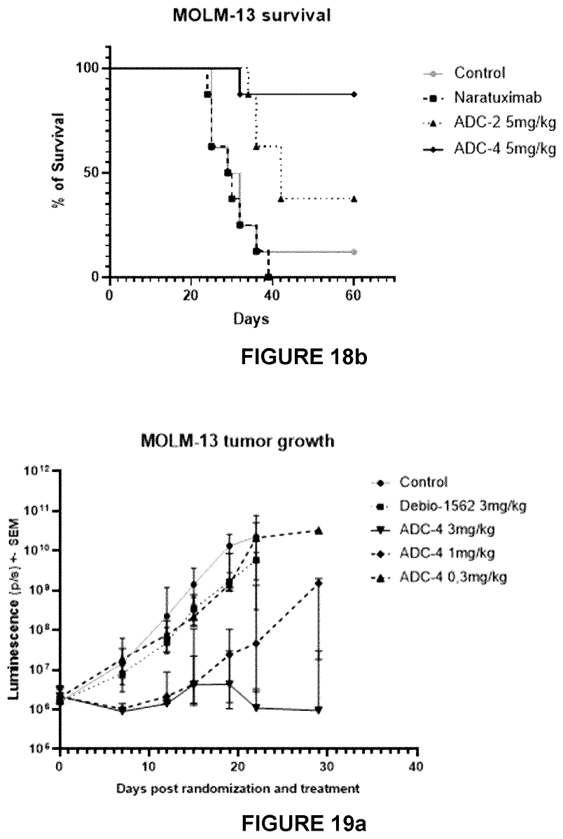

The Vehicle was PBS. The control group mice received an injection of the vehicle (5 mL/kg). After tumor cells inoculation, the animals were routinely monitored as set out above, and tumor growth was imaged twice per week after Luciferine (using an IVIS Spectrum BL from PerkinElmer). The tumor growth and % survival results are shown in FIGS. 18a and 18b.

NOD/SCID mice were inoculated with MOLM-13 Luciferase Acute Myeloid Leukemia cells in tails to allow tumor dissemination (2×10⁶ cells per mouse). Mice were randomized based on total flux value 7 days after inoculation in 5 groups of 8 mice and treatment started the day of randomization (DO). In the non-control group mice, one intravenous injection of 3 mg/kg of ADC-4, 1 mg/kg of ADC-4, 0.3 mg/kg of ADC-4, or 3 mg/kg of naratuximab emtansine (Debio-1562) was performed. The vehicle was PBS.

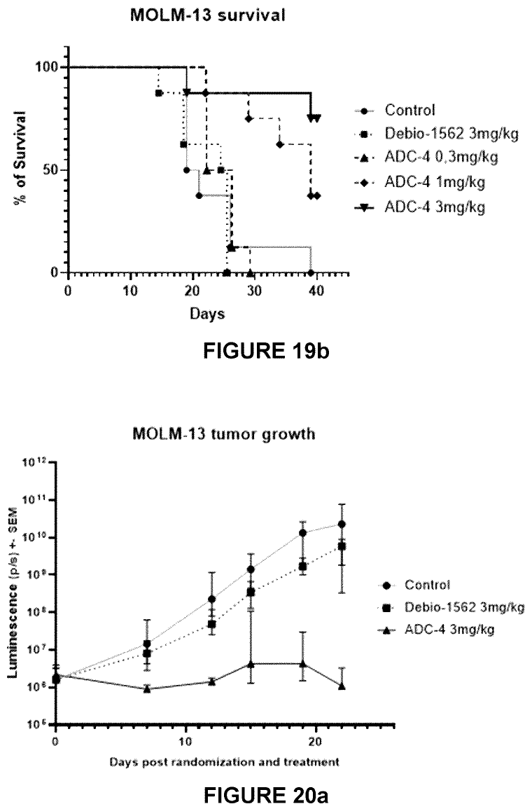

The control group mice received an injection of the vehicle (5 mL/kg). After tumor cells inoculation, the animals were routinely monitored as set out above, and tumor growth was imaged twice per week after Luciferine administration (using an IVIS Spectrum BL from PerkinElmer). The tumor growth and % survival results are shown in FIG. 19a, 19b, 20a and 20b.

### 8.6.5 Cellular Binding, Internalization and Cytotoxicity in AML Cell Lines

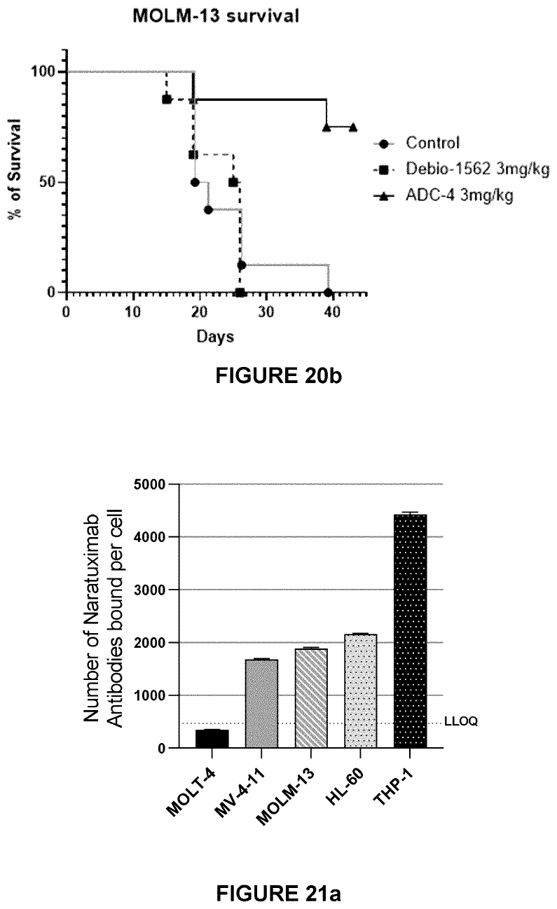

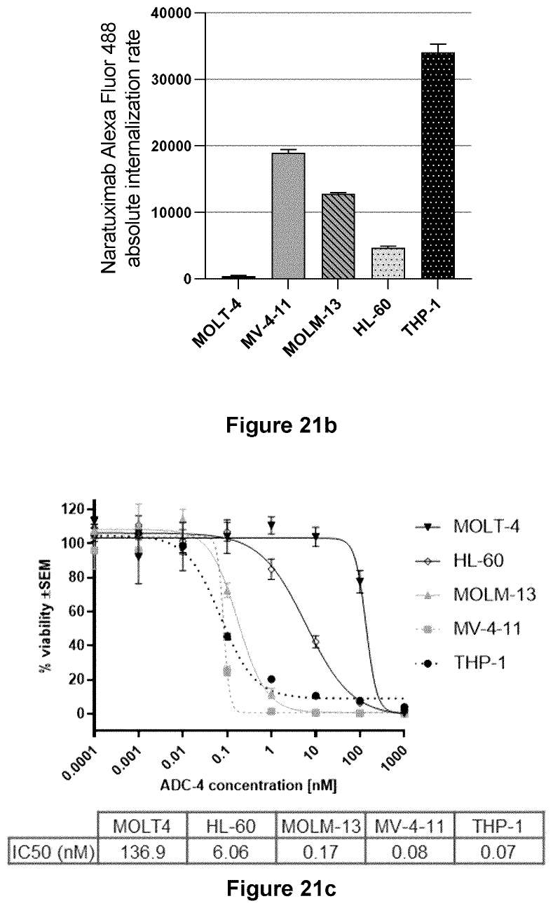

CD37 expressing AML cell lines MV-4-11, MOLM-13, HL-60 and THP-1 were labelled with Live/Dead near IR (Thermo Fisher, L10119), Fc blocked with HumanTruStain FcX (Biolegend, 422302), and incubated with Naratuximab-PE (Biolegend, 99341, lot B304638, custom conjugation) at 1 μg/mL. Phycoerythrin Fluorescence Quantitation beads (BD, 340495) were used as reference to estimate the Naratuximab antibodies bound per cells (ABC). Cells and beads were finally acquired on an Attune NxT flow cytometer. The CD37 negative ALL cell line MOLT-4 was included as negative control. Number of Naratuximab antibodies bound per cell is shown in FIG. 21a.

CD37 expressing AML cell lines MV-4-11, MOLM-13, HL-60 and THP-1 cells were labelled with Live/Dead near IR (Thermo Fisher, L10119) prior to incubation with anti-CD37-Alexa Fluor 488 (clone K₇₁₅₃A) at 0.5 μg/mL for 30 min or 2 h either on ice or at 37° C. Cells were washed twice and incubated with a quenching anti-Alexa Fluor 488 antibody (Thermo fisher, 710369) prior to fixation (Biolegend, 422101). Following fixation cells were analysed by flow cytometry. Data are represented as absolute internalization rate+/−SD (n=3), which is defined as the fluorescence of quenched samples corrected for incomplete surface quenching. The CD37 negative ALL cell line MOLT-4 was included as negative control. Naratuximab absolute internalization rate is shown in FIG. 21b.

In vitro cytotoxic assay: MOLT-4, MV-4-11, MOLM-13, HL-60 and THP-1 cells were plated and after overnight resting (12 hours), serial dilutions of ADC-4 (eight 10-fold dilutions, from 1 μM down to 0.1 μM in triplicate) were added to the cells. After 72 hours of incubation, plates were inspected under an inverted microscope to ensure growth of the controls and sterile conditions. Then, 100 μL CellTiter Glo 2.0 (Promega, G9242) was added to each well prior to luminescence reading as per the manufacturer's instructions. Relative IC50 was calculated for each cell line using GraphPad Prism. Percentage of viability over control (non treated cells) curves are shown in FIG. 21c.

### 8.6.6 in Vitro Cytotoxicity Data

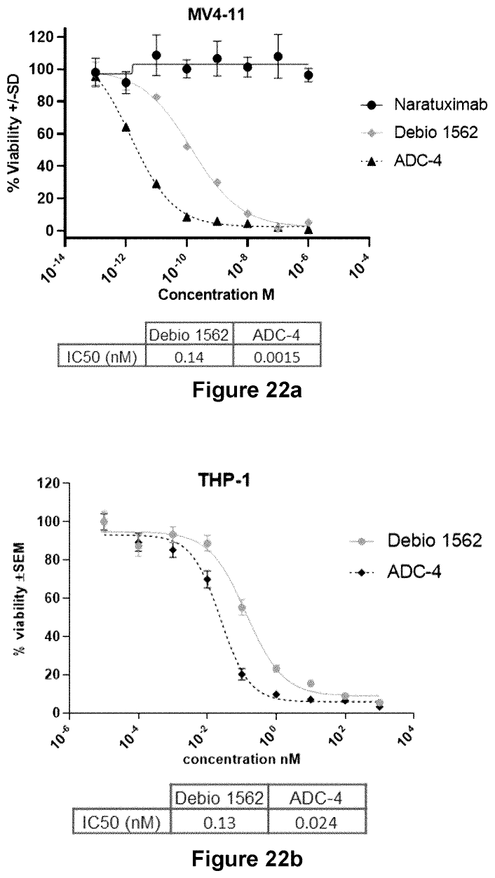

MV-4-11 cells were plated and after overnight resting (12 hours), serial dilutions of ADC-4, Debio 1562 or Naratuximab (eight 10-fold dilutions, from 1 μM down to 0.1 μM in triplicate) were added to the cells. After 72 hours of incubation, plates were inspected under an inverted microscope to ensure growth of the controls and sterile conditions. Then, 100 μL CellTiter Glo 2.0 (Promega, G9242) was added to each well prior to luminescence reading as per the manufacturer's instructions. Relative IC50 was calculated for each cell line using GraphPad Prism. Percentage of viability over control (non treated cells) curves are shown in FIG. 22a.

THP-1 cells were plated and after overnight resting (12 hours), serial dilutions of ADC-4 or Debio 1562 (nine 10-fold dilutions, from 1 μM down to 0.01 μM in triplicate) were added to the cells. After 72 hours of incubation, plates were inspected under an inverted microscope to ensure growth of the controls and sterile conditions. Then, 100 μL CellTiter Glo 2.0 (Promega, G9242) was added to each well prior to luminescence reading as per the manufacturer's instructions. Relative IC50 was calculated for each cell line using GraphPad Prism. Percentage of viability over control (non-treated cells) curves are shown in FIG. 22b.

### 8.6.7 in Vivo Efficacy Compared to AML Standard of Care

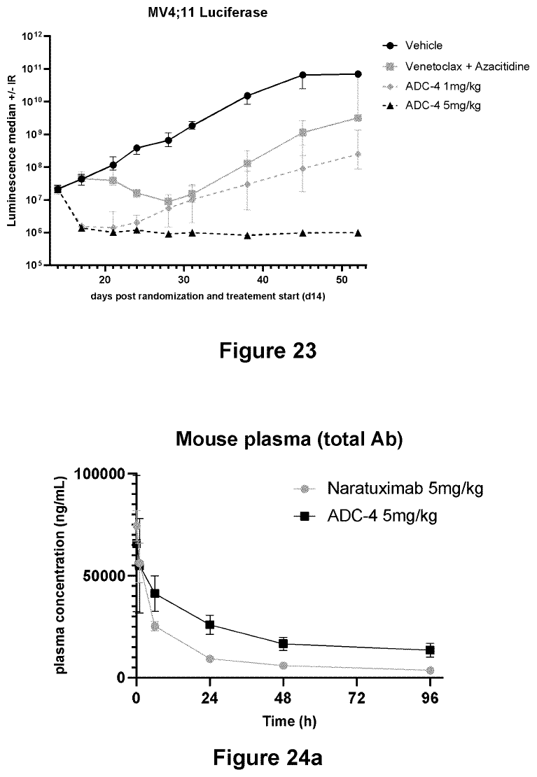

NCG mice were inoculated with MV4;11-Luc Acute Myeloid Leukemia cells in tail vein to allow tumor dissemination (2×10⁷ cells per mouse). Mice were randomized, according to the Bioluminescent result of each animal (Total Flux, photons/sec/cm2) 14 days after inoculation, in 4 groups of 8 mice and treatment started immediately after randomization (D14). In the non-control group mice, one intravenous injection of 1 mg/kg or 5 mg/kg of ADC-4 was performed, or 5 consecutive daily intravenous injection of 3.5 mg/kg Azacitidine in addition to 100 mg/kg Venetoclax administered orally on days 1 to 6 and days 9 to 14. The Vehicle was PBS except for Venetoclax which was formulated in 60% phosal 50 propylene glycol, 30% polyethylene glycol 400, and 10% ethanol. The control group mice received an injection of the vehicle (10 μL/g). After tumor cells inoculation, the animals were daily monitored for clinical signs, morbidity and mortality and body weight was measured twice per week. Tumor growth was imaged twice per week 15 minutes after D-Luciferin injection (PerkinElmer, XenoLight D-Luciferin K+ Salt) using an IVIS Spectrum BL from PerkinElmer. The tumor growth results in Luminescence signal are shown in FIG. 23.

### 8.6.8 Pharmacokinetic Profile in Mouse

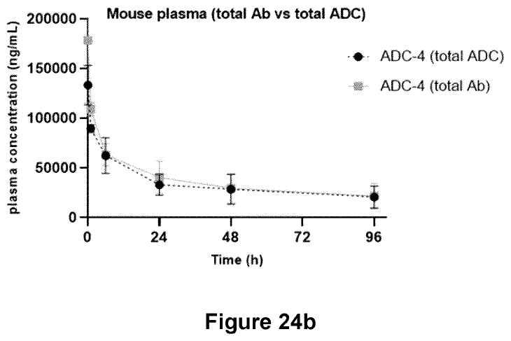

3 Swiss male mice were treated with intravenous injection of 5 mg/kg naratuximab or ADC-4 (vehicle was PBS) and blood was collected by micro-sampling with K3EDTA micro-capillaries at 5 min, 1 h, 6 h, 24 h, 48 h and 72 h posttreatment. Microcapillary blood samples were centrifuged (2000 g, 5 minutes, +4° C.) as soon as practical to allow plasma processing. Plasma samples were freezed at −80° C. and further analyzed for determination of concentration of the Total Antibody using qualified analytical immunoassay procedure. Toxicokinetic parameters were estimated using Phoenix pharmacokinetic software. PK parameters and plasma concentrations are presented in FIG. 24a.

3 CD1 female mice were treated with intravenous injection of 5 mg/kg ADC-4 (vehicle was PBS) and blood was collected by micro-sampling with K2EDTA micro-capillaries at 5 min, 1 h, 6 h, 24 h, 48 h and 96 h post treatment. Microcapillary blood samples were centrifuged (1500 g, 10 minutes, +4° C.) as soon as practical to allow plasma processing. Plasma samples were freezed at −80° C. and further analyzed for determination of concentration of the Total Antibody (Total Ab) and the Total ADC using two qualified analytical immunoassay procedures. Toxicokinetic parameters were estimated using Phoenix pharmacokinetic software. PK parameters and plasma concentrations are presented in FIG. 24b and table 4.

TABLE 4

| Test Item | ^{a}tₘₐₓ (hr) | Cₘₐₓ (ng/mL) | C₀ (ng/mL) | ^{a}tₗₐₛₜ (hr) | AUCₜₗₐₛₜ (hr*ng/mL) |
|---|---|---|---|---|---|
| ADC-4 Total ADC | 0.083 | 133000 ± | 138000 ± | 96 | 3280000 ± |
| | | 19900 | 22100 | | 1200000 |
| ADC-4 Total Ab | 0.083 | 178000 ± | 186000 ± | 96 | 3590000 ± |
| | | 63000 | 69700 | | 1350000 |

### 8.6.9 Human and Mouse Plasma Stability

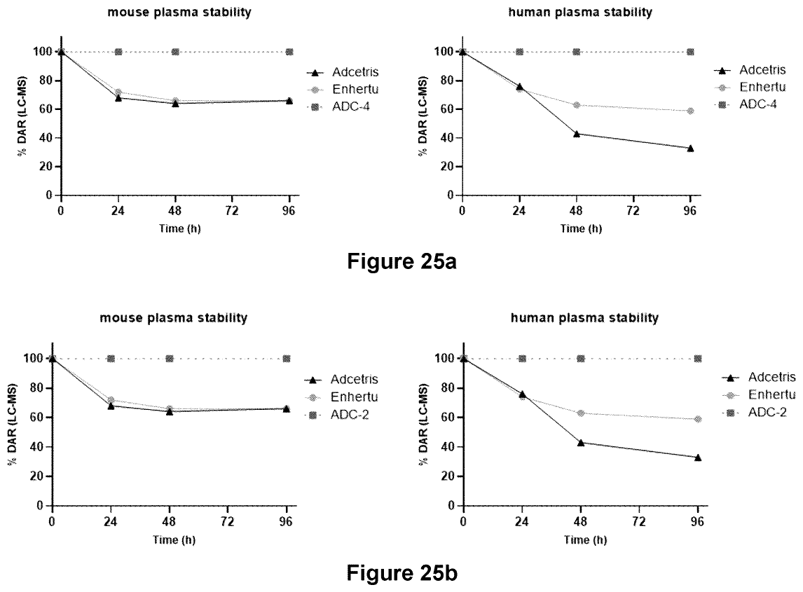

ADC-2, ADC-4, Enhertu and Adcetris (Enhertu and Adcetris used as controls) were spiked into human and mouse plasma at 1 mg/mL concentration. After 0, 24, 48 and 96 h incubation, ADCs were captured with streptavidin magnetic beads coated with biotin anti-IgG-Fc (human) conjugate (100 μL conjugate/250 μL bead slurry). For ADC-2, 40 μL of ADC-spiked plasma were mixed with 60 μL PBS and incubated with 20 μL beads for 1 h at RT, 900 rpm (Eppendorf thermomixer). For ADC-4, Enhertu and Adcetris, 20 μL of ADC-spiked plasma were mixed with 80 μL PBS and incubated with 20 μL beads for 1 h at RT, 900 rpm (Eppendorf thermomixer). Beads were then washed 3 times with 500 μL PBS and elution was performed 2 mM HCl solution and neutralized with 0.5M Ammonium Bicarbonate pH8. ADC-2 and Adcetris were deglycosulated (PNGase F) and Adcetris was reduced with DTT. Analysis of samples was performed by LC-MS (Waters BioAccord). DAR reduction of ADCs was measured and are presented in FIG. 25a (ADC-2) and FIG. 25b (ADC-4).

### 8.6.10 in Vitro Cytotoxicity on DLBCL Cell Lines Panel

A3/KAW, DOHH-2, HBL-1, KARPAS-422, OCI-LY19, OCI-LY3, OCI-LY7, Pfeiffer, U-DHL-2, SU-DHL-4, SU-DHL-5, SU-DHL-6, SU-DHL-8, Toledo, U-2932, WSU-DLCL2 and WSU-NHL cells were plated on day 0 and on day 1 serial dilutions of ADC-4, Debio 1562 or Naratuximab (nine 10-fold dilutions, from 1 μM down to 0.01 μM in triplicate) were added to the cells. After 72 hours of incubation, plates were inspected under an inverted microscope to ensure growth of the controls and sterile conditions. Then, 50 μL CellTiter Glo (Promega, G7572) were added prior to luminescence reading as per the manufacturer's instructions. Relative IC50 were calculated using GraphPad Prism. IC50 values of ADC-4, Debio 1562 and Naratuximab are shown in Table 5.

TABLE 5

| | ADC-4 | Debio 1562 | Naratuximab |
|---|---|---|---|
| Cell line | IC50 (nM) | IC50 (nM) | IC50 (nM) |
| OCI-LY3 | 0.011 | 0.352 | >1000 |
| DOHH-2 | 0.018 | 0.949 | >1000 |
| WSU-NHL | 0.019 | 0.190 | >1000 |
| SU-DHL-5 | 0.022 | 0.901 | >1000 |
| SU-DHL-4 | 0.035 | 0.983 | >1000 |
| SU-DHL-6 | 0.041 | 1.743 | >1000 |
| HBL-1 | 0.055 | 0.217 | >1000 |
| WSU-DLCL2 | 0.063 | 9.593 | >1000 |
| U-2932 | 0.113 | 3.572 | >1000 |
| SU-DHL-2 | 0.307 | 7.777 | >1000 |
| OCI-LY7 | 0.514 | 79.326 | >1000 |
| KARPAS-422 | 0.523 | 20.456 | >1000 |
| SU-DHL-8 | 1.844 | 21.503 | >1000 |
| Pfeiffer | 2.422 | 73.486 | >1000 |
| Toledo | 19.335 | 90.001 | >1000 |
| A3/KAW | 46.462 | 32.986 | >1000 |
| OCI-LY19 | 57.824 | 45.519 | >1000 |

### 8.6.11 in Vivo Efficacy in DLBCL Model

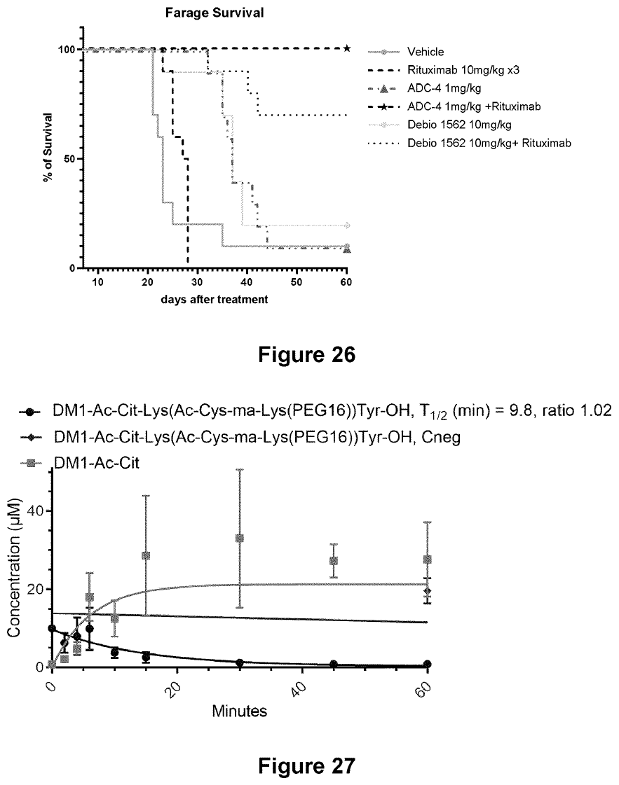

SCID Beige female mice were inoculated with Farage DLBCL cells in tails to allow tumor dissemination (2×10⁷ cells per mouse). Mice were randomized based on body weight 7 days after inoculation in 6 groups of 10 mice and treatment started the day of randomization (DO). In the non-control group mice, one intravenous injection of 1 mg/kg of ADC-4 or 10 mg/kg Debio 1562 was performed alone or in combination with 3 weekly intravenous injection of 10 mg/kg rituximab. The vehicle was PBS. The control group mice received an injection of the vehicle (5 mL/kg). After tumor cells inoculation, the animals were routinely monitored for clinical signs, morbidity and mortality and body weight was measured twice per week. Survival endpoint results are shown in FIG. 26.

8.6.12 Screening Study with ADC-4 at 5 and 50 mg/Kg

The objective of this study was to determine the potential toxicity of ADC-4 when administered intravenously (via bolus injection) as a single dose to female mice. In addition, the toxicokinetic characteristics of ADC-4, both as total antibody (TAb) and total ADC, were determined.

The study design was as follows:

TABLE 6
Experimental design

| | | | | | No. of Animals | |
|---|---|---|---|---|---|---|
| | | Dose | Dose | Dose | | Toxicokinetic |
| Group | Test | Level | Volume^{a} | Concentration | Main Study | Study |
| No. | Material | (mg/kg) | (mL/kg) | (mg/mL) | Females | Females |
| 1 | Control | 0 | 10 | 0 | 5 | 3 |
| | item | | | | | |
| 2 | ADC-4 | 5 | 10 | 0.5 | 5 | 3 |
| | Low dose | | | | | |
| 3 | ADC-4 | 50 | 10 | 5 | 5 | 3 |
| | High dose | | | | | |

(^{a}Based on the most recent body weight measurement)

The following parameters and end points were evaluated in this study: mortality, clinical observations, body weights, food consumption, ophthalmology, clinical pathology parameters (hematology and clinical chemistry), toxicokinetic parameters, organ weights, and macroscopic and microscopic examinations.

CD-1® IGS female mice were administered intravenously with a single injection of either 5 mg/kg or 50 mg/kg of ADC-4. All the animals administered with a single dose of ADC-4 at 5 or 50 mg/kg survived until termination. The median tₘₐₓ of total ADC and TAb were observed at the first sampling time (5 minutes postdose). Systemic exposure to total ADC and TAb (mean C₀, Cₘₐₓ, and/or AUCₜₗₐₛₜ) increased with increasing dose of ADC-04 from 5 to 50 mg/kg in an approximately dose proportional manner. The exposure to TAb (mean C₀, Cₘₐₓ, and AUCₜₗₐₛₜ) in plasma was slightly higher than total ADC following the dosing of ADC-4 at 5 and 50 mg/kg.

There was no ADC-4 related clinical signs in animals administered single doses of 5 mg/kg or 50 mg/kg of ADC-4. There were also no ophthalmologic findings at either of these dose levels. Ten days after administration, an increase in reticulocyte counts was noted (with or without increase in white blood cell, neutrophil, eosinophil, monocyte, and lymphocyte counts), as well as an increase in triglyceride, total protein, globulin concentrations, and calcium and/or phosphorus levels.

At necropsy, ADC-4 was found to have induced an enlargement of the spleen with an incidence related to the dose (which correlated with increased spleen weights and with splenic extramedullary hematopoiesis (EMH)). Increases in kidney and liver weights were also noted without correlates. There were no noticeable microscopic findings 10 days after injection and injection of ADC-4 was well tolerated locally.

### 8.6.13 Dose Range Finding (DRF) ADC-4—Determination of the Potential Toxicity of ADC-4.

ADC-4 was administered intravenously (2 bolus injections of the same dose (25 mg, 50 mg or 100 mg/kg/adm) were administered separately (on Day 1 and Day 8 (7 day interval))) to CD-1® IGS female mice, and the reversibility and/or delayed occurrence of any findings during a 10-day observation period was evaluated. In addition, the toxicokinetic characteristics of ADC-4 (total ADC and total antibody) were determined.

A control group of CD-1® IGS female mice received the control item: Phosphate Buffer Saline (PBS).

The study design was as follows:

TABLE 7
Experimental design

| | | | | | No. of Animals | |
|---|---|---|---|---|---|---|
| | | | Dose | Dose | Main | Toxicokinetic |
| Group | | Dose Level | Volume^{a} | Concentration | Study | Study |
| No. | Test Material | (mg/kg/adm) | (mL/kg) | (mg/mL) | Females | Females |
| 1 | Vehicle control | 0 | 11.24 | 0 | 5 | 3 |
| 2 | Test item | 25 | 10 | 2.5 | 5 | 6 |
| 3 | Test item | 50 | 10 | 5 | 5 | 6 |
| 4 | Test item | 100 | 11.24 | 8.895 | 5 | 6 |

(^{a}Based on the most recent body weight measurement)

The following parameters and end points were evaluated in this study: mortality, clinical observations, body weights, food consumption, ophthalmology, clinical pathology parameters (hematology and clinical chemistry), toxicokinetic parameters, organ weights, and macroscopic and microscopic examinations.

All the animals administered with 2 injections of 100 mg/kg/adm of ADC-4 were prematurely euthanized or found dead on Day 10 due to severe body weight losses and severe reduction of food consumption accompanied by erected fur, hunched posture, and decreased activity. There was no relevant ADC-4 related clinical signs in the animals administered 2 injections of 25 mg/kg/adm of ADC-4 or 2 injections of 50 mg/kg/adm of ADC-4, there were also no premature deaths in these groups of animals.

In the animals administered with 2 injections of 25 of ADC-4 or 2 injections of 50 mg/kg/adm of ADC-4, a higher mean body weight gain was noted (when compared with mean control values) between Day 1 and Day 17, without effect on the food consumption. There was no ophthalmologic finding.

In the mice administered 2 injections of 100 mg/kg/adm of ADC-4, two days after administration of the second injection a marked increase in total white blood cell count was noted (mainly due to an increase in neutrophil and monocyte counts). A decrease in red blood cell, reticulocytes and platelets counts, and hematocrit was also noted. Marked increases in enzyme activities (AST, ALT, and ALP), moderate increases in total bilirubin, total protein and particularly globulin (and consequently decrease in A/G ratio) concentrations, and decreases in cholesterol, triglyceride and glucose concentrations were observed. An increase in the phosphorus concentration was also noted.

In the mice administered 2 injections of 25 mg/kg/adm of ADC-4 or 2 injections of 50 mg/kg/adm of ADC-4, ten days after administration of the second injection an increase in total white blood cell counts (when compared with mean control values) was noted (mainly due to an increase in neutrophil, lymphocyte and monocyte counts). A decrease in platelet count was noted in the animals administered 2 injections of 50 mg/kg/adm of ADC-4 (this was not noted for the animals administered 2 injections of 25 mg/kg/adm of ADC-4).

Noted changes in clinical chemistry consisted of an increase in enzyme activities (AST, ALT, and ALP), total protein (particularly albumin concentrations), and a decrease in triglyceride concentration which was generally more marked in the animals administered 2 injections of the 50 mg/kg/adm of ADC-4.

Microscopically, the following findings were noted:
In animals administrations 2 injections ≥25 mg/kg/adm, ADC-4-related findings were present in the liver (diffuse hepatocellular hypertrophy, extramedullary hematopoiesis and increased mitoses, correlated with higher weight and gross enlargement), spleen (dose-related extramedullary hematopoiesis, correlated with higher weight and gross enlargement/pale discoloration, and single cell necrosis), and duodenum (single cell necrosis of epithelial cells of the mucosa and submucosal glands). At injection site, perivascular subacute inflammation reflected minor local tissue effects of the ADC-4.
In animals administrations 2 injections ≥50 mg/kg/adm, ADC-4-related findings were present in the bone marrow (single cell necrosis, increased myeloid cellularity, decreased erythroid cellularity), liver (single cell necrosis in addition of findings observed at 25 mg/kg/adm), heart (myocardial degeneration/necrosis, and extramedullary hematopoiesis), and eye (single cell necrosis in the corneal epithelium).
In animals administrations 2 injections 100 mg/kg/adm, ADC-4-related findings were present in the liver (glycogen depletion), spleen (decreased white pulp cellularity), kidney (single cell necrosis of epithelial tubular and glomerular cells), digestive tract (single cell necrosis in the stomach, jejunum and ileum, erosion/ulcer in the duodenum, jejunum and ileum, epithelial hypertrophy/hyperplasia in the duodenum and jejunum), iliac lymph nodes (single cell necrosis and extramedullary hematopoiesis), and skin (extramedullary hematopoiesis).

In conclusion:
Two 100 mg/kg/adm injections of ADC-4 administered (with a 7-day interval) in CD-1® mice were not tolerated; all the animals were prematurely euthanized or found dead on Day 10 due to weight loss, significant clinical signs, and reduced appetite. In addition, an increase in white blood cell count, a decrease in red blood cell, reticulocyte and platelet counts and hematocrit, correlating with necrosis and decreased cellularity in the spleen were noted.
Two 25 mg/kg/adm or 50 mg/kg/adm injections of ADC-4 administered (with a 7-day interval) in CD-1® mice were clinically well tolerated. The administrations induced an increase in white blood cell counts and a decrease in platelet counts after 18 days (correlated with extramedullary hematopoiesis in the spleen and in the liver). there was no effect on the food consumption, or body weight and no clinical signs.
Target organ effects were observed at all dose levels in the liver, spleen, digestive tract, kidney, bone marrow, iliac lymph nodes, heart, eye and/or skin. The severity and the extent of the findings were dose-related.

Based on the above results, the no observed adverse effect level (NOAEL) for ADC-4 was considered to be 25 mg/kg/adm in mice.

### 8.7 Cat B-Induced Cleavage Study

### 8.7.1 Preparation of DM1-Ac-Cit and of DM1-Ac-Cit-Lys(Ac-Cys-ma-Lys(PEG16))-Tyr-OH

### DM1-Ac-Cit

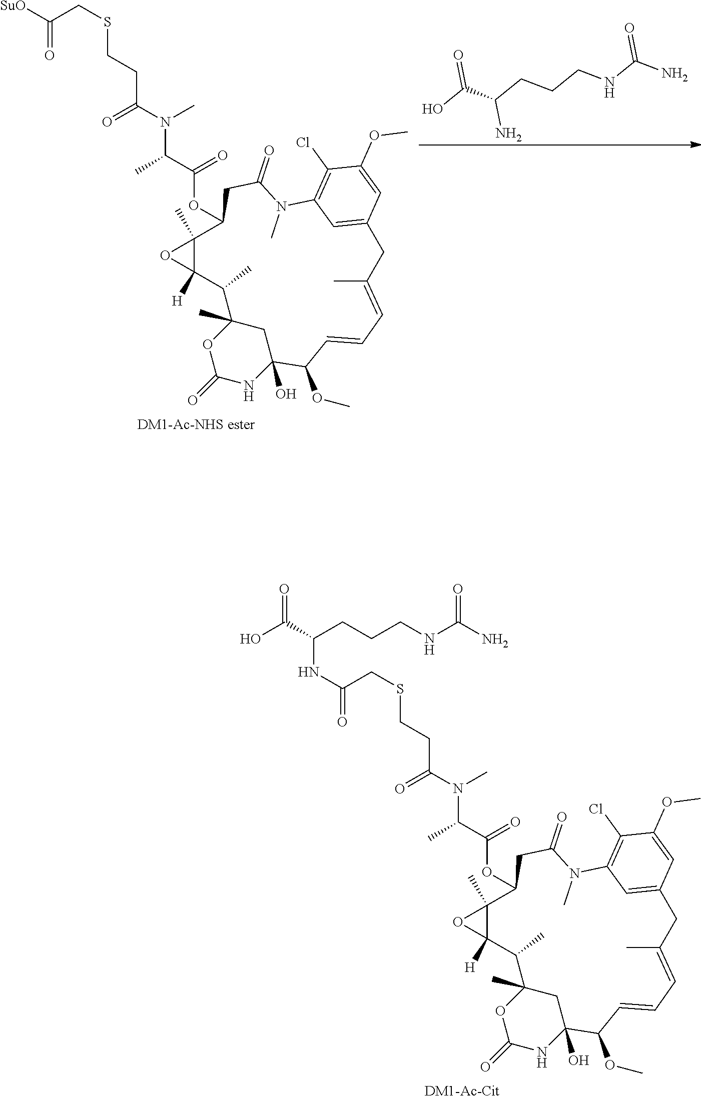

DIEA (0.14 mL, 0.83 mmol, 4.0 eq.) was added to a mixture of DM1-Ac-NHS ester (185 mg, 0.21 mmol, 1.0 eq.) and Citrulline (72.6 mg, 0.41 mmol, 2.0 eq.) in DMSO (3.7 mL) at rt. After stirring at rt for 18 h, TFA was added until acidic pH was reached. Purification by preparative HPLC (10 to 40% of ACN+0.1% TFA in water+0.1% TFA) afforded DM1-Ac-Cit (130 mg, 0.14 mmol, 100% UV purity, 66% yield) as a white powder after freeze-drying. UPLC-MS (method 1): Rt=1.33 min, m/z=952 [M−H]⁻.

### DM1-Ac-Cit-Lys(Ac-Cys-ma-Lys(PEG16))-Tyr-OH

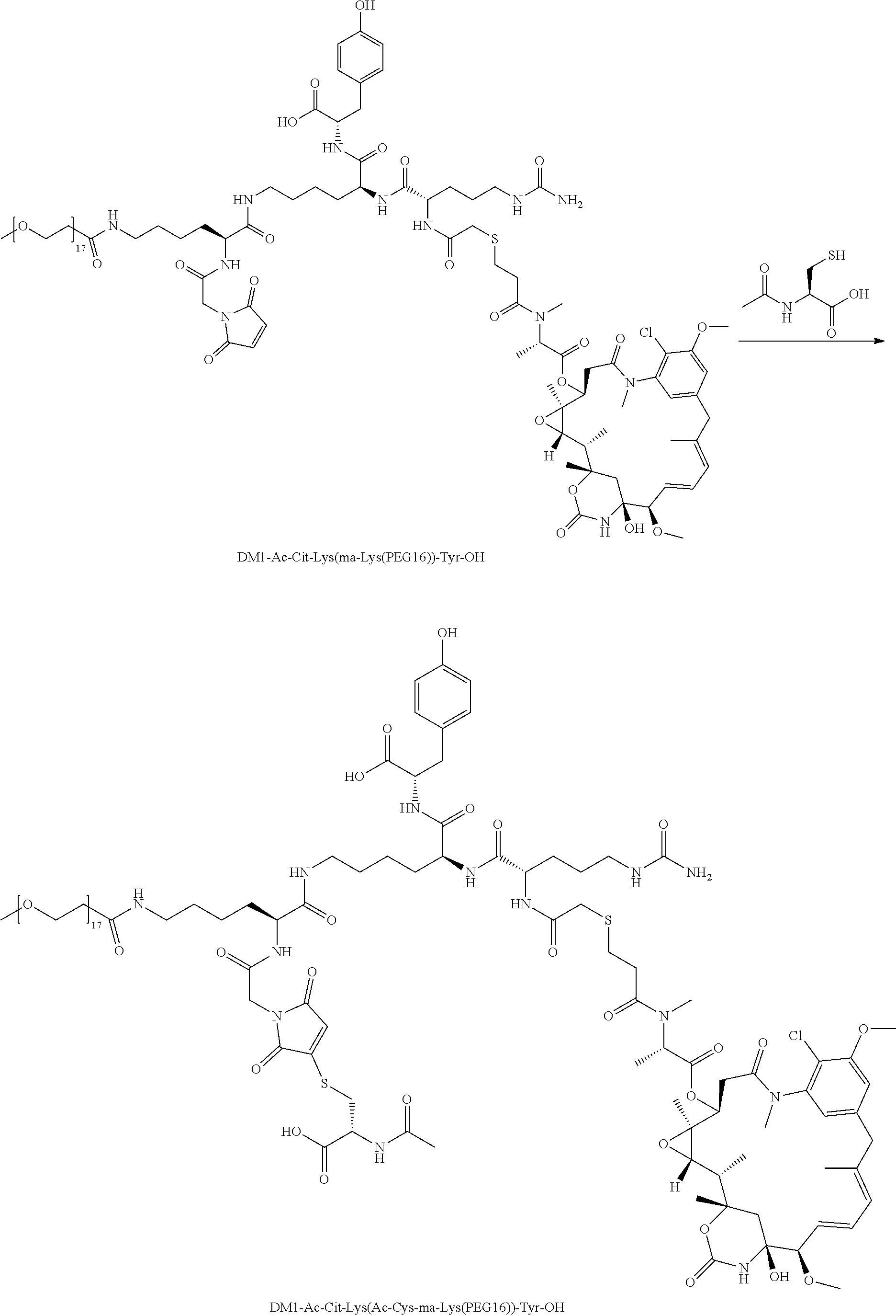

Acetyl-L-cysteine (0.32 mg, 2.17 μmol, 1.0 eq.) was added to a solution of DM1-Ac-Cit-Lys(ma-Lys(PEG16))-Tyr-OH (5.0 mg, 2.17 μmol, 1.0 eq.) in DMF (1 mL) at rt. After stirring at rt for 40 min, TFA was added until acidic pH was reached. Purification by preparative HPLC (5 to 100% of ACN+0.1% TFA in water+0.1% TFA) afforded DM1-Ac-Cit-Lys(Ac-Cys-ma-Lys(PEG16))-Tyr-OH (4.59 mg, 1.86 μmol, 99% UV purity, 94% yield) as a white powder after freeze-drying. UPLC-MS (method 4): Rt=2.41 min, m/z=1231 [M-2H]²⁻

### 8.7.2 Preparation of Exatecan-Suc-Phe-Cit and Exatecan-Suc-Phe-Cit-Lys(Ac-Cys-ma-Lys(Peg16))-Tyr-OH

### Exatecan-Suc-Phe-Cit

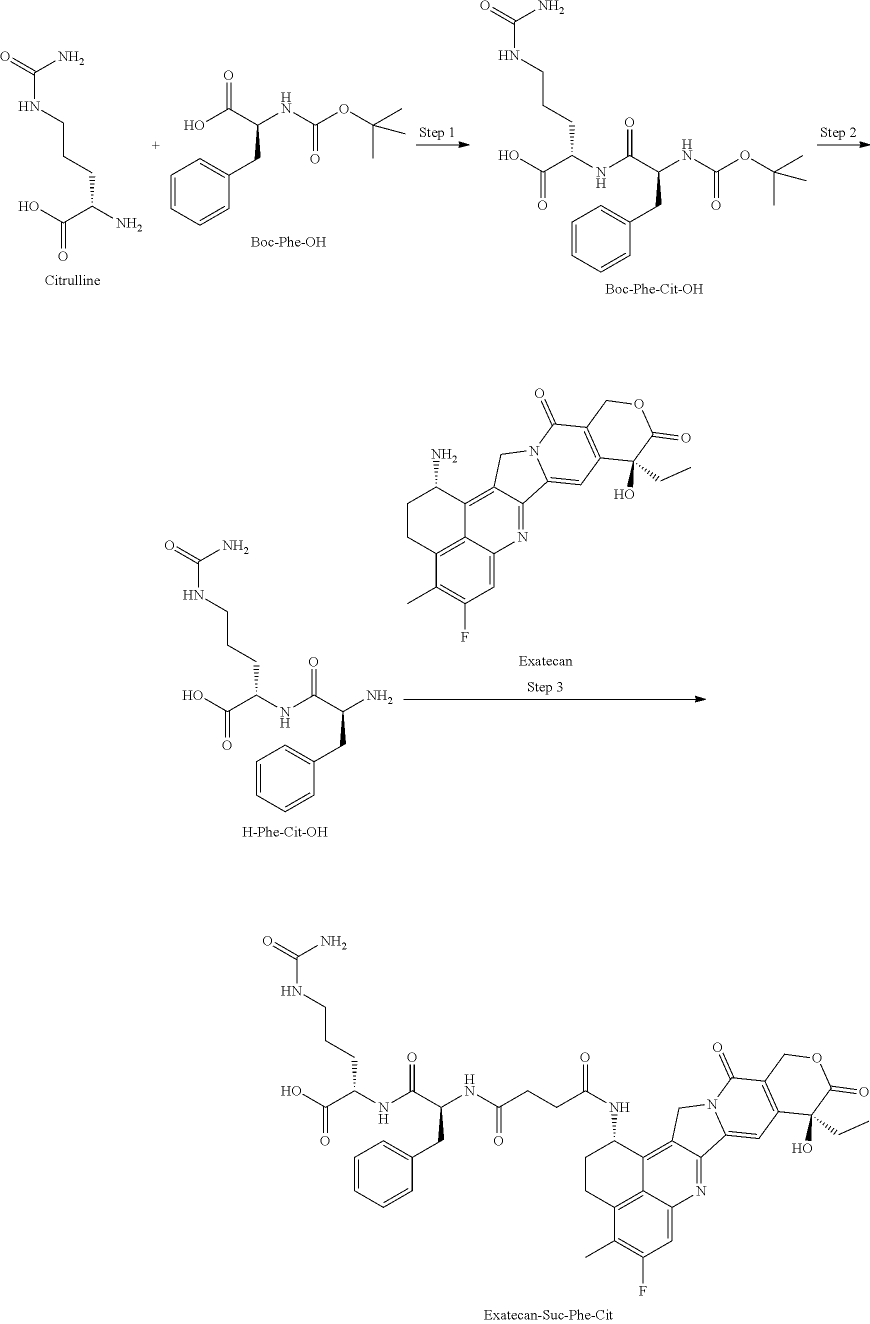

Step 1. DIEA (0.78 mL, 4.48 mmol, 4.0 eq.) was added to a mixture of Citrulline (200 mg, 1.12 mmol, 1.0 eq.) and Boc-Phe-OSu (405 mg, 1.12 mmol, 1.0 eq.) in DMF (10 mL) at rt. After stirring at rt for 16 h, the reaction mixture was filtered then concentrated under reduced pressure. A mixture of water/CAN/TFA (1:1:0.5%) was added. The mixture was filtered then freeze-dried. Purification by preparative HPLC (10 to 60% of ACN+0.1% TFA in water+0.1% TFA) afforded Boc-Phe-Cit-OH (86 mg, 0.20 mmol, 100% UV purity, 18% yield) as a white powder after freeze-drying. UPLC-MS (method 3): Rt=1.27 min, m/z=423 [M+H]⁺, 421 [M−H]⁻.

Step 2. A mixture of DCM (0.9 mL) and TFA (0.9 mL) was added to Boc-Phe-Cit-OH (60 mg, 0.14 mmol, 1.0 eq.) at rt. After stirring at rt for 20 min, the reaction mixture was concentrated. Water (10 mL) and ACN (10 mL) were added and the mixture was freeze-dried to afford H-Phe-Cit-OH (30 mg, 83.8 μmol, 90% UV purity, 59% yield) as a white powder. UPLC-MS (method 3): Rt=0.35 min, m/z=323 [M+H]⁺, 321 [M−H]⁻.

Step 3. Dihydrofuran-2,5-dione (8.4 mg, 83.5 μmol, 1.0 eq.) was added to a mixture of Exatecan (36 mg, 83.5 μmol, 1.0 eq.) and DIEA (0.17 mL, 1.00 mmol, 12 eq.) in DMF (0.8 mL) at rt. After stirring at rt for 10 min, 1-hydroxypyrrolidine-2,5-dione (9.6 mg, 83.5 μmol, 1.0 eq.) was added followed by HATU (31.7 mg, 83.5 μmol, 1.0 eq.). After stirring at rt for 15 min, H-Phe-Cit-OH (29.9 mg, 83.5 μmol, 1.0 eq.) was added to the reaction mixture. After stirring at rt for 1 h, H-Phe-Cit-OH (29.9 mg, 83.5 μmol, 1.0 eq.) was added to the reaction mixture. After stirring at rt for 1 h, TFA was added until acidic pH was reached. Purification by preparative HPLC (20 to 60% of ACN+0.1% TFA in water+0.1% TFA) afforded Exatecan-Suc-Phe-Cit (26.4 mg, 30.8 mmol, 98% UV purity, 37% yield) as a yellow powder after freeze-drying. UPLC-MS (method 2): Rt=1.39 min, m/z=840 [M+H]⁺, 838 [M−H]⁻.

### Exatecan-Suc-Phe-Cit-Lys(Ac-Cys-ma-Lys(Peg16))-Tyr-OH

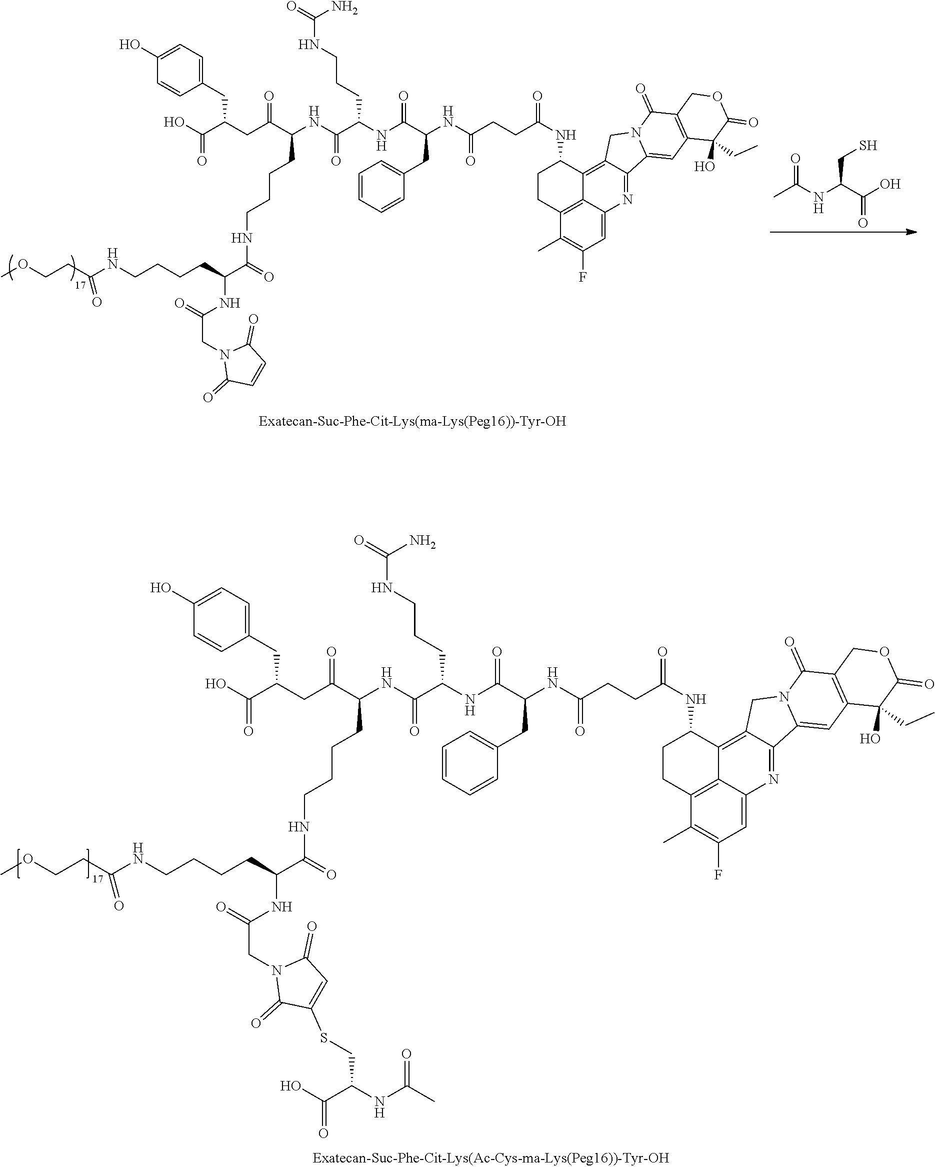

DIEA (1.2 μL, 6.8 μmol, 4.0 eq.) was added to a mixture of Exatecan-Suc-Phe-Cit-Lys(ma-C1-Lys(Peg16))-Tyr-OH (3.71 mg, 1.7 mmol, 1.0 eq.) and acetyl-L-cysteine (0.28 mg, 1.7 μmol, 1.0 eq.) in DMF (0.6 mL) at rt. After stirring at rt for 1 h, TFA was added until acidic pH was reached. Purification by preparative HPLC (20 to 60% of ACN+0.1% TFA in water+0.1% TFA) afforded Exatecan-Suc-Phe-Cit-Lys(Ac-Cys-ma-Lys(Peg16))-Tyr-OH (2.87 mg, 1.2 mmol, 100% UV purity, 72% yield) as a white powder after freeze-drying. UPLC-MS (method 4): Rt=2.37 min, m/z=1176 [M+2H]²+, 1174 [M-2H]²⁻.

### 8.7.3 Cat B-Induced Cleavage

Cat B-induced cleavage of the linker payloads (DM1-Ac-Cit-Lys(Ac-Cys-ma-Lys(PEG16))-Tyr-OH and exatecan-Suc-Phe-Cit-Lys(Ac-Cys-ma-Lys(PEG16))-Tyr-OH) (Ac-Cys quenched linkers of the present invention) were evaluated according to the in vitro enzymatic cleavage assay using recombinant human Cathepsin B (commercially acquired from R&D Systems, Bio-Techne AG, cat #. 953-CY-010 in the form of a precursor) and UHPLC-MS/MS analysis.

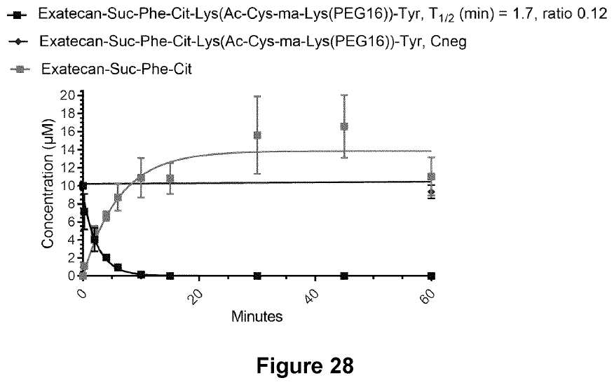

The enzyme was reconstituted in 25 mM 2-(N-morpholino) ethanesulfonic acid (MES) buffer adjusted at pH 5.0 with a 1 M NaOH solution and then activated with a 20 nM solution of Dithiothreitol (DDT) at room temperature for at least 15 min. The in vitro enzymatic assay was conducted at 37° C. with the test compounds at a concentration of 10 μM (2.5 μM when the test compound is an antibody-drug conjugate) in the presence of activated recombinant human Cathepsin B enzyme at 2 μg/mL in a 25 mM MES buffer pH 5.0. The enzymatic cleavage reaction was stopped for each defined time point by mixing a 1/10 ratio volume of acetonitrile+0.1% formic acid (FA) containing an internal standard (warfarine at 0.5 μM). Analysis was conducted using a Waters Acquity UPLC System coupled to a Waters Xevo TQ triple quad mass spectrometer. UHPLC was conducted with a HSS T3 1.8 μm 50×2.1 mm column (Waters) heated at 45° C. and fitted with 2 μm insert filter pre-column (Waters), and solvent systems A1 (H2O+0.1% FA) and B1 (acetonitrile+0.1% FA) at a flow rate of 0.6 mL/min and a 5-95% gradient of B1 over 2.0 min. MS/MS was performed using electrospray ionization (ESI) interface in positive mode and specific MRM transitions for each compound. Results are shown in FIGS. 27 and 28.

### 8.8 DRF—DM1-Ac-Cit and DM1-Ac-Cit-Lys(Cys-ma-Lys(PEG16))-Tyr-OH

The objectives of this study were to determine the potential toxicity of Multilink-DM1-Ac-Cit and DM1-Ac-Cit-Lys(Cys-ma-Lys(PEG16))-Tyr-OH when administered intravenously (via bolus injection).

DM1-Ac-Cit was prepared as set out in section 8.7.1 above.

### Preparation of DM1-Ac-Cit-Lys(Cys-ma-Lys(PEG16))-Tyr-OH

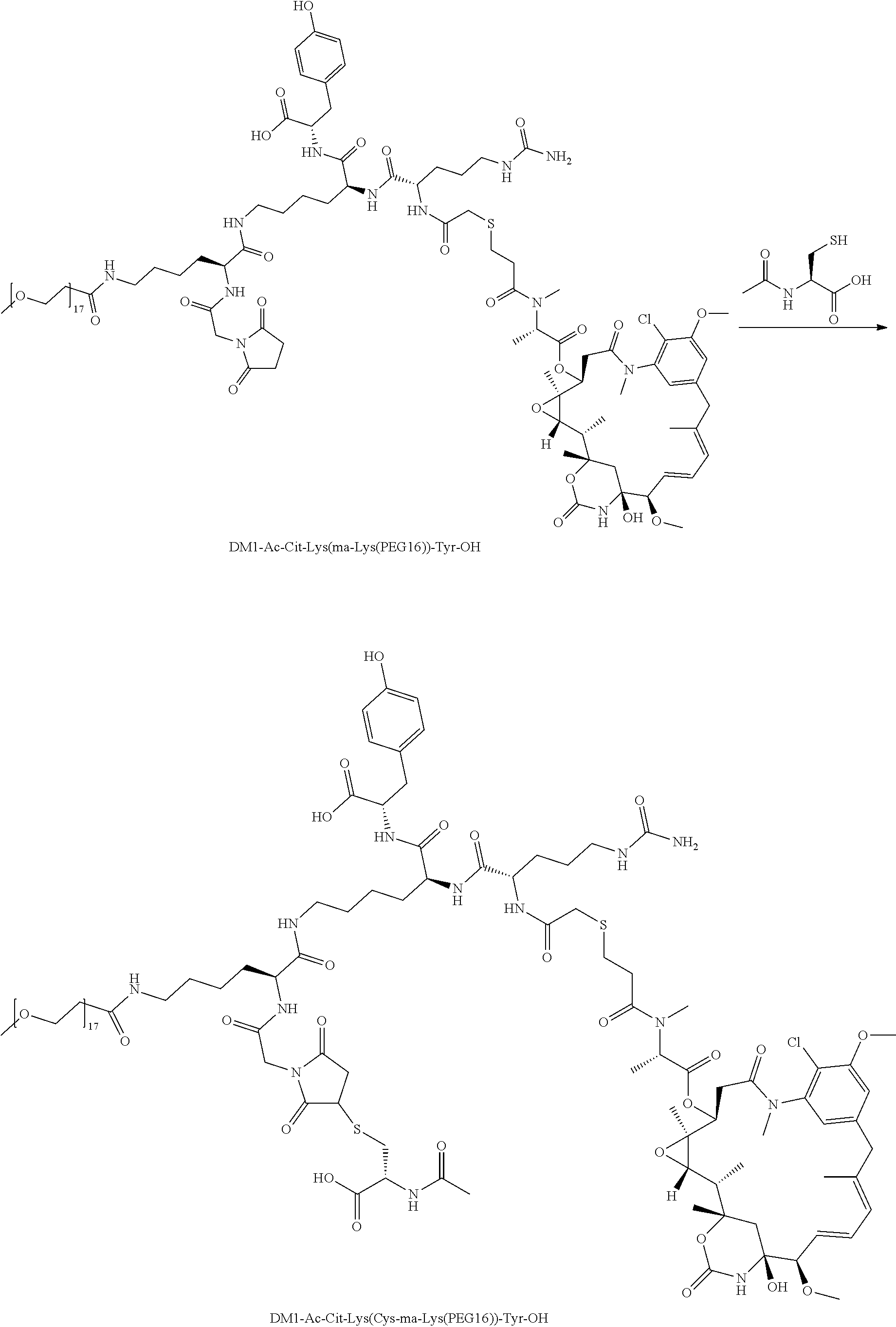

Cysteine (9.15 mg, 75.5 μmol, 2.0 eq.) was added to a solution of DM1-Ac-Cit-Lys(ma-Lys(PEG16))-Tyr-OH (86.9 mg, 37.8 μmol, 1.0 eq.) in DMF (7.6 mL) at rt. After stirring at rt for 5 h, the reaction mixture was filtered using a 33 mm 0.22 μm hydrophobic filter. Purification by preparative HPLC (10 to 50% of ACN+0.1% TFA in water+0.1% TFA) afforded DM1-Ac-Cit-Lys(Cys-ma-Lys(PEG16))-Tyr-OH (62.4 mg, 25.8 μmol, 100% UV purity, 68% yield) as a white powder after freeze-drying. UPLC-MS (method 1): Rt=1.34 min, m/z=1210 [M-2H]²⁻.

2 injections of the same dose of either DM1-Ac-Cit ((test item 1) at 0.15, 0.68 or 1.4 mg/kg/adm) or 2 injections of the same dose of DM1-Ac-Cit-Lys(Cys-ma-Lys(PEG16))-Tyr-OH ((test item 2) at 3.5 mg/kg/adm were administered separately (on Day 1 and Day 8 (7-day interval) to CD-1® IGS mice, and the reversibility and/or delayed occurrence of any findings were evaluated during a 10-day observation period. In addition, the toxicokinetic characteristics of DM1-Ac-Cit-Lys(Cys-ma-Lys(PEG16))-Tyr-OH, DM1-Ac-Cit, and the catabolite DM1 were determined.

A control group received the Vehicle Control 1: 8% Ethanol/8% Polysorbate (Tween*) 80/PBS 84%.

The study design was as follows:

TABLE 8
Experimental design

| | | | | | No. of Animals | |
|---|---|---|---|---|---|---|
| | | | Dose | Dose | Main | Toxicokinetic |
| Group | Test | Dose Level | Volume^{a} | Concentration | Study | Study |
| No. | Material | (mg/kg/adm) | (mL/kg) | (mg/mL) | Females | Females |
| 1 | Vehicle | 0 | 10 | 0 | 5 | 3 |
| | Control 1 | | | | | |
| 2 | Test Item 1 | 0.15 | 10 | 0.015 | 5 | 6 |
| 3 | Test Item 1 | 0.68 | 10 | 0.068 | 5 | 6 |
| 4 | Test Item 1 | 1.4 | 10 | 0.14 | 5 | 6 |
| 5 | Test Item 2 | 3.5 | 10 | 0.35 | 5 | 6 |

(adm = administration, ^{a}Based on the most recent body weight measurement)

The following parameters and end points were evaluated in this study: mortality, clinical observations, body weights, food consumption, ophthalmology, clinical pathology parameters (hematology and clinical chemistry), toxicokinetic parameters, organ weights, and macroscopic and microscopic examinations.

There was no DM1-Ac-Cit- or DM1-Ac-Cit-Lys(Cys-ma-Lys(PEG16))-Tyr-OH related mortalities, clinical observations or effects on body weights and food consumption.

There was no DM1-Ac-Cit- or DM1-Ac-Cit-Lys(Cys-ma-Lys(PEG16))-Tyr-OH related ophthalmic findings or changes in clinical pathology parameters.

No DM1-Ac-Cit- or DM1-Ac-Cit-Lys(Cys-ma-Lys(PEG16))-Tyr-OH related organ weight changes and no macroscopic and microscopic findings were noted.

In conclusion:
2 intravenous (bolus) injections given at 7-day interval of DM1-Ac-Cit at 0.15, 0.68, and 1.4 mg/kg/adm were well tolerated in female CD-1® mice. Based on these results, the no observed adverse effect level (NOAEL) was considered to be 1.4 mg/kg/adm.
In the same way, DM1-Ac-Cit-Lys(Cys-ma-Lys(PEG16))-Tyr-OH at 3.5 mg/kg/adm was well tolerated.

### Reference Example: Cat B-Induced Cleavage Study

The reference compounds shown in Table 9 below were prepared in the Reference Example according to the methodology set out in WO 2019/096867 A1.

TABLE 9
Reference compounds comprising a linker of formula (II)/(II′)

| Compound | Structure |
|---|---|
| 1 | AF-Arg-Lys(PEG₄-Mal-Cys-Ac)-Phe-OH |
| 2 | AF-Arg-Lys(PEG₄-Mal)-Phe-OH |
| 3 | AF-Arg-Phe-Lys(PEG₄-Mal-Cys-Ac)-OH |
| 4 | AF-Arg-Phe-Lys(PEG₄-Mal)-OH |
| 5 | DM1-Mal-Phe-Lys-Lys(PEG₄-Mal-Cys-Ac)-Phe-OH |
| 6 | DM1-Mal-Phe-Cit-Lys(PEG₄-Mal-Cys-Ac)-Phe-OH |
| 7 | DM1-Mal-Phe-Cit-Phe-Lys(PEG₄-Mal-Cys-Ac)-OH |
| 8 | AF-Cit-Lys(PEG₄-Mal-Cys-Ac)-Phe-OH |
| 9 | ACit-Lys(PEG₄-Mal-Cys-Ac)-Phe-OH |
| 10 | ACit-Phe-Lys(PEG₄-Mal-Cys-Ac)-OH |
| 11 | DM1-Mcc-Phe-Cit-Lys(PEG₅-Ma-Cys-Ac)-Tyr-OH |
| 12 | DM1-Mcc-Cit-Lys(PEG₅-Ma-Cys-Ac)-Tyr-OH |
| 13 | DM1-Mcc-Phe-Lys(PEG₅-Ma-Cys-Ac)-Tyr-OH |

The peptides were prepared by standard Fmoc-based SPPS using an Activo P-11 Automated Peptide Synthesizer (available from Activotec), and a Fmoc-Xxx-Wang resin (Xxx: C-terminal amino acid; loading: 0.60 mmol/g; Bachem).

Coupling reactions for amide bond formation were performed over 30 min at room temperature using 3 eq of Fmoc-amino-acids, Fmoc-NH-PEG₄-COOH or Fmoc-NH-PEG₅—COOH activated with HBTU (2.9 eq) in the presence of DIEA (7 eq). Fmoc deprotection was conducted with a solution of 20% piperidine in DMF. Selective removal of the Mtt side-chain protecting group (Lys) was performed using DCM/TFA/TIS (94/1/5, v/v/v).

For the synthesis of compounds 1 to 4 and 8, Auristatin F (AF) was coupled after Fmoc removal by fragment condensation (3 eq AF, 2.9 eq HBTU, 7 eq DIEA) during 30 min. For the synthesis of compounds 9 and 10, Auristatin Cit (ACit) was coupled after Fmoc removal at identical conditions (3 eq ACit, 2.9 eq HBTU, 7 eq DIEA).

For the synthesis of compounds 1 to 4 and 8 to 10, the derivative Mal-PEG₄-NHS was added on resin for 30 min (3 eq of Mal-PEG₄-NHS, 7 eq DIEA) after Mtt removal by DCM/TFA/TIS (94/1/5, v/v/v). Then, for compounds 1, 3, 8, 9 and 10, the maleimide residue on the PEG chain was reacted on resin with acetyl-cysteine (Ac-Cys-OH) via chemoselective ligation (3 eq of Ac-Cys-OH, DIEA, 7 eq) during 20 min. The peptides were cleaved from the resin under simultaneous side-chain deprotection by treatment with TFA/TIS^{a}/water (95/2.5/2.5, v/v/v; ^{a} TIS=triisopropylsilane) during 60 min. After concentration of the cleavage mixture, the crude peptides were precipitated with cold diethyl ether and centrifuged.

For the synthesis of compounds 5 to 7, the derivative Mal-PEG₄-NHS was added on resin for 30 min (3 eq of Mal-PEG₄-NHS, 7 eq DIEA) after Mtt removal by DCM/TFA/TIS (94/1/5, v/v/v). Then, the maleimide residue on the PEG chain was reacted on resin with acetyl-cysteine (Ac-Cys-OH) via chemoselective ligation between maleimide and thiol (3 eq of Ac-Cys-OH, DIEA, 7 eq) during 20 min. The Mal-derivative was inserted by adding the moiety Mal-NHS to the N-terminus of Phe after Fmoc deprotection. The peptides were cleaved from the resin under simultaneous side-chain deprotection by treatment with TFA/TIS/water (95/2.5/2.5, v/v/v) during 60 min. After concentration of the cleavage mixture, the crude peptides were precipitated with cold diethyl ether and centrifuged. Then, Mertansine (DM1, 1.45 eq) was reacted with the terminal maleimide group via chemoselective ligation in PBS buffer at pH 7.4 and acetonitrile (ratio 2:1).

For the synthesis of compounds 11 to 13, the derivative Ma-NHS was added on resin for 30 min (3 eq of Mal-NHS, 7 eq DIEA) after Fmoc removal. Then, the maleimide residue was reacted on resin with acetyl-cysteine (Ac-Cys-OH) via chemoselective ligation (3 eq of Ac-Cys-OH, DIEA, 7 eq) during 20 min. The peptides were cleaved from the resin under simultaneous side-chain deprotection by treatment with TFA/TIS/water (95/2.5/2.5, v/v/v) during 60 min. After concentration of the cleavage mixture, the crude peptides were precipitated with cold diethyl ether and centrifuged. After their purification, the derivative DM1-smcc (1.1 eq) was reacted to the N-terminus of the linker in solution in DMF and 4-methylmorpholine (6 eq) for 4 h.

The peptides were purified on a Waters Autopurification HPLC system coupled to SOD mass spectrometer with a XSelect Peptide CSH C18 OBD Prep column (130 Å, 5 μm, 19 mm×150 mm) using solvent system A (0.1% TFA in water) and B (0.1% TFA in acetonitrile) at a flow rate of 24 mL/min and a 20-60% gradient of B over 30 min.

The appropriate fractions were concentrated and lyophilized. The purity was determined on a Waters Acquity UPLC System coupled to SOD mass spectrometer with a CSH 318 column (130 Å, 11 μm, 2.1 mm×50 mm) using solvent system A (0.1% FA in water) and B (0.1% FA in acetonitrile) at a flow rate of 0.6 mL/min and a 5-85% gradient of B over 5 min or SH Floro-phenyl column (130 Å, 187 μm, 2.1 mm×50 mm) using solvent system A (0.1% FA in water) and B (0.1% FA in acetonitrile) at a flow rate of 0.9 mL/min and a 5-95% gradient of B over 2.9 min.

MS-analysis was performed using electrospray ionization (ESI) interface in positive and negative mode. The results of the analysis of the compounds obtained in the Reference Example are shown in Table 10 below.

TABLE 10
Analysis of compounds 1-13

| | | Purity | | | |
|---|---|---|---|---|---|
| Compound | Formula | (%) | Mw | [M + 2H⁺]⁺² | [M + 3H⁺]⁺³ |
| 1 | C₈₆H₁₃₉N₁₅O₂₃S | 94 | 1783.2 | 892.8 | 595.6 |
| 2 | C₇₉H₁₂₆N₁₄O₁₉ | 96 | 1575.9 | 789.0 | 526.7 |
| 3 | C₈₄H₁₃₅N₁₅O₂₂S | 90 | 1739.1 | 870.8 | 581.0 |
| 4 | C₇₉H₁₂₆N₁₄O₁₉ | 98 | 1575.9 | 789.3 | 526.7 |
| 5 | C₉₅H₁₃₂ClN₁₃O₂₉S₂ | 98 | 2019.7 | 1011.5 | 674.5 |
| 6 | C₉₅H₁₃₁ClN₁₄O₃₀S₂ | 97 | 2048.7 | 1025.6 | 683.8 |
| 7 | C₉₅H₁₃₁ClN₁₄O₃₀S₂ | 94 | 2048.7 | 1026.1 | 685.0 |
| 19 | C₈₄H₁₃₅N₁₅O₂₂S | 90 | 1739.2 | 871.9 | 581.6 |
| 20 | C₇₅H₁₂₅N₁₃O₂₂S | 96 | 1592.9 | 798.3 | 532.2 |
| 21 | C₇₅H₁₂₅N₁₃O₂₂S | 96 | 1592.9 | 798.4 | 532.1 |
| 22 | C₁₀₁H₁₄₁ClN₁₄O₃₂S₂ | 99 | 2162.9 | 1082.2 | 721.9 |
| 23 | C₉₂H₁₃₂ClN₁₃O₃₁S₂ | 89 | 2015.7 | 1008.7 | 673.0 |
| 24 | C₉₅H₁₃₀ClN₁₁O₃₀S₂ | 89 | 2005.7 | — | 669.0 |

The propensity of compounds 1-13 to be cleaved by Cathepsin B—was evaluated according to an in vitro enzymatic cleavage assay using recombinant human Cathepsin B and UHPLC-MS/MS analysis as described below.

Reference compounds Cys-MC-Val-Cit-PABC-MMAF and MMAF were used as positive controls. The enzyme was reconstituted in 25 mM MES buffer adjusted at pH 5.0 with a 1M NaOH solution and then activated with a 20 nM solution of DDT at room temperature for at least 15 min.

The in vitro enzymatic assay was conducted at 37° C. with the test compounds at a concentration of 10 μM (2.5 μM when the test compound is an antibody-drug conjugate) in the presence of activated recombinant human Cathepsin B enzyme at 2 μg/mL in a 25 mM MES buffer pH 5.0. The enzymatic cleavage reaction was stopped for each defined time point by mixing an equal volume of acetonitrile+0.1% FA containing an internal standard (warfarine at 8 μM).

Analysis was conducted using a Waters Acquity UPLC System coupled to a Waters Xevo TQ triple quad mass spectrometer. UHPLC was conducted depending on the test compounds with a BEH C8 1.7 μm 100×2.1 mm or BEH C18 1.7 μm 50×2.1 mm or HSS T3 1.7 μm 50×2.1 mm columns heated at 45° C. or 50° C. and fitted with 2 μm insert filter pre-columns (available from Waters), and solvent systems A1 (H₂O+0.1% FA) and B1 (acetonitrile+0.1% FA) at a flow rate of 0.6 mL/min and a 10-95% gradient of B1 over 1.9 min.

MS/MS was performed using electrospray ionization (ESI) interface in positive mode and specific MRM transitions for each tested compound.

The results are given in Table 11 below.

TABLE 11
Cat B-induced cleavage study of compounds 1, 3, 5, 6-8,
11 and 12 (Reference compound: Cys-MC-Val-Cit-PABC-MMAF)

| | T_{1/2 Compound} | T_{1/2 Reference} | Ratio |
|---|---|---|---|
| Compound | (min) | (min) | (T_{1/2 Compound}/T_{1/2 Reference} |
| 1 | 1.5 | 12.9 | 0.12 |
| 3 | 1.4 | 12.9 | 0.11 |
| 5 | 0.6 | 10.9 | 0.05 |
| 6 | 0.3 | 13.1 | 0.02 |
| 7 | 1.2 | 13.1 | 0.09 |
| 8 | 1.1 | 12.6 | 0.09 |
| 11 | 0.06 | 11.4 | 0.005 |
| 12 | 0.0004 | 11.4 | 0.00004 |

From these results, it is apparent that exo-Cat B cleavage and drug release (AF-Arg, AF-Cit, ACit, DM1-Mal-Phe-Lys, DM1-Mal-Phe-Cit, DM1-Mcc-Phe-Cit) in compounds 1, 3, 5, 6-8, 11 and 12 occurred simultaneously and were very fast. For instance, Cat B-induced drug release from compound 5 occurred 20 times faster as compared to the reference PABC compound Cys-MC-Val-Cit-PABC-MMAF. The fast cleavage kinetics achieved by compounds 1-8 and 11-12 demonstrates that the compounds of the invention comprising a linker of formula (II) or (II′) exhibit high selectivity and binding affinity for the exopeptidase activity of Cat B. Furthermore, it was surprisingly found that the presence of an Ac-Cys-PEG₄ moiety on the side-chain of the Lys residue (corresponding to residue Axx in formula (II) or (II′)) had no detrimental effect on the binding affinity of the compounds for Cat B. These results also indicate that, by contrast, cleavage by the endopeptidase based mechanism of Cat B as realized in the PABC linker systems (e.g., as in reference compound Cys-MC-Val-Cit-PABC-MMAF) occurs at significantly slower rates. As particular striking examples, compounds 11 and 12 are spontaneously cleaved by exo-Cat B (T_{1/2}≤1 min), demonstrating the highly favorable binding properties of substrates based on the linker of formula (II)/(II′), it is believed that the favorable interaction between the C-terminal Tyr and occluding loop of Cat B strongly contributes to the fast cleavage rate observed in these compounds.

1. A compound represented by the general formula (I): 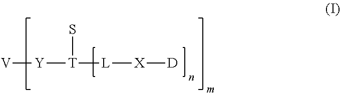
wherein,
D represents a moiety derived from a drug, the drug being selected from a carboxyl-containing drug such as auristatin F (AF), a thiol-containing drug such as mertansine (DM1) or ravtansine (DM4), an amino-containing drug such as monomethyl auristatin F (MMAF) or exatecan, and a hydroxyl-containing drug such as Maaa-1181a, preferably from a thiol-containing drug, an amino-containing drug and a hydroxyl-containing drug; if more than one (D) is present, each (D) is independently selected from a carboxyl-containing drug, a thiol-containing drug, an amino-containing drug and a hydroxyl-containing drug, the moieties (D) being preferably identical to each other;
X represents a divalent group comprising one to seven, preferably two to six, more preferably two to five, backbone atoms independently selected from C, N, O, and S; X being covalently attached to (D) via an atom selected from C, S, N and O derived from the carboxyl, thiol, amino, or hydroxyl functional group comprised in (D);
Y is a divalent group comprising one or more atoms selected from C, N, O, P and S, preferably a divalent group derived from a compound selected from maleimides, triazoles, hydrazones, carbonyl-containing compounds and derivatives thereof, more preferably a divalent group derived from maleimides and derivatives thereof such as opened hydrolyzed maleimides, and most preferably a divalent group derived from an opened hydrolyzed maleimide;
L represents a linker capable of being cleaved by Cathepsin B;
T represents a (2+n)-valent branching group;
S represents a moiety derived from a compound comprising one or more, e.g., two, three or four, solubilizing groups;
V represents a moiety derived from a vector group capable of interacting with a target cell;
n is an integer of 1 to 4, preferably 1 or 2, more preferably 1; and
m is an integer of 1 to 12, preferably 2 to 10, more preferably 4 to 8.

2. The compound of claim 1, wherein (L) is represented by the general formula (II) or (II′): 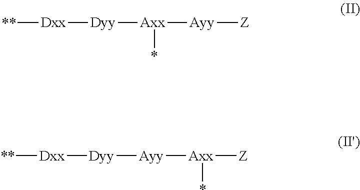
wherein,
Axx represents a moiety derived from a trifunctional amino acid, with the proviso that Axx in formula (II) is not a moiety derived from an amino acid in the (D) configuration;
Ayy represents a moiety derived from an amino acid selected from Phe, Ala, Trp, Tyr, Phenylglycine (Phg), Met, Val, His, Lys, Arg, Citrulline (Cit), 2-amino-butyric acid (Abu), Ornithine (Orn), Ser, Thr, Leu and Ile; or Ayy in formula (II) represents a moiety derived from an amino acid selected from homo-tyrosine (homo-Tyr), homo-phenylalanine (homo-Phe), beta-phenylalanine (beta-Phe) and beta-homo-phenylalanine (beta-homo-Phe), Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24; with the proviso that Ayy in formula (II′) is not a moiety derived from an amino acid in the (D) configuration;
Dxx represents a single covalent bond or a moiety derived from an amino acid having a hydrophobic side chain, preferably a single covalent bond or a moiety derived from an amino acid selected from Phe, Val, Tyr, homo-Phe and Ala, more preferably a single covalent bond or a moiety derived from Phe or Val;
Dyy represents a single covalent bond, a moiety derived from Phe or a moiety derived from an amino acid having a basic side chain, preferably a moiety derived from an amino acid selected from Arg, Lys, Cit, Orn, 2,3-diamino-propionic acid (Dap), 2,4-diamino-butyric acid (Dab), more preferably a moiety derived from Arg or Cit; with the proviso that if Dxx is a moiety derived from an amino acid having a hydrophobic side chain, Dyy is a moiety derived from Phe or a moiety derived from an amino acid having a basic side chain, and if Dxx is a single covalent bond, Dyy is a single covalent bond, a moiety derived from Phe or a moiety derived from an amino acid having a basic side chain;
Z represents a group covalently bonded to the C-terminus of Ayy or Axx selected from —OH and —N(H)(R) wherein R represents a hydrogen atom, an alkyl group or a cycloalkyl group, preferably —OH;
* indicates covalent attachment to (T); and
** indicates covalent attachment to (X).

3. The compound of claim 2, wherein at least one of Axx and Ayy is defined as follows:
Axx represents a moiety derived from an amino acid selected from Glu, 2-amino-pimelic acid (Apa), 2-amino adipic acid (Aaa), Dap, Dab, Lys, Orn, Ser, Ama, and homo-lysine (homo-Lys), preferably a moiety derived from an amino acid selected from Dap, Dab, Lys, Orn and homo-Lys, more preferably a moiety derived from Orn or Lys, most preferably a moiety derived from Lys;
Ayy in formula (II) represents a moiety derived from an amino acid selected from Phe, homo-Phe, Ala, Trp, Phg, Leu, Val, Tyr, homo-Tyr, Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24, preferably a moiety derived from Phe, homo-Phe, Tyr, homo-Tyr, Tyr(OR₁) and homo-Tyr(OR₁), more preferably a moiety derived Phe or Tyr, most preferably a moiety derived from Tyr;
Ayy in formula (II′) represents a moiety derived from an amino acid selected from Phe, homo-Phe, Ala, Trp, Phg, Leu, Val, Tyr and Ser, preferably a moiety derived from Phe, home-Phe and Ser, more preferably a moiety derived from Phe or Ser, most preferably a moiety derived from Phe.

4. The compound of any of claims 1 to 3, wherein (X) represents a divalent carbonyl- or thiocarbonyl-containing group, preferably a group represented by one of the following formulae (IIIa) to (IIIf):
***—(CH₂)ₙ₂—(C=A)-**′ (IIIa)
***—(C=A)-(CH₂)ₙ₃—**′ (IIIb)
***—(CH₂)ₙ₂—(C=A)-(CH₂)ₙ₃—**′ (IIIc)
***—(CH₂)ₙ₂—(C=A)-(C(CH₃)₂)—(CH₂)ₙ₃—**′ (IIId)
***—(CH₂)ₙ₂—(C(CH₃)₂)—(C=A)-(CH₂)ₙ₃—**′ (IIIe)
***—(C=A)-(NH)ₙ₄—(CH₂)ₙ₃—(NH)ₙ₅—(C=A)-**′ (IIIf)
wherein
n2, n3 are each independently selected from 0 to 5, preferably 0, 1 or 2, more preferably 0 or 1;
n4, n5 are each selected from 0 or 1;
each A is independently selected from O and S, preferably O;
*** represents covalent attachment to (D); and
**′ represents covalent attachment to (L).

5. The compound of any of claims 1 to 3, wherein (X) is represented by one of the following formulae (IVa) to (IVj′): 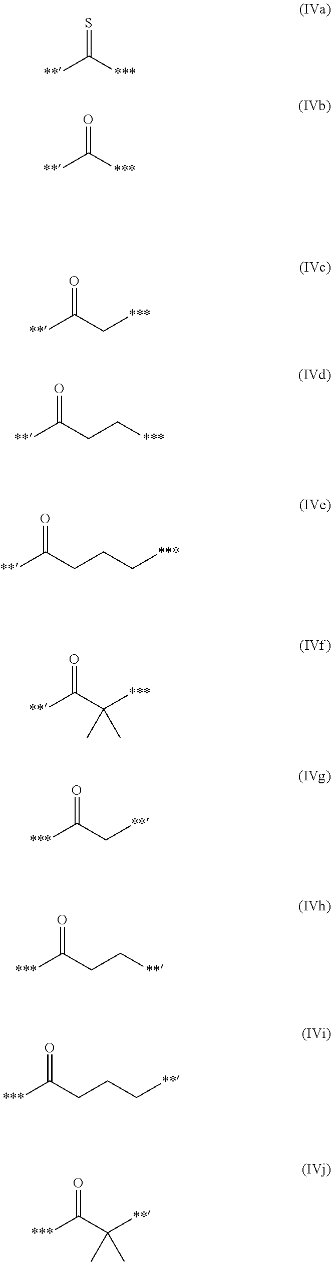 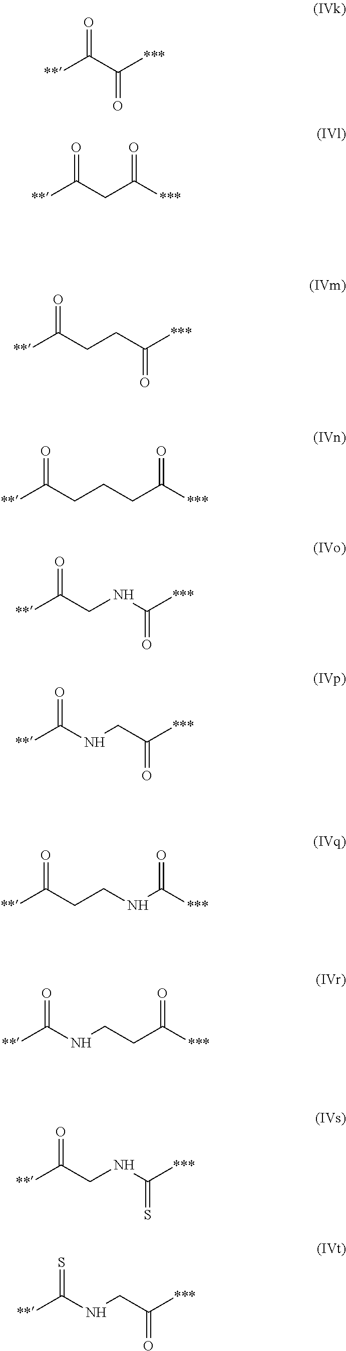 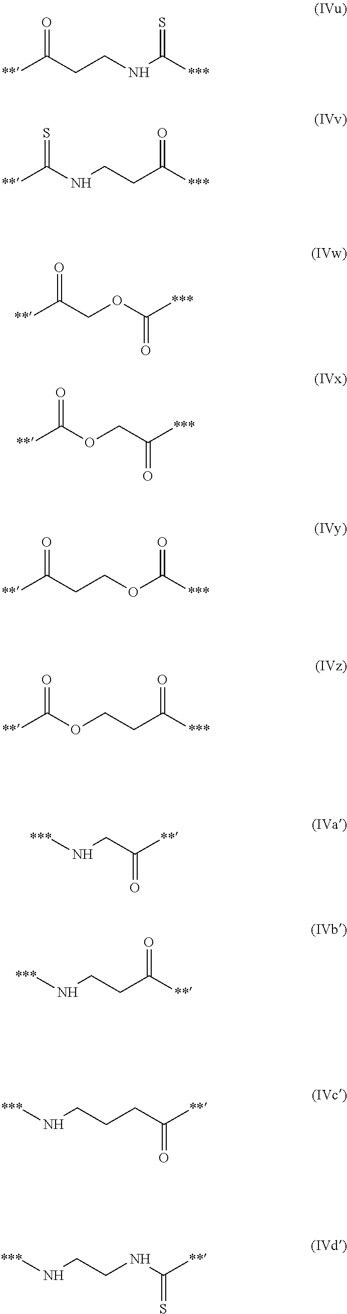 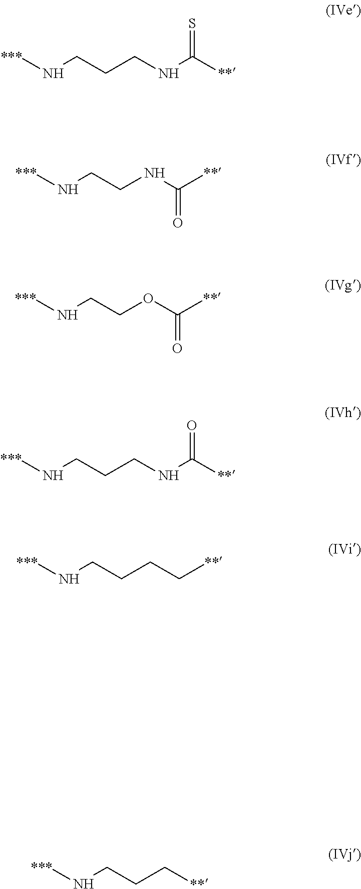
wherein,
*** represents covalent attachment to (D);
**′ represents covalent attachment to (L);
with the proviso that if (X) is represented by formula (IVg), (IVh), (IVi), (IVj), (IVk), (IV/), (IVm), (IVn), (IVp), (IVr), (IVt), (IVv), (IVw), (IVx), (IVy) or (IVz), (D) in formula (I) represents an amino-containing drug; if (X) is represented by formula (IVj), (IVq), (IVs) or (IVu), (D) in formula (I) represents an amino-containing drug or a hydroxyl-containing drug; and if (X) is represented by formula (IVa′), (IVb′), (IVc′), (IVd′), (IVe′), (IVf′), (IVg′), (IVh′), (IVi′) or (IVj′), (D) in formula (I) represents a carboxyl-containing drug.

6. The compound of claim 5, wherein (X) is represented by formula (IVb′), (IVc), (IVm), (IVn), (IVo), (IVp), (IVs) or (IVt), preferably by formula (IVc) or (IVm).

7. The compound of any of claims 1 to 6, wherein each solubilizing group comprised in (S) is independently selected from the group consisting of:
moieties comprising one or more ionic or ionizable groups, such as ammonium, guanidinium, sulfate or sulfonate groups, preferably of moieties derived from Arg, (D)-Arg, Dap, (D)-Dap, Dab, (D)-Dab, Orn, (D)-Orn, Lys, D-Lys or carnitine;
saccharide moieties selected from monosaccharides, disaccharides and linear or branched oligosaccharides, in particular linear or branched oligosaccharides having 3 to 10 monosaccharide units being linked by glycosidic bonds, wherein each of the monosaccharide units in the monosaccharide, disaccharide and oligosaccharide is independently selected from glucose, fructose, mannose, ribose, and galactose; and
polyalkylene oxide groups, preferably C₂₋₃ polyalkylene oxide groups, more preferably C₂₋₃ polyalkylene oxide groups independently comprising from 6 to 200, preferably from 10 to 150, more preferably from 12 to 80 repeating units.

8. The compound of any of claims 1 to 7, wherein (S) is a moiety derived from a compound comprising one or more polyethylene oxide groups, wherein preferably each polyethylene oxide group independently comprises from 6 to 200, more preferably from 10 to 150, most preferably from 12 to 80 repeating units;
(S) being preferably a moiety represented by the formula (V):
****—X¹—(CH₂CH₂O)ₙ₃—X² (V)
wherein,
n3 is an integer of 6 to 200, preferably 10 to 150, more preferably 12 to 80;
*** indicates covalent attachment to (T);
X¹ is selected from a single covalent bond, —(C═O)—, and —N(R)— in which R represents a hydrogen atom, an alkyl group or a cycloalkyl group;
X² represents an alkyl group having 1 to 6 carbon atoms, a carbonyl-containing group such an acetyl group or a group of formula —(CH₂)ₙ₄—CO₂H, a thiocarbonyl-containing group, a group of formula —(CH₂)ₙ₄OR, a group of formula —(CH₂)ₙ₄—SO₃H, or an amino-containing group such as a group of formula —(CH₂)ₙ₄—(C=A)-N(R)₂ or —(CH₂)ₙ₄—N(R)₂, in which A is O or S, each R is independently selected from a hydrogen atom, an alkyl group and a cycloalkyl group, and n4 is an integer of 1 to 6;
X² being preferably —CH₃, —CH₂CH₂OH, or a group represented by the following formula (VI):
—(CH₂)ₙ₅—(C=A)N(R)—(CH₂)ₙ₆—(C=A)N(H)(R) (VI)
wherein,
each A is independently selected from O and S, preferably O;
each R is independently selected from a hydrogen atom, an alkyl group and a cycloalkyl group; and
n5 and n6 are each independently an integer of 1 to 6, preferably 1 or 2;
X² being most preferably —CH₃; and
if more than one (S) is present, each (S) is preferably a moiety of formula (V) above.

9. The compound of any of claims 1 to 8, wherein (T) is represented by the following formula (VII): 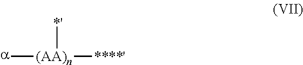
wherein,
each AA independently represents a moiety derived from a trifunctional amino acid such as a diamino-carboxylic acid, an amino dicarboxylic acid, an azido amino acid or an alkyne-containing amino acid, preferably derived from an amino acid selected from N-ε-propargyloxycarbonyl-L-Lysine (Lys(Poc)), Asp, Glu, Orn, Lys, Dab and Dap, more preferably derived from Lys(Poc), Glu, Orn or Lys, most preferably derived from Lys;
α indicates covalent attachment to (Y);
if n=1, the side chain originating from the trifunctional amino acid is covalently attached to (L) or (S), the C-terminus is covalently attached to the other moiety (S) or (L), respectively;
if n=2, 3 or 4:
*′ indicates covalent attachment to (L);
****′ indicates covalent attachment to (S); and
n is as defined in claim 1.

10. The compound of any of claims 1 to 8, wherein (T) is represented by the formula (VIII) or (IX): 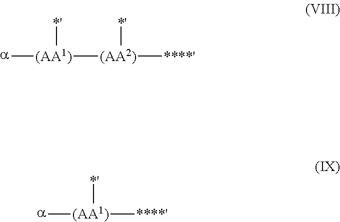
wherein,
each AA¹ and AA² is independently a moiety derived from a trifunctional amino acid, such as a diamino-carboxylic acid, an amino dicarboxylic acid, an azido amino acid or an alkyne-containing amino acid, preferably a moiety derived from an amino acid selected from Lys(Poc), Asp, Glu, Orn, Lys, Dab and Dap, more preferably a moiety derived from Lys(Poc), Glu, Orn or Lys, most preferably a moiety derived from Lys;
α indicates covalent attachment to (Y);
in formula (IX), the side chain originating from the trifunctional amino acid is covalently attached to (L) or (S), the C-terminus is covalently attached to the other moiety (S) or (L), respectively;
in formula (VIII), *′ indicates covalent attachment to (L), and ****′ indicates covalent attachment to (S).

11. The compound of any of claims 1 to 10, wherein (D) is a moiety derived from a drug selected from
(i) an antineoplastic agent such as
a DNA-alkylating agent, such as duocarmycin,
a topoisomerase inhibitor, such as doxorubicin,
an RNA-polymerase II inhibitor, such as alpha-amanitin,
a DNA cleaving agent, such as calicheamicin,
an antimitotic agent or microtubule disruptor, such as a taxane, an auristatin or a maytansinoid,
an anti-metabolite, such as derivatives of gemcitabine,
a Kinesin spindle protein inhibitor, such as Filanesib,
a kinase inhibitor, such as ipatasertib or gefitinib,
nicotinamide phosphoribosyltransferase inhibitor,
a matrix metallopeptidase 9 inhibitor,
a phosphatase inhibitor such as mycrocystin-LR,
(ii) an immunomodulatory agent, such as fluticasone
(iii) an anti-infectious disease agent, such as rifamycin, clindamycin or reptamulin, and
(iv) radioisotopes, metabolites, pharmaceutically acceptable salts, and/or prodrugs of any of the foregoing;
with the proviso that the drug selected from (i) to (iv) is a carboxyl-containing drug, a thiol-containing drug, an amino-containing drug, or a hydroxyl-containing drug; and
if more than one (D) is present, each (D) is independently selected from the aforementioned moieties (i) to (iv), the moieties (D) being preferably identical to each other.

12. The compound of any of claims 1 to 11, wherein (D) is a moiety derived from a drug selected from amanitin, duocarmycin, auristatin, auristatin F (AF), monomethyl auristatin F (MMAF), maytansine, mertansine (DM1), ravtansine (DM4), tubulysin, calicheamicin, camptothecin, SN-38, exatecan, Maaa-1181a, taxol, daunomycin, vinblastine, doxorubicin, methotrexate, pyrrolobenzodiazepine (PBD) and dimers thereof, indilinobenzodiazepine (IBD) and dimers thereof, or radioisotopes and/or pharmaceutically acceptable salts thereof; preferably a moiety derived from a drug selected from auristatin, MMAF, exatecan, maytansine, DM1 and DM4; more preferably a moiety derived from DM1 or DM4.

13. The compound of any one of claims 1 to 12, which is represented by the general formula (X) or (X′): 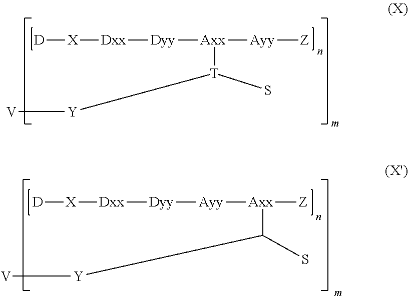
wherein,
Axx in formula (X) and in formula (X′) represents a moiety derived from an amino acid selected from Glu, Apa, Aaa, Dap, Dab, Lys, Orn, Ser, Ama and homo-Lys, preferably a moiety derived from an amino acid selected from Dap, Dab, Lys, Orn and homo-Lys, more preferably a moiety derived from a moiety derived from Lys;
Ayy in formula (X) represents a moiety derived from an amino acid selected from Phe, homo-Phe, Ala, Trp, Phg, Leu, Val, Tyr, homo-Tyr, Tyr(OR₁) and homo-Tyr(OR₁) wherein R₁ is —(CH₂CH₂O)ₙ₁—R₂, wherein R₂ is a hydrogen atom or a methyl group and n1 is an integer of 2 to 24, preferably a moiety derived from Phe, homo-Phe, Tyr, homo-Tyr, Tyr(OR₁) or homo-Tyr(OR₁), more preferably a moiety derived from Tyr;
Ayy in formula (X′) represents a moiety derived from an amino acid selected from Phe, homo-Phe, Ala, Trp, Phg, Leu, Val, Tyr and Ser, preferably a moiety derived from Phe, home-Phe or Ser, more preferably a moiety derived from Phe or Ser;
D, Dxx, Dyy, X, Y, T, S, V, Z, m and n have the same meanings as specified in any of claims 1, 2, 4, 5, 6, 7, 8, 9, 10, 11 and 12; and wherein preferably at least one, e.g., two, three, four, five, six, seven or eight, of D, Dxx, Dyy, X, Y, T, S and Z is/are defined as follows:
(a) D is a moiety derived from a drug selected from auristatin, MMAF, exatecan, maytansine, DM1 and DM4, preferably a moiety derived from DM1 or DM4;
(b) Dxx is a moiety derived from an amino acid selected from Phe, Val, Tyr, homo-Phe and Ala, preferably a moiety derived from Phe or Val;
(c) Dyy is a covalent bond or a moiety derived from an amino acid selected from Arg, Lys, Cit, Orn, Dap and Dab, preferably a covalent bond or a moiety derived from Arg or Cit;
(d) X is a group of formula (III) wherein n2 is 1 or 2, or a group represented by any of formulae (IVa) to (IVz), preferably a group represented by any of formula (IVc), (IVm), (IVn), (IVo), (IVp), (IVs) or (IVt), more preferably by formula (IVc) or (IVm);
(e) Y is a group derived from a compound selected from maleimides, triazoles, hydrazones, carbonyl-containing compounds and derivatives thereof, preferably from maleimides and derivatives thereof such as opened hydrolyzed maleimides, and more preferably derived from an opened hydrolyzed maleimide;
(f) T is a group of formula (VII), (VIII) or (IX);
(g) S is a moiety of formula (V); and
(h) Z is —OH.

14. The compound of claim 13, wherein in formula (X), each Dyy-Dxx-Axx-Ayy is independently selected from Arg-Lys-Phe wherein Dyy is a covalent bond, Arg-Lys-homoPhe wherein Dyy is a covalent bond, Arg-Lys-Tyr wherein Dyy is a covalent bond, Cit-Lys-Phe wherein Dyy is a covalent bond, Cit-Lys-Tyr wherein Dyy is a covalent bond, Arg-Lys-homoTyr wherein Dyy is a covalent bond, Cit-Lys-homoTyr wherein Dyy is a covalent bond, Phe-Cit-Lys-Phe, Phe-Cit-Lys-Tyr, Phe-Arg-Lys-Tyr, Phe-Cit-Lys-homoTyr, Phe-Lys-Lys-Phe, homoPhe-Arg-Lys-Phe, homo-Phe-Cit-Lys-Tyr; and
in formula (X′), each Dyy-Dxx-Ayy-Axx is independently selected from Arg-Phe-Lys wherein Dyy is a covalent bond, Arg-Ser-Lys wherein Dyy is a covalent bond, Cit-Phe-Lys wherein Dyy is a covalent bond, Cit-Ser-Lys wherein Dyy is a covalent bond, Cit-homoPhe-Lys wherein Dyy is a covalent bond, Phe-Cit-Phe-Lys, homoPhe-Cit-Phe-Lys, and Phe-Arg-Phe-Lys.

15. The compound of any one of claims 1 to 14, which is represented by one of the following formulae: 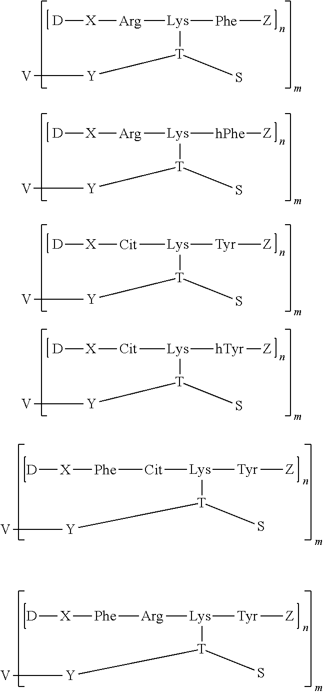
wherein D, X, Y, T, S, V, Z, m and n have the same meanings as specified in claim 1, 2, 4, 5, 6, 7, 8, 9, 10, 11 or 12; and wherein preferably at least one, e.g., two, three, four, five or six, of D, X, Y, T, S and Z is/are defined as follows:
(a) D is a moiety derived from a drug selected from auristatin, AF, MMAF, exatecan, maytansine, DM1 and DM4, preferably a moiety derived from DM1 or DM4;
(d) X is a group of formula (III), wherein n2 is 1 or 2, or a group represented by any of formulae (IVa) to (IVz), preferably a group represented by any of formula (IVc), (IVm), (IVn), (IVo), (IVp), (IVs) or (IVt), more preferably by formula (IVc) or (IVm);
(e) Y is a group derived from a compound selected from maleimides, triazoles, hydrazones, carbonyl-containing compounds and derivatives thereof, preferably from maleimides and derivatives thereof such as opened hydrolyzed maleimide derivatives, and more preferably derived from an opened hydrolyzed maleimide;
(f) T is a group of formula (VII), (VIII) or (IX);
(g) S is a moiety of formula (V); and
(h) Z is —OH.

16. The compound of any of claims 1 to 15, which is represented by one of the following formulae: 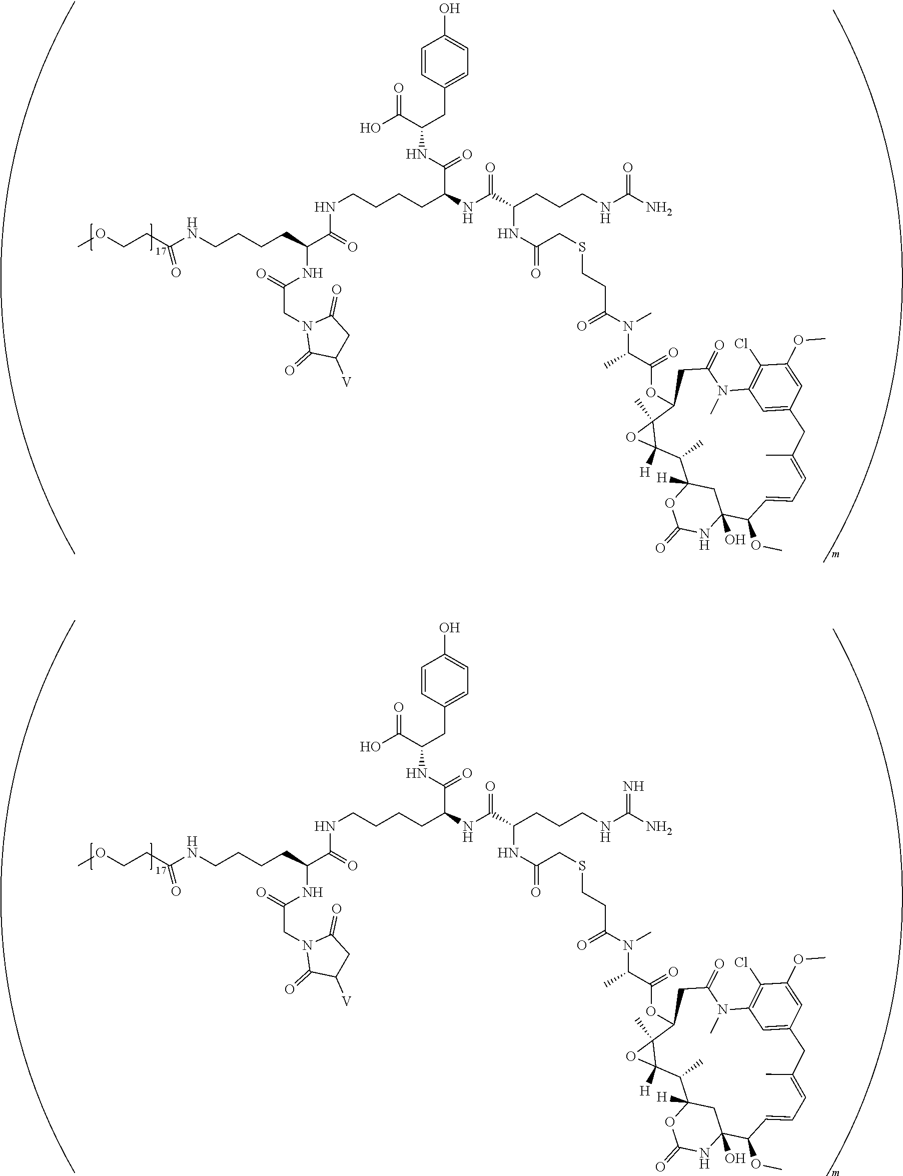 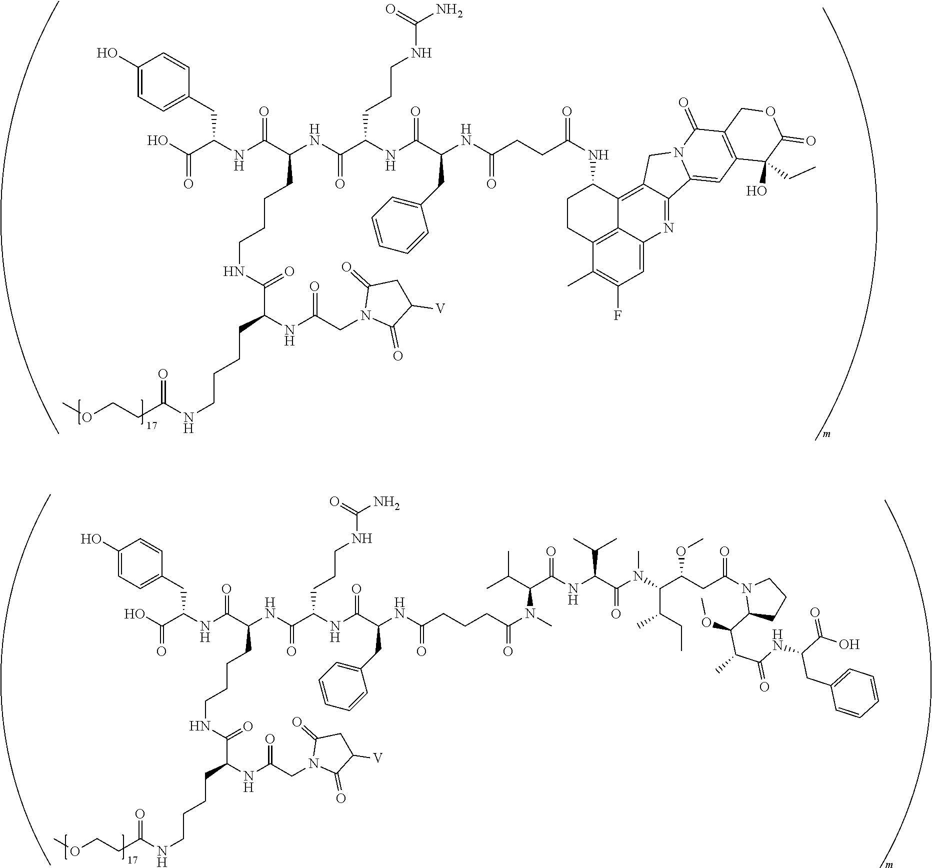
wherein m is an integer of 1 to 12, preferably 2 to 10, more preferably 4 to 8;
wherein the number of oxyethylene repeating units (17) may be replaced by 12 to 30, preferably 14 to 25, more preferably 15 to 19 oxyethylene groups; and/or wherein the maleimide attachment to (V) may be replaced by an opened hydrolyzed maleimide.

17. The compound of any of claims 1 to 16, wherein (V) represents a moiety derived from a vector group capable of interacting with a target cell, wherein the target cell is selected from tumor cells, virus infected cells, microorganism infected cells, parasite infected cells, cells involved in autoimmune diseases, activated cells, myeloid cells, lymphoid cells, melanocytes and infectious agents including bacteria, viruses, mycobacteria, fungi;
preferably the target cell is selected from lymphoma cells, myeloma cells, myeloid cells, lymphoid cells, renal cancer cells, breast cancer cells, prostate cancer cells, ovarian cancer cells, colorectal cancer cells, gastric cancer cells, squamous cancer cells, small-cell lung cancer cells, testicular cancer cells, skin cancer cells, pancreatic cancer cells, liver cancer cells, and any cells growing and dividing at an unregulated and quickened pace to cause cancers.

18. The compound of any of claims 1 to 17, wherein (V) represents a moiety derived from a vector group selected from antibodies, antibody fragments, proteins, peptides and non-peptidic molecules;
preferably a moiety derived from an antibody or an antibody fragment such as a single chain antibody, a monoclonal antibody, a single chain monoclonal antibody, a monoclonal antibody fragment, a chimeric antibody, a chimeric antibody fragment, a domain antibody or fragment thereof, a cytokine, a hormone, a growth factor, a colony stimulating factor, a neurotransmitter or a nutrient-transport molecule.

19. The compound of any of claims 1 to 18, wherein (V) represents a moiety derived from:
a monoclonal antibody, preferably from an antibody selected from the group consisting of adalimumab, aducanumab, alemtuzumab, altumomab pentetate, amivantamab, atezolizumab, anetumab, avelumab, bapineuzumab, basiliximab, bectumomab, belantamab mafadotin, bermekimab, besilesomab, bevacizumab, bezlotoxumab, brentuximab, brentuximab vedotin, brodalumab, catumaxomab, cemiplimab, cetuximab, cinpanemab, clivatuzumab, crenezumab, tetraxetan, daclizumab, daratumumab, denosumab, dinutuximab, dostarlimab, durvalumab, edrecolomab, elotuzumab, emapalumab, enfortumab, enfortumab vedotin, epcoritamab, epratuzumab, epratuzumab-SN-38, etaracizumab, gemtuzumab, gemtuzumab ozogamycin, genmab, glofitamab, girentuximab, gosuranemab, ibritumomab, inebilizumab infliximab, inotuzumab, inotuzumab ozogamicin, ipilimumab, isatuximab ixekizumab, J591 PSMA-antibody, labetuzumab, lecanemab, loncastuximab tesirin, mogamulizumab, mosunetuzumab, necitumumab, nimotuzumab, natalizumab, naratuximab, naxitamab, nivolumab, ocrelizumab, ofatumumab, olaratumab, oregovomab, panitumumab, pembrolizumab, pertuzumab, polatuzumab, polatuzumab vedotin, prasinezumab, racotumomab, ramucirumab, rituximab, sacituzumab, sacituzumab govitecan, semorinemab, siltuximab, solanezumab, tacatuzumab, tafasitamab, teprotumumab, tilavonemab, tocilizumab, tositumomab, trastuzumab, trastuzumab deruxtecan, trastuzumab emtansine, TS23, ustekinumab, vedolizumab, votumumab, zagotenemab, zalutumumab, zanolimumab, fragments and derivatives thereof; more preferably from atezolizumab, durvalumab, pembrolizumab, rituximab or trastuzumab; or
an antibody fragment incorporated into an Fc-fusion protein, which is preferably selected from belatacept, aflibercept, ziv-aflibercept, dulaglutide, rilonacept, romiplostim, abatacept and alefacept.

20. The compound or salt of any of claims 1 to 19, wherein (V) represents a moiety derived from an anti-HER2, anti-CD37, anti-PDL1 or anti-EGFR antibody, preferably from an antibody selected from trastuzumab, pembrolizumab, naratuximab, atezolizumab, durvalumab, avelumab, panitumumab and cetuximab, more preferably from naratuximab, trastuzumab, and cetuximab, and most preferably from naratuximab and trastuzumab.

21. The compound of claim 20, wherein (D) is a moiety derived from an antineoplastic agent, and preferably a moiety derived from a drug selected from amanitin, duocarmycin, auristatin, auristatin F (AF), monomethyl auristatin F (MMAF), maytansine, mertansine (DM1), ravtansine (DM4), tubulysin, calicheamicin, camptothecin, SN-38, exatecan, Maaa-1181a, taxol, daunomycin, vinblastine, doxorubicin, methotrexate, pyrrolobenzodiazepine (PBD) and dimers thereof, indilinobenzodiazepine (IBD) and dimers thereof, or radioisotopes and/or pharmaceutically acceptable salts thereof.

22. The compound of claim 21, wherein (D) represents a moiety derived from a drug selected from auristatin, MMAF, exatecan, maytansine, DM1 and DM4; more preferably a moiety derived from DM1 or DM4.

23. The compound of claim 22, wherein (D) represents a moiety derived from DM1 and wherein the compound is represented by one of the following formulae: 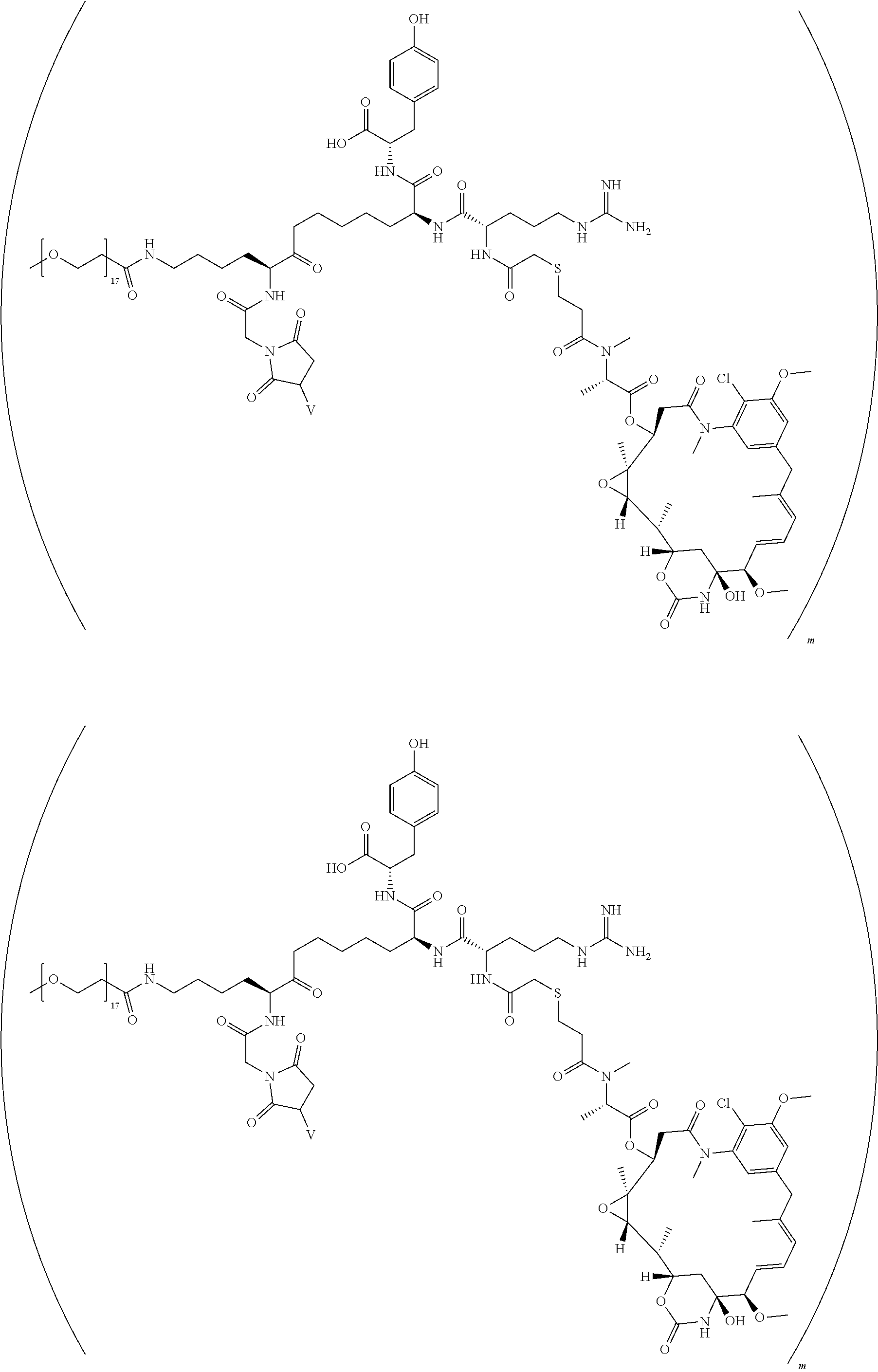
wherein m is an integer of 1 to 12, preferably 2 to 10, more preferably 4 to 8; and wherein the number of oxyethylene repeating units (17) may be replaced by 12 to 30, preferably 14 to 25, more preferably 15 to 19 oxyethylene groups and/or wherein the maleimide attachment to (V) may be replaced by an opened hydrolyzed maleimide.

24. The compound of claim 23, which is represented by the following formula: 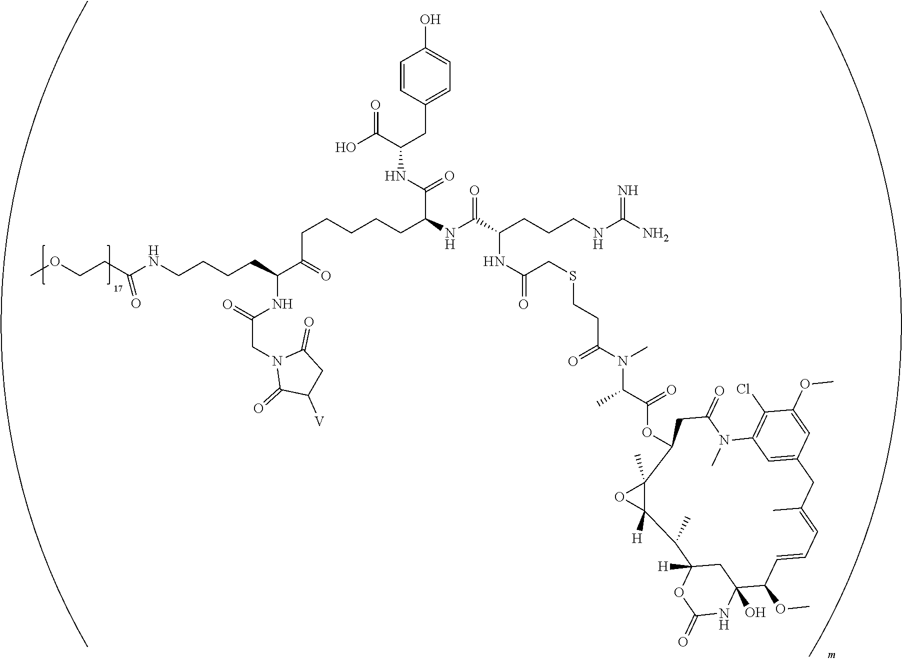
wherein m is an integer of 1 to 12, preferably 2 to 10, more preferably 4 to 8; and wherein the number of oxyethylene repeating units (17) may be replaced by 12 to 30, preferably 14 to 25, more preferably 15 to 19 oxyethylene groups and/or wherein the maleimide attachment to (V) may be replaced by an opened hydrolyzed maleimide.

25. Composition comprising a therapeutically effective amount of the compound of any of claims 1 to 24 or a pharmaceutically acceptable salt thereof, and one or more components selected from a carrier, a diluent and other excipients.

26. The compound or composition of any of claims 1 to 25 for use in a method of treating or preventing a cancer, an autoimmune disease and/or an infectious disease.

27. The compound or composition for use of claim 26 wherein the method is a method of treating a cancer of the blood and bone marrow and preferably acute myeloid leukemia (AML).

28. The compound or composition for use of item 26, wherein the compound is as defined in any of claims 20, 21, 22, 23 and 24, preferably as defined in claim 23 or 24, and most preferably as defined in claim 24.

29. The compound or composition for use of any of claims 26 to 28, wherein in the method of treating or preventing a cancer, an autoimmune disease and/or an infectious disease, the compound or composition is administered concurrently with, before or after one or more other therapeutic agents or therapies such as chemotherapeutic agents, radiation therapy, immunotherapy agents, autoimmune disorder agents, anti-infectious agents or other compounds of formula (I).

30. A compound represented by the general formula (XI): 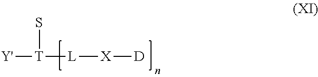
wherein,
D, X, L, T, S and n have the same meanings as specified in claim 1, 2, 4, 5, 6, 7, 8, 9, 10, 11 or 12;
Y′ represents a moiety comprising a conjugation group capable of forming a covalent attachment to a molecule capable of interacting with a target cell (V′) such as a monoclonal antibody or an antibody fragment incorporated into an Fc-fusion protein; Y′ being preferably a moiety comprising a conjugation group selected from:
an optionally substituted maleimide, preferably capable of reacting with one or two thiol groups of (V′),
an optionally substituted haloacetamide, preferably capable of reacting with a thiol group of (V′),
an ester, preferably capable of reacting with the side chain of an amino acid of (V′) such as an acyl halide, an N-hydroxy succinimide ester or a phenolic ester
a carbonate, preferably capable of reacting with the side chain of an amino acid of (V′) such as a haloformate or a carbonate comprising a leaving group such as N-hydroxy succinimide or phenol;
an isocyanate or isothiocyanate, preferably capable of reacting with the side chain of an amino acid of (V′);
an azide, preferably capable of reacting with an alkyne group comprised in (V′);
an alkyne, preferably capable of reacting with an azide group comprised in (V′);
an amino group, preferably capable of reacting with a molecule (V′) in the presence of an enzyme such as a transglutaminase;
the compound preferably having a structure as shown in claim 13 or 15, with the proviso that (Y′) replaces (Y) and m is 1.

31. Kit for the modification of a molecule capable of interacting with a target cell comprising the compound of claim 30 and optionally a buffer, said buffer having preferably a pH of from 6.0 to 10, more preferably of from 6.5 to 8.0.

32. Method for the modification of a molecule capable of interacting with a target cell comprising reacting a molecule capable of interacting with a target cell, such as a monoclonal antibody or an antibody fragment incorporated into an Fc-fusion protein, with a compound according to claim 30.
